(19) United States
(12) Patent Application Publication
(10) Pub. No.: US 2024/0207385 A1
(43) Pub. Date: Jun. 27, 2024

(54) COMPOSITIONS COMPRISING SELF-ASSEMBLING VACCINES AND METHODS OF USING THE SAME

(71) Applicant: The Wistar Institute of Anatomy and Biology, Philadelphia, PA (US)

(72) Inventors: Daniel W. KULP, Moorestown, NJ (US); David B. WEINER, Merion, PA (US); Ziyang XU, Philadelphia, PA (US); Kar MUTHUMANI, Cherry Hill, NJ (US)

(21) Appl. No.: 17/802,936

(22) PCT Filed: Feb. 26, 2021
(86) PCT No.: PCT/US21/20105

§ 371 Date: Aug. 26, 2022

Related U.S. Application Data
(60) Provisional application No. 62/982,038, filed on Feb. 26, 2020.
(60) Provisional application No. 63/047,245, filed on Jul. 1, 2020.

(51) Int. Cl. A61K 39/12; A61K 39/00; A61P 31/16; A61P 37/04

(52) U.S. Cl. CPC A61K 39/12; A61P 31/16; A61P 37/04; A61K 2039/53; A61K 2039/545; A61K 2039/55516; A61K 2039/55555

(57) ABSTRACT

The disclosure generally relates to compositions comprising self-assembling vaccines and methods of using the same. The disclosure provides compositions comprising an expressible nucleic acid sequence comprising a first nucleic acid sequence encoding a self-assembling polypeptide or a pharmaceutically acceptable salt thereof and a second nucleic acid sequence encoding a viral antigen or a pharmaceutically acceptable salt thereof. The disclosure further provides compositions comprising an expressible nucleic acid sequence comprising a first nucleic acid sequence encoding a self-assembling polypeptide or a pharmaceutically acceptable salt thereof and a second nucleic acid sequence encoding a CD40 ligand polypeptide. Methods of using any of the disclosed compositions are also provided.

### CROSS REFERENCE TO RELATED APPLICATION

This application claims priority to U.S. Provisional Application No. 62/982,038, filed Feb. 26, 2020, and U.S. Provisional Application No. 63/047,245, filed Jul. 1, 2020, the entire contents of each of the aforementioned applications are incorporated by reference herein in its entirety.

### STATEMENT REGARDING FEDERALLY SPONSORED RESEARCH

The present disclosure was made with government support under NIH IPCAVD Grant U19 A1109646-04, NIH CIVICS Grant 4270101374, NIH Grant R01GM103899 and NIH Grant R01GM129357 awarded by the National Institutes of Health. The government has certain rights in the disclosed inventions.

### SUBMISSION OF SEQUENCE LISTING

The instant application contains a Sequence Listing which has been submitted electronically via Patent Center in ASCII plain text format and is hereby incorporated by reference in its entirety. The name of the file containing the Sequence Listing is WIST-006-US-SEQ LIST_ST25. The size of the text file is 400,050 bytes and the text file was created on Dec. 23, 2023.

### FIELD OF THE INVENTION

The disclosure generally relates to compositions comprising self-assembling vaccines and methods of using the same. The disclosure provides compositions comprising an expressible nucleic acid sequence comprising a first nucleic acid sequence encoding a self-assembling polypeptide or a pharmaceutically acceptable salt thereof and a second nucleic acid sequence encoding a viral antigen or a pharmaceutically acceptable salt thereof. The disclosure further provides compositions comprising an expressible nucleic acid sequence comprising a first nucleic acid sequence encoding a self-assembling polypeptide or a pharmaceutically acceptable salt thereof and a second nucleic acid sequence encoding a CD40 ligand polypeptide. Methods of using any of the disclosed compositions are also provided.

### BACKGROUND

Vaccination is an extremely important public health measure that has demonstrated prophylactic and therapeutic utility against many infectious diseases [1-3], and impacted some forms of cancer [4]. In the past decade, advances in material engineering has allowed for the development and study of a new generation of nanoparticle vaccines [5-7]. Hepatitis B and human papillomavirus (HPV) vaccines are such examples of self-assembling virus-like particles which have impacted millions of people [8, 9]. Nanoparticles may come in several shapes and forms. Inorganic materials [10, 11], nontoxic phospholipids [12], virus-like particles (VLPs) or self-assembling protein nanoparticles (SAPN) [13-16] can all scaffold and present antigens in repetitive multimeric manners to robustly stimulate immunity in animal models [16-18].

However, some intrinsic production challenges have impeded broader translation of nano-vaccines into the clinical space [19, 20]. VLP vaccines are often produced at low yields in mammalian cell lines and are difficult to purify, requiring complex reassembly processes and additional post-hoc characterization [13, 21, 22]. Production of HPV VLPs, for example, requires three sequential purification steps of strong cation exchange chromatography, size-exclusion chromatography, and hydroxyapatite chromatography [23]. Large-scale production of liposome-based nano-vaccines is challenging, as slight variations in the methods of production result in heterogeneity of the liposomes produced [19]. Production of nano-vaccines for a global market could therefore require specialized pipelines which raise costs. In addition, regulatory approval of drugs for use in humans can be complex for development of multicomponent nano-medicines [24]. Technologies that would allow de novo nanoparticle assemblies in the hosts from materials that are inexpensive, simple and stable, which bypass these complex biochemical processes and downstream purifications, may be of interest.

In this regard, computational protein engineering is an extremely powerful tool and has facilitated the design of novel biologics as well as specific potent nano-vaccines [15, 26]. One such example is the eOD-GT8-60mer, which is a priming immunogen engineered to activate precursors of HIV-1 broadly neutralizing antibodies [27-29]. When scaffolded with the C-terminus of the lumazine synthase (LS) enzyme from Aquifex Aeolicus, eOD-GT8 can assemble into a 60-mer nanoparticle to induce stronger humoral immunity and higher frequencies of antigen-specific IgG+ memory B cells [27]. In terms of vaccine delivery, DNA vaccines have been studied for induction of humoral and cellular immunity [30-32]. Additionally, delivery of optimized DNA plasmids encoding monomeric immunogens via adaptively-controlled electroporation (EP) can result in 1000-fold enhancement of in vivo expression and longer-term in vivo production of the encoded antigens [34-36]. The newer DNA platform is also a robust method of eliciting adaptive immune responses in humans, having demonstrated immune potency in the clinic against such diseases as ZIKA, Ebola, HIV, MERS and clinical efficacy against HPV-driven cervical dysplasia [4, 37-40].

### SUMMARY OF EMBODIMENTS

While simple multimerization domains, such as heptamer domain IMX313P, have been employed to improve DNA vaccine responses [41, 42], the disclosure relates to methods of inducing both therapeutically effective B- and T-cell responses by administration of nucleic acid vaccines computationally designed to include nucleic acid sequences encoding nanoparticle monomer peptides (such as 7, 24, 60, and 180-mers) that self-assemble with a fusion of antigens.

Nanotechnologies are considered to be of growing importance to the vaccine field. Through decoration of immunogens on multivalent nanoparticles, designed nano-vaccines can elicit improved humoral immunity. However, significant practical and monetary challenges in large-scale production of nano-vaccines have impeded their widespread clinical translation. Compositions comprising self-assembling vaccines and methods of using the same have been previously described in International Application No. PCT/US2019/068444 filed on Dec. 23, 2018, which is incorporated by reference in its entirety herewith. In the present disclosure, an alternative approach integrating computational protein modeling and adaptive electroporation mediated synthetic DNA delivery thus enabling direct in vivo production of nano-vaccines is provided. eOD-GT8-60mer is currently being clinically evaluated as a recombinant protein vaccine [43], and was examined as a prototype for DNA delivery. It is demonstrated that DNA-launched nanoparticles decorated with COD-GT8 (herein referred as DLnano_LS_GT8 for DNA-Launched nanoparticle Lumazine Synthase decorated with eOD-GT8) could spontaneously self-assembled in vivo into nanoparticles. DNA-launched nano-vaccines induced stronger humoral responses than their monomeric counterparts in both mice and guinea pigs, and uniquely elicited CD8+ effector T-cell immunity as compared to recombinant protein nano-vaccines. Improvements in vaccine responses were recapitulated when DNA-launched nano-vaccines with alternative scaffolds and decorated antigen were designed and evaluated. Finally, evaluation of functional immune responses induced by DLnano-vaccines demonstrated that, in comparison to control mice or mice immunized with DNA-encoded hemagglutinin monomer, mice immunized with a DNA-launched hemagglutinin nanoparticle vaccine fully survived a lethal influenza challenge, and had substantially lower viral load, weight loss and influenza-induced lung pathology. Synthetic DNA/EP technology can, therefore, be used to direct in vivo assembly of computationally designed nano-vaccines, which elicit more potent functional immunological responses. This combination can be important for rapid development of vaccines and immunotherapies thus offering advantages for immunization against multiple disease targets.

Accordingly, the disclosure relates to a composition comprising an expressible nucleic acid sequence comprising: a) a first nucleic acid sequence encoding a scaffold domain comprising a self-assembling polypeptide; and b) a second nucleic acid sequence encoding a viral antigen, and optionally, wherein the expressible nucleic acid sequence is free of a nucleic acid sequence encoding a leader sequence.

In some embodiments, the self-assembling polypeptide encoded by the first nucleic acid sequence of the expressible nucleic acid sequence of the present disclosure is from Aquifex aeolicus, Helicobacter pylori, Pyrococcus furiosus or Thermotoga maritima. In some embodiments, the self-assembling polypeptide comprises at least 70% sequence identity to SEQ ID NO: 7, SEQ ID NO: 23, SEQ ID NO: 31 or SEQ ID NO: 26.

In some embodiments, the viral antigen encoded by the second nucleic acid sequence of the expressible nucleic acid sequence of the present disclosure is an antigen from a retrovirus, flavivirus, Nipah virus, West Nile virus, human papillomavirus, respiratory syncytial virus, filovirus, zaire ebolavirus, sudan ebolavirus, marburgvirus or influenza virus, or any virus disclosed in Table 1. In some embodiments, the viral antigen is an antigen from human immunodeficiency virus-1 (HIV-1). In some embodiments, the viral antigen comprises at least 70% sequence identity to SEQ ID NO: 9, SEQ ID NO: 45, SEQ ID NO: 47, SEQ ID NO: 49, SEQ ID NO: 51, SEQ ID NO: 53, SEQ ID NO: 55, SEQ ID NO: 65, SEQ ID NO: 66 or SEQ ID NO: 67.

In some embodiments, the expressible nucleic acid sequence of the present disclosure further comprises a third nucleic acid sequence encoding a linker domain comprising a linker peptide, said third nucleic acid sequence positioned between the first nucleic acid sequence and the second nucleic acid sequence in the 5′ to 3′ orientation. In some embodiments, the linker peptide comprises at least 70% sequence identity to SEQ ID NO: 8, SEQ ID NO: 18, SEQ ID NO: 22, SEQ ID NO: 27 or SEQ ID NO: 32.

In some embodiments, the expressible nucleic acid sequence of the present disclosure comprises at least 70% sequence identity to SEQ ID NO: 68. In other embodiments, the expressible nucleic acid sequence of the present disclosure encodes a polypeptide comprising at least 70% sequence identity to SEQ ID NO: 69.

In some embodiments, the expressible nucleic acid sequence of the present disclosure is operably linked to one or a plurality of regulatory sequences. In some embodiments, the expressible nucleic acid sequence is comprised in a nucleic acid molecule. In some embodiments, the nucleic acid molecule is a plasmid.

The disclosure further relates to a pharmaceutical composition comprising (i) any of the compositions disclosed herein, and (ii) a pharmaceutically acceptable carrier. In some embodiments, the pharmaceutical composition of the present disclosure comprises any of the disclosed compositions in the amount of from about 1 to about 100 micrograms. In some embodiments, the pharmaceutical composition of the present disclosure comprises any of the disclosed compositions in the amount of from about 1 to about 20 micrograms.

The disclosure also relates to a method of vaccinating a subject in need thereof comprising administering a therapeutically effective amount of any of the pharmaceutical compositions disclosed herein to the subject. In some embodiments, the therapeutically effective amount is from about 20 to about 2000 micrograms of the expressible nucleic acid sequence comprised in any of the pharmaceutical compositions disclosed herein. In some embodiments, the therapeutically effective dose is from about 0.3 micrograms of the composition per kilogram of subject to about 30 micrograms per kilogram of subject. In some embodiments, the subject being vaccinated is a human.

The disclosure further relates to a method of inducing an immune response in a subject in need thereof comprising administering to the subject any of the pharmaceutical compositions disclosed herein. In some embodiments, the administering comprises administering from about 1 to about 2000 micrograms of the expressible nucleic acid sequence comprised in any of the pharmaceutical compositions disclosed herein. In some embodiments, the therapeutically effective dose is from about 0.3 micrograms of the composition per kilogram of subject to about 30 micrograms per kilogram of subject. In some embodiments, the subject is a human. In some embodiments, the immune response being induced is an antigen-specific immune response. In some embodiments, the subject is diagnosed with or suspected of having an HIV-1 infection. In some embodiments, the immune response is an antigen-specific immune response against an HIV-1 antigen.

The disclosure additionally relates to a method of neutralizing one or plurality of viruses in a subject in need thereof comprising administering to the subject any of the pharmaceutical compositions disclosed herein. In some embodiments, the administering comprises administering from about 1 to about 30 micrograms of the expressible nucleic acid sequence comprised in any of the pharmaceutical compositions disclosed herein. In some embodiments, the therapeutically effective dose is from about 0.3 micrograms of the composition per kilogram of subject to about 30 micrograms per kilogram of subject. In some embodiments, the subject is a human.

In some embodiments, the administration of any of the disclosed pharmaceutical compositions in any of the disclosed methods is accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or a combination thereof.

Also disclosed is a method of stimulating a therapeutically effective antigen-specific immune response against a virus in a mammal in need thereof infected with a virus comprising administering a therapeutically effective amount of any of the pharmaceutical compositions disclosed herein. In some embodiments, the mammal is infected with a HIV virus. In some embodiments, the therapeutically effective amount is from about 0.3 micrograms of the disclosed composition per kilogram of subject to about 30 micrograms per kilogram of subject.

Further disclosed is a method of inducing expression of a self-assembling vaccine in a subject comprising administering any of the pharmaceutical compositions disclosed herein. In some embodiments, any of the disclosed methods are free of administering any polypeptide of the antigen directly to the subject.

The disclosure also relates to a vaccine comprising a polypeptide comprising: a) a scaffold domain comprising a self-assembling polypeptide; and b) an antigen domain comprising a viral antigen, and optionally, wherein the vaccine is free of a leader sequence.

In some embodiments, the self-assembling polypeptide encoded by the first nucleic acid sequence of the expressible nucleic acid sequence of the present disclosure is from Aquifex aeolicus, Helicobacter pylori, Pyrococcus furiosus or Thermotoga maritima. In some embodiments, the self-assembling polypeptide comprises at least 70% sequence identity to SEQ ID NO: 7, SEQ ID NO: 23, SEQ ID NO: 31 or SEQ ID NO: 26. In some embodiments, the self-assembling polypeptide comprises at least 80% sequence identity to SEQ ID NO: 7, SEQ ID NO: 23, SEQ ID NO: 31 or SEQ ID NO: 26. In some embodiments, the self-assembling polypeptide comprises at least 85% sequence identity to SEQ ID NO: 7, SEQ ID NO: 23, SEQ ID NO: 31 or SEQ ID NO: 26. In some embodiments, the self-assembling polypeptide comprises at least 90% sequence identity to SEQ ID NO: 7, SEQ ID NO: 23, SEQ ID NO: 31 or SEQ ID NO: 26. In some embodiments, the self-assembling polypeptide comprises at least 95% sequence identity to SEQ ID NO: 7, SEQ ID NO: 23, SEQ ID NO: 31 or SEQ ID NO: 26. In some embodiments, the self-assembling polypeptide comprises at least 96% sequence identity to SEQ ID NO: 7, SEQ ID NO: 23, SEQ ID NO: 31 or SEQ ID NO: 26. In some embodiments, the self-assembling polypeptide comprises at least 97% sequence identity to SEQ ID NO: 7, SEQ ID NO: 23, SEQ ID NO: 31 or SEQ ID NO: 26. In some embodiments, the self-assembling polypeptide comprises at least 98% sequence identity to SEQ ID NO: 7, SEQ ID NO: 23, SEQ ID NO: 31 or SEQ ID NO: 26. In some embodiments, the self-assembling polypeptide comprises at least 99% sequence identity to SEQ ID NO: 7, SEQ ID NO: 23, SEQ ID NO: 31 or SEQ ID NO: 26. In some embodiments, the self-assembling polypeptide comprises 100% sequence identity to SEQ ID NO: 7, SEQ ID NO: 23, SEQ ID NO: 31 or SEQ ID NO: 26.

In some embodiments, the viral antigen encoded by the second nucleic acid sequence of the expressible nucleic acid sequence of the present disclosure encodes an antigen from a retrovirus, Flavivirus, Nipah virus, West Nile virus, human papillomavirus, respiratory syncytial virus, filovirus, zaire ebolavirus, sudan ebolavirus, marburgvirus or influenza virus, or any virus disclosed in Table 1. In some embodiments, the viral antigen is an antigen from HIV-1. In some embodiments, the viral antigen comprises at least 70% sequence identity to SEQ ID NO: 9, SEQ ID NO: 45, SEQ ID NO: 47, SEQ ID NO: 49, SEQ ID NO: 51, SEQ ID NO: 53, SEQ ID NO: 55, SEQ ID NO: 65, SEQ ID NO: 66 or SEQ ID NO: 67.

In some embodiments, the polypeptide encoded by the expressible nucleic acid sequence of the present disclosure further comprises a linker domain comprising a linker peptide located between the scaffold domain and the antigen domain. In some embodiments, the linker peptide comprises at least 70% sequence identity to SEQ ID NO: 8, SEQ ID NO: 18, SEQ ID NO: 22, SEQ ID NO: 27 or SEQ ID NO: 32. In some embodiments, the polypeptide encoded by the expressible nucleic acid sequence of the present disclosure comprises at least 70% sequence identity to SEQ ID NO: 69.

The disclosure further relates to a DNA vaccine comprising an expressible nucleic acid sequence encoding a polypeptide comprising at least about 70% sequence identity to SEQ ID NO: 69. In some embodiments, the expressible nucleic acid sequence comprised in such DNA vaccines comprises at least about 70% sequence identity to SEQ ID NO: 68. In some embodiments, the DNA vaccine of the present disclosure further comprises a pharmaceutically acceptable excipient. In some embodiments, the pharmaceutically composition further comprises: (i) a second nucleic acid molecule encoding a vaccine adjuvant; and/or (ii) a vaccine adjuvant that is a polypeptide.

The disclosure further related to a composition comprising one or a plurality of any of the expressible nucleic acid sequences disclosed herein, the plurality of expressible nucleic acid sequences comprising a first nucleic acid sequence encoding a scaffold domain comprising a self-assembling polypeptide and a second nucleic acid sequence encoding an antigen domain comprising a viral antigen, wherein the plurality of expressible nucleic acid sequences is optionally free of a nucleic acid sequence encoding a leader sequence. In some embodiments, the self-assembling polypeptide encoded by the first nucleic acid sequence is from Aquifex aeolicus, Helicobacter pylori, Pyrococcus furiosus or Thermotoga maritima. In some embodiments, the self-assembling polypeptide comprises at least 70% sequence identity to SEQ ID NO: 7, SEQ ID NO: 23, SEQ ID NO: 31 or SEQ ID NO: 26. In some embodiments, the viral antigen encoded by the second nucleic acid sequence is an antigen from a retrovirus, flavivirus, Nipah virus, West Nile virus, human papillomavirus, respiratory syncytial virus, filovirus, zaire ebolavirus, sudan ebolavirus, marburgvirus or influenza virus, or any virus disclosed in Table 1. In some embodiments, the viral antigen is an antigen from HIV-1. In some embodiments, the viral antigen comprises at least 70% sequence identity to SEQ ID NO: 9, SEQ ID NO: 45, SEQ ID NO: 47, SEQ ID NO: 49, SEQ ID NO: 51, SEQ ID NO: 53, SEQ ID NO: 55, SEQ ID NO: 65, SEQ ID NO: 66 or SEQ ID NO: 67. In some embodiments, the plurality of expressible nucleic acid sequences further comprise a third nucleic acid sequence encoding a linker domain comprising a linker peptide located between the scaffold domain and the antigen domain. In some embodiments, the linker peptide comprises at least 70% sequence identity to SEQ ID NO: 8, SEQ ID NO: 18, SEQ ID NO: 22, SEQ ID NO: 27 or SEQ ID NO: 32. In some embodiments, the plurality of expressible nucleic acid sequences comprise at least 70% sequence identity to SEQ ID NO: 68, or wherein the plurality of expressible nucleic acid sequences encode a polypeptide comprising at least 70% sequence identity to SEQ ID NO: 69. In some embodiments, at least one of the plurality of expressible nucleic acid sequences is operably linked to at least one regulatory sequence.

The present disclosure also relates to a cell comprising any of the expressible nucleic acid sequences disclosed herein. In some embodiments, the cell of the present disclosure comprises an expressible nucleic acid sequence comprising: a) a first nucleic acid sequence encoding a scaffold domain comprising a self-assembling polypeptide; and b) a second nucleic acid sequence encoding an antigen domain comprising a viral antigen, and optionally, wherein the expressible nucleic acid sequence is free of a nucleic acid sequence encoding a leader sequence. In some embodiments, the cell is an antigen presenting cell, such as a macrophage or astrocyte.

In some embodiments, the self-assembling polypeptide encoded by the first nucleic acid sequence is from Aquifex aeolicus, Helicobacter pylori, Pyrococcus furiosus or Thermotoga maritima. In some embodiments, the self-assembling polypeptide comprises at least 70% sequence identity to SEQ ID NO: 7, SEQ ID NO: 23, SEQ ID NO: 31 or SEQ ID NO: 26.

In some embodiments, the viral antigen encoded by the second nucleic acid sequence encodes an antigen from a retrovirus, flavivirus, Nipah virus, West Nile virus, human papillomavirus, respiratory syncytial virus, filovirus, zaire ebolavirus, sudan ebolavirus, marburgvirus or influenza virus, or any virus disclosed in Table 1. In some embodiments, the viral antigen is an antigen from HIV-1. In some embodiments, the viral antigen comprises at least 70% sequence identity to SEQ ID NO: 9, SEQ ID NO: 45, SEQ ID NO: 47, SEQ ID NO: 49, SEQ ID NO: 51, SEQ ID NO: 53, SEQ ID NO: 55, SEQ ID NO: 65, SEQ ID NO: 66 or SEQ ID NO: 67.

In some embodiments, the expressible nucleic acid sequence comprised in the cell of the present disclosure further comprises a third nucleic acid sequence encoding a linker domain comprising a linker peptide, said third nucleic acid sequence positioned between the first nucleic acid sequence and the second nucleic acid sequence in the 5′ to 3′ orientation. In some embodiments, the linker peptide comprises at least 70% sequence identity to SEQ ID NO: 8, SEQ ID NO: 18, SEQ ID NO: 22, SEQ ID NO: 27 or SEQ ID NO: 32.

In some embodiments, the expressible nucleic acid sequence comprised in the cell of the present disclosure comprises at least 70% sequence identity to SEQ ID NO: 68. In some embodiments, the expressible nucleic acid sequence comprised in the cell of the present disclosure encodes a polypeptide comprising at least 70% sequence identity to SEQ ID NO: 69.

In some embodiments, the expressible nucleic acid sequence comprised in the cell of the present disclosure is operably linked to one or a plurality of regulatory sequences. In some embodiments, the expressible nucleic acid sequence is comprised within a nucleic acid molecule. In some embodiments, the nucleic acid molecule is a plasmid. In some embodiments, the disclosure relates to a plasmid comprising at least one, two, three, four or more expressible nucleic acid sequences, at least a first nucleic acid sequence comprising or consisting of: a) a first nucleic acid sequence encoding a scaffold domain comprising a self-assembling polypeptide; and b) a second nucleic acid sequence encoding an antigen domain comprising a viral antigen, and optionally, wherein the expressible nucleic acid sequence is free of a nucleic acid sequence encoding a leader sequence. In some embodiments, the cell is an antigen presenting cell, such as a macrophage or astrocyte. In some embodiments, each composition comprises at least a first expressible nucleic acid sequence further comprises a immunoglobulin leader sequence, such as an IgE leader or a IgG leader sequence.

The disclosure further relates to a pharmaceutical composition comprising: (i) any of the compositions or cells disclosed herein, and (ii) a pharmaceutically acceptable carrier. In some embodiments, the pharmaceutical composition further comprises a nucleic acid sequence that encodes an adjuvant or a protein adjuvant. In some embodiments, the pharmaceutical composition comprises from about 1 to about 100 micrograms of the disclosed expressible nucleic acid sequence. In some embodiments, the pharmaceutical composition comprises from about 1 to about 20 micrograms of the disclosed expressible nucleic acid sequence.

Further disclosed is a method of vaccinating a subject in need thereof against viral infection comprising administering a therapeutically effective amount of any of the pharmaceutical compositions disclosed herein. A method of inducing an immune response to a viral antigen in a subject in need thereof comprising administering a therapeutically effective amount of any of the pharmaceutical composition disclosed herein is also disclosed. In some embodiments, the viral infection being vaccinated against is an infection of retrovirus, flavivirus, Nipah Virus, West Nile virus, human papillomavirus, respiratory syncytial virus, filovirus, zaire ebolavirus, sudan ebolavirus, marburgvirus or influenza virus. In some embodiments, the administering is accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof. In some embodiments, the therapeutically effective amount is from about 20 to about 2000 micrograms of the disclosed expressible nucleic acid sequence. In some embodiments, the therapeutically effective amount is from about 0.3 micrograms of the disclosed expressible nucleic acid sequence per kilogram of subject to about 30 micrograms of the disclosed expressible nucleic acid sequence per kilogram of subject. In some embodiments, the subject is a human. In some embodiments, the immune response is an antigen-specific immune response against a viral antigen. In some embodiments, the immune response is a therapeutically effective antigen-specific immune response against a viral antigen that includes a CD8+ and CD4+ immune response.

The disclosure further provides a nanoparticle comprising: (i) from about 7 to about 120 monomers, each monomer comprising at least about 70% sequence identity to SEQ ID NO: 7, SEQ ID NO: 23, SEQ ID NO: 26, or SEQ ID NO: 31, or a functional fragment thereof; and (ii) a CD40L polypeptide. In some embodiments, the nanoparticle further comprises a polypeptide that is a viral antigen or cancer antigen. Also disclosed is a nucleic acid molecule comprising an expressible nucleic acid sequence encoding: (i) a nanoparticle monomer from lumen synthase or a functional fragment or variant thereof comprising at least about 70% sequence identity to SEQ ID NO: 7, SEQ ID NO: 23, SEQ ID NO: 26, or SEQ ID NO: 31; and (ii) a CD40L polypeptide. The disclosure also relates to a nucleic acid molecule comprising an expressible nucleic acid sequence encoding: (i) a self-assembling nanoparticle monomer or a functional fragment or variant thereof comprising at least about 70% sequence identity to SEQ ID NO: 2, SEQ ID NO: 13, SEQ ID NO: 14, or SEQ ID NO: 15; and (ii) a CD40L polypeptide. In some embodiments, any of the disclosed nucleic acid molecules is an RNA, DNA or RNA/DNA plasmid, cosmid, or viral vector. In some embodiments, the expressible nucleic acid sequence comprised in any of the disclosed nucleic acid molecules further comprises a nucleic acid sequence encoding a viral antigen or cancer antigen. The disclosure further provides a composition comprising an expressible nucleic acid sequence comprising: a) a first nucleic acid sequence encoding a scaffold domain comprising a self-assembling polypeptide; and b) a second nucleic acid sequence encoding a CD40L polypeptide. In some embodiments, the disclosed composition further comprises a third nucleic acid sequence encoding a protein of interest. In some embodiments, the protein of interest is a viral antigen or a cancer antigen.

In some embodiments, the CD40L polypeptide comprised in any of the disclosed nanoparticles, nucleic acid molecules and compositions comprises at least 70% sequence identity to SEQ ID NO: 103, SEQ ID NO: 104, SEQ ID NO: 105, SEQ ID NO: 106, SEQ ID NO: 108, SEQ ID NO: 109, SEQ ID NO: 110, or SEQ ID NO: 111, or a functional fragment thereof. In some embodiments, the CD40L polypeptide is encoded by a nucleic acid sequence comprising at least 70% sequence identity to SEQ ID NO: 102 or SEQ ID NO: 107, or a functional fragment thereof that comprises at least about 70%, 75%, 80%, 85%, 90%, 91%, 92%, 93%, 94%, 95%, 96%, 97%, 98% or 99% sequence identity to SEQ ID NO: 102 or SEQ ID NO: 107. In some embodiments, any of the disclosed nucleic acid molecules or expressible nucleic acid sequences comprises a nucleic acid sequence comprising at least 70% sequence identity to SEQ ID NO: 2, SEQ ID NO: 13, SEQ ID NO: 14, or SEQ ID NO: 15, or a functional fragment thereof that comprises at least about 70%, 75%, 80%, 85%, 90%, 91%, 92%, 93%, 94%, 95%, 96%, 97%, 98% or 99% sequence identity to SEQ ID NO: 2, SEQ ID NO: 13, SEQ ID NO: 14, or SEQ ID NO: 15. In some embodiments, any of the disclosed nucleic acid molecules or expressible nucleic acid sequences encodes a scaffold domain comprising at least about 70% sequence identity to SEQ ID NO: 7, SEQ ID NO: 23, SEQ ID NO: 26, or SEQ ID NO: 31, or a functional fragment thereof. In some embodiments, the nucleic acid sequences encoding the CD40L polypeptide and the monomer are contiguously connected by a nucleic acid sequence encoding a linker.

Further disclosed is a cell comprising any of the nanoparticles and nucleic acid molecules disclosed herein. In some embodiments, the cell is a eukaryotic cell. In some embodiments, the cell is an antigen presenting cell.

The disclosure also provides a pharmaceutical composition comprising a therapeutically effective amount of one or a plurality of any of the nanoparticles, nucleic acid molecules or cells disclosed herein and a pharmaceutically acceptable carrier. Also disclosed is a method of inducing an immune response in a subject comprising administering the disclosed pharmaceutical composition to the subject. The disclosure also provides a method of treating and/or preventing a viral infection or cancer in a subject in need thereof comprising administering a therapeutically effective amount of the pharmaceutical composition disclosed herein to the subject. A method of vaccinating a subject in need thereof comprising administering a therapeutically effective amount of the disclosed pharmaceutical composition to the subject is also provided. Further disclosed is a method of activating and/or enhancing an antigen-specific immune response of a vaccine in a subject in need thereof comprising administering a therapeutically effective amount of the disclosed pharmaceutical composition to the subject.

The disclosure also relates to a vaccine or vaccine adjuvant comprising a therapeutically effective amount of any of the disclosed pharmaceutical composition.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A-1I show expression and assembly of in vitro produced protein eOD-GT8-60mer and GT8-monomer and in vivo produced DLnano_LS_GT8 and DLmono_GT8. FIG. 1A: Predicted structure of eOD-GT8-60mer, LS inner scaffold is shown in dark gray (inside of the particle), decorated GT8 shown in gray and N-linked glycans are represented as light gray sticks. FIG. 1B: SECMAL trace showing the calculated molecular weight of SEC purified eOD-GT8-60mer. FIG. 1C: Negative stain electron microscopy images of purified COD-GT8-60mer. FIG. 1D: In vivo expression of DLmono_GT8 or DLnano_LS_GT8 in BALB/c mice four d.p.i, as probed by VRC01 and anti-human Alexa Fluor 488, nuclei staining with DAPI is shown in gray. FIG. 1E: Reducing SDS PAGE western analysis to determine in vivo expression of DLmono_GT8 and DLnano_LS_GT8 four d.p.i in muscle homogenates with VRC01 (in light gray); GAPDH (in gray) is used as the loading control. FIG. 1F: Pseudo-native PAGE analysis comparing migration of in vivo produced DLmono_GT8 and DLnano_LS_GT8 to in vitro produced SEC purified recombinant GT8-monomer (labelled as STD mono) and eOD-GT8-60mer (labelled as STD nano) protein standards. FIG. 1G: Murine MBL labelling of naïve mouse muscles or muscles transfected with DLmono_GT8 and DLnano_LS_GT8 seven d.p.i. FIG. 1H: Transmission electron microscopy (TEM) images of muscle sections from mice injected with DLmono_GT8 or DLnano_LS_GT8 seven d.p.i that were immunolabelled with VRC01 and gold anti-human IgG. Black arrows highlight VRC01 staining. FIG. 1I: TEM image of muscle section showing an example of high-valency GT8 nanoparticle assembled in vivo. 80 μg plasmid DNA dose of DLmono_GT8 or DLnano_LS_GT8 used in FIG. 1D-1I.

FIG. 2A-2J show characterization of in vivo trafficking of DLnano_LS_GT8 and humoral responses induced by DLnano_LS_GT8 versus DLmono_GT8. FIG. 2A: Trafficking of DLnano_LS_GT8 and DLmono_GT8 seven d.p.i in the draining lymph nodes, as determined by VRC01 staining (light gray) and anti-CD35-BV421 staining (gray) for co-localization analyses. FIG. 2B: ELISA binding against monomeric GT8 using serum from female BALB/c immunized with DLmono_GT8 or DLnano_LS_GT8 seven d.p.i. FIG. 2C: Endpoint titers to GT8 over time using serum from female BALB/c receiving two immunizations of DLmono_GT8 or DLnano_LS_GT8 three weeks apart. FIG. 2D: Frequencies of CD19+IgM-IgD-IgG+GT8 specific B-cells in the spleen of naïve female BALB/c mice or female BALB/c mice immunized with two doses of DLmono_GT8 or DLnano_LS_GT8 five weeks post the second immunization. FIG. 2E: Percentage inhibition of VRC01-GT8 binding by naïve mice sera or post-immune sera from the DLmono_GT8 or DLnano_LS_GT8 vaccinated mice at 1:200 dilution. FIG. 2F: Comparison of GT8 endpoint titers for female BALB/c mice receiving two doses of DLmono_GT8 at 25 μg dose or DLnano_LS_GT8 at 2 μg dose. FIG. 2G: Comparison of GT8 endpoint titers for male BALB/c mice receiving two doses of DLmono_GT8 or DLnano_LS_GT8 at 25 μg dose. FIG. 2H: Comparison of endpoint titers in guinea pigs receiving single 50 μg intradermal immunization of DLmono_GT8 or DLnano_LS_GT8. FIG. 2I: Comparison of humoral responses induced by protein eOD-GT8-60mer adjuvanted by Sigma Adjuvant System or DLnano_LS_GT8 as assessed in C57BL/6 mice. FIG. 2J: Humoral responses in wild-type C57BL/6, MBL KO or CR2 KO mice to protein eOD-GT8-60mer and DLnano_LS_GT8 vaccinations seven d.p.i. 80 μg of plasmid DNA used in FIG. 2A, 25 μg plasmid DNA and 10 μg recombinant protein used elsewhere in the figure unless otherwise specified. Each group except in FIG. 2J includes five animals; each group in FIG. 2J include four animals; each dot represents an animal; error bar represents standard deviation; arrow below the plot represents an immunization; two-tailed Mann-Whitney Rank Test used to compare groups; p-values were adjusted for multiple comparison where appropriate; *, p-value<0.05.

FIG. 3A-3I show characterization of cellular responses induced by DLnano_LS_GT8 versus DLmono_GT8 in BALB/c mice and by protein eOD-GT8-60mer and DLnano_LS_GT8 in C57BL/6 mice. FIG. 3A: ELIspot responses to the LS peptides and GT8 peptides in BALB/c mice immunized with two doses of DLmono_GT8 or DLnano_LS_GT8 at specified doses. FIG. 3B: Effector memory CD4+ T-cell responses (CD3+CD4+CD44+CD62L−) in immunized BALB/c mice as in FIG. 3A. FIG. 3C-3E: Effector memory CD8+ T-cell responses (CD3+CD8+CD44+CD62L−) in immunized BALB/c mice in terms of IFNγ expression in FIG. 3DA and CD107a expression in FIG. 3E. FIG. 3F: Comparison for the frequencies of CD8+ effector memory T-cell responses induced by DLmono_GT8 or DLnano_LS_GT8 immunizations in BALB/c mice. FIG. 3G: T-cell responses as determined by IFN-γ ELISpot assays for protein eOD-GT8-60mer and DLnano_LS_GT8 immunized C57BL/6 mice. FIG. 3H: CD4+ effector memory T-cell responses for protein eOD-GT8-60mer and DLnano_LS_GT8 immunized C57BL/6 mice as determined by ICS. FIG. 3I: Comparisons of CD8+ T-cell responses induced by protein COD-GT8-60mer versus DLnano_LS_GT8 vaccinations in in wild-type C57BL/6, MBL KO or CR2 KO mice. 25 μg plasmid DNA and 10 μg recombinant protein used in the figure unless otherwise specified. Each group except in FIG. 3I includes five mice; each group in FIG. 3I includes four animals; each dot represents a mouse; error bar represents standard deviation; two-tailed Mann-Whitney Rank Test used to compare groups; p-values were adjusted for multiple comparison where appropriate; *, p-value<0.05.

FIG. 4A-4H show design and evaluation of new DLnano GT8-vaccines with alternative scaffolds. FIG. 4A: nsEM image of SEC-purified fraction of in vitro produced 3BVE-GT8 nanoparticles. FIG. 4B: nsEM image of SEC-purified fraction of in vitro produced PIV-GT8 nanoparticles. FIG. 4C: In vivo expression of DLnano_3 BVE_GT8 and DLnano_PfV_GT8 in transfected mouse muscles as determined by immunofluorescence; VRC01 labelling is shown in light gray and nuclei labelling shown in gray. FIG. 4D: Reducing SDS PAGE western analysis to determine in vivo expression of DLnano_3 BVE_GT8 and DLnano_PfV_GT8 four d.p.i in muscle homogenates with VRC01 (in light gray); GAPDH (in gray) is used as the loading control. FIG. 4E: Humoral responses in BALB/c mice immunized with two 25 μg doses of DLmono_GT8, DLnano_3 BVE_GT8, DLnano_LS_GT8 and DLnano_PIV-GT8. FIG. 4F: CD8+ effector memory CD107a+ T-cell responses to GT8 domain in BALB/c mice immunized with DLmono_GT8, DLnano_3 BVE_GT8, DLnano_LS_GT8 and DLnano_PIV-GT8 as in FIG. 4E. FIG. 4G: Humoral responses in BALB/c mice immunized with 2 μg doses of DLmono_GT8, DL_GT8_IMX313P, DLnano_3 BVE_GT8, DLnano_LS_GT8 and DLnano_PfV-GT8 seven d.p.i. FIG. 4H: CD8+ effector memory CD107a+ T-cell responses to GT8 domain in BALB/c mice immunized twice with 2 μg DLmono_GT8, DL_GT8_IMX313P, DLnano_3 BVE_GT8, DLnano_LS_GT8 and DLnano_PfV-GT8 three weeks apart. 80 μg of plasmid DNA used in FIG. 4C and FIG. 4D; 25 μg plasmid DNA used elsewhere in FIG. 4E and FIG. 4F; 2 μg plasmid DNA used in FIG. 4G and FIG. 4H. Each group contains five mice; each dot represents a mouse; error bar represents standard deviation; arrow below the plot represents an immunization; two-tailed Mann-Whitney Rank Test used to compare groups; p-values were adjusted for multiple comparison where appropriate; *, p<0.05.

FIG. 5A-5F show design and evaluation of new DLnano influenza hemagglutinin vaccine. FIG. 5A: SECMAL trace of lectin and SEC purified LS_HA_NC99. FIG. 5B: nsEM image of SEC-purified fraction of in vitro produced protein LS_HA_NC99 nanoparticles. FIG. 5C: Humoral responses in BALB/c mice that received DLnano_LS_HA_NC99 or DLmono_HA_NC99 at 1 μg dose. FIG. 5D: Autologous HAI titers against the H1 NC99 strain at DO, D42 (post-dose #2) and D56 (post-dose #3) for mice treated with 1 μg DLmono_HA_NC99 or DLnano_LS_HA_NC99. FIG. 5E: Heterologous HAI titers against the H1 SI06 strain at 56 d.p.i for mice treated with 1 μg DLmono_HA_NC99 or DLnano_LS_HA_NC99. FIG. 5F: CD8+ effector memory IFNγ+ T-cell responses to NC99 HA domain in naïve BALB/c mice or mice immunized with two doses of 10 μg DLmono_HA_NC99 or DLnano_LS_HA_NC99. Each group contains five mice; each dot represents a mouse; error bar represents standard deviation; arrow below the plot represents an immunization; two-tailed Mann-Whitney Rank Test used to compare groups; p-values were adjusted for multiple comparison where appropriate; *, p<0.05.

FIG. 6A-6G show functional evaluations of DLmono_HA_CA09 versus DLnano_3 BVE_HA_CA09 in H1 A/California/07/09 lethal challenge model. FIG. 6A: Binding endpoint titers to HA(CA09) over time in BALB/c mice immunized with two 1 μg doses of pVAX, DLmono_HA_CA09 or DLnano_3 BVE_HA_CA09 three weeks apart. FIG. 6B: HAI titers to the autologous A/California/07/09 strain in BALB/c mice immunized with 1 μg pVAX, DLmono_HA_CA09 or DLnano_3 BVE_HA_CA09 five weeks from their first vaccination. FIG. 6C: Percentages of vaccinated mice surviving the lethal 10LD50 H1/A/California/07/09 challenge over two-week period. FIG. 6D: Weight changes in mice immunized with pVAX, DLmono_HA_CA09 or DLnano_3 BVE_HA_CA09 over two-week period following 10LD50 H1/A/California/07/09 challenge. FIG. 6E: Percentages of vaccinated mice surviving the lethal 10LD50 H1/A/California/07/09 challenge over seven-day period in a separate study. FIG. 6F: Lung viral load in challenged mice at seven days post challenge or at the time of euthanasia as determined by RT-qPCR. FIG. 6G: H&E stain for lung histo-pathology in mice seven days post viral challenge or at the time of euthanasia, normal lung histology is shown for comparison; scale bar represents 100 μm. Each group contained 10 mice in panels FIG. 6A and FIG. 6B; each group contained five in the remaining panels; each dot represents a mouse; error bar represents standard deviation; arrow below the plot represents an immunization; two-tailed Mann-Whitney Rank Test used to compare groups; p-values were adjusted for multiple comparison where appropriate; *, p<0.05.

FIG. 7A-7I show in vitro expression of protein eOD-GT8-60mer and GT8-monomer and in vivo expression and assembly of DLnano_LS_GT8 and DLmono_GT8. FIG. 7A: Immunofluorescence analyses of intracellular expression of protein eOD-GT8-monomer and −60mer with or without IgE leader sequence in transfected HEK293T cells as determined by staining with VRC01 (light gray) and DAPI (gray) staining. FIG. 7B: Reducing SDS-PAGE analysis of Expi293F transfection supernatants of pVAX backbone plasmid, protein GT8-monomer, protein eOD-GT8-60mer. FIG. 7C: SEC trace of lectin column purified Expi293F transfection supernatant of eOD-GT8-60mer. FIG. 7D-7E: Binding of in vitro produced protein eOD-GT8-monomer and eOD-GT8-60mer to VRC01 (FIG. 7D) and MBL (FIG. 7E) in ELISA assays. FIG. 7F-7G: Binding of in vivo expressed DLmono_GT8 and DLnano_LS_GT8 seven d.p.i to VRC01 (FIG. 7F) and MBL (FIG. 7G) in the ELISA assays. FIG. 7HA: Additional TEM images of muscle sections from mice injected with DLmono_GT8 and DLnano_LS_GT8. FIG. 7I: Quantitative determination of the frequencies of clusters of different orders in the TEM images; *, p<0.05. 80 μg of plasmid DNA used in vivo for panels FIG. 7F through FIG. 7I.

FIG. 8A-8J show humoral responses induced by DLnano_LS_GT8 versus DLmono_GT8 vaccination in female BALB/c, C57BL/6 and CDI mice. FIG. 8A: ELISA binding against monomeric GT8 using serum from BALB/c immunized with 1:1 ratio (25 μg each) of DLmono_GT8 with pVAX backbone plasmid, DLmono_GT8 with DLnano_LS(core) or DLnano_LS_GT8 with pVAX backbone seven d.p.i. FIG. 8B: Endpoint titers at seven d.p.i in BALB/c mice immunized with DLmono_GT8, DL_GT8_IMX313P, or DLnano_LS_GT8. FIG. 8C: IgM endpoint titers to GT8 over time in BALB/c mice immunized with two doses of DLmono_GT8, or DLnano_LS_GT8. FIG. 8D: Endpoint titers to GT8 over time using serum from BALB/c receiving single immunizations of DLmono_GT8 or DLnano_LS_GT8. FIG. 8E: Flow plot demonstration of gating of antigen-specific GT8-Tetramer-APC+GT8-24mer-FITC+CD19+IgM-IgD-IgG+B-cells in the spleens of BALB/c mice immunized with two doses of DLmono_GT8 or DLnano_LS_GT8 five weeks post the second immunization. FIG. 8F: ELISA data showing competition of VRC01 binding at its corresponding EC₇₀ concentration to GT8 by week five post-immune sera from mice immunized with two doses of DLmono_GT8 or DLnano_LS_GT8. FIG. 8G: Endpoint titers to GT8 using serum for BALB/c receiving two immunizations of varying doses of DLmono_GT8. FIG. 8H: Endpoint titers to GT8 using serum from BALB/c mice receiving two immunizations of varying doses of DLnano_LS_GT8. FIG. 8I: Humoral responses in C57BL/6 mice immunized with two doses of DLmono_GT8 or DLnano_LS_GT8. FIG. 8J: Humoral responses in CDI mice immunized with two doses of DLnano_LS_GT8 or DLmono_GT8. 25 μg of plasmid DNA used in these experiments unless otherwise specified. n=5 for BALB/c and C57BL/6 mice, n=10 for CDI mice; each line represents an animal; error bar represents standard deviation; arrow below the plot represents an immunization; *, p<0.05.

FIG. 9A-9L show generalizability of improved cellular responses of DLnano_LS_GT8 in BALB/c, C57BL/6 and CDI mice strains; comparison of induced CD8+ T-cell responses by protein eOD-GT8-60mer versus DLnano_LS_GT8 in C57BL/6 mice. FIG. 9A-9B: Frequencies of TNFα and IL-2 expressing CD4+ effector memory T-cells specific to either the LS and GT8 domains in female BALB/c mice immunized twice with DLmono_GT8 or DLnano_LS_GT8. FIG. 9C: Comparisons of total cellular responses to the LS and GT8 domains as assessed by IFN-γ ELISpot assay in female C57BL/6 versus BALB/c mice. FIG. 9D-9E: Comparison of CD4+(FIG. 9D) and CD8+(FIG. 9E) effector memory T-cell responses to the LS and GT8 domains in female C57BL/6 and BALB/c mice. FIG. 9F-9H: Overall T-cell (FIG. 9F), CD4+ effector memory (FIG. 9G), and CD8+ effector memory (FIG. 9H) T-cell responses in female CDI mice immunized with two doses of DLnano_LS_GT8 as compared to DLmono_GT8. FIG. 9IB: Frequencies of effector memory CD8+ T-cells in female C57BL/6 and CDI mice immunized twice with DLnano_LS_GT8 and DLmono_GT8. FIG. 9J: Comparison of frequencies of GT8-specific CD8+ T-cell responses induced by two immunizations of DLnano_LS_GT8 versus DLmono_GT8 in male BALB/c mice. FIG. 9K: CD4+ effector memory T-cell responses induced by protein eOD-GT8-60mer and DLnano_LS_GT8 in C57BL/6 mice as determined by ICS. FIG. 9L: Flow plot demonstrating induction of CD8+ effector memory T-cell responses by DLnano_LS_GT8 in comparison to protein eOD-GT8-60mer in C57BL/6 mice as determined. 25 μg plasmid DNA and 10 μg recombinant protein used in the figure unless otherwise specified. n=5 for BALB/c and C57BL/6 mice, n=10 for CDI mice; each dot represents an animal; error bar represents standard deviation; two-tailed Mann-Whitney Rank Test used to compare groups; *, p<0.05; **, p<0.0005.

FIG. 10A-10E show characterization of biophysical profiles and immune responses induced by newly designed DLnano GT8-vaccines with alternative scaffolds. FIG. 10A: SEC trace of recombinantly produced designed 3BVE-GT8. FIG. 10B: SEC trace of designed PfV_GT8 immunogen shows partial assembly into the 180-mer form. FIG. 10C: Binding of in vitro produced protein GT8-monomer, 3BVE_GT8, eOD-GT8-60mer and PIV_GT8 to VRC01 by ELISA. FIG. 10D-10E: Effector memory T-cell responses to GT8 domain in BALB/c mice immunized with two doses of 25 μg DLmono_GT8, DLnano_3 BVE_GT8, DLnano_LS_GT8 and DLnano_PfV_GT8 by IFNγ ELIspots (FIG. 10D) and ICS for CD8+ T-cells (FIG. 10E). n=5 per group; each dot represents an animal; error bar represents standard deviation; two-tailed Mann-Whitney Rank Test used to compare groups; p-values were adjusted for multiple comparison where appropriate; *, p<0.05.

FIG. 11A-11F show characterization of biophysical profiles and immune responses induced by newly designed DLnano influenza hemagglutinin-based vaccines. FIG. 11A: SEC trace of recombinantly produced designed LS_HA_NC99 nanoparticles. FIG. 11B: Binding of sera from BALB/c mice immunized with 1 μg DLnano_LS_HA_NC99 or DLmono_HA_NC99 at 56 d.p.i (post-dose #3) to heterologous recombinant H1 (SI06) hemagglutinin protein. FIG. 11C-11DA: CD8+ effector memory T-cell responses to NC99 HA domain in BALB/c mice immunized with two 10 μg doses of DLmono_HA_NC99 or DLnano_LS_HA_NC99 in terms of IFNγ (FIG. 11C) and CD107a (FIG. 11D) expression. FIG. 11E: SEC trace of lectin purified recombinantly produced LS_HA_CA09. FIG. 11F: Comparison of humoral responses induced by two 10 μg doses of DLnano_LS_HA_NC99, which homogeneously assembled and by DNA-encoded LS_HA_CA09, which did not homogeneously assemble in vitro. n=5 per group; each dot represents an animal; error bar represents standard deviation; two-tailed Mann-Whitney Rank Test used to compare groups; p-values were adjusted for multiple comparison where appropriate; *, p<0.05.

FIG. 12A-12D show improved protection from lethal H1/CA09 challenge in mice with DLnano_3 BVE_HA_CA09 vaccination. FIG. 12A: SEC trace for lectin-purified recombinantly produced 3BVE_HA_CA09 nanoparticles. FIG. 12B: nsEM image of SEC-purified 3BVE_HA_CA09 nanoparticles. FIG. 12C: Weight changes in mice immunized with pVAX, DLmono_HA_CA09 or DLnano_3 BVE_HA_CA09 over seven-day period following 10LD50 H1/A/California/07/09 challenge as in FIG. 6E. FIG. 12D: H&E stain for lung histo-pathology in remaining 12 mice seven days post viral challenge or at the time of euthanasia as in FIG. 6E. 1 μg DNA dose used in vivo for FIG. 12C and FIG. 12D. n=5 per group; each line represents an animal.

FIG. 13A-13J show comparison of immune responses induced by DLnano_LS_GT8 and protein eOD-GT8-60mer, as well as DLnano_LS_HA(NC99) versus HA(NC99)-60mer in BALB/c mice. Mice received either 25 μg DNA vaccination with EP or 10 μg RIBI adjuvanted protein vaccination without EP twice three weeks apart and were euthanized two weeks post second vaccination for cellular analysis. FIG. 13A: Endpoint titers to GT8-monomer induced by DLnano_LS_GT8 in comparison with RIBI adjuvanted protein eOD-GT8-60mer. FIG. 13B: CD4+IFNγ responses to the LS domain induced by DLnano_LS_GT8, protein eOD-GT8-60mer or in naïve mice. FIG. 13A-13D: Flow cytometry plots and combined statistics for CD8+IFNγ responses to the GT8 domain induced by DLnano_LS_GT8, protein eOD-GT8-60mer or in naïve mice. FIG. 13E-13F: IFNγ ELISpot images and combined statistics for overall T-cell responses to the GT8 domain induced by DLnano_LS_GT8, protein eOD-GT8-60mer or in naïve mice. FIG. 13G: Endpoint titers to NC99 hemagglutinin induced by DLnano_LS_HA(NC99) in comparison with RIBI adjuvanted protein HA(NC99)-60mer. FIG. 13H: HAI titers against autologous H1 A/NewCaledonia/20/1999 induced by DLnano_LS_HA(NC99), protein HA(NC99)-60mer or in naïve mice. FIG. 13I: ICS determination of CD8+IFNγ responses to the HA domain induced by DLnano_LS_HA(NC99), protein HA(NC99)-60mer or in naïve mice. FIG. 13J: IFNγ ELISpot assays for overall T-cell responses to the HA domain induced by DLnano_LS_HA(NC99), protein HA(NC99)-60mer or in naïve mice. Each group includes five mice; each dot represents an animal; error bar represents standard deviation; arrow below the plot represents an immunization; two-tailed Mann-Whitney Rank Test used to compare groups; p-values were adjusted for multiple comparison where appropriate; *, p-value<0.05.

FIG. 14A-14I show determination of the role of tissue apoptosis and APC infiltration upon DNA vaccination in C57BL/6 mice. FIG. 14A: Immunofluorescence staining for cleaved caspase 3 (light gray) or nuclei (gray) in muscle sections from naïve mice or those treated with protein eOD_GT8-60mer (without EP) or DLnano_LS_GT8 (with EP) harvested four d.p.i. FIG. 14B: TUNEL assay to determine presence of double-stranded DNA breaks (dark gray) or intact DNA (light gray) for muscle sections from naïve mice or those treated with protein eOD_GT8-60mer without EP or DLnano_LS_GT8 with EP harvested four d.p.i. FIG. 14C-14DA: Flow cytometry plots and combined statistics for frequency of muscle infiltrating CD11b+F4/80+ macrophages in naïve mice, or those treated with protein eOD-GT8-60mer or DLnano_LS_GT8 seven d.p.i. FIG. 14E: Flow determination of muscle infiltrating CD11c+MHC Class II+ DCs in naïve mice, or those treated with protein eOD-GT8-60mer or DLnano_LS_GT8 seven d.p.i. FIG. 14F: Flow determination for GT8-uptake by VRC01-FITC staining for muscle macrophages in naïve mice, or mice treated with protein eOD-GT8-60mer or DLnano_LS_GT8 seven d.p.i. FIG. 14G: Comparison for changes in frequencies of muscle infiltrating macrophages four d.p.i in mice treated with DLnano_LS_GT8; the mice also received three doses of IV clodrosome or control encapsosome on Days −3, 0 and 3 relative to DNA vaccination. FIG. 14H: CD8+ T-cell responses induced by DLnano_LS_GT8 or protein eOD-GT8-60mer in mice that did or did not receive systematic macrophage depletion with clodrosome. Mice received 25 μg DNA vaccination with EP or 10 μg RIBI-adjuvanted protein vaccination without EP, and were euthanized two weeks post-vaccination for cellular analysis. FIG. 14I: CD8+ T-cell responses in naïve mice or in C57BL/6 or BATF3KO mice vaccinated with DLnano_LS_GT8. Mice were vaccinated and euthanized with the same dose and schedule as described in FIG. 14H. Each group includes four mice in FIG. 14C-14F and five mice in FIG. 14G-14I; each dot represents an animal; error bar represents standard deviation; two-tailed Mann-Whitney Rank Test used to compare groups; p-values were adjusted for multiple comparison where appropriate; *, p-value<0.05; **, p-value<0.005.

FIG. 15A-15M show characterization of functional importance of CD8+ T-cell priming by DNA-launched versus protein nanoparticle vaccination in melanoma challenge model in C57BL/6 mice. FIG. 15A: SEC trace for designed LS_Trp2₁₈₈-60mer. FIG. 15B: nsEM image of SEC purified LS_Trp2₁₈₈-60mer nanoparticles. FIG. 15C-15D: Comparison of immunogenicity of DLnano-vaccines versus monomeric DNA vaccines. Mice were vaccinated twice with 10 μg of each DNA vaccine two weeks apart and euthanized two weeks post second vaccination for cellular analysis. FIG. 15C: Comparison of CD8+IFNγ T-cell responses to Trp2₁₈₈ peptide in naïve mice or mice immunized twice two weeks apart with 10 μg DLmono_Trp2₁₈₈ or DLnano_LS_Trp2₁₈₈. FIG. 15D: Comparison of CD8+IFNγ T-cell responses to Gp100₂₅ peptide in naïve mice or mice immunized twice two weeks apart with 10 μg DLmono_Gp100₂₅ or DLnano_LS_Gp100₂₅. FIG. 15E: Treatment and vaccination schemes used to study CD8+ T-cell responses to both Trp2₁₈₈ and Gp100₂₅ peptides in naïve mice, B16F10-tumor bearing mice that received anti-PD1 treatment alone, or anti-PD1 treatment in combination with protein (4 μg) or DNA vaccination (10 μg) of LS-GT8 scaffolded 60mer nanoparticles presenting Trp2₁₈₈ and Gp100₂₅ epitopes. FIG. 15F-15H: Induced IFNγ+, IFNγ+CD107a+, or IFNγ+ TNFα+IL-2+CD8+ T-cell responses to Trp2₁₈₈ in naïve tumor-free mice or B16-F10 bearing mice that received treatments as described in FIG. 15E, FIG. 15I and FIG. 15J, tumor growth (FIG. 15I) and overall survival (FIG. 15J) in mice challenged with 105 B16-F10 cells and then received treatments as described in FIG. 15E and FIG. 15K. IVIS to determine in vivo tumor growth in the prophylactic tumor model where mice first received two vaccinations of pVAX vector, combination of protein Trp2₁₈₈ and Gp100₂₅-60mer, or combination of DLnano_LS_Trp2₁₈₈ and DLnano_LS_Gp100₂₅ and were then challenged with 105 B16-F10-Luc cells seven days post second immunization.

FIG. 15L: Survival curves for mice shown in FIG. 15K and FIG. 15M. Survival curves for mice that first received two vaccinations two weeks apart of pVAX vector, or combination of DLnano_LS_Trp2₁₈₈ and DLnano_LS_Gp10025. The mice were then given either anti-mouse CD8 antibody or Rat IgG2b isotype control antibody and challenged with 105 B16-F10-Luc cells seven days post the second immunization. Each group includes five mice; each dot represents a mouse; arrow below the plot represents treatments; error bar represents standard deviation; two-tailed Mann-Whitney Rank Test used to compare groups; log-rank tests were used to compare survivals between two groups; p-values were adjusted for multiple comparison where appropriate; *, p-value<0.05; **, p-value<0.005.

FIG. 16A-16M show comparison of immune responses induced by DLnano_LS_GT8 versus protein eOD-GT8-60mer or DLnano_LS_HA(NC99) versus HA(NC99)-60mer in BALB/c mice. Mice were vaccinated twice three weeks apart and euthanized two weeks post the second vaccination for cellular analysis. FIG. 16A: Layout of all plasmids used in this study. FIG. 16B-16D: Immune responses induced by vaccination with either 25 μg of DLnano_LS_GT8 (with electroporation) or 10 μg protein eOD-GT8-60mer adjuvanted with either RIBI, 50 μg poly (I:C) or 20 μg CpG ODN (without electroporation). FIG. 16B: IFNγ CD8+ T-cell responses induced to the GT8 domain by ICS analysis. FIG. 16C: Total T-cell responses induced to the GT8 domain by IFNγ ELIspot analyses. FIG. 16D: IFNγ CD4+ T-cell responses induced to the LS domain by ICS analysis. FIG. 16E-16H: Immune responses induced by vaccination with either 50 μg of DLnano_LS_GT8 (with electroporation) or 50 μg RIBI-adjuvanted protein eOD-GT8-60mer (without electroporation). FIG. 16E: IFNγ CD8+ T-cell responses induced to the GT8 domain by ICS analysis. FIG. 16F: Total T-cell responses induced to the GT8 domain by IFNγ ELIspot analyses. FIG. 16G: IFNγ CD4+ T-cell responses induced to the LS domain by ICS analysis. FIG. 16H: Humoral responses induced to the GT8 by ELISA analysis. FIG. 16I-16K: Immune responses induced by vaccination with either 25 μg of DLnano_LS_GT8 with or without electroporation, or 10 μg RIBI-adjuvanted protein eOD-GT8-60mer with or without electroporation. FIG. 16I: Humoral responses induced by DLnano_LS_GT8 (with or without EP) to the GT8 by ELISA analysis. FIG. 16J: Humoral responses induced by protein eOD-GT8-60mer (with or without EP) to the GT8 by ELISA analysis. FIG. 16K: IFNγ CD8+ T-cell responses induced to the GT8 domain by ICS analysis. FIG. 16L: CD4+IFNγ responses to the LS domain induced by DLnano_LS_HA(NC99), HA(NC99)-60mer or in naïve mice. FIG. 16M: IFNγ ELIspot images for overall T-cell responses to the HA domain induced by DLnano_LS_HA(NC99), HA(NC99)-60mer or in naïve mice. Each group includes five mice; each dot represents an animal; error bar represents standard deviation; arrow below the plot represents an immunization; two-tailed Mann-Whitney Rank Test used to compare groups; p-values were adjusted for multiple comparison where appropriate; *, p-value<0.05.

FIG. 17A-17K show characterization of muscle infiltrating APC induced by DNA or protein vaccination. FIG. 17A: Immunofluorescence staining for cleaved caspase 3 (light gray) or nuclei (gray) for muscle sections from naïve mice or those treated with protein eOD_GT8-60mer (with or without EP) or DLnano_LS_GT8 (with or without EP) harvested four d.p.i. FIG. 17B: Time course immunofluorescence images showing cleaved caspase 3 expression (light gray) in muscle tissue transfected with DLnano_LS_GT8 with EP over time; images from two mice were shown at each time point; nuclei staining with DAPI is shown in gray. FIG. 17C-17D: Serum LDH and CK enzymatic activity in mice immunized with DLnano_LS_GT8, RIBI adjuvanted protein eOD-GT8-60mer or untreated mice following injection. FIG. 17E: Flow plot for determination of macrophage polarization (M1 versus M2) by staining CD11b+F4/80+ populations with CD206 and CD11c. FIG. 17F: Frequencies of M1 versus M2 macrophages in the muscles as determined by flow four days post DLnano_LS_GT8 vaccination. FIG. 17G: Flow plot by VRC01-FITC staining for determination of GT8-uptake in muscle macrophages from naïve mice, or mice treated with protein eOD-GT8-60mer or DLnano_LS_GT8 seven d.p.i. FIG. 17H: Flow plots for splenic CD11c+MHC Class II+DC populations one day upon IV treatment of clodrosome or control encapsosome. FIG. 17I: Flow plots for splenic CD11b+F4/80+ macrophage populations one day upon IV treatment of clodrosome or control encapsosome. FIG. 17J: Comparison for changes in frequencies of splenic macrophages and DCs in Naïve mice upon IV treatment of clodrosome or control encapsosome. FIG. 17K: Humoral responses to GT8 in naïve mice or C57BL/6 mice or BATF3KO mice vaccinated with 25 μg DLnano_LS_GT8 plus EP. Mice were vaccinated once and euthanized two weeks post vaccination. Each group includes five mice in FIG. 17C-17K; each dot represents an animal; error bar represents standard deviation; arrow below the plot represents an immunization; two-tailed Mann-Whitney Rank Test used to compare groups; p-values were adjusted for multiple comparison where appropriate; *, p-value<0.05.

FIG. 18A-18M show characterization of CD8+ T-cell responses induced by DLnano_LS_Trp2₁₈₈ and DLnano_LS_Gp100₂₅. FIG. 18A: SEC trace for designed LS_Gp100₂₅-60mer. FIG. 18B: nsEM image of SEC purified LS_Gp100₂₅-60mer nanoparticles. FIG. 18C: VRC01 binding of SEC purified GT8-monomer, GT8-60mer, Trp2-60mer and Gp100-60mer by an ELISA assay. FIG. 18D: Comparison of CD8+IFNγ T-cell responses to both Trp2₁₈₈ and Gp100₂₅ peptide in naïve C57BL/6 mice or mice immunized twice two weeks apart with 10 μg of both DLmono_Gp100₂₅ and DLnano_LS_Trp2₁₈₈ or DLnano_LS_Gp100₂₅ and DLnano_LS_Trp2₁₈₈. Mice were euthanized two weeks post the second vaccination for cellular analysis. FIG. 18E: B16-F10 challenge survival curves in the therapeutic model. C57BL/6 mice received subcutaneous implantation of 105 B16-F10 cells and then received treatment three days post tumor-inoculation, followed by weekly treatments for a total of four doses. Five groups of mice received either (1) 20 μg pVAX backbone alone, (2) 200 μg anti-PD1 antibody alone, (3) 10 μg DLnano_LS_Trp2₁₈₈ and 10 μg DLnano_LS_Gp100₂₅ alone, (4) 10 μg DLmono_Trp2₁₈₈, 10 μg DLmono_Gp100₂₅ and 200 μg anti-PD1 antibody, or (5) 10 μg DLnano_LS_Trp2₁₈₈, 10 μg DLnano_LS_Gp100₂₅ and 200 μg anti-PD1 antibody for each treatment. FIG. 18F-18I: Comparison of the immunogenicity of DLnano-vaccines versus CpG adjuvanted peptide vaccines in mice. Mice were vaccinated twice with either 10 μg DLnano_LS_Trp2₁₈₈/DLnano_LS_Gp100₂₅ or 10 μg Trp2₁₈₈/Gp100₂₅ peptide adjuvanted with 20 μg CpG ODN two weeks apart and euthanized two weeks post the second vaccination. FIG. 18F: Comparison of CD8+IFNγ T-cell responses to Trp2₁₈₈ peptide in naïve mice or mice immunized with either DLnano_LS_Trp2₁₈₈ or CpG adjuvanted Trp2₁₈₈ peptide by the ICS assay. FIG. 18G: Comparison of CD8+IFNγ T-cell responses to Gp100₂₅ peptide in naïve mice or mice immunized with either DLnano_LS_Gp100₂₅ or CpG adjuvanted Gp100₂₅ peptide by the ICS assay. FIG. 18H-18I: Comparison of IFNγ T-cell responses to Trp2₁₈₈ or Gp100₂₅ peptides in naïve mice or mice immunized with either DLnano_LS_Trp2₁₈₈/DLnano_LS_Gp100₂₅ or CpG adjuvanted Trp2₁₈₈/Gp100₂₅ peptides by the ELIspot assay. FIG. 18J-18L: Induced IFNγ+, IFNγ+CD107a+, or IFNγ+ TNFα+IL-2+CD8+ T-cell responses to Gp100₂₅ in naïve tumor-free mice or B16-F10 bearing mice that received anti-PD1 treatment alone, or anti-PD1 treatment in combination with 4 μg each of RIBI-adjuvanted protein Trp2₁₈₈ and Gp100₂₅-60mer or 10 μg each of DLnano_LS_Trp2₁₈₈ and DLnano_LS_Gp100₂₅. FIG. 18M: Tumor growths in mice that first received two vaccinations two weeks apart of pVAX vector, or a combination of DLnano_LS_Trp2₁₈₈ and DLnano_LS_Gp100₂₅. The mice then received either anti-mouse CD8 antibody or Rat IgG2b isotype control antibody and were then challenged with 105 B16-F10-Luc cells seven days post the second immunization. Each group includes five mice; each dot represents an animal; error bar represents standard deviation; arrow below the plot represents an immunization; two-tailed Mann-Whitney Rank Test used to compare groups; log-rank test used to compare survival between two groups; *, p-value<0.05; **, p-value<0.005.

FIG. 19 shows formulation of CD40L as a trimer or 60mers does not lead to expression of CD40L nanoparticles.

FIG. 20A depicts a schematic of a modification of LS 60mer (CD40L_GT60v1) by including an expression domain. FIG. 20B shows that the construct CD40L_GT60v1 produced about 35% nanoparticles.

FIG. 21A depicts a structural design of 3 new glycans to CD40L or 12 total N-linked glycans (CD40L_g12). FIG. 21B shows that the construct CD40L_g12_60 mer resulted in the production of about 82% of CD40L nanoparticles. FIG. 21C depicts a comparison of the nanoparticles produced by the constructs CD40L_GT60v1 and CD40L_g12_60 mer.

FIG. 22A depicts a schematic of the resulted CD40L/anti-PD1 combo nanoparticle. FIG. 22B shows that mice injected with the DNA vaccine and the CD40L/anti-PD1 combo nanoparticles exhibited better T-cell responses as compared to the mice injected with the DNA vaccine alone.

### DETAILED DESCRIPTION

The disclosed method and compositions may be understood more readily by reference to the following detailed description of particular embodiments and the examples included therein and to the figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present disclosure which will be limited only by the appended claims.

### Definitions

It must be noted that as used herein and in the appended claims, the singular forms “a”, “an,” and “the” include plural reference unless the context clearly dictates otherwise. Thus, for example, reference to “a nucleic acid sequence” includes a plurality of nucleotides that are formed, reference to “the nucleic acid sequence” is a reference to one or more nucleic acid sequences and equivalents thereof known to those skilled in the art, and so forth.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. The term “about” as used herein when referring to a measurable value such as an amount, a temporal duration, and the like, is meant to encompass variations of ±20%, ±10%, ±5%, ±1%, ±0.5%, or ±0.1% from the specified value, as such variations are appropriate to perform the disclosed methods.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” in context of an immunotherapy refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, indirect or direct ligation of cell surface moieties, even in the absence of a direct signal transduction event, may result in the reorganization of cytoskeletal structures, or in the coalescing of cell surface moieties, each of which could serve to enhance, modify, or alter subsequent cellular responses. As used herein, the terms “activating CD8+ T cells” or “CD8+ T cell activation” refer to a process (e.g., a signaling event) causing or resulting in one or more cellular responses of a CD8+ T cell (CTL), selected from: proliferation, differentiation, cytokine secretion, cytotoxic effector molecule release, cytotoxic activity, and expression of activation markers. As used herein, an “activated CD8+ T cell” refers to a CD8+ T cell that has received an activating signal, and thus demonstrates one or more cellular responses, selected from proliferation, differentiation, cytokine secretion, cytotoxic effector molecule release, cytotoxic activity, and expression of activation markers. Suitable assays to measure CD8+ T cell activation are known in the art and are described herein.

The term “combination therapy” as used herein is meant to refer to administration of one or more therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dose having a fixed ratio of each therapeutic agent or in multiple, individual doses for each of the therapeutic agents. For example, one combination of the present disclosure may comprise a pooled sample of one or more nucleic acid molecules comprising one or a plurality of expressible nucleic acid sequences and an adjuvant and/or an anti-viral agent administered at the same or different times. In some embodiments, the pharmaceutical composition of the disclosure can be formulated as a single, co-formulated pharmaceutical composition comprising one or more nucleic acid molecules comprising one or a plurality of expressible nucleic acid sequences and one or more adjuvants and/or one or more anti-viral agents. As another example, a combination of the present disclosure (e.g., DNA vaccines and anti-viral agent) may be formulated as separate pharmaceutical compositions that can be administered at the same or different time. As used herein, the term “simultaneously” is meant to refer to administration of one or more agents at the same time. For example, in certain embodiments, antiviral vaccine or immunogenic composition and antiviral agents are administered simultaneously). Simultaneously includes administration contemporaneously or immediately sequentially, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered intramuscularly only. The components may be administered in any therapeutically effective sequence. A “combination” embraces groups of compounds or non-small chemical compound therapies useful as part of a combination therapy. In some embodiments, the therapeutic agent is an anti-retroviral therapy, (such as one or a combination of efavirenz, lamivudine and tenofovir disoproxil fumarate) or anti-flu therapy (such as TamiFlu®). In some embodiments, the therapeutic agent is one or a combiantion of: abacavir/dolutegravir/lamivudine (Triumeq), dolutegravir/rilpivirine (Juluca), elvitegravir/cobicistat/emtricitabine/tenofovir disoproxil fumarate (Stribild), elvitegravir/cobicistat/emtricitabine/tenofovir alafenamide (Genvoya), efavirenz/emtricitabine/tenofovir disoproxil fumarate (Atripla), emtricitabine/rilpivirine/tenofovir disoproxil fumarate (Complera), emtricitabine/rilpivirine/tenofovir alafenamide (Odefsey), bictegravir, emtricitabine, and tenofovir alafenamide (Biktarvy). In some embodiments, the therapeutic agent is one or a combination of a reverse transcrioptase inhibitor of a retrovirus such as: efavirenz (Sustiva), etravirine (Intelence), nevirapine (Viramune), nevirapine extended-release (Viramune XR), rilpivirine (Edurant), delavirdine mesylate (Rescriptor). In some embodiments, the therapeutic agent is one or a combination of a protease inhibitor of a retrovirus, such as: atazanavir/cobicistat (Evotaz), darunavir/cobicistat (Prezcobix), lopinavir/ritonavir (Kaletra), ritonavir (Norvir), atazanavir (Reyataz), darunavir (Prezista), fosamprenavir (Lexiva), tipranavir (Aptivus).

As used herein, “expression” refers to the process by which a polynucleotide is transcribed from a DNA template (such as into and mRNA or other RNA transcript) and/or the process by which a transcribed mRNA (or administered mRNA) is translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. In some embodiments, the at least first expressible nucleic acid sequence comprises only DNA nucleotides, RNA nucleotides or comprises both RNA and DNA nucleotides. In some embodiments, the at least first expressible nucleic acid consist of RNA. In some embodiments, the at least first expressible nucleic acid consist of DNA.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based (such wild-type or full length sequences “reference sequences” or each individually a “reference sequence”). In some embodiments, the functional fragment is derived from the sequence of an organism, such as a human. In such embodiments, the functional fragment may retain about 99%, 98%, 97%, 96%, 95%, 94%, 93%, 92%, 91%, or 90% sequence identity to the wild-type human sequence upon which the sequence is derived. In some embodiments, the functional fragment may retain about 85%, 80%, 75%, 70%, 65%, or 60% sequence identity to the wild-type sequence upon which the sequence is derived.

By “fragment” is meant a portion of a polypeptide or nucleic acid molecule. This portion contains, preferably, at least about 5%, 10%, 20%, 30%, 40%, 50%, 60%, 70%, 80%, or about 90% of the entire length of the reference nucleic acid molecule or polypeptide. A fragment may contain about 5, 10, 20, 30, 40, 50, 60, 70, 80, 90, 100, 200, 300, 400, 500, 600, 700, 800, 900, 1000 or more nucleotides or amino acids.

“Optional” or “optionally” means that the subsequently described event, circumstance, or material may or may not occur or be present, and that the description includes instances where the event, circumstance, or material occurs or is present and instances where it does not occur or is not present.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in some embodiments, to A without B (optionally including elements other than B); in another embodiments, to B without A (optionally including elements other than A); in yet another embodiments, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “cither,” “one of,” “only one of,” or “exactly one of.” “Consisting essentially of,” when used in the claims, shall have its ordinary meaning as used in the field of patent law.

As used herein an “antigen” is meant to refer to any substance that elicits an immune response.

As used herein, the term “a CD40 ligand,” “CD40L” or “CD40L polypeptide” refers to a protein that is primarily expressed on activated T cells and is a member of the TNF superfamily of molecules. It binds to CD40 protein on antigen-presenting cells (APC), which leads to many effects depending on the target cell type. In some embodiments, the CD40L polypeptide is a human CD40 ligand comprising the amino acid sequence of SEQ ID NO: 103 encoded by the nucleic acid sequence of SEQ ID NO: 102.

catgctgcctctgccaccttctctgccagaagataccatttcaactttaacacagcatgatcgaaacatacaaccaaacttctccccgatct
gcggccactggactgcccatcagcatgaaaatttttatgtatttacttactgtttttcttatcacccagatgattgggtcagcactttttgctgt
gtatcttcatagaaggctggacaagatagaagatgaaaggaatcttcatgaagattttgtattcatgaaaacgatacagagatgcaacac
aggagaaagatccttatccttactgaactgtgaggagattaaaagccagtttgaaggctttgtgaaggatataatgttaaacaaagagga
gacgaagaaagaaaacagctttgaaatgcaaaaaggtgatcagaatcctcaaattgcggcacatgtcataagtgaggccagcagtaa
aacaacatctgtgttacagtgggctgaaaaaggatactacaccatgagcaacaacttggtaaccctggaaaatgggaaacagctgacc
gttaaaagacaaggactctattatatctatgcccaagtcaccttctgttccaatcgggaagcttcgagtcaagctccatttatagccagcct
ctgcctaaagtcccccggtagattcgagagaatcttactcagagctgcaaatacccacagttccgccaaaccttgcgggcaacaatcc
attcacttgggaggagtatttgaattgcaaccaggtgcttcggtgtttgtcaatgtgactgatccaagccaagtgagccatggcactggct
tcacgtcctttggcttactcaaactctgaacagtgtcaccttgcaggctgtggtggacgtgacgctgggagtcttcataatacagcacag
cggttaagcccaccccctgttaactgcctatttataaccctaggatcctccttatggagaactatttattatacactccaaggcatgtagaac
tgtaataagtgaattacaggtcacatgaaaccaaaacgggccctgctccataagagcttatatatctgaagcagcaaccccactgatgc
agacatccagagagtcctatgaaaagacaaggccattatgcacaggttgaattctgagtaaacagcagataacttgccaagttcagtttt
gtttctttgcgtgcagtgtctttccatggataatgcatttgatttatcagtgaagatgcagaagggaaatggggagcctcagctcacattca
gttatggttgactctgggttcctatggccttgttggagggggccaggctctagaacgtctaacacagtggagaaccgaaacccccccc
ccccccgccaccctctcggacagttattcattctctttcaatctctctctctccatctctctctttcagtctctctctctcaacctctttcttcca
atctctctttctcaatctctctgtttccctttgtcagtctcttccctcccccagtctctcttctcaatccccctttctaacacacacacacacaca
cacacacacacacacacacacacacacacacacacacacacacagagtcaggccgttgctagtcagttctcttctttccaccctgtcccta
tctctaccactatagatgagggtgaggagtagggagtgcagccctgagcctgcccactcctcattacgaaatgactgtatttaaaggaa
atctattgtatctacctgcagtctccattgtttccagagtgaacttgtaattatcttgttatttattttttgaataataaagacctcttaacatt
(SEQ ID NO: 102; X68550.1 H.sapiens TRAP mRNA for ligand of CD40)
MIETYNQTSPRSAATGLPISMKIFMYLLTVFLITQMIGSALFAVYLHRRLDKIEDERNL
HEDFVFMKTIQRCNTGERSLSLLNCEEIKSQFEGFVKDIMLNKEETKKENSFEMQKG
DQNPQIAAHVISEASSKTTSVLQWAEKGYYTMSNNLVTLENGKQLTVKRQGLYYIY
AQVTFCSNREASSQAPFIASLCLKSPGRFERILLRAANTHSSAKPCGQQSIHLGGVFEL
QPGASVFVNVTDPSQVSHGTGFTSFGLLKL (SEQ ID NO: 103;
P29965|CD40L_HUMAN CD40 ligand OS = Homo sapiens)

In some embodiments, the CD40L polypeptide is a fragment of the human CD40 ligand comprising the amino acid sequence of SEQ ID NO: 104, SEQ ID NO: 105, or SEQ ID NO: 106.

(SEQ ID NO: 104)
DKIEDERNLHEDFVFMKTIQRCNTGERSLSLLNCEEIKSQFEGFVKDIM
LNKEETKKENSFEMQKGDQNPQIAAHVISEASSKTTSVLQWAEKGYYTM
SNNLVTLENGKQLTVKRQGLYYIYAQVTFCSNREASSQAPFIASLCLKS
PGRFERILLRAANTHSSAKPCGQQSIHLGGVFELQPGASVFVNVTDPSQ
VSHGTGFTSFGLLKL
(SEQ ID NO: 105)
MQKGDQNPQIAAHVISEASSKTTSVLQWAEKGYYTMSNNLVTLENGKQL
TVKRQGLYYIYAQVTFCSNREASSQAPFIASLCLKSPGRFERILLRAAN
THSSAKPCGQQSIHLGGVFELQPGASVFVNVTDPSQVSHGTGFTSFGLL
KL
(SEQ ID NO: 106)
IAAHVISEASSKTTSVLQWAEKGYYTMSNNLVTLENGKQLTVKRQGLYY
IYAQVTFCSNREASSQAPFIASLCLKSPGRFERILLRAANTHSSAKPCG
QQSIHLGGVFELQPGASVFVNVTDPSQVSHGTGFTSFGLLKL

In some embodiments, the CD40L polypeptide is a mouse CD40 ligand comprising the amino acid sequence of SEQ ID NO: 108 encoded by the nucleic acid sequence of SEQ ID NO: 107.

ctttcagtcagcatgatagaaacatacagccaaccttcccccagatccgtggcaactggacttccagcgagcatgaagatttttatgtatt
tacttactgttttccttatcacccaaatgattggatctgtgctttttgctgtgtatcttcatagaagattggataaggtcgaagaggaagtaaa
ccttcatgaagattttgtattcataaaaaagctaaagagatgcaacaaaggagaaggatctttatccttgctgaactgtgaggagatgaga
aggcaatttgaagaccttgtcaaggatataacgttaaacaaagaagagaaaaaagaaaacagctttgaaatgcaaagaggtgatgag
gatcctcaaattgcagcacacgttgtaagcgaagccaacagtaatgcagcatccgttctacagtgggccaagaaaggatattataccat
gaaaagcaacttggtaatgcttgaaaatgggaaacagctgacggttaaaagagaaggactctattatgtctacactcaagtcaccttctg
ctctaatcgggagccttcgagtcaacgcccattcatcgtcggcctctggctgaagcccagcagtggatctgagagaatcttactcaagg
cggcaaatacccacagttcctcccagctttgcgagcagcagtctgttcacttgggcggagtgtttgaattacaagctggtgcttctgtgttt
gtcaacgtgactgaagcaagccaagtgatccacagagttggcttctcatcttttggcttactcaaactctgaacagtgcgctgtcctaggc
tgcagcagggctgatgctggcagtcttccctatacagcaagtcagttaggacctgccctgtgttgaactgcctatttataaccctaggatc
ctcctcatggagaactatttattatgtacccccaaggcacatagagctggaataagagaattacagggcaggcaaaaatcccaaggga
ccctgctccctaagaacttacaatctgaaacagcaaccccactgattcagacaaccagaaaagacaaagccataatacacagatgaca
gagctctgatgaaacaacagataactaatgagcacagttttgttgttttatgggtgtgtcgttcaatggacagtgtacttgacttaccaggg
aagatgcagaagggcaactgtgagcctcagctcacaatctgttatggttgacctgggctccctgcggccctagtagg (SEQ ID
NO: 107; NM_011616.2 Mus musculus CD40 ligand (Cd40lg), mRNA)
MIETYSQPSPRSVATGLPASMKIFMYLLTVFLITQMIGSVLFAVYLHRRLDKVEEE
VNLHEDFVFIKKLKRCNKGEGSLSLLNCEEMRRQFEDLVKDITLNKEEKKENSFE
MQRGDEDPQIAAHVVSEANSNAASVLQWAKKGYYTMKSNLVMLENGKQLTVK
REGLYYVYTQVTFCSNREPSSQRPFIVGLWLKPSSGSERILLKAANTHSSSQLCEQ
QSVHLGGVFELQAGASVFVNVTEASQVIHRVGFSSFGLLKL (SEQ ID NO: 108;
P27548|CD40L_MOUSE CD40 ligand OS = Mus musculus)

In some embodiments, the CD40L polypeptide is a fragment of the mouse CD40 ligand comprising the amino acid sequence of SEQ ID NO: 109, SEQ ID NO: 110, or SEQ ID NO: 111.

(SEQ ID NO: 109)
DKVEEEVNLHEDFVFIKKLKRCNKGEGSLSLLNCEEMRRQFEDLVKDIT
LNKEEKKENSFEMQRGDEDPQIAAHVVSEANSNAASVLQWAKKGYYTMK
SNLVMLENGKQLTVKREGLYYVYTQVTFCSNREPSSQRPFIVGLWLKPS
SGSERILLKAANTHSSSQLCEQQSVHLGGVFELQAGASVFVNVTEASQV
IHRVGFSSFGLLKL
(SEQ ID NO: 110)
MQRGDEDPQIAAHVVSEANSNAASVLQWAKKGYYTMKSNLVMLENGKQL
TVKREGLYYVYTQVTFCSNREPSSQRPFIVGLWLKPSSGSERILLKAAN
THSSSQLCEQQSVHLGGVFELQAGASVFVNVTEASQVIHRVGFSSFGLL
KL
(SEQ ID NO: 111)
IAAHVVSEANSNAASVLQWAKKGYYTMKSNLVMLENGKQLTVKREGLYY
VYTQVTFCSNREPSSQRPFIVGLWLKPSSGSERILLKAANTHSSSQLCE
QQSVHLGGVFELQAGASVFVNVTEASQVIHRVGFSSFGLLKL

In some embodiments, the “CD40L” or “CD40L polypeptide,” as used herein, comprises at least about 70%, 75%, 80%, 85%, 90%, 91%, 92%, 93%, 94%, 95%, 96%, 97%, 98%, 99% or 100% sequence identity to SEQ ID NO: 103, SEQ ID NO: 104, SEQ ID NO: 105, SEQ ID NO: 106, SEQ ID NO: 108, SEQ ID NO: 109, SEQ ID NO: 110, or SEQ ID NO: 111. In some embodiments, the “CD40L” or “CD40L polypeptide,” as used herein, is encoded by a nucleic acid comprising at least about 70%, 75%, 80%, 85%, 90%, 91%, 92%, 93%, 94%, 95%, 96%, 97%, 98%, 99% or 100% sequence identity to SEQ ID NO: 102 or SEQ ID NO: 107. In some embodiments, the “CD40L” or “CD40L polypeptide,” as used herein, refers to a CD40L polypeptide comprising at least about 1 N-linked glycan. In some embodiments, the “CD40L” or “CD40L polypeptide,” as used herein, refers to a CD40L polypeptide comprising at least about 2 N-linked glycan. In some embodiments, the “CD40L” or “CD40L polypeptide,” as used herein, refers to a CD40L polypeptide comprising at least about 3 N-linked glycan. In some embodiments, the “CD40L” or “CD40L polypeptide,” as used herein, refers to a CD40L polypeptide comprising at least about 4 N-linked glycan. In some embodiments, the “CD40L” or “CD40L polypeptide,” as used herein, refers to a CD40L polypeptide comprising at least about 5 N-linked glycan. In some embodiments, the “CD40L” or “CD40L polypeptide,” as used herein, refers to a CD40L polypeptide comprising more than about 5 N-linked glycan.

As used herein, the term “protein of interest” refers to any protein. In some embodiments, the term “protein of interest” refers to any protein that can be expressed in any of the constructs disclosed herein. In some embodiments, the “protein of interest” is a viral antigen. In some embodiments, the “protein of interest” is any of the viral antigens disclosed herein. In some embodiments, the “protein of interest” is a cancer antigen. In some embodiments, the “protein of interest” is a cancer antigen disclosed herein. In some embodiments, the “protein of interest” is any of the protein antigens disclosed herein.

As used herein, the term “electroporation,” “electro-permeabilization,” or “electro-kinetic enhancement” (“EP”), are used interchangeably and are meant to refer to the use of a transmembrane electric field pulse to induce microscopic pathways (pores) in a bio-membrane; their presence allows biomolecules such as plasmids, oligonucleotides, siRNA, drugs, ions, and/or water to pass from one side of the cellular membrane to the other. In some of the disclosed methods of treatment or prevention, the method comprises a step of electroporation of a subject's tissue for a sufficient time and with a sufficient electrical field capable of inducing uptake of the pharmaceutical compositions disclosed herein into the antigen-presenting cells. In some embodiments, the cells are antigen presenting cells.

The term “pharmaceutically acceptable excipient,” “pharmaceutically acceptable carrier” or “pharmaceutically acceptable diluent” as used herein is meant to refer to an excipient, carrier or diluent that can be administered to a subject, together with an agent or the pharmaceutical compositions disclosed herein, and which is inert or fails to eliminate the pharmacological activity of the active agent of the pharmaceutical composition. In some embodiments, the pharmaceutically acceptable carrier does fails to destroy or is incapable of eliminating the pharmacological activity of an active agent/vaccine and is nontoxic when administered in doses sufficient to deliver a therapeutic amount of the active agent. The term “pharmaceutically acceptable salt” of nucleic acids as used herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, suifanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenyiacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled viral specific antigens or polynucleotides provided herein, including those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing,” “prevention,” “prophylactic treatment,” and the like, are meant to refer to reducing the probability of developing a disease or condition in a subject, who does not have, but is at risk of or susceptible to developing a disease or condition.

As used herein, the term “purified” means that the polynucleotide or polypeptide or fragment, variant, or derivative thereof is substantially free of other biological material with which it is naturally associated, or free from other biological materials derived, e.g., from a recombinant host cell that has been genetically engineered to express the polypeptide of the present disclosure. That is, e.g., a purified polypeptide of the present disclosure is a polypeptide that is at least from about 70 to 100% pure, i.e., the polypeptide is present in a composition wherein the polypeptide constitutes from about 70 to about 100% by weight of the total composition. In some embodiments, the purified polypeptide of the present disclosure is from about 75% to about 99% by weight pure, from about 80% to about 99% by weight pure, from about 90 to about 99% by weight pure, or from about 95% to about 99% by weight pure.

As used herein, the terms “subject,” “individual,” “host,” and “patient,” are used interchangeably herein and refer to a vertebrate individual, including but not limited to a mammal or human, for whom diagnosis, treatment or therapy is desired, particularly humans. Mammals include, but are not limited to, murines, simians, humans, farm animals, cows, pigs, goats, sheep, horses, dogs, sport animals, and pets. Tissues, cells and their progeny obtained in vivo or cultured in vitro are also encompassed by the definition of the term “subject.” The methods described herein are applicable to both human therapy and veterinary applications. In some instances in the description of the present disclosure, the term “patient” refers to human patients suffering from a particular disease or disorder. In some embodiments, the subject may be a human suspected of having or being identified as at risk to develop a viral infection. In some embodiments, the subject may be diagnosed as having human immunodeficiency virus-1 (HIV-1) and of having or being identified as at risk to develop autoimmune deficiency syndrome or AIDS. In some embodiments, the subject is a mammal, and, in other embodiments, the subject is a human.

The term “therapeutic effect” as used herein is meant to refer to some extent of relief of one or more of the symptoms of a disorder (e.g., HIV infection) or its associated pathology. A “therapeutically effective amount” as used herein is meant to refer to an amount of an agent which is effective, upon single or multiple dose administration (such as a first, second and/or third booster) to the cell or subject, in prolonging the survivability of the patient with such a disorder, reducing one or more signs or symptoms of the disorder, preventing or delaying, and the like beyond that expected in the absence of such treatment. A “therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the present disclosure employed in a pharmaceutical composition at levels lower than that required in order to achieve the desired therapeutic effect and gradually increase the dosage until the desired effect is achieved.

The terms “treat,” “treated,” “treating,” “treatment,” and the like as used herein are meant to refer to reducing or ameliorating a disorder and/or symptoms associated therewith (e.g., a viral infection). “Treating” can refer to administration of the DNA vaccines described herein to a subject after the onset, or suspected onset, of a viral infection. “Treating” includes the concepts of “alleviating,” which refers to lessening the frequency of occurrence or recurrence, or the severity, of any symptoms or other ill effects related to a virus and/or the side effects associated with viral therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

For any therapeutic agent described herein the therapeutically effective amount may be initially determined from preliminary in vitro studies and/or animal models. A therapeutically effective dose may also be determined from human data. The applied dose can be adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below. Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained. Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Thus, ther term “expressible nucleic acid” or “expressible nucleic acid sequence” as used herein refers to expressible DNA or RNA molecules or expressible DNA or RNA sequences.

The nucleic acid molecule and/or sequences of each embodiment can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. A nucleic acid will generally contain phosphodiester bonds, although nucleic acid analogs maybe included that may have at least one different linkage, e.g., phosphoramidate, phosphorothioate, phosphorodithioate, or o-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties. Nucleic acids containing one or more non-naturally occurring or modified nucleotides are also included within one definition of nucleic acids. The modified nucleotide analog may he located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; 0- and N-alkylated nucleotides, e.g. N6-methyl adenosine are suitable. The 2′-OH-group may be replaced by a group selected from H, OR, R, halo, SH, SR, NH₂, NHR, N₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in U.S. Pat. No. 20020115080, which is incorporated herein by reference. Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made. In some embodiments, the expressible nucleic acid sequence is in the form of DNA. In some embodiments, the expressible nucleic acid is in the form of RNA with a sequence that encodes the polypeptide sequences disclosed herein and, in some embodiments, the expressible nucleic acid sequence is an RNA/DNA hybrid molecule that encodes any one or plurality of polypeptide sequences disclosed herein.

As used herein, the term “nucleic acid molecule” is a molecule that comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also includes a plasmid containing one or more nucleotide sequences that encode one or a plurality of viral antigens. In some embodiments, the disclosure relates to a pharmaceutical composition comprising a first, second, third or more nucleic acid molecule, each of which encoding one or a plurality of viral antigens and at least one of each plasmid comprising one or more of the compositions disclosed herein. In some embodiments, the compositions can comprise a nucleic acid molecule that comprises a first, second, third or more expressible nucleic acid sequences, wherein at least one of the first, second or third expressible nucleic acid sequences comprise the domains disclosed herein.

The terms “polypeptide,” “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may he performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The term “hybridization” or “hybridizes” as used herein refers to the formation of a duplex between nucleotide sequences that are sufficiently complementary to form duplexes via Watson-Crick base pairing. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G and C of one sequence is then aligned with a T(U), A, C and G, respectively, of the other sequence. RNA sequences can also include complementary G=U or U=G base pairs. Thus, two sequences need not have perfect homology to be “complementary.” Usually two sequences are sufficiently complementary when at least about 90% (preferably at least about 95%) of the nucleotides share base pair organization over a defined length of the molecule.

By “substantially identical” is meant nucleic acid molecule (or polypeptide) exhibiting at least about 50% identity to a reference amino acid sequence (for example, any one of the amino acid sequences described herein) or nucleic acid sequence (for example, any one of the nucleic acid sequences described herein). Preferably, such a sequence is at least about 60%, more preferably about 80% or 85%, and more preferably about 90%, 91%, 92%, 93%, 94%, 95%, 96%, 97%, 98% or even 99% identical at the amino acid level or nucleic acid level to the sequence used for comparison.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), comprising additional, exogenous DNA, RNA or hybrid DNA or RNA molecules that can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide. The disclosure relates to any one or plurality of vectors that comprise nucleic acid sequences encoding any one or plurality of amino acid sequence disclosed herein. In some embodiments, the expression vector includes from about 30 to about 100,000 nucleotides (e.g., from about 30 to about 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from about 1,000 to about 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from about 2,000 to about 70,000, and from 2,000 to 100,000).

The term “vaccine” as used herein is meant to refer to a composition for generating immunity for the prophylaxis and/or treatment of diseases (e.g., viral infections). Accordingly, vaccines are medicaments which comprise antigens in protein and/or nucleic acid forms and are in animals for generating specific defense and protective substance by vaccination. A “vaccine composition” or a “DNA vaccine composition” can include a pharmaceutically acceptable excipient, carrier or diluent. A “DNA vaccine composition” as used herein can comprise a DNA vaccine, a RNA vaccine or a combination thereof.

“Variants” are intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring or endogenous nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present disclosure is not entitled to antedate such disclosure by virtue of prior disclosure. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### A. Nucleic Acid Compositions

Disclosed are expressible nucleic acid sequences, nucleic acid molecules comprising one or plurality of such expressible nucleic acid sequences, and compositions comprising one or plurality of such expressible nucleic acid sequences and/or nucleic acid molecules. The expressible nucleic acid sequence according to the present disclosure comprises a first nucleic acid sequence encoding a scaffold domain comprising a self-assembling polypeptide, and a second nucleic acid sequence encoding an antigen domain comprising a viral antigen, and optionally, wherein the expressible nucleic acid sequence is free of a nucleic acid sequence encoding a leader sequence. In some embodiments, the expressible nucleic acid sequence further comprises a third nucleic acid sequence encoding a linker domain comprising a linker peptide, wherein the third nucleic acid sequence is positioned between the first nucleic acid sequence and the second nucleic acid sequence in the 5′ to 3′ orientation. Thus, also disclosed are compositions comprising an expressible nucleic acid sequence comprising a first nucleic acid sequence encoding a scaffold domain comprising a self-assembling polypeptide, a second nucleic acid sequence encoding an antigen domain comprising a viral antigen, and a third nucleic acid sequence encoding a linker domain comprising a linker peptide, wherein the third nucleic acid sequence is positioned between the first nucleic acid sequence and the second nucleic acid sequence in the 5′ to 3′ orientation, and optionally, wherein the expressible nucleic acid sequence is free of a nucleic acid sequence encoding a leader sequence. In some embodiments, the expressible nucleic acid is operably linked to at least one regulatory sequence and/or forms part of a nucleic acid molecule, such as a plasmid.

In some embodiments, compositions of the disclosure relate to a composition comprising one or a plurality of expressible nucleic acid sequences disclosed herein. In some embodiments, the self-assembling polypeptide is a self-assembling peptide that is expressed to envelope an antigen. Transformed or transfected cells exposed to the vaccine can produce the self-assembling peptide that envelopes the viral antigens, thereby stimulating an antigen-specific immune response against the antigen. In some embodiments, the antigen-specific immune response is a therapeutically effective immune response against the virus from which the antigen amino acid sequence is derived.

### 1. Self-Assembling Polypeptide

The disclosure relates to an expressible nucleic acid sequence comprising a first nucleic acid sequence encoding a scaffold domain comprising a self-assembling polypeptide. Self-assembling polypeptide are polypeptides capable of undergoing spontaneous assembling into ordered nanostructures. Effectively self-assembling polypeptides can act as building blocks to form the scaffold domain of the present disclosure. Any self-assembling polypeptide can be used. In some embodiments, the self assembling polypeptide is from Aquifex aeolicus, Helicobacter pylori, Pyrococcus furiosus or Thermotoga maritima.

A non-limiting example of a self-assembling polypeptide is the lumazine synthase of hyperthermophilic bacterium Aquifex aeolicus having the amino acid sequence of SEQ ID NO: 7 (LS scaffold) encoded by the nucleic acid sequence of SEQ ID NO: 2.

(SEQ ID NO: 2)
atgcagatctacgaaggaaaactgaccgctgagggactgaggttcggaattgtcgcaagccgcgcgaatcacgcactggtgg
ataggctggtggaaggcgctatcgacgcaattgtccggcacggcgggagagaggaagacatcacactggtgagagtctgcg
gcagctgggagattcccgtggcagctggagaactggctcgaaaggaggacatcgatgccgtgatcgctattggggtcctgtgc
cgaggagcaactcccagcttcgactacatcgcctcagaagtgagcaaggggctggctgatctgtccctggagctgaggaaacc
tatcacttttggcgtgattactgccgacaccctggaacaggcaatcgaggcggccggcacctgccatggaaacaaaggctggg
aagcagccctgtgcgctattgagatggcaaatctgttcaaatctctgcga
(SEQ ID NO: 7)
MQIYEGKLTAEGLRFGIVASRANHALVDRLVEGAIDAIVRHGGREEDITLVRVCG
SWEIPVAAGELARKEDIDAVIAIGVLCRGATPSFDYIASEVSKGLADLSLELRKPIT
FGVITADTLEQAIEAAGTCHGNKGWEAALCAIEMANLFKSLR

Another non-limiting example of a self-assembling polypeptide is ferritin from Helicobacter pylori having the amino acid sequence of SEQ ID NO: 23 (3BVE scaffold) encoded by the nucleic acid sequence of SEQ ID NO: 13.

(SEQ ID NO: 13)
gggctgagtaaggacattatcaagctgctgaacgaacaggtgaacaaagagatgcagtctagcaacctgtacatgtccatgagc
tcctggtgctatacccactctctggacggagcaggcctgttcctgtttgatcacgccgccgaggagtacgagcacgccaagaag
ctgatcatcttcctgaatgagaacaatgtgcccgtgcagctgacctctatcagcgcccctgagcacaagttcgagggcctgacac
agatctttcagaaggcctacgagcacgagcagcacatctccgagtctatcaacaatatcgtggaccacgccatcaagtccaagg
atcacgccacattcaactttctgcagtggtacgtggccgagcagcacgaggaggaggtgtgtttaaggacatcctggataagat
cgagctgatcggcaatgagaaccacgggctgtacctggcagatcagtatgtcaagggcatcgctaagtcaaggaaaagc
(SEQ ID NO: 23)
GLSKDIIKLLNEQVNKEMQSSNLYMSMSSWCYTHSLDGAGLFLFDHAAEEYEHA
KKLIIFLNENNVPVQLTSISAPEHKFEGLTQIFQKAYEHEQHISESINNIVDHAIKSK
DHATFNFLQWYVAEQHEEEVLFKDILDKIELIGNENHGLYLADQYVKGIAKSRK
S

A yet another non-limiting example of a self-assembling polypeptide is PfV viral cage from Pyrococcus furiosus (2e0z) having the amino acid sequence of SEQ ID NO: 31 (RBE scaffold) encoded by the nucleic acid sequence of SEQ ID NO: 14.

(SEQ ID NO: 14)
ctgagcattgcccccacactgattaaccgggacaaaccctacaccaaagaggaactgatggagattctgagactggctattatcg
ctgagctggacgccatcaacctgtacgagcagatggcccggtattctgaggacgagaatgtgcgcaagatcctgctggatgtg
gccagggaggagaaggcacacgtgggagagttcatggccctgctgctgaacctggaccccgagcaggtgaccgagctgaa
gggcggctttgaggaggtgaaggagctgacaggcatcgaggcccacatcaacgacaataagaaggaggagagcaacgtgg
agtatttcgagaagctgagatccgccctgctggatggcgtgaataagggcaggagcctgctgaagcacctgcctgtgaccagg
atcgagggccagagcttcagagtggacatcatcaagtttgaggatggcgtgcgcgtggtgaagcaggagtacaagcccatccc
tctgctgaagaagaagttctacgtgggcatcagggagctgaacgacggcacctacgatgtgagcatcgccacaaaggccggc
gagctgctggtgaaggacgaggagtccctggtcatccgcgagatcctgtctacagagggcatcaagaagatgaagctgagctc
ctgggacaatccagaggaggccctgaacgatctgatgaatgccctgcaggaggcatctaacgcaagcgccggaccattcggc
ctgatcatcaatcccaagagatacgccaagctgctgaagatctatgagaagtccggcaagatgctggtggaggtgctgaagga
gatcttccggggcggcatcatcgtgaccctgaacatcgatgagaacaaagtgatcatctttgccaacacccctgccgtgctggac
gtggtggtgggacaggatgtgacactgcaggagctgggaccagagggcgacgatgtggcctttctggtgtccgaggccatcg
gcatcaggatcaagaatccagaggcaatcgtggtgctggag
(SEQ ID NO: 31)
LSIAPTLINRDKPYTKEELMEILRLAIIAELDAINLYEQMARYSEDENVRKILLDVA
REEKAHVGEFMALLLNLDPEQVTELKGGFEEVKELTGIEAHINDNKKEESNVEYF
EKLRSALLDGVNKGRSLLKHLPVTRIEGQSFRVDIIKFEDGVRVVKQEYKPIPLLK
KKFYVGIRELNDGTYDVSIATKAGELLVKDEESLVIREILSTEGIKKMKLSSWDNP
EEALNDLMNALQEASNASAGPFGLIINPKRYAKLLKIYEKSGKMLVEVLKEIFRG
GIMQDIFIEDLNIDENKVIIFANTPAVLDVVVGQDVTLQELGPEGDDVAFLVSEAI
GIRIKNPEAIVVLE

A further non-limiting example of a self-assembling polypeptide is the self-assembling polypeptide from Thermotoga maritima having the amino acid sequence of SEQ ID NO: 26 (13 scaffold) encoded by the nucleic acid sequence of SEQ ID NO: 15.

(SEQ ID NO: 15)
gagaaagcagccaaagcagaggaagcagcacggaagatggaagaactgttcaagaagcacaagatcgtggccgtgctgag
ggccaactccgtggaggaggccaagaagaaggccctggccgtgttcctgggcggcgtgcacctgatcgagatcacctttaca
gtgcccgacgccgataccgtgatcaaggagctgtctttcctgaaggagatgggagcaatcatcggagcaggaaccgtgacaa
gcgtggagcagtgcagaaaggccgtggagagcggcgccgagtttatcgtgtcccctcacctggacgaggagatctctcagttc
tgtaaggagaagggcgtgttttacatgccaggcgtgatgacccccacagagctggtgaaggccatgaagctgggccacacaat
cctgaagctgttccctggcgaggtggtgggcccacagtttgtgaaggccatgaagggccccttccctaatgtgaagtttgtgccc
accggcggcgtgaacctggataacgtgtgcgagtggttcaaggcaggcgtgctggcagtgggcgtgggcagcgccctggtg
aagggcacacccgtggaagtcgctgagaaggcaaaggcattcgtggaaaagattagggggtgtactgag
(SEQ ID NO: 26)
EKAAKAEEAARKMEELFKKHKIVAVLRANSVEEAKKKALAVFLGGVHLIEITFT
VPDADTVIKELSFLKEMGAIIGAGTVTSVEQCRKAVESGAEFIVSPHLDEEISQFC
KEKGVFYMPGVMTPTELVKAMKLGHTILKLFPGEVVGPQFVKAMKGPFPNVKF
VPTGGVNLDNVCEWFKAGVLAVGVGSALVKGTPVEVAEKAKAFVEKIRGCTE

Accordingly, in some embodiments, the self-assembling polypeptide encoded by the expressible nucleic acid sequence of the present disclosure comprises at least about 60%, 65%, 70%, 75%, 80%, 85%, 90%, 95%, or 99% sequence identity to SEQ ID NO: 7, SEQ ID NO: 23, SEQ ID NO: 26, or SEQ ID NO: 31, or a functional fragment thereof. In some embodiments, the self-assembling polypeptide comprises the amino acid sequence of SEQ ID NO: 7, SEQ ID NO: 23, SEQ ID NO: 26, or SEQ ID NO: 31, or a functional fragment thereof. In some embodiments, the nucleic acid sequence encoding the self-assembling polypeptide comprises at least about 60%, 65%, 70%, 75%, 80%, 85%, 90%, 95%, or 99% sequence identity to SEQ ID NO: 2, SEQ ID NO: 13, SEQ ID NO: 14, or SEQ ID NO: 15, or a functional fragment thereof. In some embodiments, the nucleic acid sequence encoding the self-assembling polypeptide comprises the nucleotide sequence of SEQ ID NO: 2, SEQ ID NO: 13, SEQ ID NO: 14, or SEQ ID NO: 15, or a functional fragment thereof. In other embodiments, the nucleic acid sequence encoding the self-assembling polypeptide comprises at least about 60%, 65%, 70%, 75%, 80%, 85%, 90%, 95%, or 99% sequence identity to a nucleic acid sequence that is complementary to SEQ ID NO: 2, SEQ ID NO: 13, SEQ ID NO: 14, or SEQ ID NO: 15, or a functional fragment thereof. In some embodiments, the nucleic acid sequence encoding the self-assembling polypeptide comprises a nucleic acid sequence that is complementary to SEQ ID NO: 2, SEQ ID NO: 13, SEQ ID NO: 14, or SEQ ID NO: 15, or a functional fragment thereof.

In some embodiments, the compostions or pharmaceutical compositions of the disclosure comprises a nucleic acid molecule comprising an expressible nucleic acid that encodes a self-assembling polypeptide that is 60%, 65%, 70%, 75%, 80%, 85%, 90%, 95%, or 99% sequence identity to SEQ ID NO: 2, SEQ ID NO: 13, SEQ ID NO: 14, or SEQ ID NO: 15, or a functional fragment thereof; or an expressible nucleic acid that encodes a self-assembling polypeptide that is 60%, 65%, 70%, 75%, 80%, 85%, 90%, 95%, or 99% sequence identity to the complementary sequence of SEQ ID NO: 2, SEQ ID NO: 13, SEQ ID NO: 14, or SEQ ID NO: 15, or a functional fragment thereof. In some embodiments, composition or pharmaceutical composition of the disclosure relate to a vector or a nucleic acid molecule comprising an expressible RNA sequence that encodes a self-assembling polypeptide that is optionally in sequence with one or more additional expressible RNA sequences that encode a viral antigen. In some embodiments, the expressible DNA or RNA sequence that encodes a self-assembling polypeptide is from about 300 to 500 nucletides in length. In some embodiments, the expressible DNA or RNA sequence that encodes a self-assembling polypeptide is from about 350 to 480 nucletides in length. In some embodiments, the expressible DNA or RNA sequence that encodes a self-assembling polypeptide is from about 350 to 460 nucletides in length. In some embodiments, the expressible DNA or RNA sequence that encodes a self-assembling polypeptide is from about 300 to 500 nucletides in length. In some embodiments, the expressible DNA or RNA sequence that encodes a self-assembling polypeptide is from about 400 to 500 nucletides in length. In some embodiments, the expressible DNA or RNA sequence that encodes a self-assembling polypeptide is from about 390 to 410 nucletides in length. In some embodiments, the expressible DNA or RNA sequence that encodes a self-assembling polypeptide is from about 300 to 410 nucletides in length. In some embodiments, the expressible DNA or RNA sequence that encodes a self-assembling polypeptide is from about 300 to 500 nucletides in length.

### 2. Viral Antigens

The disclose relates to an expressible nucleic acid sequence comprising a first nucleic acid sequence encoding a scaffold domain comprising a self-assembling polypeptide, and a second nucleic acid sequence encoding an antigen domain comprising a viral antigen. In some embodiments, the nucleic acid molecule encodes a fusion peptide comprising one or a plurality of self-assembling peptides and one or a plurality of viral antigens. In some embodiments, upon administration to a subject, the composition comprising a nucleic acid comprising the expressible nucleic acid sequence of the present disclosure is transfected or transduced into an antigen presenting cell which encodes the expressible nucleic acid sequence. Upon expression, the self-assembling peptides assemble into a nanoparticle comprising the one or plurality of viral antigens. Antigen presenting cells expressing the one or plurality of viral antigens can elicit a therapeutically effective antigen-specific immune response against the virus in a subject. Any viral antigen may be used. In some embodiments, the viral antigen can be an antigen from a retrovirus, flavivirus, Nipah virus, West Nile virus, human papillomavirus, respiratory syncytial virus, filovirus, zaire ebolavirus, sudan ebolavirus, marburgvirus or influenza virus, or any virus disclosed in Table 1 below. For example, in some embodiments, the viral antigen can be an antigen from human immunodeficiency virus-1 (HIV-1). In other embodiments, the viral antigen can be an antigen from influenza virus.

Viral antigens are known for several genera of viruses and viral strains. A non-limiting example of a viral antigen is a fragment of gp120 having the amino acid sequence of SEQ ID NO: 9 (GT8) encoded by the nucleic acid sequence of SEQ ID NO: 4.

(SEQ ID NO: 4)
gacaccatcacactgccatgccgccctgcaccacctccacattgtagctccaacatcaccggcctgattctgacaagacagggg
ggatatagtaacgataataccgtgattttcaggccctcaggaggggactggagggacatcgcacgatgccagattgctggaaca
gtggtctctactcagctgtttctgaacggcagtctggctgaggaagaggtggtcatccgatctgaagactggcgggataatgcaa
agtcaatttgtgtgcagctgaacacaagcgtcgagatcaattgcactggcgcagggcactgtaacatttctcgggccaaatggaa
caataccctgaagcagatcgccagtaaactgagagagcagtacggcaataagacaatcatcttcaagccttctagtggaggcga
cccagagttcgtgaaccatagctttaattgcgggggagagttcttttattgtgattccacacagctgttcaacagcacttggtttaatt
ccacc
(SEQ ID NO: 9)
DTITLPCRPAPPPHCSSNITGLILTRQGGYSNDNTVIFRPSGGDWRDIARCQIAGTV
VSTQLFLNGSLAEEEVVIRSEDWRDNAKSICVQLNTSVEINCTGAGHCNISRAKW
NNTLKQIASKLREQYGNKTIIFKPSSGGDPEFVNHSFNCGGEFFYCDSTQLFNSTW
FNST

Other non-limiting examples of viral antigens are provided in Table 1.

TABLE 1
West Nile Virus capsid (SEQ ID NO: 41)
atgtctaaga aaccaggagg gcccggcaag agccgggctg tcaatatgct aaaacgcggaatgccccgcg tgttgtcctt
gattggactg aagagggcta tgttgaacct gatcgacggcaaggggccaa tacgatttgt gttggctctc ttggcgttct
tcaggttcac agcaattgctccgacccgag cagtgctgga tcgatggaga ggtgtgaaca aacaaacagc
gatgaaacaccttttgagtt ttaagaagga actagggacc ttgaccagtg ctatcaatcg goggagctcaaaacaaaaga
aaaga
HPV major capsid (SEQ ID NO: 42)
atgtcacttt ggcttccatc agaagctact gtttaccttc caccagttcc agtttcaaaa gttgtttcaa ctgatgaata
cgttgctagg actaatattt actaccatgc tggaacttca aggcttcttg ctgttggaca tccatacttt ccaattaaaa
aaccaaataa taataaaatt cttgttccaa aagtttcagg acttcaatac agggttttta ggattcatct tccagatcca
aataaatttg gatttccaga tacttcattt tacaatccag atactcaaag gcttgtttgggcttgtgttg gagttgaagt
tggaagggga caaccacttg gagttggaat ttcaggacat ccacttctta ataaacttga tgatactgaa aatgcttcag
cttacgctgc taatgctgga gttgataata gggaatgtat ttcaatggat tacaaacaaa ctcaactttg tcttattgga
tgtaaaccac caattggaga acattgggga aaaggatcac catgtactaa tgttgctgtt aatccaggag attgtccacc
acttgaactt attaatactg ttattcaaga tggagatatggttgatactg gatttggagc tatggatttt actactcttc
aagctaataa atcagaagtt ccacttgata tctgtacttc aatttgtaaa tacccagatt acattaaaat ggtttcagaa
ccatacggag attcactttt tttttacctt aggagggaac aaatgtttgt taggcatctt tttaataggg ctggagctgt
tggagaaaat gttccagatg atctttacat taaaggatca ggatcaactg ctaatcttgc ttcatcaaat tactttccaa
ctccatcagg atcaatggttacttcagatg ctcaaatttt taataaacca tactggcttc aaagggctca aggacataat
aatggaattt gttggggaaa tcaacttttt gttactgttg ttgatactac taggtcaact aatatgtcac tttgtgctgc
tatttcaact tcagaaacta cttacaaaaa tactaatttt aaagaatacc ttaggcatgg agaagaatac gatcttcaat
ttatttttca actttgtaaa attactctta ctgctgatgt tatgacttac attcattcaa tgaattcaac tattcttgaagattggaatt
ttggacttca accaccacca ggaggaactc ttgaagatac ttacaggttt gttacttcac aagctattgc ttgtcaaaaa
catactccac cagctccaaa agaggatcca cttaaaaaat acactttttg ggaagttaat cttaaagaaa aattttcagc
agatcttgat caatttccac ttggaaggaa atttcttctt caagctggac ttaaagctaa accaaaattt actcttggaa
aaaggaaagc tactccaact acttcatcaa cttcaactac tgctaaaaggaaaaaaagga aactttga
HPV minor capsid (SEQ ID NO: 43)
atgaggcaca agaggagcgc caagaggacc aagagggcca gcgccaccca gctgtacaagacctgcaagc
aggccggcac ctgccccccc gacatcatcc ccaaggtgga gggcaagaccatcgccgacc agatcctgca
gtacggcagc atgggcgtgt tcttcggcgg cctgggcatcggcaccggca gcggcaccgg cggcaggacc
ggctacatcc ccctgggcac caggcccccc accgccaccg acaccctggc ccccgtgagg ccccccctga
ccgtggaccc cgtgggccccagcgacccca gcatcgtgag cctggtggag gagaccagct tcatcgacgc
cggcgcccccaccagcgtgc ccagcatccc ccccgacgtg agcggcttca gcatcaccac
cagcaccgacaccacccccg ccatcctgga catcaacaac accgtgacca ccgtgaccac ccacaacaac
cccaccttca ccgaccccag cgtgctgcag ccccccaccc ccgccgagac cggcggccacttcaccctga
gcagcagcac catcagcacc cacaactacg aggagatccc catggacaccttcatcgtga gcaccaaccc
caacaccgtg accagcagca cccccatccc cggcagcaggcccgtggcca ggctgggcct gtacagcagg
accacccagc aggtgaaggt ggtggacccc gccttcgtga ccacccccac caagctgatc acctacgaca
accccgccta cgagggcatcgacgtggaca acaccctgta cttcagcagc aacgacaaca gcatcaacat
cgcccccgaccccgacttcc tggacatcgt ggccctgcac aggcccgccc tgaccagcag
gaggaccggcatcaggtaca gcaggatcgg caacaagcag accctgagga ccaggagcgg caagagcatc
ggcgccaagg tgcactacta ctacgacctg agcaccatcg accccgccga ggagatcgagctgcagacca
tcacccccag cacctacacc accaccagcc acgccgccag ccccaccagcatcaacaacg gcctgtacga
catctacgcc gacgacttca tcaccgacac cagcaccacccccgtgccca gcgtgcccag caccagcctg
agcggctaca tccccgccaa caccaccatc cccttcggtg gcgcctacaa catccccctg gtgagcggcc
ccgacatccc catcaacatcaccgaccagg cccccagcct gatccccatc gtgcccggca gcccccagta
caccatcatcgccgacgccg gcgacttcta cctgcacccc agctactaca tgctgaggaa gaggaggaagaggctgccct
acttcttcag cgacgtgagc ctggccgcct ga

Influenza HA protein (from past patent US20180344842A1, which is incorporated by reference in its entirety)

The accession numbers are as follows: GQ323579.1 (ACS72657.1), GQ323564.1 (ACS72654.1), GQ323551.1 (ACS72652.1), GQ323530.1 (ACS72651.1), GQ323520.1 (ACS72650.1), GQ323495.1 (ACS72648.1), GQ323489.1 (ACS72647.1), GQ323486.1 (ACS72646.1), GQ323483.1 (ACS72645.1), GQ323455.1 (ACS72641.1), GQ323451.1 (ACS72640.1), GQ323443.1 (ACS72638.1), GQ293077.1 (ACS68822.1), GQ288372.1 (ACS54301.1), GQ287625.1 (ACS54262.1), GQ287627.1 (ACS54263.1), GQ287623.1 (ACS54261.1), GQ287621.1 (ACS54260.1), GQ286175.1 (ACS54258.1), GQ283488.1 (ACS50088.1), GQ280797.1 (ACS45035.1), GQ280624.1 (ACS45017.1), GQ280121.1 (ACS45189.1), GQ261277.1 (ACS34968.1), GQ253498.1 (ACS27787.1), GQ323470.1 (ACS72643.1), GQ253492.1 (ACS27780.1), FJ981613.1 (ACQ55359.1), FJ971076.1 (ACP52565.1), FJ969540.1 (ACP44189.1), FJ969511.1 (ACP44150.1), FJ969509.1 (ACP44147.1), GQ255900.1 (ACS27774.1), GQ255901.1 (ACS27775.1), FJ966974.1 (ACP41953.1), GQ261275.1 (ACS34967.1), FJ966960.1 (ACP41935.1), FJ966952.1 (ACP41926.1), FJ966082.1 (ACP41105.1), GQ255897.1 (ACS27770.1), CY041645.1 (ACS27249.1), CY041637.1 (ACS27239.1), CY041629 (ACS27229.1), GQ323446.1 (ACS72639.1), CY041597.1 (ACS27189.1), CY041581.1 (ACS14726.1), CY040653.1 (ACS14666.1), CY041573.1 (ACS14716.1), CY041565.1 (ACS14706.1), CY041541.1 (ACS14676.1), GQ258462.1 (ACS34667.1), CY041557.1 (ACS14696.1), CY041549.1 (ACS14686.1), GQ283484.1 (ACS50084.1), GQ283493.1 (ACS50095.1), GQ303340.1 (ACS71656.1), GQ287619.1 (ACS54259.1), GQ267839.1 (ACS36632.1), GQ268003.1 (ACS36645.1), CY041621.1 (ACS27219.1), CY041613.1 (ACS27209.1), CY041605.1 (ACS27199.1), FJ966959.1 (ACP41934.1), FJ966982.1 (ACP41963.1), CY039527.2 (ACQ45338.1), FJ981612.1 (ACQ55358.1), FJ981615.1 (ACQ55361.1), FJ982430.1 (ACQ59195.1), FJ998208.1 (ACQ73386.1), GQ259909.1 (ACS34705.1), GQ261272.1 (ACS34966.1), GQ287621.1 (ACS54260.1), GQ290059.1 (ACS66821.1), GQ323464.1 (ACS72642.1), GQ323473.1 (ACS72644.1), GQ323509.1 (ACS72649.1). GQ323560.1 (ACS72653.1). GQ323574.1 (ACS72655.1), and GQ323576.1 (ACS72656.1).

Hemagglutinin (partial) from Influenza A virus (A/New Caledonia/20/1999(H1N1))
(SEQ ID NO: 65)
FTATYADTICIGYHANNSTDTVDTVLEKNVTVTHSVNLLEDSHNGKLCLLKGIAPLQ
LGNCSVAGWILGNPECELLISKESWSYIVETPNPENGTCYPGYFADYEELREQLSSVS
SFERFEIFPKESSWPNHTVTGVSASCSHNGKSSFYRNLLWLTGKNGLYPNLSKSYVN
NKEKEVLVLWGVHHPPNIGNQRALYHTENAYVSVVSSHYSRRFTPEIAKRPKVRDQ
EGRINYYWTLLEPGDTIIFEANGNLIAPWYAFALSRGFGSGIITSNAPMDECDAKCQT
PQGAINSSLPFQNVHPVTIGECPKYVRSAKLRMVTGLRNIH
Influenza A virus (A/West Virginia/01/2009(H1N1)) segment 4 hemagglutinin (HA)
(SEQ ID NO: 66)
MKAILVVLLYTFATANADTLCIGYHANNSTDTVDTVLEKNVTVTHSVNLLEDKHNG
KLCKLRGVAPLHLGKCNIAGWILGNPECESLSTASSWSYIVETSSSDNGTCYPGDFID
YEELREQLSSVSSFERFEIFPKTSSWPNHDSNKGVTAACPHAGAKSFYKNLIWLVKK
GNSYPKLSKSYINDKGKEVLVLWGIHHPSTSADQQSLYQNADAYVFVGTSRYSKKF
KPEIAIRPKVRDQEGRMNYYWTLVEPGDKITFEATGNLVVPRYAFAMERNAGSGIIIS
DTPVHDCNTTCQTPKGAINTSLPFQNIHPITIGKCPKYVKSTKLRLATGLRNVPSIQSR
GLFGAIAGFIEGGWTGMVDGWYGYHHQNEQGSGYAADLKSTQNAIDEITNKVNSVI
EKMNTQFTAVGKEFNHLEKRIENLNKKVDDGFLDIWTYNAELLVLLENERTLDYHD
SNVKNLYEKVRSQLKNNAKEIGNGCFEFYHKCDNTCMESVKNGTYDYPKYSEEAKL
NREEIDGVKLESTRIYQILAIYSTVASSLVLVVSLGAISFWMCSNGSLQCRICI
Hemagglutinin [Influenza A virus (A/California/04/2009(H1N1))]
(SEQ ID NO: 67)
MKAILVVLLYTFATANADTLCIGYHANNSTDTVDTVLEKNVTVTHSVNLLEDKHNG
KLCKLRGVAPLHLGKCNIAGWILGNPECESLSTASSWSYIVETPSSDNGTCYPGDFID
YEELREQLSSVSSFERFEIFPKTSSWPNHDSNKGVTAACPHAGAKSFYKNLIWLVKK
GNSYPKLSKSYINDKGKEVLVLWGIHHPSTSADQQSLYQNADTYVFVGSSRYSKKFK
PEIAIRPKVRDQEGRMNYYWTLVEPGDKITFEATGNLVVPRYAFAMERNAGSGIIISD
TPVHDCNTTCQTPKGAINTSLPFQNIHPITIGKCPKYVKSTKLRLATGLRNIPSIQSRGL
FGAIAGFIEGGWTGMVDGWYGYHHQNEQGSGYAADLKSTQNAIDEITNKVNSVIEK
MNTQFTAVGKEFNHLEKRIENLNKKVDDGFLDIWTYNAELLVLLENERTLDYHDSN
VKNLYEKVRSQLKNNAKEIGNGCFEFYHKCDNTCMESVKNGTYDYPKYSEEAKLN
REEIDGVKLESTRIYQILAIYSTVASSLVLVVSLGAISFWMCSNGSLQCRICI
RSV (from US20180346522A1)
F immunogen DNA sequence
(SEQ ID NO: 44)
gagctgcccatcctgaaaacaaacgccatcaccaccatcctggccgccgtgaccctgtgcttcgccagcagccagaacatcaccga
ggaattctaccagagcacctgtagcgccgtgtccaagggctacctgtctgccctgcggaccggctggtacaccagcgtgatcaccatc
gagctgagcaacatcaaagaaaacaagtgcaacggcaccgacgccaaagtgaagctgatcaagcaggaactggacaagtacaag
aacgccgtgaccgagctgcagctgctgatgcagagcacccctgccgccaacaacagagccagacgcgagctgccccggttcatga
actacaccctgaacaacaccaagaacaccaacgtgaccctgagcaagaagcggaagcggcggttcctgggattcctgctgggcgtg
ggcagcgccattgcctctggaatcgctgtgtctaaggtgctgcacctggaaggcgaagtgaacaagatcaagtccgccctgctgagc
accaacaaggccgtggtgtccctgagcaacggcgtgtccgtgctgaccagcaaggtgctggatctgaagaactacatcgacaagca
gctgctgcctatcgtgaacaagcagagctgcagcatcagcaacatcgagacagtgatcgagttccagcagaagaacaaccggctgct
ggaaatcacccgcgagttcagcgtgaacgccggcgtgaccacccccgtgtccacctacatgctgaccaacagcgagctgctgagcc
tgatcaacgacatgcccatcaccaacgaccagaaaaagctgatgagcaacaacgtgcagatcgtgcggcagcagagctactccatc
atgtccatcatcaaagaagaggtgctggcctacgtggtgcagctgcccctgtacggcgtgatcgacaccccctgctggaagctgcac
accagccccctgtgcaccaccaacaccaaagagggcagcaacatctgcctgacccggaccgaccggggctggtactgcgataatg
ccggcagcgtgtcattctttccacaggccgagacatgcaaggtgcagagcaaccgggtgttctgcgacaccatgaacagcctgaccc
tgccctccgaagtgaacctgtgcaacatcgacatcttcaaccctaagtacgactgcaagatoatgacctccaagaccgacgtgtccag
ctccgtgatcacctccctgggcgccatcgtgtcctgctacggcaagaccaagtgcaccgccagcaacaagaaccggggcatcatca
agaccttcagcaacggctgcgactacgtgtccaacaagggggtggacaccgtgtccgtgggcaacaccctgtactacgtgaacaaa
caggaaggcaagagcctgtacgtgaagggcgagcccatcatcaacttctacgaccccctggtgttccccagcgacgagttcgacgc
cagcatcagccaggtgaacgagaagatcaaccagagcctggccttcatcagaaagagcgacgagctgctgcacaatgtgaatgccg
gcaagagcaccaccaatatcatgatcaccacaatcatcatcgtgatcattgtgatcctgctgtccctgatcgccgtgggcctgctgctgt
actgcaaggcccggtccacccctgtgaccctgtccaaggaccagctgagcggaatcatcaacaatatcgccttctccaactqa
Encoded protein sequence
(SEQ ID NO: 45)
MQSTPAANNRARRELPRFMNYTLNNTKNTNVTLSKKRKRRFLGFLLGVGSAIASGIA
VSKVLHLEGEVNKIKSALLSTNKAVVSLSNGVSVLTSKVLDLKNYIDKQLLPIVNKQ
SCSISNIETVIEFQQKNNRLLEITREFSVNAGVTTPVSTYMLTNSELLSLINDMPITNDQ
KKLMSNNVQIVRQQSYSIMSIIKEEVLAYVVQLPLYGVIDTPCWKLHTSPLCTTNTKE
GSNICLTRTDRGWYXDNAGSVSFFPQXETCKVQSNRVFCDTMNSLTLPSEVNLCNID
IFNPKYDCKXMTSKTDVSSSVITSLGAIVSCYGKTKCTASNKNRGIIKTFSNGCDYVS
NKGVDTVSVGNTLYYVNKQEGKSLYVKGEPIINFYDPLVFPSDEFDASISQVNEKINQ
SLAFIRKSDELLHNVNAGKSTTNIMITTIIIVIIVILLSLIAVGLLLYCKARSTPVTLSKD
QLSGIINNIAFSN
RSV Ga
DNA sequence of Ga
(SEQ ID NO: 46)
atgtccaagaataaggatcagaggaccgcgaaaacgcttgagaggacgtgggacacgctgaaccacctcctgttcatctcctcgtgtc
tctacaagctcaaccttaagtccatcgcgcagatcaccttgagcattctcgccatgatcatctccaccagccttatcattgccgcaatcatc
ttcatcgcatccgccaaccataaggtgacattgactacagcgattatccaagacgctactagccagatcaagaataccacgccgaccta
tttgacgcaaaatcctcagttgggaattagcttctcgaatctctcggaaaccacgtcgcagccgactacaattcttgcgtcaacgactcca
tcggccaaatcaacaccacaatcgactaccgtaaaaacgaagaacacgactacaacacagattcagccttcaaagcccacgaccaaa
cagagacagaataagccgcccaacaagcccaacaatgattttcacttcgaggtgtttaacttcgtgccctgttcgatttgcagcaataac
cccacgtgctgggcgatttgcaagcgaatcccgaataagaagcccgggaaaaagaccacgacgaaaccgacaaagaagccgaca
atcaagacaacgaaaaaggatcttaaacctcagacgacaaagcctaaggaagtcttgacaacgaagcctacggaaaaacccactatc
aatactaccaagactaacatccggacaacactgctgacgagcaataccacgggaaacccggagctcacatcgcagaaagagacact
ccattcgacatcctccgagggtaacccttcgcccagccaggtgtatacgacgtcagaataccctagccaaccctcatcgccctcaaata
cgacccggcaatga
Protein sequence for Ga
(SEQ ID NO: 47)
MSKNKDQRTAKTLERTWDTLNHLLFISSCLYKLNLKSIAQITLSILAMIISTSLIIAAIIFI
ASANHKVTLTTAIIQDATSQIKNTTPTYLTQNPQLGISFSNLSETTSQPTTILASTTPSA
KSTPQSTTVKTKNTTTTQIQPSKPTTKQRQNKPPNKPNNDFHFEVFNFVPCSICSNNPT
CWAICKRIPNKKPGKKTTTKPTKKPTIKTTKKDLKPQTTKPKEVLTTKPTEKPTINTT
KTNIRTTLLTSNTTGNPELTSQKETLHSTSSEGNPSPSQVYTTSEYPSQPSSPSNTTRQ
RSV Gb
DNA sequence of Gb
(SEQ ID NO: 48)
atgagcaaaaacaaaaaccaaaggacggctcggacgottgagaaaacatgggacacgcttaatcaccttattgtgatctcatcgtgttt
gtaccggttgaatctcaagagcatcgcccagattgcgctgtcagtcctggccatgattatctcgacatcactcatcatcgcagocatcat
ctttatcatttcagcgaatcacaaggtaacgcttacaacagtcacggtgcagaccatcaagaatcataccgaaaagaatatcacaaccta
cctcacccaagtcagoccggagagagtaagcccctcaaaacagcctactacgacacctcccatccacacgaactcggcgaccatct
caccgaataccaaatcagaaacgcatcatacgaccgcacagacaaagggacgaaccactacacccacacagaacaacaaacccag
caccaagccgaggccaaagaatccgcccaagaagccgaaagatgactatcactttgaagtgttcaacttcgtaccgtgttcgatttgcg
ggaataatcagttgtgcaaatccatttgcaagacgatcccatccaacaaaccgaagaagaaacctaccatcaagcccacaaacaagc
caacgacaaaaacaacgaacaagcgcgatcccaaaacgctcgcgaaaacgttgaagaaggaaacgacgacaaaccctacgaaga
aacccacgcccaagaccactgagagagacacctccacctcgcaatcgacggtacttgacacgactacgagcaagcacactatccag
caacagtccctgcactcaaccacgcccgagaatacaccaaactcaacacagactccgacagcttcagagccttccacttcgaattcca
catga
Protein sequence of Gb
(SEQ ID NO: 49)
MSKNKNQRTARTXEKTWDTXNHLIVISSCLYRLNLKSIAQIALSVLAMIISTSLIIAXII
FIISANHKVTLTTVTVQTIKNHTEKNITTYLTQVXPERVSPSKQPTTTPPIHTNSATISP
NTKSETHHTTAQTKGRTTTPTQNNKPSTKPRPKNPPKKPKDDYHFEVFNFVPCSICGN
NQLCKSICKTIPSNKPKKKPTIKPTNKPTTKTTNKRDPKTLAKTLKKETTTNPTKKPTP
KTTERDTSTSQSTVLDTTTSKHTIQQQSLHSTTPENTPNSTQTPTASEPSTSNST

Filoviruses (from US20180344840A1. which is incorporated by reference in its entirety)

DNA sequence of Zaire ebolavirus glycoprotein consensus
(SEQ ID NO: 50)
atgggggtcactgggattctgcagctgcctagagatcgcttcaagcgaacctctttctttctgtgggtcatcattctgttccagaggactttt
agtatccctctgggcgtcattcacaattctaccctgcaggtgagtgacgtcgataagctggtgtgtcgggacaaactgagctccaccaa
ccagctgagatctgtcggcctgaatctggaggggaacggagtggctaccgatgtcccaagtgcaacaaagagatgggggtttcgctc
aggagtgccccctaaagtggtcaattacgaggccggggaatgggctgagaattgctataacctggaaatcaagaaacccgacggatc
agagtgtctgccagccgctcccgatgggattcgcggattccctagatgcagatacgtgcacaaggtcagcggcaccgggccatgtgc
aggagacttcgcctttcataaagaaggcgccttctttctgtacgatagactggcttccaccgtgatctatagggggaccacattcgccga
gggagtggtcgcttttctgattctgcctcaggccaagaaagacttcttttctagtcatcctctgcgggaaccagtgaacgctaccgagga
ccccagcagcggctactattccactaccatcagataccaggccacaggattcggcaccaatgagacagaatacctgtttgaagtggac
aacctgacatatgtccagctggagtctaggttcactccccagtttctgctgcagctgaatgaaactatctataccagtggcaagcgctca
aatacaactgggaagctgatttggaaagtgaaccctgagatcgataccacaattggcgaatgggccttttgggagaccaagaaaaacc
tgacacggaagatcagaagcgaggaactgtccttcaccgcagtgagtaatagggccaaaaacatttcaggccagagcccagcacga
acttcctctgaccccgggaccaatactaccacagaagatcacaagatcatggccagcgagaacagttcagctatggtgcaggtccact
cccagggaagggaggcagccgtgtctcatctgactaccctggccacaatctctactagtccccagagccccacaactaagcccggg
cctgacaatagcacccataacacacctgtgtacaaactggatatctccgaagccacccaggtcgagcagcaccatcggagaacagac
aatgattccactgcatctgacacccctccagcaaccacagctgcaggaccccccaaggctgagaatactaacaccagcaaaagcacc
gacctgctggaccccgcaactaccacatcaccacagaaccacagcgagacagccgggaacaataacactcaccatcaggacaccg
gagaggaatccgccagctccggcaagctggggctgatcacaaatactattgctggagtggcaggactgatcacaggcgggaggcg
aactcgacgagaagctattgtgaacgcacagcccaaatgcaatcctaacctgcactattggactacccaggacgagggagcagctat
cggactggcatggattccatactttgggcccgcagccgaaggaatctataccgagggcctgatgcataatcaggatggactgatctgt
ggcctgcggcagctggctaacgaaacaactcaggcactgcagctgttcctgcgagctaccacagagctgcggacctttagcatcctg
aatcgcaaggcaattgacttcctgctgcagcgatggggaggcacatcccacatcctgggaccagactgctgtattgagcctcatgattg
gacaaagaacatcactgacaaaattgatcagatcattcacgacttcgtggataaaacactgccagatcagggggacaatgataactggt
ggactggatggagacagtggattcccgccggcattggcgtcaccggcgtcattattgccgtcattgctctgttctgtatttgtaagttcgtg
ttctgataa
Protein sequence of Zaire ebolavirus glycoprotein consensus
(SEQ ID NO: 51)
MGVTGILQLPRDRFKRTSFFLWVIILFQRTFSIPLGVIHNSTLQVSDVDKLVCRDKLSS
TNQLRSVGLNLEGNGVATDVPSATKRWGFRSGVPPKVVNYEAGEWAENCYNLEIK
KPDGSECLPAAPDGIRGFPRCRYVHKVSGTGPCAGDFAFHKEGAFFLYDRLASTVIY
RGTTFAEGVVAFLILPQAKKDFFSSHPLREPVNATEDPSSGYYSTTIRYQATGXGTNE
TEYLFEVDNLTYVQLESRFTPQFLLQLNETIYTSGKRSNTTGKLIWKVNPEIDTTIGEW
AFWETKKNLTRKIRSEELSFTAVSNRAKNISGQSPARTSSDPGTNTTTEDHKIMASEN
SSAMVQVHSQGREAAVSHLTTLATISTSPQSPTTKPGPDNSTHNTPVYKLDISEATQV
EQHHRRTDNDSTASDTPPATTAAGPPKAENTNTSKSTDLLDPATTTSPQNHSETAGN
NNTHHQDTGEESASSGKLGLITNTIAGVAGLITGGRXTRREAIVNAQPKCNPNLHYW
TTQDEGAAIGLAWIPYFGPAAEGIYTEGLMHNQDGLICGLRQLANETTQALQLFLRA
TTELRTFSXLNRKAIDFLLQRWGGTXHILGPDCCIEPHDWTKNITDKIDQIIHDFVDKT
LPDQGDNDNWWTGWRQWIPAGIGVTGVIIAVIALFCICKFVF
Sudan Ebolavirus Glycoprotein consensus
DNA sequence
(SEQ ID NO: 52)
atggagggactgtcactgctgcagctgcctagagataagttcaggaaaagctccttctttgtgtgggtcatcattctgttccagaaggcct
tttcaatgcccctgggcgtggtcactaatagcaccctggaagtgacagagatcgatcagctggtctgtaaggaccacctggcttcaact
gatcagctgaaaagcgtggggctgaacctggagggatcaggcgtcagcactgatattccttctgcaaccaagagatggggatttcgc
agcggagtgccccctaaagtggtctcctacgaagcaggggagtgggccgaaaattgctataacctggagatcaagaaaccagatgg
cagcgaatgtctgccaccccctccagacggggtgcgcggattccccagatgcagatacgtccacaaggcccaggggaccggacct
tgtccaggagactatgcctttcataaagatggcgctttctttctgtacgaccgcctggctagtacagtgatctatcgaggcgtcaatttcgc
cgagggcgtgatcgcttttctgattctggcaaagccaaaagaaaccttcctgcagagccctcccattagggaggccgtgaactacaca
gaaaacacttctagttactacgctacatcctacctggagtatgaaatcgagaactttggcgctcagcactctaccacactgttcaagatta
acaataacacctttgtgctgctggatcgccctcatacaccacagttcctgtttcagctgaacgacactatccacctgcatcagcagctga
gcaatactaccggaaaactgatttggacactggacgctaatatcaacgcagatattggcgagtgggccttctgggaaaataagaaaaa
cctgtccgagcagctgcggggagaggaactgagctttgaaacactgtccctgaatgaaactgaggacgatgacgccacctcaagcc
gaacaactaagggccggatctctgatcgggctaccagaaagtacagtgatctggtgccaaaagactctcccggcatggtgagtctgc
acgtccctgaaggggagaccacactgccatcccagaactctactgagggccggagagtggacgtcaatacccaggagactatcacc
gaaactaccgcaacaatcattggcactaacgggaataacatgcagatcagcaccattggcacagggctgtcctctagtcagattctgtc
aagctcccctaccatggccccctcccctgagacacagacttctacaacttatacacccaagctgcctgtgatgaccacagaggaaccc
actaccccacccagaaacagtcctgggtcaacaactgaggcacccaccctgaccacacctgaaaatatcactaccgccgtgaaaaca
gtcctgcctcaggagtctactagtaacggactgatcaccagcacagtgactggaattctgggcagtctggggctgcgcaagcgatcaa
ggcgccaagtgaatactcgggctaccggcaaatgcaatccaaacctgcactactggaccgcacaggagcagcataacgccgctgg
gatcgcttggattccttacttcggaccaggcgcagaggggatctataccgaaggactgatgcataatcagaacgccctggtgtgtggc
ctgagacagctggcaaatgagacaactcaggccctgcagctgttcctgagagcaaccacagaactgaggacctatacaatcctgaac
cggaaggccattgattttctgctgcgacgatggggcgggacctgcagaatcctgggaccagactgctgtattgagccccacgattgga
ccaagaacatcacagacaagatcaaccagatcattcatgatttcatcgacaacccactgcccaatcaggacaacgatgacaattggtg
gaccggatggcgacagtggattcccgcaggaattggaatcaccggaattattattgccattattgctctgctgtgtgtctgtaagctgctg
tgttgataa
Protein sequence
(SEQ ID NO: 53)
MEGLSLLQLPRDKFRKSSFFVWVIILFQKAFSMPLGVVINSTLEVTEIDQLVCKDHLA
STDQLKSVGXNLEGSGVSTDIPSATKRWGFRSGVPPKVVSYEAGEWAENCYNLEIKK
PDGSECLPPPPDGVRGFPRCRYVHKXQGTGPCPGDYAFHKDGAFFLYDRLASTVXY
RGVNFAEGVIAFLILAKPKETFLQSPPIREAVNYTENTSSYYATSYLEYEIENFGAQHS
TTLFKINNNXFVLLDRPHTPQFLFQLNDTIHLHQQLSNTTGKLIWTLDANINADIGEW
AFWENKKNLSEQLRGEELSFETLSLNETEDDDATSSRTTKGRISDRATRKYSDLVPK
DSXGMVSLHVPEGETTLPSQNSTEGRRVDVNTQETITETTATIIGTNGNNMQISTIGTG
LSSSQILSSSPTMAPSPETQTSTTYTPKLPVMTTEEPTTPPRNSPGSTTEAPTLTTPENIT
TAVKTVLPQESTSNGLITSTVTGILGSLGLRKRSRRQVNTRATGKCNPNLHYWTAQE
QHNAAGIAWIPYFGPGAEGIYTEGLMHNQNALVCGLRQLANETTQALQLFLRATTE
LRTYTILNRKAIDFLLRRWGGTCRILGPDCCIEPHDWTKNITDKINQIIHDFIDNPLPNQ
DNDDNWWTGWRQWIPAGIGITGIIIAIIALLCVCKLLC
Marburgvirus glycoprotein consensus
DNA sequence
(SEQ ID NO: 54)
atgaaaaccacttgtctgctpatctcactgattctgattcagggcgtcaaaacactgcccattctggaaattgcctctaacatccagccac
agaacgtggactccgtctgttctgggaccctgcagaagacagaggatgtgcacctgatgggcttcaccctgagcgggcagaaggtc
gcagactcacccctggaagccagcaaacgatgggcatttcgggccggagtgccccctaagaacgtcgagtacaccgaaggcgagg
aagccaaaacatgctataatatctccgtgactgatcctagtggcaagtcactgctgctggacccacccaccaacattagggattacccta
agtgtaaaacaatccaccatattcagggccagaatccacacgctcaggggatcgcactgcatctgtggggagccttctttctgtacgac
aggattgctagcaccacaatgtatcgcgggaaagtgttcaccgagggaaacatcgccgctatgattgtgaataagacagtccacaaaa
tgatcttttctcgccagggccaggggtaccgacatatgaacctgaccagtacaaataagtattggaccagctccaacggcactcagac
caatgacactgggtgcttcggaaccctgcaggagtacaacagtactaaaaatcagacctgtgctccatcaaagaaaccactgccactg
cctaccgcacacccagaggtgaagctgacaagtacttcaaccgacgccacaaaactgaacactaccgaccccaatagtgacgatga
agatctgacaactagcggatccggctctggggagcaggaaccttataccacatccgatgcagccaccaagcagggcctgtctagtac
aatgcctccaactccatctccccagcctagtactccccagcaggggggaacaataccaaccattcccagggcgtggtcacagagcc
agggaagactaacactaccgcccagccctctatgccccctcacaatacaactaccatctccaccaacaatacatctaaacataacctga
gcacaccttccgtgccaatccagaacgctactaactacaacactcagtctaccgcacccgagaatgaacagacttctgcccctagtaa
gacaactctgctgcccaccgagaaccctaccacagccaagtcaacaaatagcactaaatcccctactaccacagtgccaaacactac
caataagtacagtacctcaccaagccccacccctaactccacagcacagcacctggtctatttccggagaaaaagaaatatcctgtgg
agggagggcgacatgttcccttttctggatgggctgatcaacgctccaattgacttcgatccagtgcccaatacaaagactatctttgac
gaatcaagctcctctggcgcctctgctgaggaagatcagcacgcctcacccaacattagcctgacactgtcctactttcctaaagtgaac
gagaatactgcccatagcggggagaacgaaaatgactgcgatgctgagctgcggatctggagcgtccaggaagacgatctggctgc
aggactgtcctggatcccattctttggacccggcattgagggactgtataccgccggcctgattaagaaccagaacaacctggtgtgca
gactgaggcgcctggccaatcagaccgctaaatcactggaactgctgctgcgggtcacaactgaggaaagaacattcagcctgatca
accgacatgctattgactttctgctggcacgctggggaggcacctgcaaggtgctgggaccagactgctgtatcggcattgaggatct
gtctcgcaatatcagtgaacagatcgaccagattaagaaagatgagcagaaggaaggaaccggatggggactgggcggcaagtgg
tggaccagcgattggggcgtgctgacaaacctgggaatcctgctgctgctgtccatcgccgtcctgattgctctgtcctgtatttgtcgga
ttttcactaagtatattgggtgataa
Protein sequence
(SEQ ID NO: 55)
MKTTCLXISLILIQGVKTLPILEIASNIQPQNVDSVCSGTLQKTEDVHLMGFTLSGQKV
ADSPLEASKRWAFRAGVPPKNVEYTEGEEAKTCYNISVTDPSGKSLLLDPPTNIRDYP
KCKTIHHIQGQNPHAQGIALHLWGAFFLYDRIASTTMYRGKVFTEGNIAAMIVNKTV
HKMIFSRQGQGYRHMNLTSTNKYWTXSNGTQTNDTGCFGTLQEYNSTKNQTCAPS
KKPLPLPTAHPEVKLTSTSTDATKLNTTDPNSDDEDLTTSGSGSGEQEPYTTSDAATK
QGLSSTMPPTPSPQPSTPQQGGNNTNHSQGVVTEPGKTNTTAQPSMPPHNTTTISTNN
TSKHNLSTPSVPIQNATNYNTQSTAPENEQTSAPSKTTLLPTENPTTAKSTNSTKSPTT
TVPNTTNKYSTSPSPTPNSTAQHLVYFRRKRNILWREGDMFPFLDGLINAPIDFDPVP
NTKTIFDESSSSGASAEEDQHASPNISLTLSYFPKVNENTAHSGENENDCDAELRIWS
VQEDDLAAGLSWIPFFGPGIEGLYTAGLIKNQNNLVCRLRRLANQTAKSLELLLRVT
TEERTFSLINRHAIDFLLARWGGTCKVLGPDCCIGIEDLSRNISEQIDQIKKDEQKEGT
GWGLGGKWWTSDWGVLTNLGILLLLSIAVLIALSCICRIFTKYIG

Accordingly, in some embodiments, the viral antigen encoded by the expressible nucleic acid sequence of the present disclosure comprises at least about 60%. 65%. 70%. 75%. 80%. 85%. 90%, 95%. or 99% sequence identity to SEQ ID NO: 9. SEQ ID NO: 45. SEQ ID NO: 47, SEQ ID NO: 49, SEQ ID NO: 51, SEQ ID NO: 53, SEQ ID NO: 55, SEQ ID NO: 65, SEQ ID NO: 66 or SEQ ID NO: 67, or a functional fragment thereof. In some embodiments, the viral antigen comprises the amino acid sequence of SEQ ID NO: 9, SEQ ID NO: 45, SEQ ID NO: 47, SEQ ID NO: 49, SEQ ID NO: 51, SEQ ID NO: 53, SEQ ID NO: 55, SEQ ID NO: 65, SEQ ID NO: 66 or SEQ ID NO: 67, or a functional fragment thereof. In some embodiments, the nucleic acid sequence encoding the viral antigen comprises at least about 60%, 65%, 70%, 75%, 80%, 85%, 90%, 95%, or 99% sequence identity to SEQ ID NO: 4, SEQ ID NO: 41, SEQ ID NO: 42, SEQ ID NO: 43, SEQ ID NO: 44, SEQ ID NO: 46, SEQ ID NO: 48, SEQ ID NO: 50, SEQ ID NO: 52 or SEQ ID NO: 54 or a functional fragment thereof. In some embodiments, the nucleic acid sequence encoding the viral antigen comprises the nucleotide sequence of SEQ ID NO: 4, SEQ ID NO: 41, SEQ ID NO: 42, SEQ ID NO: 43, SEQ ID NO: 44, SEQ ID NO: 46, SEQ ID NO: 48, SEQ ID NO: 50, SEQ ID NO: 52 or SEQ ID NO: 54 or a functional fragment thereof. In other embodiments, the nucleic acid sequence encoding the viral antigen comprises at least about 60%, 65%, 70%, 75%, 80%, 85%, 90%, 95%, or 99% sequence identity to a nucleic acid sequence that is complementary to SEQ ID NO: 4, SEQ ID NO: 41, SEQ ID NO: 42, SEQ ID NO: 43, SEQ ID NO: 44, SEQ ID NO: 46, SEQ ID NO: 48, SEQ ID NO: 50, SEQ ID NO: 52 or SEQ ID NO: 54 or a functional fragment thereof. In some embodiments, the nucleic acid sequence encoding the viral antigen comprises a nucleic acid sequence that is complementary to SEQ ID NO: 4, SEQ ID NO: 41, SEQ ID NO: 42, SEQ ID NO: 43, SEQ ID NO: 44, SEQ ID NO: 46, SEQ ID NO: 48, SEQ ID NO: 50, SEQ ID NO: 52 or SEQ ID NO: 54 or a functional fragment thereof. In some embodiments, the antigens for the present disclosure comprise at least about 60%, 65%, 70%, 75%, 80%, 85%, 90%, 95%, or 99% sequence identity to any antigen sequences disclosed in the Examples disclosed herein, particularly Examples 8-11.

In some embodiments, the composition or pharmaceutical composition of the disclosure comprises an expressible DNA or RNA sequence that encodes a viral antigen is from about 1100 to 1300 nucletides in length. In some embodiments, the composition or pharmaceutical composition of the disclosure comprises an expressible DNA or RNA sequence that encodes a viral antigen is from about 1100 to 1200 nucletides in length. In some embodiments, the composition or pharmaceutical composition of the disclosure comprises an expressible DNA or RNA sequence that encodes a viral antigen is from about 1100 to 1300 nucletides in length.

In some embodiments, the composition or pharmaceutical composition of the disclosure comprises an expressible DNA or RNA sequence that encodes a viral antigen is from about 1200 to 1210 nucletides in length. In some embodiments, the composition or pharmaceutical composition of the disclosure comprises an expressible DNA or RNA sequence that encodes a viral antigen is from about 1180 to 1220 nucletides in length. In some embodiments, the composition or pharmaceutical composition of the disclosure comprises an expressible DNA or RNA sequence that encodes a viral antigen is from about 1180 to 1215 nucletides in length. In some embodiments, the composition or pharmaceutical composition of the disclosure comprises an expressible DNA or RNA sequence that encodes a viral antigen is from about 1180 to 1210 nucletides in length. In some embodiments, the composition or pharmaceutical composition of the disclosure comprises an expressible DNA or RNA sequence that encodes a viral antigen is from about 1180 to 1200 nucletides in length. In some embodiments, the composition or pharmaceutical composition of the disclosure comprises an expressible DNA or RNA sequence that encodes a viral antigen is from about 1220 to 1230 nucletides in length.

In some embodiments, the composition or pharmaceutical composition of the disclosure comprises an expressible DNA or RNA sequence that encodes a viral antigen is from about 1100 to 1300 nucletides in length. In some embodiments, the composition or pharmaceutical composition of the disclosure comprises an expressible DNA or RNA sequence that encodes a viral antigen is from about 1100 to 1300 nucletides in length.

### 3. Linker

The disclosure relates, in some embodiments, to an expressible nucleic acid sequence comprising, in addition to the first nucleic acid sequence encoding a scaffold domain comprising a self-assembling polypeptide and the second nucleic acid sequence encoding an antigen domain comprising a viral antigen described above, a third nucleic acid sequence encoding a linker domain comprising a linker peptide, wherein the third nucleic acid sequence is positioned between the first nucleic acid sequence and the second nucleic acid sequence in the 5′ to 3′ orientation. Any type of linker or linker peptide can be used. The term “linker” or “linker peptide” is used interchangeable herein.

In some embodiments, each linker or linker peptide is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural amino acids in length.

In some embodiments, each linker or linker peptide is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length. In some embodiments, each linker or linker peptide is independently selectable from a linker or linker peptide that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length. In some embodiments, each linker or linker peptide is about 21 natural or non-natural amino acids in length.

In some embodiments, the length of each linker or linker peptide is different. For example, in some embodiments, the length of a first linker or linker peptide is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length, and the length of a second linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length, where the length of the first linker is different from the length of the second linker. Various configurations can be envisioned by the present disclosure, where the linker domain comprises 1, 2, 3, 4, 5, 6, 7, 8, 9, 10 or more linkers or linker peptides wherein the linkers or linker peptides are of similar or different lengths.

In some embodiments, two linkers or linker peptides can be used together. Accordingly, in some embodiments, the first linker or linker peptide is independently selectable from about 0 to about 25 natural or non-natural amino acids in length, about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural amino acids in length. In some embodiments, the second linker or linker peptide is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural amino acids in length. In some embodiments, the first linker or linker peptide is independently selectable from a linker or linker peptide that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length. In some embodiments, the second linker or linker peptide is independently selectable from a linker or linker peptide that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length.

A non-limiting example of a linker peptide may comprise the amino acid sequence of GGSGGSGGSGGSGGG (SEQ ID NO: 8) encoded by the nucleic acid sequence of GGAGGCTCCGGAGGATCTGGAGGGAGTGGAGGCTCAGGAGGAGGC (SEQ ID NO: 3).

A linker or lilnker peptide can be either flexible or rigid or a combination thereof. An example of a flexible linker is a GGS repeat. In some embodiments, the GGS can be repeated about 1, 2, 3, 4, 5, 6, 7, 8, 9, or 10 times. Non-limiting examples of such linker peptides may comprise the amino acid sequence of GGSGGSGGS (SEQ ID NO: 22), GGSGGSGGSGGS (SEQ ID NO: 27), or GGSGGSGGSGGSGGGGSGGGSGGG (SEQ ID NO: 32). An example of a rigid linker is 4QTL-115 Angstroms, single chain 3-helix bundle represented by the sequence:

(SEQ ID NO: 18)
NEDDMKKLYKQMVQELEKARDRMEKLYKEMVELIQKAIELMRKIFQEVK
QEVEKAIEEMKKLYDEAKKKIEQMIQQIKQGGDKQKMEELLKRAKEEMK
KVKDKMEKLLEKLKQIMQEAKQKMEKLLKQLKEEMKKMKEKMEKLLKEM
KQRMEEVKKKMDGDDELLEKIKKNIDDLKKIAEDLIKKAEENIKEAKKI
AEQLVKRAKQLIEKAKQVAEELIKKILQLIEKAKEIAEKVLKGLE.

Other non-limiting examples of linker peptides may be encoded by the nucleic acid sequence of:

(SEQ ID NO: 16)
GGCGGCTCTGGCGGAAGTGGCGGAAGTGGGGGAAGTGGAGGCGGCGGAA
GCGGGGGAGGCAGCGGGGGAGGG,
(SEQ ID NO: 17)
GGCGGAAGCGGCGGAAGCGGCGGGTCT,
(SEQ ID NO: 19)
GGCGGCAGCGGCGGCAGCGGCGGGAGCGGAGGAAGT,
or
(SEQ ID NO: 29)
GGCGGCTCTGGCGGAAGTGGCGGAAGTGGGGGAAGTGGAGGCGGCGGAA
GCGGGGGAGGCAGCGGGGGAGGG.

Accordingly, in some embodiments, the linker peptide encoded by the expressible nucleic acid sequence of the present disclosure comprises at least about 60%, 65%, 70%, 75%, 80%, 85%, 90%, 95%, or 99% sequence identity to SEQ ID NO: 8, SEQ ID NO: 18, SEQ ID NO: 22, SEQ ID NO: 27 or SEQ ID NO: 32, or a functional fragment thereof. In some embodiments, the linker peptide comprises the amino acid sequence of SEQ ID NO: 8, SEQ ID NO: 18, SEQ ID NO: 22, SEQ ID NO: 27 or SEQ ID NO: 32, or a functional fragment thereof. In some embodiments, the nucleic acid sequence encoding the linker peptide comprises at least about 60%, 65%, 70%, 75%, 80%, 85%, 90%, 95%, or 99% sequence identity to SEQ ID NO: 3, SEQ ID NO: 16, SEQ ID NO: 17, SEQ ID NO: 19 or SEQ ID NO: 29, or a functional fragment thereof. In some embodiments, the nucleic acid sequence encoding the linker peptide comprises the nucleotide sequence of SEQ ID NO: 3, SEQ ID NO: 16, SEQ ID NO: 17, SEQ ID NO: 19 or SEQ ID NO: 29, or a functional fragment thereof. In other embodiments, the nucleic acid sequence encoding the linker peptide comprises at least about 60%, 65%, 70%, 75%, 80%, 85%, 90%, 95%, or 99% sequence identity to a nucleic acid sequence that is complementary to SEQ ID NO: 3, SEQ ID NO: 16, SEQ ID NO: 17, SEQ ID NO: 19 or SEQ ID NO: 29, or a functional fragment thereof. In some embodiments, the nucleic acid sequence encoding the linker peptide comprises a nucleic acid sequence that is complementary to SEQ ID NO: 3, SEQ ID NO: 16, SEQ ID NO: 17, SEQ ID NO: 19 or SEQ ID NO: 29, or a functional fragment thereof.

### 4. Leader Sequence

The expressible nucleic acid sequence of the present disclosure is optionally free of a nucleic acid sequence encoding a leader sequence. A “leader sequence” may be from time to time refers to a “signal peptide” and thus, the terms “leader sequence” and “signal peptide” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences, when present, are linked at the N terminus of the protein.

Although the expressible nucleic acid sequence of the present disclosure is optionally free of a nucleic acid sequence encoding a leader sequence, if the presence of such a leader sequence is required for proper secretion of the protein produced by the cell, it may nonetheless be included in the polypeptide encoded by the expressible nucleic acid sequence of the present disclosure.

A non-limiting example of the leader sequence is the amino acid sequence of MDWTWILFLVAAATRVHS (SEQ ID NO: 6) encoded by the nucleic acid sequence of atggactggacctggattctgttcctggtggccgccgccacaagggtgcacagc (SEQ ID NO: 1).

Another non-limiting example of the leader sequence is the amino acid sequence of MDWTWRILFLVAAATGTHA (SEQ ID NO: 40) encoded by the nucleic acid sequence of atggactggacctggagaatcctgttcctggtggccgccgccaccggcacacacgccgatacacacttccccatctgcatcttttg ctgtggctgttgccataggtccaagtgtgggatgtgctgcaaaact (SEQ ID NO: 39).

A yet another non-limiting example of the leader sequence is the amino acid sequence of MRRMQLLLLIALSLALVTNS (SEQ ID NO: 101).

Thus, in some embodiments when the leader sequence is present, the leader sequence may comprise at least about 60%, 65%, 70%, 75%, 80%, 85%, 90%, 95%, or 99% sequence identity to SEQ ID NO: 6, SEQ ID NO: 40, or SEQ ID NO: 101, or a functional fragment thereof. In some embodiments when the leader sequence is present, the leader sequence may comprise the amino acid sequence of SEQ ID NO: 6, SEQ ID NO: 40, or SEQ ID NO: 101, or a functional fragment thereof. In some embodiments when the leader sequence is present, the leader sequence may be encoded by a nucleic acid sequence comprising at least about 60%, 65%, 70%, 75%, 80%, 85%, 90%, 95%, or 99% sequence identity to SEQ ID NO: 1 or SEQ ID NO: 39, or a functional fragment thereof. In some embodiments when the leader sequence is present, the leader sequence may be encoded by the nucleic acid sequence of SEQ ID NO: 1 or SEQ ID NO: 39, or a functional fragment thereof. In other embodiments when the leader sequence is present, the leader sequence may be encoded by a nucleic acid sequence comprising at least about 60%, 65%, 70%, 75%, 80%, 85%, 90%, 95%, or 99% sequence identity to a nucleic acid sequence that is complementary to SEQ ID NO: 1 or SEQ ID NO: 39, or a functional fragment thereof. In some embodiments when the leader sequence is present, the leader sequence may be encoded by a nucleic acid sequence that is complementary to SEQ ID NO: 1 or SEQ ID NO: 39, or a functional fragment thereof.

### 5. Regulatory Sequences

In some embodiments, the expressible nucleic acid sequence can be operably linked to one or a plurality of regulatory sequences. Examples of regulatory sequences include, but not limited to, promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

### 6. Expressible RNA Sequences

In some embodiments, the expressible nucleic acid sequence comprised in the composition of the present disclosure is an RNA molecule or RNA transcript. In such embodiments therefore, the expressible nucleic acid sequence comprises a first RNA sequence encoding a scaffold domain comprising any of the self-assembling polypeptides disclosed herein and a second RNA sequence encoding one or more of any of the viral antigens disclosed herein, and optionally, wherein the expressible RNA sequence is free of a RNA sequence encoding a leader sequence. In some embodiments, the expressible RNA sequence may comprise a third RNA sequence encoding a linker domain comprising any of the linker peptides disclosed herein, wherein the third RNA sequence is positioned between the first RNA sequence and the second RNA sequence in the 5′ to 3′ orientation.

In some embodiments, the first RNA sequence of the expressible RNA sequence of the present disclosure comprises an RNA sequence comprising at least about 60%, 65%, 70%, 75%, 80%, 85%, 90%, 95%, or 99% sequence identity to SEQ ID NO: 2, SEQ ID NO: 13, SEQ ID NO: 14, or SEQ ID NO: 15, or a complement thereof. In some embodiments, the first RNA sequence of the expressible RNA sequence of the present disclosure comprises an RNA sequence comprising SEQ ID NO: 2, SEQ ID NO: 13, SEQ ID NO: 14, or SEQ ID NO: 15, or a complement thereof. In some embodiments, the first RNA sequence of the expressible RNA sequence of the present disclosure comprises an RNA sequence encoding a self-assembling polypeptide comprising at least about 60%, 65%, 70%, 75%, 80%, 85%, 90%, 95%, or 99% sequence identity to SEQ ID NO: 7, SEQ ID NO: 23, SEQ ID NO: 31 or SEQ ID NO: 26, or a functional fragment thereof. In some embodiments, the first RNA sequence of the expressible RNA sequence of the present disclosure comprises an RNA sequence encoding a self-assembling polypeptide comprising SEQ ID NO: 7, SEQ ID NO: 23, SEQ ID NO: 31 or SEQ ID NO: 26, or a functional fragment thereof.

In some embodiments, the second RNA sequence of the expressible RNA sequence of the present disclosure comprises an RNA sequence comprising at least about 60%, 65%, 70%, 75%, 80%, 85%, 90%, 95%, or 99% sequence identity to SEQ ID NO: 4, SEQ ID NO: 41, SEQ ID NO: 42, SEQ ID NO: 43, SEQ ID NO: 44, SEQ ID NO: 46, SEQ ID NO: 48, SEQ ID NO: 50, SEQ ID NO: 52 or SEQ ID NO: 54 or a complement thereof. In some embodiments, the second RNA sequence of the expressible RNA sequence of the present disclosure comprises an RNA sequence comprising SEQ ID NO: 4, SEQ ID NO: 41, SEQ ID NO: 42, SEQ ID NO: 43, SEQ ID NO: 44, SEQ ID NO: 46, SEQ ID NO: 48, SEQ ID NO: 50, SEQ ID NO: 52 or SEQ ID NO: 54 or a complement thereof. In some embodiments, the second RNA sequence of the expressible RNA sequence of the present disclosure comprises an RNA sequence encoding a viral antigen comprising at least about 60%, 65%, 70%, 75%, 80%, 85%, 90%, 95%, or 99% sequence identity to SEQ ID NO: 9, SEQ ID NO: 45, SEQ ID NO: 47, SEQ ID NO: 49, SEQ ID NO: 51, SEQ ID NO: 53, SEQ ID NO: 55, SEQ ID NO: 65, SEQ ID NO: 66 or SEQ ID NO: 67, or a functional fragment thereof. In some embodiments, the second RNA sequence of the expressible RNA sequence of the present disclosure comprises an RNA sequence encoding a viral antigen comprising SEQ ID NO: 9, SEQ ID NO: 45, SEQ ID NO: 47, SEQ ID NO: 49, SEQ ID NO: 51, SEQ ID NO: 53, SEQ ID NO: 55, SEQ ID NO: 65, SEQ ID NO: 66 or SEQ ID NO: 67, or a functional fragment thereof.

In some embodiments, the third RNA sequence of the expressible RNA sequence of the present disclosure comprises an RNA sequence comprising at least about 60%, 65%, 70%, 75%, 80%, 85%, 90%, 95%, or 99% sequence identity to SEQ ID NO: 3, SEQ ID NO: 16, SEQ ID NO: 17, SEQ ID NO: 19 or SEQ ID NO: 29, or a complement thereof. In some embodiments, the third RNA sequence of the expressible RNA sequence of the present disclosure comprises an RNA sequence comprising SEQ ID NO: 3, SEQ ID NO: 16, SEQ ID NO: 17, SEQ ID NO: 19 or SEQ ID NO: 29, or a complement thereof. In some embodiments, the third RNA sequence of the expressible RNA sequence of the present disclosure comprises an RNA sequence encoding a linker peptide comprising at least about 60%, 65%, 70%, 75%, 80%, 85%, 90%, 95%, or 99% sequence identity to SEQ ID NO: 8, SEQ ID NO: 18, SEQ ID NO: 22, SEQ ID NO: 27 or SEQ ID NO: 32, or a functional fragment thereof. In some embodiments, the third RNA sequence of the expressible RNA sequence of the present disclosure comprises an RNA sequence encoding a linker peptide comprising SEQ ID NO: 8, SEQ ID NO: 18, SEQ ID NO: 22, SEQ ID NO: 27 or SEQ ID NO: 32, or a functional fragment thereof.

In some embodiments, the expressible RNA sequence of the present disclosure comprises an RNA sequence comprising at least about 60%, 65%, 70%, 75%, 80%, 85%, 90%, 95%, or 99% sequence identity to SEQ ID NO: 68, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97 or SEQ ID NO: 99, or a complement thereof. In some embodiments, the expressible RNA sequence of the present disclosure comprises an RNA sequence comprising SEQ ID NO: 68, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97 or SEQ ID NO: 99, or a complement thereof. In some embodiments, the expressible RNA sequence of the present disclosure comprises an RNA sequence encoding a polypeptide comprising at least about 60%, 65%, 70%, 75%, 80%, 85%, 90%, 95%, or 99% sequence identity to SEQ ID NO: 69, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98 or SEQ ID NO: 100, or a functional fragment thereof. In some embodiments, the expressible RNA sequence of the present disclosure comprises an RNA sequence encoding a polypeptide comprising SEQ ID NO: 69, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98 or SEQ ID NO: 100, or a functional fragment thereof.

### B. Nucleic Acid Molecule

In one aspect, the present disclosure also relates to a nucleic acid molecule that comprises any of the disclosed expressible nucleic acid sequences. For example, the expressible nucleic acid sequence disclosed herein can be part of a plasmid and thus the nucleic acid molecule is a plasmid comprising such an expressible nucleic acid sequence. Provided herein is a vector or plasmid that is capable of expressing at least a monomer of a self-assembling nanoparticle and a viral antigen construct or constructs in the cell of a mammal in a quantity effective to elicit an immune response in the mammal. The vector or plasmid may comprise heterologous nucleic acid encoding the one or more viral antigens (such as HIV-1 antigens). The vector may be a plasmid. The plasmid may be useful for transfecting cells with nucleic acid encoding a viral antigen, which the transformed host cell is cultured and maintained under conditions wherein expression of the viral antigen takes place and wherein the structure of the nanoparticle with the antigen elicits an immune response of a magnitude greater than and/or more therapeutically effective than the immune response elicited by the antigen alone. The plasmid may further comprise an initiation codon, which may be upstream of the expressible sequence, and a stop codon, which may be downstream of the coding sequence. The initiation and termination codon may be in frame with the expressible sequence.

The plasmid may also comprise a promoter that is operably linked to the coding sequence. The promoter operably linked to the coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, or human metalothionein. The promoter may also be a tissue specific promoter, such as a muscle or skin specific promoter, natural or synthetic. Examples of such promoters are described in US patent application publication no. US20040175727, the contents of which are incorporated herein in its entirety. The plasmid may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

The plasmid may also comprise an enhancer upstream of the coding sequence. The enhancer may be human actin, human myosin, human hemoglobin, human muscle creatine or a viral enhancer such as one from CMV, FMDV, RSV or EBV. Polynucleotide function enhancers are described in U.S. Pat. Nos. 5,593,972, 5,962,428, and WO94/016737, the contents of each are fully incorporated by reference. The plasmid may also comprise a mammalian origin of replication in order to maintain the plasmid extrachromosomally and produce multiple copies of the plasmid in a cell. The plasmid may be pVAX1, pCEP4 or pREP4 from ThermoFisher Scientific (San Diego, CA), which may comprise the Epstein Barr virus origin of replication and nuclear antigen EBNA-1 coding region, which may produce high copy episomal replication without integration.

In some embodiments, the vector can be pVAX1 or a pVax1 variant with changes such as the variant plasmid described herein. The variant pVax1 plasmid is a 2998 basepair variant of the backbone vector plasmid pVAX1 (Invitrogen, Carlsbad CA). The CMV promoter is located at bases 137-724. The T7 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993. The vaccine may comprise the consensus antigens and plasmids at quantities of from about 1 nanogram to 100 milligrams; about 1 microgram to about 10 milligrams; or preferably about 0.1 microgram to about 10 milligrams; or more preferably about 1 milligram to about 2 milligram. In some embodiments, pharmaceutical compositions according to the present disclosure comprise from about 1 nanogram to about 1000 micrograms of DNA. The nucleic acid sequence for the pVAX1 backbone sequence is as follows:

(SEQ ID NO: 56)
gactcttcgcgatgtacgggccagatatacgcgttgacattgattattgactagttattaatagtaatcaattacggggtcattagttcatag
cccatatatggagttccgcgttacataacttacggtaaatggcccgcctggctgaccgcccaacgacccccgcccattgacgtcaataa
tgacgtatgttcccatagtaacgccaatagggactttccattgacgtcaatgggtggactatttacggtaaactgcccacttggcagtaca
tcaagtgtatcatatgccaagtacgccccctattgacgtcaatgacggtaaatggcccgcctggcattatgcccagtacatgaccttatg
ggactttcctacttggcagtacatctacgtattagtcatcgctattaccatggtgatgcggttttggcagtacatcaatgggcgtggatagc
ggtttgactcacggggatttccaagtctccaccccattgacgtcaatgggagtttgttttggcaccaaaatcaacgggactttccaaaatg
tcgtaacaactccgccccattgacgcaaatgggcggtaggcgtgtacggtgggaggtctatataagcagagctctctggctaactaga
gaacccactgcttactggcttatcgaaattaatacgactcactatagggagacccaagctggctagcgtttaaacttaagcttggtaccg
agctcggatccactagtccagtgtggtggaattctgcagatatccagcacagtggcggccgctcgagtctagagggcccgtttaaacc
cgctgatcagcctcgactgtgccttctagttgccagccatctgttgtttgcccctcccccgtgccttccttgaccctggaaggtgccactc
ccactgtcctttcctaataaaatgaggaaattgcatcgcattgtctgagtaggtgtcattctattctggggggtggggtggggcaggaca
gcaagggggaggattgggaagacaatagcaggcatgctggggatgcggtgggctctatggcttctactgggcggttttatggacagc
aagcgaaccggaattgccagctggggcgccctctggtaaggttgggaagccctgcaaagtaaactggatggctttctcgccgccaag
gatctgatggcgcaggggatcaagctctgatcaagagacaggatgaggatcgtttcgcatgattgaacaagatggattgcacgcaggt
tctccggccgcttgggtggagaggctattcggctatgactgggcacaacagacaatcggctgctctgatgccgccgtgttccggctgt
cagcgcaggggcgcccggttctttttgtcaagaccgacctgtccggtgccctgaatgaactgcaagacgaggcagcgcggctatcgt
ggctggccacgacgggcgttccttgcgcagctgtgctcgacgttgtcactgaagcgggaagggactggctgctattgggcgaagtgc
cggggcaggatctcctgtcatctcaccttgctcctgccgagaaagtatccatcatggctgatgcaatgcggcggctgcatacgcttgat
ccggctacctgcccattcgaccaccaagcgaaacatcgcatcgagcgagcacgtactcggatggaagccggtcttgtcgatcaggat
gatctggacgaagagcatcaggggctcgcgccagccgaactgttcgccaggctcaaggcgagcatgcccgacggcgaggatctcg
tcgtgacccatggcgatgcctgcttgccgaatatcatggtggaaaatggccgcttttctggattcatcgactgtggccggctgggtgtgg
cggaccgctatcaggacatagcgttggctacccgtgatattgctgaagagcttggcggcgaatgggctgaccgcttcctcgtgctttac
ggtatcgccgctcccgattcgcagcgcatcgccttctatcgccttcttgacgagttcttctgaattattaacgcttacaatttcctgatgcgg
tattttctccttacgcatctgtgcggtatttcacaccgcatacaggtggcacttttcggggaaatgtgcgcggaacccctatttgtttatttttc
taaatacattcaaatatgtatccgctcatgagacaataaccctgataaatgcttcaataatagcacgtgctaaaacttcatttttaatttaaaa
ggatctaggtgaagatcctttttgataatctcatgaccaaaatcccttaacgtgagttttcgttccactgagcgtcagaccccgtagaaaag
atcaaaggatcttcttgagatcctttttttctgcgcgtaatctgctgcttgcaaacaaaaaaaccaccgctaccagcggtggtttgtttgccg
gatcaagagctaccaactctttttccgaaggtaactggcttcagcagagcgcagataccaaatactgtccttctagtgtagccgtagttag
gccaccacttcaagaactctgtagcaccgcctacatacctcgctctgctaatcctgttaccagtggctgctgccagtggcgataagtcgt
gtcttaccgggttggactcaagacgatagttaccggataaggcgcagcggtcgggctgaacggggggttcgtgcacacagcccagc
ttggagcgaacgacctacaccgaactgagatacctacagcgtgagctatgagaaagcgccacgcttcccgaagggagaaaggcgg
acaggtatccggtaagcggcagggtcggaacaggagagcgcacgagggagcttccagggggaaacgcctggtatctttatagtcct
gtcgggtttcgccacctctgacttgagcgtcgatttttgtgatgctcgtcaggggggcggagcctatggaaaaacgccagcaacgcgg
cctttttacggttcctgggcttttgctggccttttgctcacatgttctt

Other vectors or plasmids that can be used herein to produce the vaccine of the present disclosure include, but not limited to, pcDNA3.1(+), pCI mammalian expression vector, pSI vector, pZeoSV2(+), phCMV1, pTCP and pIRES with their respective backbone sequence as follows.

The pcDNA3.1(+) backbone sequence:

(SEQ ID NO: 76)
gacggatcgggagatctcccgatcccctatggtgcactctcagtacaatctgctctgatgccgcatagttaagccagtatctgctccctg
cttgtgtgttggaggtcgctgagtagtgcgcgagcaaaatttaagctacaacaaggcaaggcttgaccgacaattgcatgaagaatctg
cttagggttaggcgttttgcgctgcttcgcgatgtacgggccagatatacgcgttgacattgattattgactagttattaatagtaatcaatta
cggggtcattagttcatagcccatatatggagttccgcgttacataacttacggtaaatggcccgcctggctgaccgcccaacgacccc
cgcccattgacgtcaataatgacgtatgttcccatagtaacgccaatagggactttccattgacgtcaatgggtggagtatttacggtaaa
ctgcccacttggcagtacatcaagtgtatcatatgccaagtacgccccctattgacgtcaatgacggtaaatggcccgcctggcattatg
cccagtacatgaccttatgggactttcctacttggcagtacatctacgtattagtcatcgctattaccatggtgatgcggttttggcagtaca
tcaatgggcgtggatagcggtttgactcacggggatttccaagtctccaccccattgacgtcaatgggagtttgttttggcaccaaaatca
acgggactttccaaaatgtcgtaacaactccgccccattgacgcaaatgggcggtaggcgtgtacggtgggaggtctatataagcaga
gctctctggctaactagagaacccactgcttactggcttatcgaaattaatacgactcactatagggagacccaagctggctagcgttta
aacttaagcttggtaccgagctcggatccactagtccagtgtggtggaattctgcagatatccagcacagtggcggccgctcgagtcta
gagggcccgtttaaacccgctgatcagcctcgactgtgccttctagttgccagccatctgttgtttgcccctcccccgtgccttccttgac
cctggaaggtgccactcccactgtcctttcctaataaaatgaggaaattgcatcgcattgtctgagtaggtgtcattctattctggggggtg
gggtggggcaggacagcaagggggaggattgggaagacaatagcaggcatgctggggatgcggtgggctctatggcttctgagg
cggaaagaaccagctggggctctagggggtatccccacgcgccctgtagcggcgcattaagcgcggcgggtgtggtggttacgcg
cagcgtgaccgctacacttgccagcgccctagcgcccgctcctttcgctttcttcccttcctttctcgccacgttcgccggctttccccgtc
aagctctaaatcgggggctccctttagggttccgatttagtgctttacggcacctcgaccccaaaaaacttgattagggtgatggttcacg
tagtgggccatcgccctgatagacggtttttcgccctttgacgttggagtccacgttctttaatagtggactcttgttccaaactggaacaa
cactcaaccctatctcggtctattcttttgatttataagggattttgccgatttcggcctattggttaaaaaatgagctgatttaacaaaaattt
aacgcgaattaattctgtggaatgtgtgtcagttagggtgtggaaagtccccaggctccccagcaggcagaagtatgcaaagcatgcat
ctcaattagtcagcaaccaggtgtggaaagtccccaggctccccagcaggcagaagtatgcaaagcatgcatctcaattagtcagcaa
ccatagtcccgcccctaactccgcccatcccgcccctaactccgcccagttccgcccattctccgccccatggctgactaattttttttattt
atgcagaggccgaggccgcctctgcctctgagctattccagaagtagtgaggaggcttttttggaggcctaggcttttgcaaaaagctc
ccgggagcttgtatatccattttcggatctgatcaagagacaggatgaggatcgtttcgcatgattgaacaagatggattgcacgcaggt
tctccggccgcttgggtggagaggctattcggctatgactgggcacaacagacaatcggctgctctgatgccgccgtgttccggctgt
cagcgcaggggcgcccggttctttttgtcaagaccgacctgtccggtgccctgaatgaactgcaggacgaggcagcgcggctatcgt
ggctggccacgacgggcgttccttgcgcagctgtgctcgacgttgtcactgaagcgggaagggactggctgctattgggcgaagtgc
cggggcaggatctcctgtcatctcaccttgctcctgccgagaaagtatccatcatggctgatgcaatgcggcggctgcatacgcttgat
ccggctacctgcccattcgaccaccaagcgaaacatcgcatcgagcgagcacgtactcggatggaagccggtcttgtcgatcaggat
gatctggacgaagagcatcaggggctcgcgccagccgaactgttcgccaggctcaaggcgcgcatgcccgacggcgaggatctcg
tcgtgacccatggcgatgcctgcttgccgaatatcatggtggaaaatggccgcttttctggattcatcgactgtggccggctgggtgtgg
cggaccgctatcaggacatagcgttggctacccgtgatattgctgaagagcttggcggcgaatgggctgaccgcttcctcgtgctttac
ggtatcgccgctcccgattcgcagcgcatcgccttctatcgccttcttgacgagttcttctgagcgggactctggggttcgaaatgaccg
accaagcgacgcccaacctgccatcacgagatttcgattccaccgccgccttctatgaaaggttgggcttcggaatcgttttccgggac
gccggctggatgatcctccagcgcggggatctcatgctggagttcttcgcccaccccaacttgtttattgcagcttataatggttacaaat
aaagcaatagcatcacaaatttcacaaataaagcatttttttcactgcattctagttgtggtttgtccaaactcatcaatgtatcttatcatgtc
tgtataccgtcgacctctagctagagcttggcgtaatcatggtcatagctgtttcctgtgtgaaattgttatccgctcacaattccacacaac
atacgagccggaagcataaagtgtaaagcctggggtgcctaatgagtgagctaactcacattaattgcgttgcgctcactgcccgctttc
cagtcgggaaacctgtcgtgccagctgcattaatgaatcggccaacgcgcggggagaggcggtttgcgtattgggcgctcttccgctt
cctcgctcactgactcgctgcgctcggtcgttcggctgcggcgagcggtatcagctcactcaaaggcggtaatacggttatccacaga
atcaggggataacgcaggaaagaacatgtgagcaaaaggccagcaaaaggccaggaaccgtaaaaaggccgcgttgctggcgttt
ttccataggctccgcccccctgacgagcatcacaaaaatcgacgctcaagtcagaggtggcgaaacccgacaggactataaagatac
caggcgtttccccctggaagctccctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctgggctgtgtgcacgaaccccccg
ttcagcccgaccgctgcgccttatccggtaactatcgtcttgagtccaacccggtaagacacgacttatcgccactggcagcagccact
ggtaacaggattagcagagcgaggtatgtaggcggtgctacagagttcttgaagtggtggcctaactacggctacactagaagaaca
gtatttggtatctgcgctctgctgaagccagttaccttcggaaaaagagttggtagctcttgatccggcaaacaaaccaccgctggtagc
ggtttttttgtttgcaagcagcagattacgcgcagaaaaaaaggatctcaagaagatcctttgatcttttctacggggtctgacgctcagtg
gaacgaaaactcacgttaagggattttggtcatgagattatcaaaaaggatcttcacctagatccttttaaattaaaaatgaagttttaaatc
aatctaaagtatatatgagtaaacttggtctgacagttaccaatgcttaatcagtgaggcacctatctcagcgatctgtctatttcgttcatcc
atagttgcctgactccccgtcgtgtagataactacgatacgggagggcttaccatctggccccagtgctgcaatgataccgcgagacc
cacgctcaccggctccagatttatcagcaataaaccagccagccggaagggccgagcgcagaagtggtcctgcaactttatccgcct
ccatccagtctattaattgttgccgggaagctagagtaagtagttcgccagttaatagtttgcgcaacgttgttgccattgctacaggcatc
gtggtgtcacgctcgtcgtttggtatggcttcattcagctccggttcccaacgatcaaggcgagttacatgatcccccatgttgtgcaaaa
aagcggttagctccttcggtcctccgatcgttgtcagaagtaagttggccgcagtgttatcactcatggttatggcagcactgcataattct
cttactgtcatgccatccgtaagatgcttttctgtgactggtgagtactcaaccaagtcattctgagaatagtgtatgcggcgaccgagttg
ctcttgcccggcgtcaatacgggataataccgcgccacatagcagaactttaaaagtgctcatcattggaaaacgttcttcggggcgaa
aactctcaaggatcttaccgctgttgagatccagttcgatgtaacccactcgtgcacccaactgatcttcagcatcttttactttcaccagc
gtttctgggtgagcaaaaacaggaaggcaaaatgccgcaaaaaagggaataagggcgacacggaaatgttgaatactcatactcttc
ctttttcaatattattgaagcatttatcagggttattgtctcatgagcggatacatatttgaatgtatttagaaaaataaacaaataggggttcc
gcgcacatttccccgaaaagtgccacctgacgtc

The pCI mammalian expression vector backbone sequence:

(SEQ ID NO: 77)
tcaatattggccattagccatattattcattggttatatagcataaatcaatattggctattggccattgcatacgttgtatctatatcataata
tgtacatttatattggctcatgtccaatatgaccgccatgttggcattgattattgactagttattaatagtaatcaattacggggtcattagtt
catagcccatatatggagttccgcgttacataacttacggtaaatggcccgcctggctgaccgcccaacgacccccgcccattgacgtcaat
aatgacgtatgttcccatagtaacgccaatagggactttccattgacgtcaatgggtggagtatttacggtaaactgcccacttggcagta
catcaagtgtatcatatgccaagtccgccccctattgacgtcaatgacggtaaatggcccgcctggcattatgcccagtacatgacctta
cgggactttcctacttggcagtacatctacgtattagtcatcgctattaccatggtgatgcggttttggcagtacaccaatgggcgtggata
gcggtttgactcacggggatttccaagtctccaccccattgacgtcaatgggagtttgttttggcaccaaaatcaacgggactttccaaaa
tgtcgtaataaccccgccccgttgacgcaaatgggcggtaggcgtgtacggtgggaggtctatataagcagagctcgtttagtgaacc
gtcagatcactagaagctttattgcggtagtttatcacagttaaattgctaacgcagtcagtgcttctgacacaacagtctcgaacttaagc
tgcagaagttggtcgtgaggcactgggcaggtaagtatcaaggttacaagacaggtttaaggagaccaatagaaactgggcttgtcga
gacagagaagactcttgcgtttctgataggcacctattggtcttactgacatccactttgcctttctctccacaggtgtccactcccagttca
attacagctcttaaggctagagtacttaatacgactcactataggctagcctcgagaattcacgcgtggtacctctagagtcgacccggg
cggccgcttcgagcagacatgataagatacattgatgagtttggacaaaccacaactagaatgcagtgaaaaaaatgctttatttgtgaa
atttgtgatgctattgctttatttgtaaccattataagctgcaataaacaagttaacaacaacaattgcattcattttatgtttcaggttcaggg
ggagatgtgggaggttttttaaagcaagtaaaacctctacaaatgtggtaaaatcgataaggatccgggctggcgtaatagcgaagagg
cccgcaccgatcgcccttcccaacagttgcgcagcctgaatggcgaatggacgcgccctgtagcggcgcattaagcgcggcgggtg
tggtggttacgcgcagcgtgaccgctacacttgccagcgccctagcgcccgctcctttcgctttcttcccttcctttctcgccacgttcgc
cggctttccccgtcaagctctaaatcgggggctccctttagggttccgatttagtgctttacggcacctcgaccccaaaaaacttgattag
ggtgatggttcacgtagtgggccatcgccctgatagacggtttttcgccctttgacgttggagtccacgttctttaatagtggactcttgttc
caaactggaacaacactcaaccctatctcggtctattcttttgatttataagggattttgccgatttcggcctattggttaaaaaatgagctga
tttaacaaaaatttaacgcgaattttaacaaaatattaacgcttacaatttcctgatgcggtattttctccttacgcatctgtgcggtatttcac
accgcatatggtgcactctcagtacaatctgctctgatgccgcatagttaagccagccccgacacccgccaacacccgctgacgcgccc
tgacgggcttgtctgctcccggcatccgcttacagacaagctgtgaccgtctccgggagctgcatgtgtcagaggttttcaccgtcatca
ccgaaacgcgcgagacgaaagggcctcgtgatacgcctatttttataggttaatgtcatgataataatggtttcttagacgtcaggtggca
cttttcggggaaatgtgcgcggaacccctatttgtttatttttctaaatacattcaaatatgtatccgctcatgagacaataaccctgataaat
gcttcaataatattgaaaaaggaagagtatgagtattcaacatttccgtgtcgcccttattcccttttttgcggcattttgccttcctgtttttg
ctcacccagaaacgctggtgaaagtaaaagatgctgaagatcagttgggtgcacgagtgggttacatcgaactggatctcaacagcggt
aagatccttgagagttttcgccccgaagaacgttttccaatgatgagcacttttaaagttctgctatgtggcgcggtattatcccgtattgac
gccgggcaagagcaactcggtcgccgcatacactattctcagaatgacttggttgagtactcaccagtcacagaaaagcatcttacgg
atggcatgacagtaagagaattatgcagtgctgccataaccatgagtgataacactgcggccaacttacttctgacaacgatcggagga
ccgaaggagctaaccgcttttttgcacaacatgggggatcatgtaactcgccttgatcgttgggaaccggagctgaatgaagccatacc
aaacgacgagcgtgacaccacgatgcctgtagcaatggcaacaacgttgcgcaaactattaactggcgaactacttactctagcttccc
ggcaacaattaatagactggatggaggcggataaagttgcaggaccacttctgcgctcggcccttccggctggctggtttattgctgat
aaatctggagccggtgagcgtgggtctcgcggtatcattgcagcactggggccagatggtaagccctcccgtatcgtagttatctacac
gacggggagtcaggcaactatggatgaacgaaatagacagatcgctgagataggtgcctcactgattaagcattggtaactgtcagac
caagtttactcatatatactttagattgatttaaaacttcatttttaatttaaaaggatctaggtgaagatcctttttgataatctcatgaccaa
aatcccttaacgtgagttttcgttccactgagcgtcagaccccgtagaaaagatcaaaggatcttcttgagatcctttttttctgcgcgtaatct
gctgcttgcaaacaaaaaaaccaccgctaccagcggtggtttgtttgccggatcaagagctaccaactctttttccgaaggtaactggct
tcagcagagcgcagataccaaatactgttcttctagtgtagccgtagttaggccaccacttcaagaactctgtagcaccgcctacatacc
tcgctctgctaatcctgttaccagtggctgctgccagtggcgataagtcgtgtcttaccgggttggactcaagacgatagttaccggata
aggcgcagcggtcgggctgaacggggggttcgtgcacacagcccagcttggagcgaacgacctacaccgaactgagatacctaca
gcgtgagctatgagaaagcgccacgcttcccgaagggagaaaggcggacaggtatccggtaagcggcagggtcggaacaggag
agcgcacgagggagcttccagggggaaacgcctggtatctttatagtcctgtcgggtttcgccacctctgacttgagcgtcgatttttgt
gatgctcgtcaggggggcggagcctatggaaaaacgccagcaacgcggcctttttacggttcctggccttttgctggccttttgctcac
atggctcgacagatct

The pSI vector backbone sequence:

(SEQ ID NO: 78)
gcgcagcaccatggcctgaaataacctctgaaagaggaacttggttaggtaccttctgaggcggaaagaaccagctgtggaatgtgtg
tcagttagggtgtggaaagtccccaggctccccagcaggcagaagtatgcaaagcatgcatctcaattagtcagcaaccaggtgtgg
aaagtccccaggctccccagcaggcagaagtatgcaaagcatgcatctcaattagtcagcaaccatagtcccgcccctaactccgcc
catcccgcccctaactccgcccagttccgcccattctccgccccatggctgactaattttttttatttatgcagaggccgaggccgcctcg
gcctctgagctattccagaagtagtgaggaggcttttttggaggcctaggcttttgcaaaaagcttgattcttctgacacaacagtctcga
acttaagctgcagaagttggtcgtgaggcactgggcaggtaagtatcaaggttacaagacaggtttaaggagaccaatagaaactgg
gcttgtcgagacagagaagactcttgcgtttctgataggcacctattggtcttactgacatccactttgcctttctctccacaggtgtccact
cccagttcaattacagctcttaaggctagagtacttaatacgactcactataggctagcctcgagaattcacgcgtggtacctctagagtc
gacccgggcggccgcttcgagcagacatgataagatacattgatgagtttggacaaaccacaactagaatgcagtgaaaaaaatgctt
tatttgtgaaatttgtgatgctattgctttatttgtaaccattataagctgcaataaacaagttaacaacaacaattgcattcattttatgtttc
aggttcagggggaggtgtgggaggttttttaaagcaagtaaaacctctacaaatgtggtaaaatcgataaggatccgggctggcgtaatag
cgaagaggcccgcaccgatcgcccttcccaacagttgcgcagcctgaatggcgaatggacgcgccctgtagcggcgcattaagcg
cggcgggtgtggtggttacgcgcagcgtgaccgctacacttgccagcgccctagcgcccgctcctttcgctttcttcccttcctttctcgc
cacgttcgccggctttccccgtcaagctctaaatcgggggctccctttagggttccgatttagtgctttacggcacctcgaccccaaaaa
acttgattagggtgatggttcacgtagtgggccatcgccctgatagacggtttttcgccctttgacgttggagtccacgttctttaatagtg
gactcttgttccaaactggaacaacactcaaccctatctcggtctattcttttgatttataagggattttgccgatttcggcctattggttaaaa
aatgagctgatttaacaaaaatttaacgcgaattttaacaaaatattaacgcttacaatttcctgatgcggtattttctccttacgcatctgtgc
ggtatttcacaccgcatatggtgcactctcagtacaatctgctctgatgccgcatagttaagccagccccgacacccgccaacacccgc
tgacgcgccctgacgggcttgtctgctcccggcatccgcttacagacaagctgtgaccgtctccgggagctgcatgtgtcagaggtttt
caccgtcatcaccgaaacgcgcgagacgaaagggcctcgtgatacgcctatttttataggttaatgtcatgataataatggtttcttagac
gtcaggtggcacttttcggggaaatgtgcgcggaacccctatttgtttatttttctaaatacattcaaatatgtatccgctcatgagacaata
accctgataaatgcttcaataatattgaaaaaggaagagtatgagtattcaacatttccgtgtcgcccttattcccttttttgcggcattttgcc
ttcctgtttttgctcacccagaaacgctggtgaaagtaaaagatgctgaagatcagttgggtgcacgagtgggttacatcgaactggatc
tcaacagcggtaagatccttgagagttttcgccccgaagaacgttttccaatgatgagcacttttaaagttctgctatgtggcgcggtatta
tcccgtattgacgccgggcaagagcaactcggtcgccgcatacactattctcagaatgacttggttgagtactcaccagtcacagaaaa
gcatcttacggatggcatgacagtaagagaattatgcagtgctgccataaccatgagtgataacactgcggccaacttacttctgacaac
gatcggaggaccgaaggagctaaccgcttttttgcacaacatgggggatcatgtaactcgccttgatcgttgggaaccggagctgaat
gaagccataccaaacgacgagcgtgacaccacgatgcctgtagcaatggcaacaacgttgcgcaaactattaactggcgaactactt
actctagcttcccggcaacaattaatagactggatggaggcggataaagttgcaggaccacttctgcgctcggcccttccggctggctg
gtttattgctgataaatctggagccggtgagcgtgggtctcgcggtatcattgcagcactggggccagatggtaagccctcccgtatcgt
agttatctacacgacggggagtcaggcaactatggatgaacgaaatagacagatcgctgagataggtgcctcactgattaagcattggt
aactgtcagaccaagtttactcatatatactttagattgatttaaaacttcatttttaatttaaaaggatctaggtgaagatcctttttgataat
ctcatgaccaaaatcccttaacgtgagttttcgttccactgagcgtcagaccccgtagaaaagatcaaaggatcttcttgagatcctttttttct
gcgcgtaatctgctgcttgcaaacaaaaaaaccaccgctaccagcggtggtttgtttgccggatcaagagctaccaactctttttccgaa
ggtaactggcttcagcagagcgcagataccaaatactgttcttctagtgtagccgtagttaggccaccacttcaagaactctgtagcacc
gcctacatacctcgctctgctaatcctgttaccagtggctgctgccagtggcgataagtcgtgtcttaccgggttggactcaagacgata
gttaccggataaggcgcagcggtcgggctgaacggggggttcgtgcacacagcccagcttggagcgaacgacctacaccgaactg
agatacctacagcgtgagctatgagaaagcgccacgcttcccgaagggagaaaggcggacaggtatccggtaagcggcagggtcg
gaacaggagagcgcacgagggagcttccagggggaaacgcctggtatctttatagtcctgtcgggtttcgccacctctgacttgagcg
tcgatttttgtgatgctcgtcaggggggcggagcctatggaaaaacgccagcaacgcggcctttttacggttcctggccttttgctggcc
ttttgctcacatggctcgacagatct

The pZeoSV2(+) backbone sequence:

(SEQ ID NO: 79)
ggatcgatccggctgtggaatgtgtgtcagttagggtgtggaaagtccccaggctccccagcaggcagaagtatgcaaagcatgcat
ctcaattagtcagcaaccaggtgtggaaagtccccaggctccccagcaggcagaagtatgcaaagcatgcatctcaattagtcagcaa
ccatagtcccgcccctaactccgcccatcccgcccctaactccgcccagttccgcccattctccgccccatggctgactaattttttttattt
atgcagaggccgaggccgcctcggcctctgagctattccagaagtagtgaggaggcttttttggaggcctaggcttttgcaaaaagctc
tctggctaactagagaacccactgcttactggcttatcgaaattaatacgactcactatagggagacccaagctggctagcgtttaaactt
aagcttggtaccgagctcggatccactagtccagtgtggtggaattctgcagatatccagcacagtggcggccgctcgagtctagagg
gcccgtttaaacccgctgatcagcctcgactgtgccttctagttgccagccatctgttgtttgcccctcccccgtgccttccttgaccctgg
aaggtgccactcccactgtcctttcctaataaaatgaggaaattgcatcgcattgtctgagtaggtgtcattctattctggggggtggggt
ggggcaggacagcaagggggaggattgggaagacaatagcaggcatgctggggatgcggtgggctctatggcttctgaggcgga
aagaaccagcatgtgagcaaaaggccagcaaaaggccaggaaccgtaaaaaggccgcgttgctggcgtttttccataggctccgcc
cccctgacgagcatcacaaaaatcgacgctcaagtcagaggtggcgaaacccgacaggactataaagataccaggcgtttccccctg
gaagctccctcgtgcgctctcctgttccgaccctgccgcttaccggatacctgtccgcctttctcccttcgggaagcgtggcgctttctca
tagctcacgctgtaggtatctcagttcggtgtaggtcgttcgctccaagctgggctgtgtgcacgaaccccccgttcagcccgaccgct
gcgccttatccggtaactatcgtcttgagtccaacccggtaagacacgacttatcgccactggcagcagccactggtaacaggattagc
agagcgaggtatgtaggcggtgctacagagttcttgaagtggtggcctaactacggctacactagaagaacagtatttggtatctgcgc
tctgctgaagccagttaccttcggaaaaagagttggtagctcttgatccggcaaacaaaccaccgctggtagcggtggtttttttgtttgc
aagcagcagattacgcgcagaaaaaaaggatctcaagaagatcctttgatcttttctacggggtctgacgctcagtggaacgaaaactc
acgttaagggattttggtcatgacattaacctataaaaataggcgtatcacgaggccctttcgtctcgcgcgtttcggtgatgacggtgaa
aacctctgacacatgcagctcccggagacggtcacagcttgtctgtaagcggatgccgggagcagacaagcccgtcagggcgcgtc
agcgggtgttggcgggtgtcggggctggcttaactatgcggcatcagagcagattgtactgagagtgcaccatatgcggtgtgaaata
ccgcacagatgcgtaaggagaaaataccgcatcaggacgcgccctgtagcggcgcattaagcgcggcgggtgtggtggttacgcg
cagcgtgaccgctacacttgccagcgccctagcgcccgctcctttcgctttcttcccttcctttctcgccacgttcgccggctttccccgtc
aagctctaaatcgggggctccctttagggttccgatttagtgctttacggcacctcgaccccaaaaaacttgattagggtgatggttcacg
tagtgggccatcgccctgatagacggtttttcgccctttgacgttggagtccacgttctttaatagtggactcttgttccaaactggaacaa
cactcaaccctatctcggtctattcttttgatttataagggattttgccgatttcggcctattggttaaaaaatgagctgatttaacaaaaatt
taacgcgaattttaacaaaatattaacgcttacaatttccattcgccattcaggctgaactagatctagagtccgttacataacttacggtaaa
tggcccgcctggctgaccgcccaacgacccccgcccattgacgtcaataatgacgtatgttcccatagtaacgccaatagggactttcc
attgacgtcaatgggtggagtatttacggtaaactgcccacttggcagtacatcaagtgtatcatatgccaagtacgccccctattgacgt
caatgacggtaaatggcccgcctggcattatgcccagtacatgaccttatgggactttcctacttggcagtacatctacgtattagtcatc
gctattaccatggtgatgcggttttggcagtacatcaatgggcgtggatagcggtttgactcacggggatttccaagtctccaccccattg
acgtcaatgggagtttgttttggcaccaaaatcaacgggactttccaaaatgtcgtaacaactccgccccattgacgcaaatgggcggt
aggcgtgtacggtgggaggtctatataagcagagctcgtttagtgaaccgtcagatcgcctggagacgccatccacgctgttttgacct
ccatagaagacaccgggaccgatccagcctccgcggccgggaacggtgcattggaacggaccgtgttgacaattaatcatcggcat
agtatatcggcatagtataatacgacaaggtgaggaactaaaccatggccaagttgaccagtgccgttccggtgctcaccgcgcgcga
cgtcgccggagcggtcgagttctggaccgaccggctcgggttctcccgggacttcgtggaggacgacttcgccggtgtggtccggg
acgacgtgaccctgttcatcagcgcggtccaggaccaggtggtgccggacaacaccctggcctgggtgtgggtgcgcggcctgga
cgagctgtacgccgagtggtcggaggtcgtgtccacgaacttccgggacgcctccgggccggccatgaccgagatcggcgagcag
ccgtgggggcgggagttcgccctgcgcgacccggccggcaactgcgtgcacttcgtggccgaggagcaggactgacactcgacct
cgaaacttgtttattgcagcttataatggttacaaataaagcaatagcatcacaaatttcacaaataaagcatttttttcactgcattctagtt
gtggtttgtccaaactcatcaatgtatcttatcatgtct

The phCMV1 backbone sequence:

(SEQ ID NO: 80)
tagttattaatagtaatcaattacggggtcattagttcatagcccatatatggagttccgcgttacataacttacggtaaatggcccgcctgg
ctgaccgcccaacgacccccgcccattgacgtcaataatgacgtatgttcccatagtaacgccaatagggactttccattgacgtcaatg
ggtggagtatttacggtaaactgcccacttggcagtacatcaagtgtatcatatgccaagtacgccccctattgacgtcaatgacggtaa
atggcccgcctggcattatgcccagtacatgaccttatgggactttcctacttggcagtacatctacgtattagtcatcgctattaccatggt
gatgcggttttggcagtacatcaatgggcgtggatagcggtttgactcacggggatttccaagtctccaccccattgacgtcaatggga
gtttgttttggcaccaaaatcaacgggactttccaaaatgtcgtaacaactccgccccattgacgcaaatgggcggtaggcgtgtacggt
gggaggtctatataagcagagctcgtttagtgaaccgtcagatcgcctggagacgccatccacgctgttttgacctccatagaagacac
cgggaccgatccagcctccgcggccgggaacggtgcattggaacgcggattccccgtgccaagagtgacgtaagtaccgcctatag
actctataggcacacccctttggctcttatgcatgaattaatacgactcactatagggagacagactgttcctttcctgggtcttttctgcag
gcaccgtcgtcgacttaacagatctcgagctcaagcttcgaattctgcagtcgacggtaccgcgggcccgggatccaccgggtacaa
gtaaagcggccgcgactctagatcataatcagccataccacatttgtagaggttttacttgctttaaaaaacctcccacacctccccctga
acctgaaacataaaatgaatgcaattgttgttgttaacttgtttattgcagcttataatggttacaaataaagcaatagcatcacaaatttcac
aaataaagcatttttttcactgcattctagttgtggtttgtccaaactcatcaatgtatcttaaggcgtaaattgtaagcgttaatattttgtta
aaattcgcgttaaatttttgttaaatcagctcattttttaaccaataggccgaaatcggcaaaatcccttataaatcaaaagaatagaccgagat
agggttgagtgttgttccagtttggaacaagagtccactattaaagaacgtggactccaacgtcaaagggcgaaaaaccgtctatcagg
gcgatggcccactacgtgaaccatcaccctaatcaagttttttggggtcgaggtgccgtaaagcactaaatcggaaccctaaagggag
cccccgatttagagcttgacggggaaagccggcgaacgtggcgagaaaggaagggaagaaagcgaaaggagcgggcgctagg
gcgctggcaagtgtagcggtcacgctgcgcgtaaccaccacacccgccgcgcttaatgcgccgctacagggcgcgtcaggtggca
cttttcggggaaatgtgcgcggaacccctatttgtttatttttctaaatacattcaaatatgtatccgctcatgagacaataaccctgataaat
gcttcaataatattgaaaaaggaagagtcctgaggcggaaagaaccagctgtggaatgtgtgtcagttagggtgtggaaagtccccag
gctccccagcaggcagaagtatgcaaagcatgcatctcaattagtcagcaaccaggtgtggaaagtccccaggctccccagcaggc
agaagtatgcaaagcatgcatctcaattagtcagcaaccatagtcccgcccctaactccgcccatcccgcccctaactccgcccagttc
cgcccattctccgccccatggctgactaattttttttatttatgcagaggccgaggccgcctcggcctctgagctattccagaagtagtga
ggaggcttttttggaggcctaggcttttgcaaagatcgatcaagagacaggatgaggatcgtttcgcatgattgaacaagatggattgca
cgcaggttctccggccgcttgggtggagaggctattcggctatgactgggcacaacagacaatcggctgctctgatgccgccgtgttc
cggctgtcagcgcaggggcgcccggttctttttgtcaagaccgacctgtccggtgccctgaatgaactgcaagacgaggcagcgcg
gctatcgtggctggccacgacgggcgttccttgcgcagctgtgctcgacgttgtcactgaagcgggaagggactggctgctattgggc
gaagtgccggggcaggatctcctgtcatctcaccttgctcctgccgagaaagtatccatcatggctgatgcaatgcggcggctgcatac
gcttgatccggctacctgcccattcgaccaccaagcgaaacatcgcatcgagcgagcacgtactcggatggaagccggtcttgtcgat
caggatgatctggacgaagagcatcaggggctcgcgccagccgaactgttcgccaggctcaaggcgagcatgcccgacggcgag
gatctcgtcgtgacccatggcgatgcctgcttgccgaatatcatggtggaaaatggccgcttttctggattcatcgactgtggccggctg
ggtgtggcggaccgctatcaggacatagcgttggctacccgtgatattgctgaagagcttggcggcgaatgggctgaccgcttcctcg
tgctttacggtatcgccgctcccgattcgcagcgcatcgccttctatcgccttcttgacgagttcttctgagcgggactctggggttcgaa
atgaccgaccaagcgacgcccaacctgccatcacgagatttcgattccaccgccgccttctatgaaaggttgggcttcggaatcgttttc
cgggacgccggctggatgatcctccagcgcggggatctcatgctggagttcttcgcccaccctagggggaggctaactgaaacacg
gaaggagacaataccggaaggaacccgcgctatgacggcaataaaaagacagaataaaacgcacggtgttgggtcgtttgttcataa
acgcggggttcggtcccagggctggcactctgtcgataccccaccgagaccccattggggccaatacgcccgcgtttcttccttttccc
caccccaccccccaagttcgggtgaaggcccagggctcgcagccaacgtcggggggcaggccctgccatagcctcaggttactc
atatatactttagattgatttaaaacttcatttttaatttaaaaggatctaggtgaagatcctttttgataatctcatgaccaaaatcccttaa
cgtgagttttcgttccactgagcgtcagaccccgtagaaaagatcaaaggatcttcttgagatcctttttttctgcgcgtaatctgctgcttgc
aaacaaaaaaaccaccgctaccagcggtggtttgtttgccggatcaagagctaccaactctttttccgaaggtaactggcttcagcagagc
gcagataccaaatactgtccttctagtgtagccgtagttaggccaccacttcaagaactctgtagcaccgcctacatacctcgctctgcta
atcctgttaccagtggctgctgccagtggcgataagtcgtgtcttaccgggttggactcaagacgatagttaccggataaggcgcagcg
gtcgggctgaacggggggttcgtgcacacagcccagcttggagcgaacgacctacaccgaactgagatacctacagcgtgagctat
gagaaagcgccacgcttcccgaagggagaaaggcggacaggtatccggtaagcggcagggtcggaacaggagagcgcacgag
ggagcttccagggggaaacgcctggtatctttatagtcctgtcgggtttcgccacctctgacttgagcgtcgatttttgtgatgctcgtcag
gggggcggagcctatggaaaaacgccagcaacgcggcctttttacggttcctggccttttgctggccttttgctcacatgttctttcctgc
gttatcccctgattctgtggataaccgtattaccgccatgcat

The pTCP backbone sequence:

(SEQ ID NO: 81)
tagttattaatagtaatcaattacggggtcattagttcatagcccatatatggagttccgcgttacataacttacggtaaatggcccgcctgg
ctgaccgcccaacgacccccgcccattgacgtcaataatgacgtatgttcccatagtaacgccaatagggactttccattgacgtcaatg
ggtggagtatttacggtaaactgcccacttggcagtacatcaagtgtatcatatgccaagtacgccccctattgacgtcaatgacggtaa
atggcccgcctggcattatgcccagtacatgaccttatgggactttcctacttggcagtacatctacgtattagtcatcgctattaccatggt
gatgcggttttggcagtacatcaatgggcgtggatagcggtttgactcacggggatttccaagtctccaccccattgacgtcaatggga
gtttgttttggcaccaaaatcaacgggactttccaaaatgtcgtaacaactccgccccattgacgcaaatgggggtaggcgtgtacggt
gggaggtctatataagcagagctggtttagtgaaccgtggatcccgtcgcttaccgattcagaatggttgatatccgccattctgaatcg
gtaagcgacgaagcttaataaaggatcttttattttcattggatctgtgtgttggttttttgtgtgcggccgccctcgactgtgccttctagaa
gacaatagcaggcatgctggggatgcggtgggctctatggcttctgaggcggaaagaaccagctggggctctagggggtatcccca
cgcgccctgtagcggcgcattaagcgcggcgggtgtggtggttacgcgcagcgtgaccgctacacttgccagcgccctagcgcccg
ctcctttcgctttcttcccttcctttctcgccacgttcgccggctttccccgtcaagctctaaatcgggggctccctttagggttccgatttag
tgctttacggcacctcgaccccaaaaaacttgattagggtgatggttcacgtagtgggccatcgccctgatagacggtttttcgccctttga
cgttggagtccacgttctttaatagtggactcttgttccaaactggaacaacactcaaccctatctcggtctattcttttgatttataagggat
tttgccgatttcggcctattggttaaaaaatgagctgatttaacaaaaatttaacgcgaattaattctgtggaatgtgtgtcagttagggtgtg
gaaagtccccaggctccccagcaggcagaagtatgcaaagcatgcatctcaattagtcagcaaccaggtgtggaaagtccccaggct
ccccagcaggcagaagtatgcaaagcatgcatctcaattagtcagcaaccatagtcccgcccctaactccgcccatcccgcccctaac
tccgcccagttccgcccattctccgccccatggctgactaattttttttatttatgcagaggccgaggccgcctctgcctctgagctattcc
agaagtagtgaggaggcttttttggaggcctaggcttttgcaaaaagctcccgggatgaccgagtacaagcccacggtgcgcctcgc
cacccgcgacgacgtcccgcgggccgtacgcaccctcgccgccgcgttcgccgactaccccgccacgcgccacaccgtcgaccc
ggaccgccacatcgagcgggtcaccgagctgcaagaactcttcctcacgcgcgtcgggctcgacatcggcaaggtgtgggtcgcg
gacgacggcgccgcggtggcggtctggaccacgccggagagcgtcgaagcgggggcggtgttcgccgagatcggcccgcgcat
ggccgagttgagcggttcccggctggccgcgcagcaacagatggaaggcctcctggcgccgcaccggcccaaggagcccgcgtg
gttcctggccaccgtcggcgtctcgcccgaccaccagggcaagggtctgggcagcgccgtcgtgctccccggagtggaggcggcc
gagcgcgccggggtgcccgccttcctggagacctccgcgccccgcaacctccccttctacgagcggctcggcttcaccgtcaccgc
cgacgtcgaggtgcccgaaggaccgcgcacctggtgcatgacccgcaagcccggtgcctgattcgaatgaccgaccaagcgacgc
ccaacctgccatcacgagatttcgattccaccgccgccttctatgaaaggttgggcttcggaatcgttttccgggacgccggctggatg
atcctccagcgcggggatctcatgctggagttcttcgcccaccccaacttgtttattgcagcttataatggttacaaataaagcaatagcat
cacaaatttcacaaataaagcatttttttcactgcattctagttgtggtttgtccaaactcatcaatgtatcttatcatgtctgtataccgtcg
acctctagctagagcttggcgtaatcatggtcatagctgtttcctgtgtgaaattgttatccgctcacaattccacacaacatacgagccggaa
gcataaagtgtaaagcctggggtgcctaatgagtgagctaactcacattaattgcgttgcgctcactgcccgctttccagtcgggaaacc
tgtcgtgccagctgcattaatgaatcggccaacgcgcggggagaggcggtttgcgtattgggcgctcttccgcttcctcgctcactgac
tcgctgcgctcggtcgttcggctgcggcgagcggtatcagctcactcaaaggcggtaatacggttatccacagaatcaggggataac
gcaggaaagaacatgtgagcaaaaggccagcaaaaggccaggaaccgtaaaaaggccgcgttgctggcgtttttccataggctccg
cccccctgacgagcatcacaaaaatcgacgctcaagtcagaggtggcgaaacccgacaggactataaagataccaggcgtttcccc
ctggaagctccctcgtgcgctctcctgttccgaccctgccgcttaccggatacctgtccgcctttctcccttcgggaagcgtggcgctttc
tcatagctcacgctgtaggtatctcagttcggtgtaggtcgttcgctccaagctgggctgtgtgcacgaaccccccgttcagcccgacc
gctgcgccttatccggtaactatcgtcttgagtccaacccggtaagacacgacttatcgccactggcagcagccactggtaacaggatt
agcagagcgaggtatgtaggcggtgctacagagttcttgaagtggtggcctaactacggctacactagaagaacagtatttggtatctg
cgctctgctgaagccagttaccttcggaaaaagagttggtagctcttgatccggcaaacaaaccaccgctggtagcggtttttttgtttgc
aagcagcagattacgcgcagaaaaaaaggatctcaagaagatcctttgatcttttctacggggtctgacgctcagtggaacgaaaactc
acgttaagggattttggtcatgagattatcaaaaaggatcttcacctagatccttttaaattaaaaatgaagttttaaatcaatctaaagtata
tatgagtaaacttggtctgacagttaccaatgcttaatcagtgaggcacctatctcagcgatctgtctatttcgttcatccatagttgcctgac
tccccgtcgtgtagataactacgatacgggagggcttaccatctggccccagtgctgcaatgataccgcgagacccacgctcaccggc
tccagatttatcagcaataaaccagccagccggaagggccgagcgcagaagtggtcctgcaactttatccgcctccatccagtctatta
attgttgccgggaagctagagtaagtagttcgccagttaatagtttgcgcaacgttgttgccattgctacaggcatcgtggtgtcacgctc
gtcgtttggtatggcttcattcagctccggttcccaacgatcaaggcgagttacatgatcccccatgttgtgcaaaaaagcggttagctcc
ttcggtcctccgatcgttgtcagaagtaagttggccgcagtgttatcactcatggttatggcagcactgcataattctcttactgtcatgcca
tccgtaagatgcttttctgtgactggtgagtactcaaccaagtcattctgagaatagtgtatgcggcgaccgagttgctcttgcccggcgt
caatacgggataataccgcgccacatagcagaactttaaaagtgctcatcattggaaaacgttcttcggggcgaaaactctcaaggatc
ttaccgctgttgagatccagttcgatgtaacccactcgtgcacccaactgatcttcagcatcttttactttcaccagcgtttctgggtgagca
aaaacaggaaggcaaaatgccgcaaaaaagggaataagggcgacacggaaatgttgaatactcatactcttcctttttcaatattattga
agcatttatcagggttattgtctcatgagcggatacatatttgaatgtatttagaaaaataaacaaataggggttccgcgcacatttccccg
aaaagtgccacctgacgtcgacggatcgggagatctcccgatcccctatggtgcactctcagtacaatctgctctgatgccgcatagtt
aagccagtatctgctccctgcttgtgtgttggaggtcgctgagtagtgcgcgagcaaaatttaagctacaacaaggcaaggcttgaccg
acaattgcatgaagaatctgcttagggttaggcgttttgcgctgcttcgcgatgtacgggccagatatacgcgttgacattgattattgac

The pIRES backbone sequence:

(SEQ ID NO: 82)
tcaatattggccattagccatattattcattggttatatagcataaatcaatattggctattggccattgcatacgttgtatctatatcataata
tgtacatttatattggctcatgtccaatatgaccgccatgttggcattgattattgactagttattaatagtaatcaattacggggtcattagtt
catagcccatatatggagttccgcgttacataacttacggtaaatggcccgcctggctgaccgcccaacgacccccgcccattgacgtcaat
aatgacgtatgttcccatagtaacgccaatagggactttccattgacgtcaatgggtggagtatttacggtaaactgcccacttggcagta
catcaagtgtatcatatgccaagtccgccccctattgacgtcaatgacggtaaatggcccgcctggcattatgcccagtacatgacctta
cgggactttcctacttggcagtacatctacgtattagtcatcgctattaccatggtgatgcggttttggcagtacaccaatgggcgtggata
gcggtttgactcacggggatttccaagtctccaccccattgacgtcaatgggagtttgttttggcaccaaaatcaacgggactttccaaaa
tgtcgtaacaactgcgatcgcccgccccgttgacgcaaatgggcggtaggcgtgtacggtgggaggtctatataagcagagctcgttt
agtgaaccgtcagatcactagaagctttattgcggtagtttatcacagttaaattgctaacgcagtcagtgcttctgacacaacagtctcga
acttaagctgcagtgactctcttaaggtagccttgcagaagttggtcgtgaggcactgggcaggtaagtatcaaggttacaagacaggt
ttaaggagaccaatagaaactgggcttgtcgagacagagaagactcttgcgtttctgataggcacctattggtcttactgacatccacttt
gcctttctctccacaggtgtccactcccagttcaattacagctcttaaggctagagtacttaatacgactcactataggctagcctcgagaa
ttcacgcgtcgagcatgcatctagggggccaattccgcccctctcccccccccccctctccctcccccccccctaacgttactggccg
aagccgcttggaataaggccggtgtgcgtttgtctatatgttattttccaccatattgccgtcttttggcaatgtgagggcccggaaacctg
gccctgtcttcttgacgagcattcctaggggtctttcccctctcgccaaaggaatgcaaggtctgttgaatgtcgtgaaggaagcagttcc
tctggaagcttcttgaagacaaacaacgtctgtagcgaccctttgcaggcagcggaaccccccacctggcgacaggtgcctctgcgg
ccaaaagccacgtgtataagatacacctgcaaaggcggcacaaccccagtgccacgttgtgagttggatagttgtggaaagagtcaa
atggctctcctcaagcgtattcaacaaggggctgaaggatgcccagaaggtaccccattgtatgggatctgatctggggcctcggtgc
acatgctttacatgtgtttagtcgaggttaaaaaaacgtctaggccccccgaaccacggggacgtggttttcctttgaaaaacacgatgat
aagcttgccacaacccgggatcctctagagtcgacccgggcggccgcttccctttagtgagggttaatgcttcgagcagacatgataa
gatacattgatgagtttggacaaaccacaactagaatgcagtgaaaaaaatgctttatttgtgaaatttgtgatgctattgctttatttgtaac
cattataagctgcaataaacaagttaacaacaacaattgcattcattttatgtttcaggttcagggggagatgtgggaggttttttaaagcaa
gtaaaacctctacaaatgtggtaaaatccgataaggatcgatccgggctggcgtaatagcgaagaggcccgcaccgatcgcccttcc
caacagttgcgcagcctgaatggcgaatggacgcgccctgtagcggcgcattaagcgcggcgggtgtggtggttacgcgcagcgtg
accgctacacttgccagcgccctagcgcccgctcctttcgctttcttcccttcctttctcgccacgttcgccggctttccccgtcaagctct
aaatcgggggctccctttagggttccgatttagtgctttacggcacctcgaccccaaaaaacttgattagggtgatggttcacgtagtgg
gccatcgccctgatagacggtttttcgccctttgacgttggagtccacgttctttaatagtggactcttgttccaaactggaacaacactca
accctatctcggtctattcttttgatttataagggattttgccgatttcggcctattggttaaaaaatgagctgatttaacaaaaatttaacgcg
aattttaacaaaatattaacgcttacaatttcctgatgcggtattttctccttacgcatctgtgcggtatttcacaccgcatacgcggatctgc
gcagcaccatggcctgaaataacctctgaaagaggaacttggttaggtaccttctgaggcggaaagaaccagctgtggaatgtgtgtc
agttagggtgtggaaagtccccaggctccccagcaggcagaagtatgcaaagcatgcatctcaattagtcagcaaccaggtgtggaa
agtccccaggctccccagcaggcagaagtatgcaaagcatgcatctcaattagtcagcaaccatagtcccgcccctaactccgcccat
cccgcccctaactccgcccagttccgcccattctccgccccatggctgactaattttttttatttatgcagaggccgaggccgcctcggcc
tctgagctattccagaagtagtgaggaggcttttttggaggcctaggcttttgcaaaaagcttgattcttctgacacaacagtctcgaactt
aaggctagagccaccatgattgaacaagatggattgcacgcaggttctccggccgcttgggtggagaggctattcggctatgactggg
cacaacagacaatcggctgctctgatgccgccgtgttccggctgtcagcgcaggggcgcccggttctttttgtcaagaccgacctgtcc
ggtgccctgaatgaactgcaggacgaggcagcgcggctatcgtggctggccacgacgggcgttccttgcgcagctgtgctcgacgtt
gtcactgaagcgggaagggactggctgctattgggcgaagtgccggggcaggatctcctgtcatctcaccttgctcctgccgagaaa
gtatccatcatggctgatgcaatgcggcggctgcatacgcttgatccggctacctgcccattcgaccaccaagcgaaacatcgcatcg
agcgagcacgtactcggatggaagccggtcttgtcgatcaggatgatctggacgaagagcatcaggggctcgcgccagccgaactg
ttcgccaggctcaaggcgcgcatgcccgacggcgaggatctcgtcgtgacccatggcgatgcctgcttgccgaatatcatggtggaa
aatggccgcttttctggattcatcgactgtggccggctgggtgtggcggaccgctatcaggacatagcgttggctacccgtgatattgct
gaagagcttggcggcgaatgggctgaccgcttcctcgtgctttacggtatcgccgctcccgattcgcagcgcatcgccttctatcgcctt
cttgacgagttcttctgagcgggactctggggttcgaaatgaccgaccaagcgacgcccaacctgccatcacgatggccgcaataaa
atatctttattttcattacatctgtgtgttggttttttgtgtgaatcgatagcgataaggatccgcgtatggtgcactctcagtacaatctgctc
tgatgccgcatagttaagccagccccgacacccgccaacacccgctgacgcgccctgacgggcttgtctgctcccggcatccgcttac
agacaagctgtgaccgtctccgggagctgcatgtgtcagaggttttcaccgtcatcaccgaaacgcgcgagacgaaagggcctcgtg
atacgcctatttttataggttaatgtcatgataataatggtttcttagacgtcaggtggcacttttcggggaaatgtgcgcggaacccctattt
gtttatttttctaaatacattcaaatatgtatccgctcatgagacaataaccctgataaatgcttcaataatattgaaaaaggaagagtatgag
tattcaacatttccgtgtcgcccttattcccttttttgcggcattttgccttcctgtttttgctcacccagaaacgctggtgaaagtaaaagatg
ctgaagatcagttgggtgcacgagtgggttacatcgaactggatctcaacagcggtaagatccttgagagttttcgccccgaagaacgt
tttccaatgatgagcacttttaaagttctgctatgtggcgcggtattatcccgtattgacgccgggcaagagcaactcggtcgccgcatac
actattctcagaatgacttggttgagtactcaccagtcacagaaaagcatcttacggatggcatgacagtaagagaattatgcagtgctg
ccataaccatgagtgataacactgcggccaacttacttctgacaacgatcggaggaccgaaggagctaaccgcttttttgcacaacatg
ggggatcatgtaactcgccttgatcgttgggaaccggagctgaatgaagccataccaaacgacgagcgtgacaccacgatgcctgta
gcaatggcaacaacgttgcgcaaactattaactggcgaactacttactctagcttcccggcaacaattaatagactggatggaggcgga
taaagttgcaggaccacttctgcgctcggcccttccggctggctggtttattgctgataaatctggagccggtgagcgtgggtctcgcg
gtatcattgcagcactggggccagatggtaagccctcccgtatcgtagttatctacacgacggggagtcaggcaactatggatgaacg
aaatagacagatcgctgagataggtgcctcactgattaagcattggtaactgtcagaccaagtttactcatatatactttagattgatttaaa
acttcatttttaatttaaaaggatctaggtgaagatcctttttgataatctcatgaccaaaatcccttaacgtgagttttcgttccactgagcgt
cagaccccgtagaaaagatcaaaggatcttcttgagatcctttttttctgcgcgtaatctgctgcttgcaaacaaaaaaaccaccgctacc
agcggtggtttgtttgccggatcaagagctaccaactctttttccgaaggtaactggcttcagcagagcgcagataccaaatactgttctt
ctagtgtagccgtagttaggccaccacttcaagaactctgtagcaccgcctacatacctcgctctgctaatcctgttaccagtggctgctg
ccagtggcgataagtcgtgtcttaccgggttggactcaagacgatagttaccggataaggcgcagcggtcgggctgaacggggggtt
cgtgcacacagcccagcttggagcgaacgacctacaccgaactgagatacctacagcgtgagctatgagaaagcgccacgcttccc
gaagggagaaaggcggacaggtatccggtaagcggcagggtcggaacaggagagcgcacgagggagcttccagggggaaacg
cctggtatctttatagtcctgtcgggtttcgccacctctgacttgagcgtcgatttttgtgatgctcgtcaggggggcggagcctatggaaa
aacgccagcaacgcggcctttttacggttcctggccttttgctggccttttgctcacatggctcgacagatct

In some embodiments therefore, the disclosure relates to a composition comprising a nucleic acid molecule comprising at least about 70%, 75%, 80%, 85%, 90%, 91%, 92%, 93%, 94%, 95%, 96%, 97%, 98% or 99% sequence identity to SEQ ID NO: 56, SEQ ID NO: 76, SEQ ID NO: 77, SEQ ID NO: 78, SEQ ID NO: 79, SEQ ID NO:80, SEQ ID NO: 81 or SEQ ID NO: 82, or a functional fragment thereof. In some embodiments, the disclosure relates to a composition comprising a nucleic acid molecule comprising the nucleotide sequence of SEQ ID NO: 56, SEQ ID NO: 76, SEQ ID NO: 77, SEQ ID NO: 78, SEQ ID NO: 79, SEQ ID NO:80, SEQ ID NO: 81 or SEQ ID NO: 82, or a functional fragment thereof. In some embodiments, the disclosure relates to a composition comprising a nucleic acid molecule that is a pVax variant.

In some embodiments, the disclosure relates to a composition comprising a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 56, SEQ ID NO: 76, SEQ ID NO: 77, SEQ ID NO: 78, SEQ ID NO: 79, SEQ ID NO:80, SEQ ID NO: 81 or SEQ ID NO: 82, or a functional fragment thereof, and an expressible nucleic acid sequence comprising a first nucleic acid sequence encoding a scaffold domain comprising any of the self-assembling polypeptides disclosed herein and a second nucleic acid sequence encoding an antigen domain comprising any of the viral antigens disclosed herein. In some embodiments therefore, the composition of the present disclosure comprises a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 56, SEQ ID NO: 76, SEQ ID NO: 77, SEQ ID NO: 78, SEQ ID NO: 79, SEQ ID NO:80, SEQ ID NO: 81 or SEQ ID NO: 82, or a functional fragment thereof, and an expressible nucleic acid sequence comprising a first nucleic acid sequence comprising at least about 70%, 75%, 80%, 85%, 90%, 91%, 92%, 93%, 94%, 95%, 96%, 97%, 98%, 99% or 100% sequence identity to SEQ ID NO: 2, SEQ ID NO: 13, SEQ ID NO: 14, or SEQ ID NO: 15, or a functional fragment thereof, and a second nucleic acid sequence comprising at least about 70%, 75%, 80%, 85%, 90%, 91%, 92%, 93%, 94%, 95%, 96%, 97%, 98%, 99% or 100% sequence identity to SEQ ID NO: 4, SEQ ID NO: 41, SEQ ID NO: 42, SEQ ID NO: 43, SEQ ID NO: 44, SEQ ID NO: 46, SEQ ID NO: 48, SEQ ID NO: 50, SEQ ID NO: 52 or SEQ ID NO: 54 or a functional fragment thereof. In some embodiments, the composition of the present disclosure comprises a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 56, SEQ ID NO: 76, SEQ ID NO: 77, SEQ ID NO: 78, SEQ ID NO: 79, SEQ ID NO:80, SEQ ID NO: 81 or SEQ ID NO: 82, or a functional fragment thereof, and an expressible nucleic acid sequence comprising a first nucleic acid sequence encoding a self-assembling polypeptide comprising at least about 70%, 75%, 80%, 85%, 90%, 91%, 92%, 93%, 94%, 95%, 96%, 97%, 98%, 99% or 100% sequence identity to SEQ ID NO: 7, SEQ ID NO: 23, SEQ ID NO: 31 or SEQ ID NO: 26, or a functional fragment thereof, and a second nucleic acid sequence encoding a viral antigen comprising at least about 70%, 75%, 80%, 85%, 90%, 91%, 92%, 93%, 94%, 95%, 96%, 97%, 98%, 99% or 100% sequence identity to SEQ ID NO: 9, SEQ ID NO: 45, SEQ ID NO: 47, SEQ ID NO: 49, SEQ ID NO: 51, SEQ ID NO: 53, SEQ ID NO: 55, SEQ ID NO: 65, SEQ ID NO: 66 or SEQ ID NO: 67, or a functional fragment thereof.

In some embodiments, the expressible nucleic acid sequences comprised in such nucleic acid molecules or plasmids further comprise a third nucleic acid sequence encoding a linker domain comprising any of the linker peptides disclosed herein. In some embodiments therefore, the composition of the present disclosure comprises a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 56, SEQ ID NO: 76, SEQ ID NO: 77, SEQ ID NO: 78, SEQ ID NO: 79, SEQ ID NO:80, SEQ ID NO: 81 or SEQ ID NO: 82, or a functional fragment thereof, and an expressible nucleic acid sequence comprising a first nucleic acid sequence comprising at least about 70%, 75%, 80%, 85%, 90%, 91%, 92%, 93%, 94%, 95%, 96%, 97%, 98%, 99% or 100% sequence identity to SEQ ID NO: 2, SEQ ID NO: 13, SEQ ID NO: 14, or SEQ ID NO: 15, or a functional fragment thereof, a second nucleic acid sequence comprising at least about 70%, 75%, 80%, 85%, 90%, 91%, 92%, 93%, 94%, 95%, 96%, 97%, 98%, 99% or 100% sequence identity to SEQ ID NO: 4, SEQ ID NO: 41, SEQ ID NO: 42, SEQ ID NO: 43, SEQ ID NO: 44, SEQ ID NO: 46, SEQ ID NO: 48, SEQ ID NO: 50, SEQ ID NO: 52 or SEQ ID NO: 54, or a functional fragment thereof, and a third nucleic acid sequence comprising at least about 70%, 75%, 80%, 85%, 90%, 91%, 92%, 93%, 94%, 95%, 96%, 97%, 98%, 99% or 100% sequence identity to SEQ ID NO: 3, SEQ ID NO: 16, SEQ ID NO: 17, SEQ ID NO: 19 or SEQ ID NO: 29, or a functional fragment thereof. In some embodiments, the composition of the present disclosure comprises a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 56, SEQ ID NO: 76, SEQ ID NO: 77, SEQ ID NO: 78, SEQ ID NO: 79, SEQ ID NO:80, SEQ ID NO: 81 or SEQ ID NO: 82, or a functional fragment thereof, and an expressible nucleic acid sequence comprising a first nucleic acid sequence encoding a self-assembling polypeptide comprising at least about 70%, 75%, 80%, 85%, 90%, 91%, 92%, 93%, 94%, 95%, 96%, 97%, 98%, 99% or 100% sequence identity to SEQ ID NO: 7, SEQ ID NO: 23, SEQ ID NO: 31 or SEQ ID NO: 26, or a functional fragment thereof, a second nucleic acid sequence encoding a viral antigen comprising at least about 70%, 75%, 80%, 85%, 90%, 91%, 92%, 93%, 94%, 95%, 96%, 97%, 98%, 99% or 100% sequence identity to SEQ ID NO: 9, SEQ ID NO: 45, SEQ ID NO: 47, SEQ ID NO: 49, SEQ ID NO: 51, SEQ ID NO: 53, SEQ ID NO: 55, SEQ ID NO: 65, SEQ ID NO: 66 or SEQ ID NO: 67, or a functional fragment thereof, and a third nucleic acid sequence encoding a linker comprising at least about 70%, 75%, 80%, 85%, 90%, 91%, 92%, 93%, 94%, 95%, 96%, 97%, 98%, 99% or 100% sequence identity to SEQ ID NO: 8, SEQ ID NO: 18, SEQ ID NO: 22, SEQ ID NO: 27 or SEQ ID NO: 32, or a functional fragment thereof.

In some embodiments, the expressible nucleic acid sequences comprised in such nucleic acid molecules or plasmids are free of a nucleic acid sequence encoding a leader sequence.

In some embodiments, the expressible nucleic acid sequence comprised in the nucleic acid molecule or plasmid of the present disclosure comprises at least about 70%, 75%, 80%, 85%, 90%, 91%, 92%, 93%, 94%, 95%, 96%, 97%, 98% or 99% sequence identity to SEQ ID NO: 68, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97 or SEQ ID NO: 99, or a functional fragment thereof. In some embodiments, the expressible nucleic acid sequence of the present disclosure comprises the nucleic acid sequence of SEQ ID NO: 68, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97 or SEQ ID NO: 99, or a functional fragment thereof. In some embodiments, the expressible nucleic acid sequence comprised in the nucleic acid molecule or plasmid of the present disclosure encodes a polypeptide comprising at least about 70%, 75%, 80%, 85%, 90%, 91%, 92%, 93%, 94%, 95%, 96%, 97%, 98% or 99% sequence identity to SEQ ID NO: 69, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98 or SEQ ID NO: 100, or a functional fragment thereof. In some embodiments, the expressible nucleic acid sequence of the present disclosure encodes a polypeptide comprising the amino acid sequence of SEQ ID NO: 69, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98 or SEQ ID NO: 100, or a functional fragment thereof.

In some embodiments, the disclosure relates to a composition comprising a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 56, SEQ ID NO: 76, SEQ ID NO: 77, SEQ ID NO: 78, SEQ ID NO: 79, SEQ ID NO:80, SEQ ID NO: 81 or SEQ ID NO: 82, or a functional fragment thereof, and an expressible nucleic acid sequence comprising at least about 70%, 75%, 80%, 85%, 90%, 91%, 92%, 93%, 94%, 95%, 96%, 97%, 98%, 99% or 100% sequence identity to SEQ ID NO: 68, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97 or SEQ ID NO: 99, or a functional fragment thereof. In some embodiments, the disclosure relates to a composition comprising a nucleic acid molecule comprising the nucleotide sequence of SEQ ID NO: 56, SEQ ID NO: 76, SEQ ID NO: 77, SEQ ID NO: 78, SEQ ID NO: 79, SEQ ID NO:80, SEQ ID NO: 81 or SEQ ID NO: 82, or a functional fragment thereof, and an expressible nucleic acid sequence comprising the nucleotide sequence of SEQ ID NO: 68, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97 or SEQ ID NO: 99, or a functional fragment thereof. In some embodiments, the disclosure relates to a composition comprising a nucleic acid molecule comprising at least about 70%, 75%, 80%, 85%, 90%, 91%, 92%, 93%, 94%, 95%, 96%, 97%, 98%, 99% or 100% sequence identity to SEQ ID NO: 56, SEQ ID NO: 76, SEQ ID NO: 77, SEQ ID NO: 78, SEQ ID NO: 79, SEQ ID NO:80, SEQ ID NO: 81 or SEQ ID NO: 82, or a functional fragment thereof, and an expressible nucleic acid sequence encoding a polypeptide comprising at least about 70%, 75%, 80%, 85%, 90%, 91%, 92%, 93%, 94%, 95%, 96%, 97%, 98%, 99% or 100% sequence identity to SEQ ID NO: 69, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98 or SEQ ID NO: 100, or a functional fragment thereof. In some embodiments, the disclosure relates to a composition comprising a nucleic acid molecule comprising the nucleotide sequence of SEQ ID NO: 56, SEQ ID NO: 76, SEQ ID NO: 77, SEQ ID NO: 78, SEQ ID NO: 79, SEQ ID NO:80, SEQ ID NO: 81 or SEQ ID NO: 82, or a functional fragment thereof, and an expressible nucleic acid sequence encoding a polypeptide comprising the amino acid sequence of SEQ ID NO: 69, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98 or SEQ ID NO: 100, or a functional fragment thereof.

In some embodiments, the disclosure relates to nucleic acid molecules comprising a plasmid comprising a regulatory sequence operably linked one or more expressible nucleic acid sequences, wherein the expressible nucleic acid sequences comprise at least a first nucleic acid sequence encoding a scaffold domain comprising a self-assembling polypeptide, and a second nucleic acid sequence encoding an antigen domain comprising any one or plurality of viral antigens disclosed herein. In some embodiments, the first and second nucleic acids are linked by a linker peptide disclosed herein. In some embodiments, the first and second nucleic acids are in a 5′ to 3′ orientation and free of an IgE or IgG linker positioned 5′ of the 5′ end of the first and/or second nucleic acid sequence. In some embodiments, the disclosure relates to a composition comprising a nucleic acid molecule comprising at least about 70%, 75%, 80%, 85%, 90%, 91%, 92%, 93%, 94%, 95%, 96%, 97%, 98%, 99% or 100% sequence identity to SEQ ID NO: 56, or a functional fragment thereof, and positioned within a multiple cloning site are one or more expressible nucleic acid sequences of the present disclosure.

In some embodiments, the disclosed compositions can be vectors comprising a DNA backbone with an expressible insert comprising one or more of the nucleic acid sequences encoding a self-assembling polypeptide, linker and one or a plurality of viral antigens. In some embodiments, the disclosure relates to a viral vector comprising a DNA or RNA sequence that comprises one or more of the nucleic acid sequences encoding a self-assembling polypeptide, linker and one or a plurality of viral antigens.

### C. Polypeptide Sequences

Disclosed are the polypeptide sequences encoded by the disclosed nucleic acid sequences. In some embodiments, the disclosure relates to compositions comprising polypeptide sequences encoded by the expressible nucleic acid molecule of the present disclosure comprising a scaffold domain comprising a self-assembling polypeptide and an antigen domain comprising a viral antigen, and optionally comprising a linker domain comprising a linker peptide. The disclosure also relates to cells expressing one or more such polypeptides disclosed herein.

In some embodiments, the polypeptide encoded by the expressible nucleic acid molecule of the present disclosure comprises at least about 70%, 75%, 80%, 85%, 90%, 91%, 92%, 93%, 94%, 95%, 96%, 97% or 99% sequence identity to SEQ ID NO: 69, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98 or SEQ ID NO: 100, or a functional fragment thereof. In some embodiments, the polypeptide encoded by the expressible nucleic acid molecule of the present disclosure comprises the amino acid sequence of SEQ ID NO: 69, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98 or SEQ ID NO: 100, or a functional fragment thereof. The sequences of SEQ ID NO: 68 and SEQ ID NO: 69 also refer to in the present disclosure as GT8_180 mer_pVAX or DLnano_PfV_GT8.

In some embodiments, the self-assembling polypeptide comprises at least about 70%, 75%, 80%, 85%, 90%, 91%, 92%, 93%, 94%, 95%, 96%, 97% or 99% sequence identity to SEQ ID NO: 7, SEQ ID NO: 23, SEQ ID NO: 31 or SEQ ID NO: 26, or a functional fragment thereof. In some embodiments, the self-assembling polypeptide comprises the amino acid sequence of SEQ ID NO: 7, SEQ ID NO: 23, SEQ ID NO: 31 or SEQ ID NO: 26, or a functional fragment thereof.

In some embodiments, the viral antigen encoded by the expressible nucleic acid sequence of the present disclosure comprises at least about 60%, 65%, 70%, 75%, 80%, 85%, 90%, 95%, or 99% sequence identity to SEQ ID NO: 9, SEQ ID NO: 45, SEQ ID NO: 47, SEQ ID NO: 49, SEQ ID NO: 51, SEQ ID NO: 53, SEQ ID NO: 55, SEQ ID NO: 65, SEQ ID NO: 66 or SEQ ID NO: 67, or a functional fragment thereof. In some embodiments, the viral antigen comprises the amino acid sequence of SEQ ID NO: 9, SEQ ID NO: 45, SEQ ID NO: 47, SEQ ID NO: 49, SEQ ID NO: 51, SEQ ID NO: 53, SEQ ID NO: 55, SEQ ID NO: 65, SEQ ID NO: 66 or SEQ ID NO: 67, or a functional fragment thereof.

In some embodiments, the linker peptide encoded by the expressible nucleic acid sequence of the present disclosure comprises at least about 60%, 65%, 70%, 75%, 80%, 85%, 90%, 95%, or 99% sequence identity to SEQ ID NO: 8, SEQ ID NO: 18, SEQ ID NO: 22, SEQ ID NO: 27 or SEQ ID NO: 32, or a functional fragment thereof. In some embodiments, the linker peptide comprises the amino acid sequence of SEQ ID NO: 8, SEQ ID NO: 18, SEQ ID NO: 22, SEQ ID NO: 27 or SEQ ID NO: 32, or a functional fragment thereof.

### Recitation of Sequences:

IgE-GLT1-3BVE-Entire Expressible Nucleic Acid Sequence expressing 3BVE (SEQ ID
NO: 20) (Linker sequence (SEQ ID NO: 17): bold and italic; 3BVE (Forms 24mer-SEQ ID
NO: 13): underlined)
atggactggacctggattctgttcctggtggccgccgccacaagggtgcacagcgacaccatcacactgccatgccgccctgcacca
cctccacattgtagctccaacatcaccggcctgattctgacaagacaggggggatatagtaacgataataccgtgattttcaggccctca
ggaggggactggagggacatcgcacgatgccagattgctggaacagtggtctctactcagctgtttctgaacggcagtctggctgag
gaagaggtggtcatccgatctgaagactggcgggataatgcaaagtcaatttgtgtgcagctgaacacaagcgtcgagatcaattgca
ctggcgcagggcactgtaacatttctcgggccaaatggaacaataccctgaagcagatcgccagtaaactgagagagcagtacggc
aataagacaatcatcttcaagccttctagtggaggcgacccagagttcgtgaaccatagctttaattgcgggggagagttcttttattgtg
attccacacagctgttcaacagcacttggtttaattccaccggcggaagcggcggaagcggcgggtctgggctgagtaaggacatta
tcaagctgctgaacgaacaggtgaacaaagagatgcagtctagcaacctgtacatgtccatgagctcctggtgctatacccactctctg
gacggagcaggcctgttcctgtttgatcacgccgccgaggagtacgagcacgccaagaagctgatcatcttcctgaatgagaacaat
gtgcccgtgcagctgacctctatcagcgcccctgagcacaagttcgagggcctgacacagatctttcagaaggcctacgagcacgag
cagcacatctccgagtctatcaacaatatcgtggaccacgccatcaagtccaaggatcacgccacattcaactttctgcagtggtacgtg
gccgagcagcacgaggaggaggtgctgtttaaggacatcctggataagatcgagctgatcggcaatgagaaccacgggctgtacct
ggcagatcagtatgtcaagggcatcgctaagtcaaggaaaagctgataa
Entire Expressed amino acid sequence (SEQ ID NO: 21) (Linker: bold and italic; 3BVE
Scaffold: underlined)
MDWTWILFLVAAATRVHSDTITLPCRPAPPPHCSSNITGLILTRQGGYSNDNTVIFRPS
GGDWRDIARCQIAGTVVSTQLFLNGSLAEEEVVIRSEDWRDNAKSICVQLNTSVEIN
CTGAGHCNISRAKWNNTLKQIASKLREQYGNKTIIFKPSSGGDPEFVNHSFNCGGEFF
YCDSTQLFNSTWENSTGGSGGSGGSGLSKDIIKLLNEQVNKEMQSSNLYMSMSSWC
YTHSLDGAGLFLFDHAAEEYEHAKKLIIFLNENNVPVQLTSISAPEHKFEGLTQIFQKA
YEHEQHISESINNIVDHAIKSKDHATFNFLQWYVAEQHEEEVLFKDILDKIELIGNENH
GLYLADQYVKGIAKSRKS
IgE-GLT1-13-Entire Expressible Nucleic Acid Sequence expressing I3 (SEQ ID NO: 24)
(Linker (SEQ ID NO: 19): bold and italic; I3 Scaffold (SEQ ID NO: 15): underlined)
atggactggacctggattctgttcctggtggccgccgccacaagggtgcacagcgacaccatcacactgccatgccgccctgcacca
cctccacattgtagctccaacatcaccggcctgattctgacaagacaggggggatatagtaacgataataccgtgattttcaggccctca
ggaggggactggagggacatcgcacgatgccagattgctggaacagtggtctctactcagctgtttctgaacggcagtctggctgag
gaagaggtggtcatccgatctgaagactggcgggataatgcaaagtcaatttgtgtgcagctgaacacaagcgtcgagatcaattgca
ctggcgcagggcactgtaacatttctcgggccaaatggaacaataccctgaagcagatcgccagtaaactgagagagcagtacggc
aataagacaatcatcttcaagccttctagtggaggcgacccagagttcgtgaaccatagctttaattgcgggggagagttcttttattgtg
attccacacagctgttcaacagcacttggtttaattccaccggcggcagcggcggcagcggcgggagcggaggaagtgagaaagc
agccaaagcagaggaagcagcacggaagatggaagaactgttcaagaagcacaagatcgtggccgtgctgagggccaactccgt
ggaggaggccaagaagaaggccctggccgtgttcctgggcggcgtgcacctgatcgagatcacctttacagtgcccgacgccgata
ccgtgatcaaggagctgtctttcctgaaggagatgggagcaatcatcggagcaggaaccgtgacaagcgtggagcagtgcagaaag
gccgtggagagcggcgccgagtttatcgtgtcccctcacctggacgaggagatctctcagttctgtaaggagaagggcgtgttttacat
gccaggcgtgatgacccccacagagctggtgaaggccatgaagctgggccacacaatcctgaagctgttccctggcgaggtggtgg
gcccacagtttgtgaaggccatgaagggccccttccctaatgtgaagtttgtgcccaccggcggcgtgaacctggataacgtgtgcga
gtggttcaaggcaggcgtgctggcagtgggcgtgggcagcgccctggtgaagggcacacccgtggaagtcgctgagaaggcaaa
ggcattcgtggaaaagattagggggtgtactgagtgataa
Entire Expressed Amino Acid sequence with 13-linker- Antigen (SEQ ID NO: 25) (Linker
(SEQ ID NO: 27): bold and italic; 13 Scaffold (SEQ ID NO: 26): underlined)
MDWTWILFLVAAATRVHSDTITLPCRPAPPPHCSSNITGLILTRQGGYSNDNTVIFRPS
GGDWRDIARCQIAGTVVSTQLFLNGSLAEEEVVIRSEDWRDNAKSICVQLNTSVEIN
CTGAGHCNISRAKWNNTLKQIASKLREQYGNKTIIFKPSSGGDPEFVNHSFNCGGEFF
YCDSTQLFNSTWENSTGGSGGSGGSGGSEKAAKAEEAARKMEELFKKHKIVAVLRA
NSVEEAKKKALAVFLGGVHLIEITFTVPDADTVIKELSFLKEMGAIIGAGTVTSVEQC
RKAVESGAEFIVSPHLDEEISQFCKEKGVFYMPGVMTPTELVKAMKLGHTILKLFPGE
VVGPQFVKAMKGPFPNVKFVPTGGVNLDNVCEWFKAGVLAVGVGSALVKGTPVEV
AEKAKAFVEKIRGCTE
IgE-GLT1-RBE-Entire Expressible Nucleic Acid Sequence expressing RBE (SEQ ID NO:
28) (Linker (SEQ ID NO: 29): bold and italic; RBE Scaffold (SEQ ID NO: 14): underlined)
atggactggacctggattctgttcctggtggccgccgccacaagggtgcacagcctgagcattgcccccacactgattaaccgggac
aaaccctacaccaaagaggaactgatggagattctgagactggctattatcgctgagctggacgccatcaacctgtacgagcagatgg
cccggtattctgaggacgagaatgtgcgcaagatcctgctggatgtggccagggaggagaaggcacacgtgggagagttcatggcc
ctgctgctgaacctggaccccgagcaggtgaccgagctgaagggcggctttgaggaggtgaaggagctgacaggcatcgaggccc
acatcaacgacaataagaaggaggagagcaacgtggagtatttcgagaagctgagatccgccctgctggatggcgtgaataagggc
aggagcctgctgaagcacctgcctgtgaccaggatcgagggccagagcttcagagtggacatcatcaagtttgaggatggcgtgcg
cgtggtgaagcaggagtacaagcccatccctctgctgaagaagaagttctacgtgggcatcagggagctgaacgacggcacctacg
atgtgagcatcgccacaaaggccggcgagctgctggtgaaggacgaggagtccctggtcatccgcgagatcctgtctacagagggc
atcaagaagatgaagctgagctcctgggacaatccagaggaggccctgaacgatctgatgaatgccctgcaggaggcatctaacgc
aagcgccggaccattcggcctgatcatcaatcccaagagatacgccaagctgctgaagatctatgagaagtccggcaagatgctggt
ggaggtgctgaaggagatcttccggggcggcatcatcgtgaccctgaacatcgatgagaacaaagtgatcatctttgccaacacccct
gccgtgctggacgtgotggtgggacaggatgtgacactgcaggagctgggaccagagggcgacgatgtggcctttctggtgtccga
ggccatcggcatcaggatcaagaatccagaggcaatcgtggtgctggagggcggctctggcggaagtggcggaagtgggggaa
gtggaggcggcggaagcgggggaggcagcgggggaggggacaccatcacactgccatgccgccctgcaccacctccacattgt
agctccaacatcaccggcctgattctgacaagacaggggggatatagtaacgataataccgtgattttcaggccctcaggaggggact
ggagggacatcgcacgatgccagattgctggaacagtggtctctactcagctgtttctgaacggcagtctggctgaggaagaggtggt
catccgatctgaagactggcgggataatgcaaagtcaatttgtgtgcagctgaacacaagcgtcgagatcaattgcactggcgcaggg
cactgtaacatttctcgggccaaatggaacaataccctgaagcagatcgccagtaaactgagagagcagtacggcaataagacaatc
atcttcaagccttctagtggaggcgacccagagttcgtgaaccatagctttaattgcgggggagagttcttttattgtgattccacacagct
gttcaacagcacttggtttaattccacc
Protein sequence IgE leader-RBE- linker-HIV antigen (SEQ ID NO: 30) (Linker (SEQ ID
NO: 32): bold and italic; RBE Scaffold (SEQ ID NO: 31): underlined)
MDWTWILFLVAAATRVHSLSIAPTLINRDKPYTKEELMEILRLAIIAELDAINLYEQM
ARYSEDENVRKILLDVAREEKAHVGEFMALLLNLDPEQVTELKGGFEEVKELTGIEA
HINDNKKEESNVEYFEKLRSALLDGVNKGRSLLKHLPVTRIEGQSFRVDIIKFEDGVR
VVKQEYKPIPLLKKKFYVGIRELNDGTYDVSIATKAGELLVKDEESLVIREILSTEGIK
KMKLSSWDNPEEALNDLMNALQEASNASAGPFGLIINPKRYAKLLKIYEKSGKMLV
EVLKEIFRGGIMQDIFIEDLNIDENKVIIFANTPAVLDVVVGQDVTLQELGPEGDDVAF
LVSEAIGIRIKNPEAIVVLEGGSGGSGGSGGSGGGGSGGGSGGGDTITLPCRPAPPPHC
SSNITGLILTRQGGYSNDNTVIFRPSGGDWRDIARCQIAGTVVSTQLFLNGSLAEEEV
VIRSEDWRDNAKSICVQLNTSVEINCTGAGHCNISRAKWNNTLKQIASKLREQYGNK
TIIFKPSSGGDPEFVNHSFNCGGEFFYCDSTQLFNSTWENST
Nipah virus-Construct 1. NivFtop_stab2_gMax_Nt_60 mer; Entire Expressible Nucleic Acid
Sequence for Nipah Virus antigen with 60 mer Self-Assembly (SEQ ID NO: 33)
atggactggacctggattctgttcctggtggccgccgccacaagggtgcacagcatgcagatctacgaaggaaaactgaccgctgag
ggactgaggttcggaattgtcgcaagccgcgcgaatcacgcactggtggataggctggtggaaggcgctatcgacgcaattgtccgg
cacgggggagagaggaagacatcacactggtgagagtctgcggcagctgggagattcccgtggcagctggagaactggctcgaa
aggaggacatcgatgccgtgatcgctattggggtcctgtgccgaggagcaactcccagcttcgactacatcgcctcagaagtgagca
aggggctggctgatctgtccctggagctgaggaaacctatcacttttggcgtgattactgccgacaccctggaacaggcaatcgaggc
ggccggcacctgccatggaaacaaaggctgggaagcagccctgtgcgctattgagatggcaaatctgttcaaatctctgcgaggagg
ctccggaggatctggagggagtggaggctcaggaggaggcggggtcacttgtgccggacgagccatcggaaatgctaccgccgc
ccagattactgccggagtcgccctgtatgaagccatgaagaatgccgacaacatcaataagctgaagagctccatcgagagcaccaa
cgaggccgtggtgaagctgcaggagacagccgagaagacagtgtacgtgctgacagccctgcaggactatatcaacaccaatctgg
tgcccacaatcgataagatcagctgcaagcagaccgaggcatccctggacgccgccctgtccaagtacctgtctgatctgctgtacgt
gttcggccccaacctgagcgaccccgtgagcaattctatgcctatccaggccatctctcaggccttcggcggcaactacagcaccctg
ctgaggacactgggctatgccccagaggactttgacgatctgctggagagcgattccatcacaggccagatcatctacgtggacctgt
ctagctactatatcatcgtgagagtgtattttccaaatggctccggccccctgaccaaggatatcgtgatcaagatgatccccaacgtgtc
taatatgagccagtgtacaggctctgtgatggagaactacaagaccaggctgaatggcatcctgacacctatcaagggcgccctgga
gatctataagaataactgtcacgatggatgataa
NivFtop_stab2_gMax (SEQ ID NO: 34)
ggggtcacttgtgccggacgagccatcggaaatgctaccgccgcccagattactgccggagtcgccctgtatgaagccatgaagaat
gccgacaacatcaataagctgaagagctccatcgagagcaccaacgaggccgtggtgaagctgcaggagacagccgagaagaca
gtgtacgtgctgacagccctgcaggactatatcaacaccaatctggtgcccacaatcgataagatcagctgcaagcagaccgaggcat
ccctggacgccgccctgtccaagtacctgtctgatctgctgtacgtgttcggccccaacctgagcgaccccgtgagcaattctatgcct
atccaggccatctctcaggccttcggcggcaactacagcaccctgctgaggacactgggctatgccccagaggactttgacgatctgc
tggagagcgattccatcacaggccagatcatctacgtggacctgtctagctactatatcatcgtgagagtgtattttccaaatggctccgg
ccccctgaccaaggatatcgtgatcaagatgatccccaacgtgtctaatatgagccagtgtacaggctctgtgatggagaactacaaga
ccaggctgaatggcatcctgacacctatcaagggcgccctggagatctataagaataactgtcacgatggatgataa
Entire Expressed IgE-Self Assembly-Linker-Viral Antigen sequence (SEQ ID NO: 35)
MDWTWILFLVAAATRVHSMQIYEGKLTAEGLRFGIVASRANHALVDRLVEGAIDAI
VRHGGREEDITLVRVCGSWEIPVAAGELARKEDIDAVIAIGVLCRGATPSFDYIASEV
SKGLADLSLELRKPITFGVITADTLEQAIEAAGTCHGNKGWEAALCAIEMANLFKSLR
GGSGGSGGSGGSGGGGVTCAGRAIGNATAAQITAGVALYEAMKNADNINKLKSSIE
STNEAVVKLQETAEKTVYVLTALQDYINTNLVPTIDKISCKQTEASLDAALSKYLSDL
LYVFGPNLSDPVSNSMPIQAISQAFGGNYSTLLRTLGYAPEDFDDLLESDSITGQIIYV
DLSSYYIIVRVYFPNGSGPLTKDIVIKMIPNVSNMSQCTGSVMENYKTRLNGILTPIKG
ALEIYKNNCHDG
NivFtop_stab2_gMax expressed amino acid sequence (SEQ ID NO: 36)
GVTCAGRAIGNATAAQITAGVALYEAMKNADNINKLKSSIESTNEAVVKLQETAEKT
VYVLTALQDYINTNLVPTIDKISCKQTEASLDAALSKYLSDLLYVFGPNLSDPVSNSM
PIQAISQAFGGNYSTLLRTLGYAPEDFDDLLESDSITGQIIYVDLSSYYIIVRVYFPNGS
GPLTKDIVIKMIPNVSNMSQCTGSVMENYKTRLNGILTPIKGALEIYKNNCHDG
Construct 2-NivFtop_stab2_gMax_Ct_60 mer; Entire Expressible Nucleic Acid Sequence
for Nipah Virus Antigen with 60 mer Self-Assembly (SEQ ID NO: 37)
atggactggacctggattctgttcctggtggccgccgccacaagggtgcacagcggggtcacttgtgccggacgagccatcggaaat
gctaccgccgcccagattactgccggagtcgccctgtatgaagccatgaagaatgccgacaacatcaataagctgaagagctccatc
gagagcaccaacgaggccgtggtgaagctgcaggagacagccgagaagacagtgtacgtgctgacagccctgcaggactatatca
acaccaatctggtgcccacaatcgataagatcagctgcaagcagaccgaggcatccctggacgccgccctgtccaagtacctgtctg
atctgctgtacgtgttcggccccaacctgagcgaccccgtgagcaattctatgcctatccaggccatctctcaggccttcggcggcaac
tacagcaccctgctgaggacactgggctatgccccagaggactttgacgatctgctggagagcgattccatcacaggccagatcatct
acgtggacctgtctagctactatatcatcgtgagagtgtattttccaaatggctccggccccctgaccaaggatatcgtgatcaagatgat
ccccaacgtgtctaatatgagccagtgtacaggctctgtgatggagaactacaagaccaggctgaatggcatcctgacacctatcaag
ggcgccctggagatctataagaataactgtcacgatggaggaggctccggaggatctggagggagtggaggctcaggaggaggca
tgcagatctacgaaggaaaactgaccgctgagggactgaggttcggaattgtcgcaagccgcgcgaatcacgcactggtggatagg
ctggtggaaggcgctatcgacgcaattgtccggcacggcgggagagaggaagacatcacactggtgagagtctgcggcagctggg
agattcccgtggcagctggagaactggctcgaaaggaggacatcgatgccgtgatcgctattggggtcctgtgccgaggagcaactc
ccagcttcgactacatcgcctcagaagtgagcaaggggctggctgatctgtccctggagctgaggaaacctatcacttttggcgtgatt
actgccgacaccctggaacaggcaatcgaggcggccggcacctgccatggaaacaaaggctgggaagcagccctgtgcgctattg
agatggcaaatctgttcaaatctctgcgatgataa
Entire Expressible Amino Acid Sequence for Nipah Virus Antigen with 60 mer Self-
Assembly (SEQ ID NO: 38)
MDWTWILFLVAAATRVHSGVTCAGRAIGNATAAQITAGVALYEAMKNADNINKLK
SSIESTNEAVVKLQETAEKTVYVLTALQDYINTNLVPTIDKISCKQTEASLDAALSKY
LSDLLYVFGPNLSDPVSNSMPIQAISQAFGGNYSTLLRTLGYAPEDFDDLLESDSITGQ
IIYVDLSSYYIIVRVYFPNGSGPLTKDIVIKMIPNVSNMSQCTGSVMENYKTRLNGILT
PIKGALEIYKNNCHDGGGSGGSGGSGGSGGGMQIYEGKLTAEGLRFGIVASRANHAL
VDRLVEGAIDAIVRHGGREEDITLVRVCGSWEIPVAAGELARKEDIDAVIAIGVLCRG
ATPSFDYIASEVSKGLADLSLELRKPITFGVITADTLEQAIEAAGTCHGNKGWEAALC
AIEMANLFKSLR
Influenza Construct 1-NC99_60 mer_pVax (DLnano_LS_HA(NC99); Entire expressible
nucleic acid sequence (SEQ ID NO: 57)
ggatccgccaccatggactggacctggattctgttcctggtggccgccgccacaagggtgcacagcatgcagatctacgaaggaaaa
ctgaccgctgagggactgaggttcggaattgtcgcaagccgcgcgaatcacgcactggtggataggctggtggaaggcgctatcga
cgcaattgtccggcacggcgggagagaggaagacatcacactggtgagagtctgcggcagctgggagattcccgtggcagctgga
gaactggctcgaaaggaggacatcgatgccgtgatcgctattggggtcctgtgccgaggagcaactcccagcttcgactacatcgcct
cagaagtgagcaaggggctggctgatctgtccctggagctgaggaaacctatcacttttggcgtgattactgccgacaccctggaaca
ggcaatcgaggcggccggcacctgccatggaaacaaaggctgggaagcagccctgtgcgctattgagatggcaaatctgttcaaat
ctctgcgaggaggctccggaggatctggagggagtggaggctcaggaggaggcgcccctctgcagctgggaaactgctccgtggc
aggatggattctgggcaatccagagtgtgagctgctgatctctaaggagtcctggtcttacatcgtggagaccccaaaccccgagaat
ggcacatgctttcccggctacttcgccgactatgaggagctgagggagcagctgagctccgtgtctagcttcgagagatttgagatctt
ccctaaggagtcctcttggccaaaccacaccgtgacaggcgtgagcgcctcctgttctcacaacggcaagagctccttttataggaatc
tgctgtggctgaccggcaagaacggcctgtaccctaatctgagcaagtcctatgtgaacaataaggagaaggaggtgctggtgctgtg
gggcgtgcaccaccctcccaacatcggcaatcagagggccctgtaccacaccgagaacgcctacgtgagcgtggtgtctagccact
acagcaggagattcacacccgagatcgccaagaggcctaaggtgcgcgaccaggagggacggatcaattactattggaccctgctg
gagccaggcgatacaatcatctttgaggccaacggcaatctgatcgccccctggtatgccttcgccctgtcccgcggctgataactcga
g
Entire expressible amino acid sequence (SEQ ID NO: 58)
MDWTWILFLVAAATRVHSMQIYEGKLTAEGLRFGIVASRANHALVDRLVEGAIDAI
VRHGGREEDITLVRVCGSWEIPVAAGELARKEDIDAVIAIGVLCRGATPSFDYIASEV
SKGLADLSLELRKPITFGVITADTLEQAIEAAGTCHGNKGWEAALCAIEMANLFKSLR
GGSGGSGGSGGSGGGAPLQLGNCSVAGWILGNPECELLISKESWSYIVETPNPENGT
CFPGYFADYEELREQLSSVSSFERFEIFPKESSWPNHTVTGVSASCSHNGKSSFYRNLL
WLTGKNGLYPNLSKSYVNNKEKEVLVLWGVHHPPNIGNQRALYHTENAYVSVVSS
HYSRRFTPEIAKRPKVRDQEGRINYYWTLLEPGDTIIFEANGNLIAPWYAFALSRG
Influenza Construct 2-NC99_g6_60 mer_pVax; Entire expressible nucleic acid sequence
(SEQ ID NO: 59)
ggatccgccaccatggactggacctggattctgttcctggtggccgccgccacaagggtgcacagcatgcagatctacgaaggaaaa
ctgaccgctgagggactgaggttcggaattgtcgcaagccgcgcgaatcacgcactggtggataggctggtggaaggcgctatcga
cgcaattgtccggcacggcgggagagaggaagacatcacactggtgagagtctgcggcagctgggagattcccgtggcagctgga
gaactggctcgaaaggaggacatcgatgccgtgatcgctattggggtcctgtgccgaggagcaactcccagcttcgactacatcgcct
cagaagtgagcaaggggctggctgatctgtccctggagctgaggaaacctatcacttttggcgtgattactgccgacaccctggaaca
ggcaatcgaggcggccggcacctgccatggaaacaaaggctgggaagcagccctgtgcgctattgagatggcaaatctgttcaaat
ctctgcgaggaggctccggaggatctggagggagtggaggctcaggaggaggcgcccctctgcagctgggaaactgcagcgtgg
caggatggattctgggcaatccagagtgtgagctgctgatctccaaggagtcctggtcttacatcgtggagaccccaaaccccgagaa
tggcacatgctttcccggcaacttctctgactatgaggagctgagggagcagctgagctccgtgtctagcttcgagagatttgagatctt
ccctaaggagtcctcttggccaaatcacaccgtgacaggcgtgagcgcctcctgttctcacaacggcaagagctccttttacaggaatc
tgctgtggctgaccggcaagaacggcctgtaccctaatctgagcaagtcctataacaatacaaaggagaaggaggtgctggtgctgtg
gggcgtgcaccaccctcccaacatcggcaatcagagggccctgtaccacaccgagaacgcctacgtgagcgtggtgtctagccact
actctaggagattcacacccaacatcagcaagaggcctaaggtgcgcgaccaggagggacggatcaattactattggaccctgctgg
agccaggcgatacaatcatctttgaggccaacggcaatctgatcgccccctggtatgccttcgccctgtctcgcggcaacggcagctg
ataactcgag
Entire expressible amino acid sequence (SEQ ID NO: 60)
MDWTWILFLVAAATRVHSMQIYEGKLTAEGLRFGIVASRANHALVDRLVEGAIDAI
VRHGGREEDITLVRVCGSWEIPVAAGELARKEDIDAVIAIGVLCRGATPSFDYIASEV
SKGLADLSLELRKPITFGVITADTLEQAIEAAGTCHGNKGWEAALCAIEMANLFKSLR
GGSGGSGGSGGSGGGAPLQLGNCSVAGWILGNPECELLISKESWSYIVETPNPENGT
CFPGNFSDYEELREQLSSVSSFERFEIFPKESSWPNHTVTGVSASCSHNGKSSFYRNLL
WLTGKNGLYPNLSKSYNNTKEKEVLVLWGVHHPPNIGNQRALYHTENAYVSVVSS
HYSRRFTPNISKRPKVRDQEGRINYYWTLLEPGDTIIFEANGNLIAPWYAFALSRGNG
S
Influenza Construct 3-CA09(175L)_Ferritin_pVax (DLnano_3BVE_HA(CA09)); Entire
expressible nucleic acid sequence (SEQ ID NO: 61)
atggactggacttggattctgtttctggtcgccgctgccactcgcgtgcattctgccccactgcacctgggcaagtgcaacatcgccgg
ctggattctgggcaatcccgagtgtgagagcctgtccaccgccagctcctggagctacatcgtggagaccccttctagcgacaacgg
cacatgctttccaggcgacttcatcgattatgaggagctgagggagcagctgtcctctgtgagctccttcgagagatttgagatcttcccc
aagacctctagctggcctaaccacgattccaataagggagtgacagcagcatgtcctcacgcaggcgccaagagcttttacaagaac
ctgatctggctggtgaagaagggcaattcctacccaaagctgtctaagagctatatcaacgacaagggcaaggaggtgctggtgctgt
ggggcatccaccacccatccacctctgccgaccagcagtctctgtaccagaatgccgatacatacgtgttcgtgggctcctctcggtac
tccaagaagttcaagccagagatcgccatcaggcccaaggtgagagaccaggagggccgcatgaattactattggacactggtgga
gcccggcgataagatcacctttgaggccacaggcaacctggtggtgcctcggtatgccttcgccatggagcgcaatgcaagcgggg
aaagccaggtgcgacagcagttctccaaagacatcgaaaagctgctgaatgaacaggtcaacaaggaaatgcagagcagcaacctg
tacatgtccatgagctcctggtgctatacccactctctggacggagcaggcctgttcctgtttgatcacgccgccgaggagtacgagca
cgccaagaagctgatcatcttcctgaatgagaacaatgtgcccgtgcagctgacctctatcagcgcccctgagcacaagttcgagggc
ctgacacagatctttcagaaggcctacgagcacgagcagcacatctccgagtctatcaacaatatcgtggaccacgccatcaagtcca
aggatcacgccacattcaactttctgcagtggtacgtggccgagcagcacgaggaggaggtgctgtttaaggacatcctggataagat
cgagctgatcggcaacgagaatcacgggctgtatctggccgaccagtatgtgaagggcatcgctaaaagcaggaaatcaggaagc
Entire expressible amino acid sequence (SEQ ID NO: 62)
MDWTWILFLVAAATRVHSAPLHLGKCNIAGWILGNPECESLSTASSWSYIVETPSSD
NGTCFPGDFIDYEELREQLSSVSSFERFEIFPKTSSWPNHDSNKGVTAACPHAGAKSF
YKNLIWLVKKGNSYPKLSKSYINDKGKEVLVLWGIHHPSTSADQQSLYQNADTYVF
VGSSRYSKKFKPEIAIRPKVRDQEGRMNYYWTLVEPGDKITFEATGNLVVPRYAFAM
ERNASGESQVRQQFSKDIEKLLNEQVNKEMQSSNLYMSMSSWCYTHSLDGAGLFLF
DHAAEEYEHAKKLIIFLNENNVPVQLTSISAPEHKFEGLTQIFQKAYEHEQHISESINNI
VDHAIKSKDHATFNFLQWYVAEQHEEEVLFKDILDKIELIGNENHGLYLADQYVKGI
AKSRKSGS
Influenza Construct 4-H1_CA04/09_FL_HA_3BVE_pVAX; Entire expressible nucleic acid
sequence (SEQ ID NO: 63)
atggactggacttggattctgttcctggtcgccgccgcaacccgcgtgcattctatgaaggctattctggtcgtgctgctgtatactttcgc
caccgccaacgccgacacactgtgcatcggctaccacgccaacaattctaccgacacagtggataccgtgctggagaagaatgtgac
cgtgacacacagcgtgaacctgctggaggataagcacaatggcaagctgtgcaagctgaggggagtggcaccactgcacctgggc
aagtgcaacatcgccggctggattctgggcaatcccgagtgtgagtccctgtctacagccagctcctggtcctacatcgtggagacac
cctctagcgacaacggcacatgctaccctggcgactttatcgattatgaggagctgcgggagcagctgagcagcgtgagcagcttcg
agaggttcgagatcttccccaagacctctagctggcctaaccacgatagcaataagggagtgacagcagcatgtccacacgcaggcg
ccaagagcttctataagaacctgatctggctggtgaagaagggcaattcctaccctaagctgagcaagtcctatatcaacgacaaggg
caaggaggtgctggtgctgtggggcatccaccacccatctaccagcgccgaccagcagtccctgtaccagaatgccgatacatacgt
gttcgtgggctcctctcggtactctaagaagttcaagccagagatcgccatcaggccaaaggtgagggaccaggagggacgcatga
actactattggaccctggtggagcccggcgataagatcacctttgaggccacaggcaacctggtggtgcctagatatgccttcgccatg
gagagaaatgccggctccggcatcatcatctctgacacccctgtgcacgattgcaacaccacatgtcagaccccaaagggcgccatc
aacacatccctgccttttcagaatatccacccaatcacaatcggcaagtgccctaagtacgtgaagagcaccaagctgaggctggcaa
caggcctgcgcaatatcccatctatccagagcaggggcctgtttggagcaatcgcaggcttcatcgagggaggatggaccggaatgg
tggacggctggtacggctatcaccaccagaacgagcagggcagcggatatgcagcagacctgaagtccacccagaatgccatcgat
gagatcacaaacaaggtcaattccgtgatcgagaagatgaacacccagtttacagccgtgggcaaggagttcaatcacctggagaag
agaatcgagaacctgaataagaaggtggacgatggcttcctggacatctggacctacaacgccgagctgctggtgctgctggagaat
gagaggacactggactaccacgattccaacgtgaagaatctgtatgagaaggtgagatctcagctgaagaacaatgccaaggagatc
ggcaacggctgcttcgagttttaccacaagtgcgacaacacctgtatggagagcgtgaagaatggcacatacgattatcctaagtattc
cgaggaggccaagctgaaccgcgaggagatcgactctggcggcgatatcatcaagctgctgaacgagcaagtgaataaggagatg
cagagctccaatctgtacatgtctatgtctagctggtgttatacccacagcctggacggagcaggcctgttcctgtttgatcacgccgcc
gaggagtacgagcacgccaagaagctgatcatctttctgaacgagaacaatgtgccagtgcagctgacctccatctctgcccccgag
cacaagtttgagggcctgacacagatcttccagaaggcctacgagcacgagcagcacatcagcgagtccatcaacaatatcgtggac
cacgccatcaagagcaaggatcacgccaccttcaactttctgcagtggtacgtggccgagcagcacgaggaggaggtgctgttcaag
gacatcctggataagatcgagctgatcggcaacgagaatcacgggctgtacctggcagaccagtatgtcaagggcatcgcaaagtca
cggaagagcgggagc
Entire expressible amino acid sequence (SEQ ID NO: 64)
MDWTWILFLVAAATRVHSMKAILVVLLYTFATANADTLCIGYHANNSTDTVDTVLE
KNVTVTHSVNLLEDKHNGKLCKLRGVAPLHLGKCNIAGWILGNPECESLSTASSWS
YIVETPSSDNGTCYPGDFIDYEELREQLSSVSSFERFEIFPKTSSWPNHDSNKGVTAAC
PHAGAKSFYKNLIWLVKKGNSYPKLSKSYINDKGKEVLVLWGIHHPSTSADQQSLY
QNADTYVFVGSSRYSKKFKPEIAIRPKVRDQEGRMNYYWTLVEPGDKITFEATGNLV
VPRYAFAMERNAGSGIIISDTPVHDCNTTCQTPKGAINTSLPFQNIHPITIGKCPKYVKS
TKLRLATGLRNIPSIQSRGLFGAIAGFIEGGWTGMVDGWYGYHHQNEQGSGYAADL
KSTQNAIDEITNKVNSVIEKMNTQFTAVGKEFNHLEKRIENLNKKVDDGFLDIWTYN
AELLVLLENERTLDYHDSNVKNLYEKVRSQLKNNAKEIGNGCFEFYHKCDNTCMES
VKNGTYDYPKYSEEAKLNREEIDSGGDIIKLLNEQVNKEMQSSNLYMSMSSWCYTH
SLDGAGLFLFDHAAEEYEHAKKLIIFLNENNVPVQLTSISAPEHKFEGLTQIFQKA YEH
EQHISESINNIVDHAIKSKDHATFNFLQWYVAEQHEEEVLFKDILDKIELIGNENHGLY
LADQYVKGIAKSRKSGS
HIV antigen-GT8_180 mer_pVAX (DLnano_PfV_GT8); Entire expressible nucleic acid
sequence (SEQ ID NO: 68)
ctgagcattgcccccacactgattaaccgggacaaaccctacaccaaagaggaactgatggagattctgagactggctattatcgctg
agctggacgccatcaacctgtacgagcagatggcccggtattctgaggacgagaatgtgcgcaagatcctgctggatgtggccagg
gaggagaaggcacacgtgggagagttcatggccctgctgctgaacctggaccccgagcaggtgaccgagctgaagggcggctttg
aggaggtgaaggagctgacaggcatcgaggcccacatcaacgacaataagaaggaggagagcaacgtggagtatttcgagaagct
gagatccgccctgctggatggcgtgaataagggcaggagcctgctgaagcacctgcctgtgaccaggatcgagggccagagcttca
gagtggacatcatcaagtttgaggatggcgtgcgcgtggtgaagcaggagtacaagcccatccctctgctgaagaagaagttctacgt
gggcatcagggagctgaacgacggcacctacgatgtgagcatcgccacaaaggccggcgagctgctggtgaaggacgaggagtc
cctggtcatccgcgagatcctgtctacagagggcatcaagaagatgaagctgagctcctgggacaatccagaggaggccctgaacg
atctgatgaatgccctgcaggaggcatctaacgcaagcgccggaccattcggcctgatcatcaatcccaagagatacgccaagctgct
gaagatctatgagaagtccggcaagatgctggtggaggtgctgaaggagatcttccggggcggcatcatcgtgaccctgaacatcga
tgagaacaaagtgatcatctttgccaacacccctgccgtgctggacgtggtggtgggacaggatgtgacactgcaggagctgggacc
agagggcgacgatgtggcctttctggtgtccgaggccatcggcatcaggatcaagaatccagaggcaatcgtggtgctggagggcg
gctctggcggaagtggcggaagtgggggaagtggaggcggcggaagcgggggaggcagcgggggaggggacaccatcacac
tgccatgccgccctgcaccacctccacattgtagctccaacatcaccggcctgattctgacaagacaggggggatatagtaacgataat
accgtgattttcaggccctcaggaggggactggagggacatcgcacgatgccagattgctggaacagtggtctctactcagctgtttct
gaacggcagtctggctgaggaagaggtggtcatccgatctgaagactggcgggataatgcaaagtcaatttgtgtgcagctgaacac
aagcgtcgagatcaattgcactggcgcagggcactgtaacatttctcgggccaaatggaacaataccctgaagcagatcgccagtaa
actgagagagcagtacggcaataagacaatcatcttcaagccttctagtggaggcgacccagagttcgtgaaccatagctttaattgcg
ggggagagttcttttattgtgattccacacagctgttcaacagcacttggtttaattccacc
Entire expressible amino acid sequence (SEQ ID NO: 69)
LSIAPTLINRDKPYTKEELMEILRLAIIAELDAINLYEQMARYSEDENVRKILLDVARE
EKAHVGEFMALLLNLDPEQVTELKGGFEEVKELTGIEAHINDNKKEESNVEYFEKLR
SALLDGVNKGRSLLKHLPVTRIEGQSFRVDIIKFEDGVRVVKQEYKPIPLLKKKFYVG
IRELNDGTYDVSIATKAGELLVKDEESLVIREILSTEGIKKMKLSSWDNPEEALNDLM
NALQEASNASAGPFGLIINPKRYAKLLKIYEKSGKMLVEVLKEIFRGGIMQDIFIEDLN
IDENKVIIFANTPAVLDVVVGQDVTLQELGPEGDDVAFLVSEAIGIRIKNPEAIVVLEG
GSGGSGGSGGSGGGGSGGGSGGGDTITLPCRPAPPPHCSSNITGLILTRQGGYSNDNT
VIFRPSGGDWRDIARCQIAGTVVSTQLFLNGSLAEEEVVIRSEDWRDNAKSICVQLNT
SVEINCTGAGHCNISRAKWNNTLKQIASKLREQYGNKTIIFKPSSGGDPEFVNHSFNC
GGEFFYCDSTQLFNSTWFNST
LS_HA_CA09 (DLnano_CD4MutLS_GT8)-Entire expressible nucleic acid sequence (SEQ
ID NO: 70)
atgcagatctacgaaggaaaactgaccgctgagggactgaggttcggaattgtcgcaagccgcgcgaatcacgcactggtggatag
gctggtggaaggcgctatcgacgcaattgtccggcacggcgggagagaggaagacatcacactggtgagagtctgcggcagctgg
gagattcccgtggcagctggagaactggctcgaaaggaggacatcgatgccgtgatcgctattggggtcctgtgccgaggagcaact
cccagcttcgactacatcgcctcagaagtgagcaaggggctggctgatctgtccctggagctgaggaaacctatcacttttggcgtgat
tactgccgacaccctggaacaggcaatcgaggcggccggcacctgccatggaaacaaaggctgggaagcagccctgtgcgctatt
gagatggcaaatctgttcaaatctctgcgaggaggctccggaggatctggagggagtggaggctcaggaggaggcgccccactgc
acctgggcaagtgcaacatcgccggctggattctgggcaatcccgagtgtgagagcctgtccaccgccagctcctggagctacatcg
tggagaccccttctagcgacaacggcacatgctttccaggcgacttcatcgattatgaggagctgagggagcagctgtcctctgtgag
ctccttcgagagatttgagatcttccccaagacctctagctggcctaaccacgattccaataagggagtgacagcagcatgtcctcacg
caggcgccaagagcttttacaagaacctgatctggctggtgaagaagggcaattcctacccaaagctgtctaagagctatatcaacga
caagggcaaggaggtgctggtgctgtggggcatccaccacccatccacctctgccgaccagcagtctatctaccagaatgccgatac
atacgtgttcgtgggctcctctcggtactccaagaagttcaagccagagatcgccatcaggcccaaggtgagagaccaggagggcc
gcatgaattactattggacactggtggagcccggcgataagatcacctttgaggccacaggcaacctggtggtgcctcggtatgccttc
gccatggagcgcaat
Entire expressible amino acid sequence (SEQ ID NO: 71)
MQIYEGKLTAEGLRFGIVASRANHALVDRLVEGAIDAIVRHGGREEDITLVRVCGSW
EIPVAAGELARKEDIDAVIAIGVLCRGATPSFDYIASEVSKGLADLSLELRKPITFGVIT
ADTLEQAIEAAGTCHGNKGWEAALCAIEMANLFKSLRGGSGGSGGSGGSGGGAPLH
LGKCNIAGWILGNPECESLSTASSWSYIVETPSSDNGTCFPGDFIDYEELREQLSSVSSF
ERFEIFPKTSSWPNHDSNKGVTAACPHAGAKSFYKNLIWLVKKGNSYPKLSKSYIND
KGKEVLVLWGIHHPSTSADQQSIYQNADTYVFVGSSRYSKKFKPEIAIRPKVRDQEG
RMNYYWTLVEPGDKITFEATGNLVVPRYAFAMERN
GT8_7 mer-pVAX (DL_GT8_IMX313P)-Entire expressible nucleic acid sequence (SEQ ID
NO: 72)
atggactggacctggattctgttcctggtggccgccgccacaagggtgcacagcgacaccatcacactgccatgccgccctgcacca
cctccacattgtagctccaacatcaccggcctgattctgacaagacaggggggatatagtaacgataataccgtgattttcaggccctca
ggaggggactggagggacatcgcacgatgccagattgctggaacagtggtctctactcagctgtttctgaacggcagtctggctgag
gaagaggtggtcatccgatctgaagactggcgggataatgcaaagtcaatttgtgtgcagctgaacacaagcgtcgagatcaattgca
ctggcgcagggcactgtaacatttctcgggccaaatggaacaataccctgaagcagatcgccagtaaactgagagagcagtacggc
aataagacaatcatcttcaagccttctagtggaggcgacccagagttcgtgaaccatagctttaattgcgggggagagttcttttattgtg
attccacacagctgttcaacagcacttggtttaattccaccggcgggagcggagggagtggcggatctggcgggagcggcggaggc
aaaaaacagggggatgctgatgtctgcggagaggtggcttatatccagagcgtggtgtccgactgccacgtgccaaccgcagagct
gcggacactgctggagatcagaaaactgttcctggagattcagaaactgaaagtcgagctgcaggggctgtcaaaagaa
Entire expressible amino acid sequence (SEQ ID NO: 73)
MDWTWILFLVAAATRVHSMDTITLPCRPAPPPHCSSNITGLILTRQGGYSNDNTVIFR
PSGGDWRDIARCQIAGTVVSTQLFLNGSLAEEEVVIRSEDWRDNAKSICVQLNTSVEI
NCTGAGHCNISRAKWNNTLKQIASKLREQYGNKTIIFKPSSGGDPEFVNHSFNCGGEF
FYCDSTQLFNSTWFNSTGGSGGSGGSGGSGGGKKQGDADVCGEVAYIQSVVSDCHV
PTAELRTLLEIRKLFLEIQKLKVELQGLSKE
GT8_CD4Mut1LS-60 mer (DLnano_CD4MutLS_GT8)-Entire expressible nucleic acid
sequence (SEQ ID NO: 74)
Ggatccgccaccatggactggacctggattctgttcctggtggccgccgccacaagggtgcacagcatgcagatctacgaaggaaa
actgaccgctgagggactgaagttcggcatcgtgggcagcaggtttaaccacggcctggtggataggctggtggaaggcgctatcg
acgcaattgtccggcacggcgggagagaggaagacatcacactggtgagagtctgcggcagctgggagattcccgtggcagctgg
agaactggctcgaaaggaggacatcgatgccgtgatcgctattggggtcctgtgccgaggagcaactcccagcttcgactacatcgc
ctcagaagtgagcaaggggctggctgatctgtccctggagctgaggaaacctatcacttttggcgtgattactgccgacaccctggaa
caggcaatcgaggcggccggcacctgccatggaaacaaaggctgggaagcagccctgtgcgctattgagatggcaaatctgttcaa
atctctgcgaggaggctccggaggatctggagggagtggaggctcaggaggaggcgacaccatcacactgccatgccgccctgca
ccacctccacattgtagctccaacatcaccggcctgattctgacaagacaggggggatatagtaacgataataccgtgattttcaggcc
ctcaggaggggactggagggacatcgcacgatgccagattgctggaacagtggtctctactcagctgtttctgaacggcagtctggct
gaggaagaggtggtcatccgatctgaagactggcgggataatgcaaagtcaatttgtgtgcagctgaacacaagcgtcgagatcaatt
gcactggcgcagggcactgtaacatttctcgggccaaatggaacaataccctgaagcagatcgccagtaaactgagagagcagtac
ggcaataagacaatcatcttcaagccttctagtggaggcgacccagagttcgtgaaccatagctttaattgcgggggagagttcttttatt
gtgattccacacagctgttcaacagcacttggtttaattccacctgataactcgag
Entire expressible amino acid sequence (SEQ ID NO: 75)
MDWTWILFLVAAATRVHSMQIYEGKLTAEGLKFGIVGSRFNHGLVDRLVEGAIDAI
VRHGGREEDITLVRVCGSWEIPVAAGELARKEDIDAVIAIGVLCRGATPSEDYIASEV
SKGLADLSLELRKPITFGVITADTLEQAIEAAGTCHGNKGWEAALCAIEMANLFKSLR
GGSGGSGGSGGSGGGDTITLPCRPAPPPHCSSNITGLILTRQGGYSNDNTVIFRPSGGD
WRDIARCQIAGTVVSTQLFLNGSLAEEEVVIRSEDWRDNAKSICVQLNTSVEINCTGA
GHCNISRAKWNNTLKQIASKLREQYGNKTIIFKPSSGGDPEFVNHSFNCGGEFFYCDS
TQLFNSTWFNST
Construct LS_GT8 without IgE-Entire expressible nucleic acid sequence (SEQ ID NO: 83)
atgcagatctacgaaggaaaactgaccgctgagggactgaggttcggaattgtcgcaagccgcgcgaatcacgcactggtggatag
gctggtggaaggcgctatcgacgcaattgtccggcacggcgggagagaggaagacatcacactggtgagagtctgcggcagctgg
gagattcccgtggcagctggagaactggctcgaaaggaggacatcgatgccgtgatcgctattggggtcctgtgccgaggagcaact
cccagcttcgactacatcgcctcagaagtgagcaaggggctggctgatctgtccctggagctgaggaaacctatcacttttggcgtgat
tactgccgacaccctggaacaggcaatcgaggcggccggcacctgccatggaaacaaaggctgggaagcagccctgtgcgctatt
gagatggcaaatctgttcaaatctctgcgaggaggctccggaggatctggagggagtggaggctcaggaggaggcgacaccatca
cactgccatgccgccctgcaccacctccacattgtagctccaacatcaccggcctgattctgacaagacaggggggatatagtaacga
taataccgtgattttcaggccctcaggaggggactggagggacatcgcacgatgccagattgctggaacagtggtctctactcagctgt
ttctgaacggcagtctggctgaggaagaggtggtcatccgatctgaagactggcgggataatgcaaagtcaatttgtgtgcagctgaa
cacaagcgtcgagatcaattgcactggcgcagggcactgtaacatttctcgggccaaatggaacaataccctgaagcagatcgccag
taaactgagagagcagtacggcaataagacaatcatcttcaagccttctagtggaggcgacccagagttcgtgaaccatagctttaatt
gcgggggagagttcttttattgtgattccacacagctgttcaacagcacttggtttaattccacctgataa
Entire expressible amino acid sequence (SEQ ID NO: 84)
MQIYEGKLTAEGLRFGIVASRANHALVDRLVEGAIDAIVRHGGREEDITLVRVCGSW
EIPVAAGELARKEDIDAVIAIGVLCRGATPSFDYIASEVSKGLADLSLELRKPITFGVIT
ADTLEQAIEAAGTCHGNKGWEAALCAIEMANLFKSLRGGSGGSGGSGGSGGGDTIT
LPCRPAPPPHCSSNITGLILTRQGGYSNDNTVIFRPSGGDWRDIARCQIAGTVVSTQLF
LNGSLAEEEVVIRSEDWRDNAKSICVQLNTSVEINCTGAGHCNISRAKWNNTLKQIA
SKLREQYGNKTIIFKPSSGGDPEFVNHSFNCGGEFFYCDSTQLFNSTWENST
Construct GLT1-3BVE-Entire expressible nucleic acid sequence (SEQ ID NO: 85)
gacaccatcacactgccatgccgccctgcaccacctccacattgtagctccaacatcaccggcctgattctgacaagacaggggggat
atagtaacgataataccgtgattttcaggccctcaggaggggactggagggacatcgcacgatgccagattgctggaacagtggtctc
tactcagctgtttctgaacggcagtctggctgaggaagaggtggtcatccgatctgaagactggcgggataatgcaaagtcaatttgtgt
gcagctgaacacaagcgtcgagatcaattgcactggcgcagggcactgtaacatttctcgggccaaatggaacaataccctgaagca
gatcgccagtaaactgagagagcagtacggcaataagacaatcatcttcaagccttctagtggaggcgacccagagttcgtgaaccat
agctttaattgcgggggagagttcttttattgtgattccacacagctgttcaacagcacttggtttaattccaccggcggaagcggcggaa
gcggcgggtctgggctgagtaaggacattatcaagctgctgaacgaacaggtgaacaaagagatgcagtctagcaacctgtacatgt
ccatgagctcctggtgctatacccactctctggacggagcaggcctgttcctgtttgatcacgccgccgaggagtacgagcacgccaa
gaagctgatcatcttcctgaatgagaacaatgtgcccgtgcagctgacctctatcagcgcccctgagcacaagttcgagggcctgaca
cagatctttcagaaggcctacgagcacgagcagcacatctccgagtctatcaacaatatcgtggaccacgccatcaagtccaaggatc
acgccacattcaactttctgcagtggtacgtggccgagcagcacgaggaggaggtgctgtttaaggacatcctggataagatcgagct
gatcggcaatgagaaccacgggctgtacctggcagatcagtatgtcaagggcatcgctaagtcaaggaaaagctgataa
Entire expressible amino acid sequence (SEQ ID NO: 86)
DTITLPCRPAPPPHCSSNITGLILTRQGGYSNDNTVIFRPSGGDWRDIARCQIAGTVVS
TQLFLNGSLAEEEVVIRSEDWRDNAKSICVQLNTSVEINCTGAGHCNISRAKWNNTL
KQIASKLREQYGNKTIIFKPSSGGDPEFVNHSFNCGGEFFYCDSTQLFNSTWENSTGG
SGGSGGSGLSKDIIKLLNEQVNKEMQSSNLYMSMSSWCYTHSLDGAGLFLFDHAAE
EYEHAKKLIIFLNENNVPVQLTSISAPEHKFEGLTQIFQKAYEHEQHISESINNIVDHAI
KSKDHATFNFLQWYVAEQHEEEVLFKDILDKIELIGNENHGLYLADQYVKGIAKSRK
S
Construct GLT1-13-Entire expressible nucleic acid sequence (SEQ ID NO: 87)
gacaccatcacactgccatgccgccctgcaccacctccacattgtagctccaacatcaccggcctgattctgacaagacaggggggat
atagtaacgataataccgtgattttcaggccctcaggaggggactggagggacatcgcacgatgccagattgctggaacagtggtctc
tactcagctgtttctgaacggcagtctggctgaggaagaggtggtcatccgatctgaagactggcgggataatgcaaagtcaatttgtgt
gcagctgaacacaagcgtcgagatcaattgcactggcgcagggcactgtaacatttctcgggccaaatggaacaataccctgaagca
gatcgccagtaaactgagagagcagtacggcaataagacaatcatcttcaagccttctagtggaggcgacccagagttcgtgaaccat
agctttaattgcgggggagagttcttttattgtgattccacacagctgttcaacagcacttggtttaattccaccggcggcagcggcggca
gcggcgggagcggaggaagtgagaaagcagccaaagcagaggaagcagcacggaagatggaagaactgttcaagaagcacaa
gatcgtggccgtgctgagggccaactccgtggaggaggccaagaagaaggccctggccgtgttcctgggcggcgtgcacctgatc
gagatcacctttacagtgcccgacgccgataccgtgatcaaggagctgtctttcctgaaggagatgggagcaatcatcggagcagga
accgtgacaagcgtggagcagtgcagaaaggccgtggagagcggcgccgagtttatcgtgtcccctcacctggacgaggagatctc
tcagttctgtaaggagaagggcgtgttttacatgccaggcgtgatgacccccacagagctggtgaaggccatgaagctgggccacac
aatcctgaagctgttccctggcgaggtggtgggcccacagtttgtgaaggccatgaagggccccttccctaatgtgaagtttgtgccca
ccggcggcgtgaacctggataacgtgtgcgagtggttcaaggcaggcgtgctggcagtgggcgtgggcagcgccctggtgaaggg
cacacccgtggaagtcgctgagaaggcaaaggcattcgtggaaaagattagggggtgtactgagtgataa
Entire expressible amino acid sequence (SEQ ID NO: 88)
DTITLPCRPAPPPHCSSNITGLILTRQGGYSNDNTVIFRPSGGDWRDIARCQIAGTVVS
TQLFLNGSLAEEEVVIRSEDWRDNAKSICVQLNTSVEINCTGAGHCNISRAKWNNTL
KQIASKLREQYGNKTIIFKPSSGGDPEFVNHSFNCGGEFFYCDSTQLFNSTWENSTGG
SGGSGGSGGSEKAAKAEEAARKMEELFKKHKIVAVLRANSVEEAKKKALAVFLGG
VHLIEITFTVPDADTVIKELSFLKEMGAIIGAGTVTSVEQCRKAVESGAEFIVSPHLDE
EISQFCKEKGVFYMPGVMTPTELVKAMKLGHTILKLFPGEVVGPQFVKAMKGPFPN
VKFVPTGGVNLDNVCEWFKAGVLAVGVGSALVKGTPVEVAEKAKAFVEKIRGCTE
Construct NivFtop_stab2_gMax_Nt_60 mer without IgE-Entire expressible nucleic acid
sequence (SEQ ID NO: 89)
atgcagatctacgaaggaaaactgaccgctgagggactgaggttcggaattgtcgcaagccgcgcgaatcacgcactggtggatag
gctggtggaaggcgctatcgacgcaattgtccggcacggcgggagagaggaagacatcacactggtgagagtctgcggcagctgg
gagattcccgtggcagctggagaactggctcgaaaggaggacatcgatgccgtgatcgctattggggtcctgtgccgaggagcaact
cccagcttcgactacatcgcctcagaagtgagcaaggggctggctgatctgtccctggagctgaggaaacctatcacttttggcgtgat
tactgccgacaccctggaacaggcaatcgaggcggccggcacctgccatggaaacaaaggctgggaagcagccctgtgcgctatt
gagatggcaaatctgttcaaatctctgcgaggaggctccggaggatctggagggagtggaggctcaggaggaggcggggtcacttg
tgccggacgagccatcggaaatgctaccgccgcccagattactgccggagtcgccctgtatgaagccatgaagaatgccgacaacat
caataagctgaagagctccatcgagagcaccaacgaggccgtggtgaagctgcaggagacagccgagaagacagtgtacgtgctg
acagccctgcaggactatatcaacaccaatctggtgcccacaatcgataagatcagctgcaagcagaccgaggcatccctggacgcc
gccctgtccaagtacctgtctgatctgctgtacgtgttcggccccaacctgagcgaccccgtgagcaattctatgcctatccaggccatc
tctcaggccttcggcggcaactacagcaccctgctgaggacactgggctatgccccagaggactttgacgatctgctggagagcgatt
ccatcacaggccagatcatctacgtggacctgtctagctactatatcatcgtgagagtgtattttccaaatggctccggccccctgaccaa
ggatatcgtgatcaagatgatccccaacgtgtctaatatgagccagtgtacaggctctgtgatggagaactacaagaccaggctgaatg
gcatcctgacacctatcaagggcgccctggagatctataagaataactgtcacgatggatgataa
Entire expressible amino acid sequence (SEQ ID NO: 90)
MQIYEGKLTAEGLRFGIVASRANHALVDRLVEGAIDAIVRHGGREEDITLVRVCGSW
EIPVAAGELARKEDIDAVIAIGVLCRGATPSFDYIASEVSKGLADLSLELRKPITFGVIT
ADTLEQAIEAAGTCHGNKGWEAALCAIEMANLFKSLRGGSGGSGGSGGSGGGGVTC
AGRAIGNATAAQITAGVALYEAMKNADNINKLKSSIESTNEAVVKLQETAEKTVYV
LTALQDYINTNLVPTIDKISCKQTEASLDAALSKYLSDLLYVFGPNLSDPVSNSMPIQA
ISQAFGGNYSTLLRTLGYAPEDFDDLLESDSITGQIIYVDLSSYYIIVRVYFPNGSGPLT
KDIVIKMIPNVSNMSQCTGSVMENYKTRLNGILTPIKGALEIYKNNCHDG
Construct NivFtop_stab2_gMax_Ct_60 mer without IgE-Entire expressible nucleic acid
sequence (SEQ ID NO: 91)
ggggtcacttgtgccggacgagccatcggaaatgctaccgccgcccagattactgccggagtcgccctgtatgaagccatgaagaat
gccgacaacatcaataagctgaagagctccatcgagagcaccaacgaggccgtggtgaagctgcaggagacagccgagaagaca
gtgtacgtgctgacagccctgcaggactatatcaacaccaatctggtgcccacaatcgataagatcagctgcaagcagaccgaggcat
ccctggacgccgccctgtccaagtacctgtctgatctgctgtacgtgttcggccccaacctgagcgaccccgtgagcaattctatgcct
atccaggccatctctcaggccttcggcggcaactacagcaccctgctgaggacactgggctatgccccagaggactttgacgatctgc
tggagagcgattccatcacaggccagatcatctacgtggacctgtctagctactatatcatcgtgagagtgtattttccaaatggctccgg
ccccctgaccaaggatatcgtgatcaagatgatccccaacgtgtctaatatgagccagtgtacaggctctgtgatggagaactacaaga
ccaggctgaatggcatcctgacacctatcaagggcgccctggagatctataagaataactgtcacgatggaggaggctccggaggat
ctggagggagtggaggctcaggaggaggcatgcagatctacgaaggaaaactgaccgctgagggactgaggttcggaattgtcgc
aagccgcgcgaatcacgcactggtggataggctggtggaaggcgctatcgacgcaattgtccggcacggcgggagagaggaaga
catcacactggtgagagtctgcggcagctgggagattcccgtggcagctggagaactggctcgaaaggaggacatcgatgccgtga
tcgctattggggtcctgtgccgaggagcaactcccagcttcgactacatcgcctcagaagtgagcaaggggctggctgatctgtccct
ggagctgaggaaacctatcacttttggcgtgattactgccgacaccctggaacaggcaatcgaggcggccggcacctgccatggaaa
caaaggctgggaagcagccctgtgcgctattgagatggcaaatctgttcaaatctctgcgatgataa
Entire expressible amino acid sequence (SEQ ID NO: 92)
GVTCAGRAIGNATAAQITAGVALYEAMKNADNINKLKSSIESTNEAVVKLQETAEKT
VYVLTALQDYINTNLVPTIDKISCKQTEASLDAALSKYLSDLLYVFGPNLSDPVSNSM
PIQAISQAFGGNYSTLLRTLGYAPEDFDDLLESDSITGQIIYVDLSSYYIIVRVYFPNGS
GPLTKDIVIKMIPNVSNMSQCTGSVMENYKTRLNGILTPIKGALEIYKNNCHDGGGSG
GSGGSGGSGGGMQIYEGKLTAEGLRFGIVASRANHALVDRLVEGAIDAIVRHGGREE
DITLVRVCGSWEIPVAAGELARKEDIDAVIAIGVLCRGATPSFDYIASEVSKGLADLSL
ELRKPITFGVITADTLEQAIEAAGTCHGNKGWEAALCAIEMANLFKSLR
Construct NC99_60 mer_pVax without IgE-Entire expressible nucleic acid sequence (SEQ
ID NO: 93)
atgcagatctacgaaggaaaactgaccgctgagggactgaggttcggaattgtcgcaagccgcgcgaatcacgcactggtggatag
gctggtggaaggcgctatcgacgcaattgtccggcacggcgggagagaggaagacatcacactggtgagagtctgcggcagctgg
gagattcccgtggcagctggagaactggctcgaaaggaggacatcgatgccgtgatcgctattggggtcctgtgccgaggagcaact
cccagcttcgactacatcgcctcagaagtgagcaaggggctggctgatctgtccctggagctgaggaaacctatcacttttggcgtgat
tactgccgacaccctggaacaggcaatcgaggcggccggcacctgccatggaaacaaaggctgggaagcagccctgtgcgctatt
gagatggcaaatctgttcaaatctctgcgaggaggctccggaggatctggagggagtggaggctcaggaggaggcgcccctctgca
gctgggaaactgctccgtggcaggatggattctgggcaatccagagtgtgagctgctgatctctaaggagtcctggtcttacatcgtgg
agaccccaaaccccgagaatggcacatgctttcccggctacttcgccgactatgaggagctgagggagcagctgagctccgtgtcta
gcttcgagagatttgagatcttccctaaggagtcctcttggccaaaccacaccgtgacaggcgtgagcgcctcctgttctcacaacggc
aagagctccttttataggaatctgctgtggctgaccggcaagaacggcctgtaccctaatctgagcaagtcctatgtgaacaataagga
gaaggaggtgctggtgctgtggggcgtgcaccaccctcccaacatcggcaatcagagggccctgtaccacaccgagaacgcctac
gtgagcgtggtgtctagccactacagcaggagattcacacccgagatcgccaagaggcctaaggtgcgcgaccaggagggacgga
tcaattactattggaccctgctggagccaggcgatacaatcatctttgaggccaacggcaatctgatcgccccctggtatgccttcgccc
tgtcccgcggctga
Entire expressible amino acid sequence (SEQ ID NO: 94)
MQIYEGKLTAEGLRFGIVASRANHALVDRLVEGAIDAIVRHGGREEDITLVRVCGSW
EIPVAAGELARKEDIDAVIAIGVLCRGATPSFDYIASEVSKGLADLSLELRKPITFGVIT
ADTLEQAIEAAGTCHGNKGWEAALCAIEMANLFKSLRGGSGGSGGSGGSGGGAPLQ
LGNCSVAGWILGNPECELLISKESWSYIVETPNPENGTCFPGYFADYEELREQLSSVSS
FERFEIFPKESSWPNHTVTGVSASCSHNGKSSFYRNLLWLTGKNGLYPNLSKSYVNN
KEKEVLVLWGVHHPPNIGNQRALYHTENAYVSVVSSHYSRRFTPEIAKRPKVRDQE
GRINYYWTLLEPGDTIIFEANGNLIAPWYAFALSRG
Construct NC99_g6_60 mer_pVax without IgE-Entire expressible nucleic acid sequence
(SEQ ID NO: 95)
atgcagatctacgaaggaaaactgaccgctgagggactgaggttcggaattgtcgcaagccgcgcgaatcacgcactggtggatag
gctggtggaaggcgctatcgacgcaattgtccggcacggcgggagagaggaagacatcacactggtgagagtctgcggcagctgg
gagattcccgtggcagctggagaactggctcgaaaggaggacatcgatgccgtgatcgctattggggtcctgtgccgaggagcaact
cccagcttcgactacatcgcctcagaagtgagcaaggggctggctgatctgtccctggagctgaggaaacctatcacttttggcgtgat
tactgccgacaccctggaacaggcaatcgaggcggccggcacctgccatggaaacaaaggctgggaagcagccctgtgcgctatt
gagatggcaaatctgttcaaatctctgcgaggaggctccggaggatctggagggagtggaggctcaggaggaggcgcccctctgca
gctgggaaactgcagcgtggcaggatggattctgggcaatccagagtgtgagctgctgatctccaaggagtcctggtcttacatcgtg
gagaccccaaaccccgagaatggcacatgctttcccggcaacttctctgactatgaggagctgagggagcagctgagctccgtgtcta
gcttcgagagatttgagatcttccctaaggagtcctcttggccaaatcacaccgtgacaggcgtgagcgcctcctgttctcacaacggc
aagagctccttttacaggaatctgctgtggctgaccggcaagaacggcctgtaccctaatctgagcaagtcctataacaatacaaagga
gaaggaggtgctggtgctgtggggcgtgcaccaccctcccaacatcggcaatcagagggccctgtaccacaccgagaacgcctac
gtgagcgtggtgtctagccactactctaggagattcacacccaacatcagcaagaggcctaaggtgcgcgaccaggagggacggat
caattactattggaccctgctggagccaggcgatacaatcatctttgaggccaacggcaatctgatcgccccctggtatgccttcgccct
gtctcgcggcaacggcagctga
Entire expressible amino acid sequence (SEQ ID NO: 96)
MQIYEGKLTAEGLRFGIVASRANHALVDRLVEGAIDAIVRHGGREEDITLVRVCGSW
EIPVAAGELARKEDIDAVIAIGVLCRGATPSFDYIASEVSKGLADLSLELRKPITFGVIT
ADTLEQAIEAAGTCHGNKGWEAALCAIEMANLFKSLRGGSGGSGGSGGSGGGAPLQ
LGNCSVAGWILGNPECELLISKESWSYIVETPNPENGTCFPGNFSDYEELREQLSSVSS
FERFEIFPKESSWPNHTVTGVSASCSHNGKSSFYRNLLWLTGKNGLYPNLSKSYNNT
KEKEVLVLWGVHHPPNIGNQRALYHTENAYVSVVSSHYSRRFTPNISKRPKVRDQE
GRINYYWTLLEPGDTIIFEANGNLIAPWYAFALSRGNGS
Construct CA09(175L)_Ferritin_pVax without IgE-Entire expressible nucleic acid
sequence (SEQ ID NO: 97)
gccccactgcacctgggcaagtgcaacatcgccggctggattctgggcaatcccgagtgtgagagcctgtccaccgccagctcctgg
agctacatcgtggagaccccttctagcgacaacggcacatgctttccaggcgacttcatcgattatgaggagctgagggagcagctgt
cctctgtgagctccttcgagagatttgagatcttccccaagacctctagctggcctaaccacgattccaataagggagtgacagcagcat
gtcctcacgcaggcgccaagagcttttacaagaacctgatctggctggtgaagaagggcaattcctacccaaagctgtctaagagctat
atcaacgacaagggcaaggaggtgctggtgctgtggggcatccaccacccatccacctctgccgaccagcagtctctgtaccagaat
gccgatacatacgtgttcgtgggctcctctcggtactccaagaagttcaagccagagatcgccatcaggcccaaggtgagagaccag
gagggccgcatgaattactattggacactggtggagcccggcgataagatcacctttgaggccacaggcaacctggtggtgcctcgg
tatgccttcgccatggagcgcaatgcaagcggggaaagccaggtgcgacagcagttctccaaagacatcgaaaagctgctgaatga
acaggtcaacaaggaaatgcagagcagcaacctgtacatgtccatgagctcctggtgctatacccactctctggacggagcaggcct
gttcctgtttgatcacgccgccgaggagtacgagcacgccaagaagctgatcatcttcctgaatgagaacaatgtgcccgtgcagctg
acctctatcagcgcccctgagcacaagttcgagggcctgacacagatctttcagaaggcctacgagcacgagcagcacatctccgag
tctatcaacaatatcgtggaccacgccatcaagtccaaggatcacgccacattcaactttctgcagtggtacgtggccgagcagcacga
ggaggaggtgctgtttaaggacatcctggataagatcgagctgatcggcaacgagaatcacgggctgtatctggccgaccagtatgtg
aagggcatcgctaaaagcaggaaatcaggaagc
Entire expressible amino acid sequence (SEQ ID NO: 98)
APLHLGKCNIAGWILGNPECESLSTASSWSYIVETPSSDNGTCFPGDFIDYEELREQLS
SVSSFERFEIFPKTSSWPNHDSNKGVTAACPHAGAKSFYKNLIWLVKKGNSYPKLSK
SYINDKGKEVLVLWGIHHPSTSADQQSLYQNADTYVFVGSSRYSKKFKPEIAIRPKVR
DQEGRMNYYWTLVEPGDKITFEATGNLVVPRYAFAMERNASGESQVRQQFSKDIEK
LLNEQVNKEMQSSNLYMSMSSWCYTHSLDGAGLFLFDHAAEEYEHAKKLIIFLNEN
NVPVQLTSISAPEHKFEGLTQIFQKAYEHEQHISESINNIVDHAIKSKDHATFNFLQWY
VAEQHEEEVLFKDILDKIELIGNENHGLYLADQYVKGIAKSRKSGS
Construct H1_CA04/09_FL_HA_3BVE_pVAX without IgE-Entire expressible nucleic acid
sequence (SEQ ID NO: 99)
atgaaggctattctggtcgtgctgctgtatactttcgccaccgccaacgccgacacactgtgcatcggctaccacgccaacaattctacc
gacacagtggataccgtgctggagaagaatgtgaccgtgacacacagcgtgaacctgctggaggataagcacaatggcaagctgtg
caagctgaggggagtggcaccactgcacctgggcaagtgcaacatcgccggctggattctgggcaatcccgagtgtgagtccctgtc
tacagccagctcctggtcctacatcgtggagacaccctctagcgacaacggcacatgctaccctggcgactttatcgattatgaggagc
tgcgggagcagctgagcagcgtgagcagcttcgagaggttcgagatcttccccaagacctctagctggcctaaccacgatagcaata
agggagtgacagcagcatgtccacacgcaggcgccaagagcttctataagaacctgatctggctggtgaagaagggcaattcctacc
ctaagctgagcaagtcctatatcaacgacaagggcaaggaggtgctggtgctgtggggcatccaccacccatctaccagcgccgac
cagcagtccctgtaccagaatgccgatacatacgtgttcgtgggctcctctcggtactctaagaagttcaagccagagatcgccatcag
gccaaaggtgagggaccaggagggacgcatgaactactattggaccctggtggagcccggcgataagatcacctttgaggccaca
ggcaacctggtggtgcctagatatgccttcgccatggagagaaatgccggctccggcatcatcatctctgacacccctgtgcacgattg
caacaccacatgtcagaccccaaagggcgccatcaacacatccctgccttttcagaatatccacccaatcacaatcggcaagtgccct
aagtacgtgaagagcaccaagctgaggctggcaacaggcctgcgcaatatcccatctatccagagcaggggcctgtttggagcaatc
gcaggcttcatcgagggaggatggaccggaatggtggacggctggtacggctatcaccaccagaacgagcagggcagcggatatg
cagcagacctgaagtccacccagaatgccatcgatgagatcacaaacaaggtcaattccgtgatcgagaagatgaacacccagtttac
agccgtgggcaaggagttcaatcacctggagaagagaatcgagaacctgaataagaaggtggacgatggcttcctggacatctgga
cctacaacgccgagctgctggtgctgctggagaatgagaggacactggactaccacgattccaacgtgaagaatctgtatgagaagg
tgagatctcagctgaagaacaatgccaaggagatcggcaacggctgcttcgagttttaccacaagtgcgacaacacctgtatggagag
cgtgaagaatggcacatacgattatcctaagtattccgaggaggccaagctgaaccgcgaggagatcgactctggcggcgatatcat
caagctgctgaacgagcaagtgaataaggagatgcagagctccaatctgtacatgtctatgtctagctggtgttatacccacagcctgg
acggagcaggcctgttcctgtttgatcacgccgccgaggagtacgagcacgccaagaagctgatcatctttctgaacgagaacaatgt
gccagtgcagctgacctccatctctgcccccgagcacaagtttgagggcctgacacagatcttccagaaggcctacgagcacgagca
gcacatcagcgagtccatcaacaatatcgtggaccacgccatcaagagcaaggatcacgccaccttcaactttctgcagtggtacgtg
gccgagcagcacgaggaggaggtgctgttcaaggacatcctggataagatcgagctgatcggcaacgagaatcacgggctgtacct
ggcagaccagtatgtcaagggcatcgcaaagtcacggaagagcgggagc
Entire expressible amino acid sequence (SEQ ID NO: 100)
MKAILVVLLYTFATANADTLCIGYHANNSTDTVDTVLEKNVTVTHSVNLLEDKHNG
KLCKLRGVAPLHLGKCNIAGWILGNPECESLSTASSWSYIVETPSSDNGTCYPGDFID
YEELREQLSSVSSFERFEIFPKTSSWPNHDSNKGVTAACPHAGAKSFYKNLIWLVKK
GNSYPKLSKSYINDKGKEVLVLWGIHHPSTSADQQSLYQNADTYVFVGSSRYSKKFK
PEIAIRPKVRDQEGRMNYYWTLVEPGDKITFEATGNLVVPRYAFAMERNAGSGIIISD
TPVHDCNTTCQTPKGAINTSLPFQNIHPITIGKCPKYVKSTKLRLATGLRNIPSIQSRGL
FGAIAGFIEGGWTGMVDGWYGYHHQNEQGSGYAADLKSTQNAIDEITNKVNSVIEK
MNTQFTAVGKEFNHLEKRIENLNKKVDDGFLDIWTYNAELLVLLENERTLDYHDSN
VKNLYEKVRSQLKNNAKEIGNGCFEFYHKCDNTCMESVKNGTYDYPKYSEEAKLN
REEIDSGGDIIKLLNEQVNKEMQSSNLYMSMSSWCYTHSLDGAGLFLFDHAAEEYEH
AKKLIIFLNENNVPVQLTSISAPEHKFEGLTQIFQKAYEHEQHISESINNIVDHAIKSKD
HATFNFLQWYVAEQHEEEVLFKDILDKIELIGNENHGLYLADQYVKGIAKSRKSGS

### D. Cells

The present disclosure further relates to one or a plurality of cells that comprise any of the disclosed expressible nucleic acid sequences and/or any of the disclosed nucleic acid molecules or plasmids. In some embodiments, the cells of the present disclosure are cultured cells comprising one or more of the disclosed expressible nucleic acid sequences and/or one or more of the disclosed nucleic acid molecules or plasmids. In such embodiments, the cells may include, but not limited to, bacterial cells, fungal cells, insect cells, mammalian cells, or human cells. Any method routinely used by one of ordinary skill in the art for transforming or transfecting cells and maintaining transformed or transfected cells can be used to generate the cells of the present disclosure. In some embodiments, the cells of the present disclosure are cells present in a living subject which has been adminstered with a composition comprising one or a plurality of the disclosed expressible nucleic acid sequences and/or one or more of the disclosed nucleic acid molecules or plasmids.

### E. Pharmaceutical Compositions

Disclosed are pharmaceutical compositions comprising any one or more of the disclosed compositions and a pharmaceutically acceptable carrier.

In some embodiments, any of the disclosed compositions is from about 1 to about 30 micrograms. For example, any of the disclosed compositions can be from about 1 to about 5 micrograms. In some preferred embodiments, the pharmaceutical compositions contain from about 5 nanograms to about 800 micrograms of DNA. In some preferred embodiments, the pharmaceutical compositions contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of the consensus antigen or plasmid thereof. The pharmaceutical compositions can comprise from about 5 nanograms to about 10 mg of the vaccine DNA. In some embodiments, pharmaceutical compositions according to the present disclosure comprise from about 25 nanogram to about 5 mg of vaccine DNA. In some embodiments, the pharmaceutical compositions contain from about 50 nanograms to about 1 mg of DNA. In some embodiments, the pharmaceutical compositions contain about from about 0.1 to about 500 micrograms of DNA. In some embodiments, the pharmaceutical compositions contain from about 1 to about 350 micrograms of DNA. In some embodiments, the pharmaceutical compositions contain from about 5 to about 250 micrograms of DNA. In some embodiments, the pharmaceutical compositions contain from about 10 to about 200 micrograms of DNA. In some embodiments, the pharmaceutical compositions contain from about 15 to about 150 micrograms of DNA. In some embodiments, the pharmaceutical compositions contain about 20 to about 100 micrograms of DNA. In some embodiments, the pharmaceutical compositions contain about 25 to about 75 micrograms of DNA. In some embodiments, the pharmaceutical compositions contain about 30 to about 50 micrograms of DNA. In some embodiments, the pharmaceutical compositions contain about 35 to about 40 micrograms of DNA. In some embodiments, the pharmaceutical compositions contain about 100 to about 200 microgram DNA. In some embodiments, the pharmaceutical compositions comprise about 10 microgram to about 100 micrograms of DNA. In some embodiments, the pharmaceutical compositions comprise about 20 micrograms to about 80 micrograms of DNA. In some embodiments, the pharmaceutical compositions comprise about 25 micrograms to about 60 micrograms of DNA. In some embodiments, the pharmaceutical compositions comprise about 30 nanograms to about 50 micrograms of DNA. In some embodiments, the pharmaceutical compositions comprise about 35 nanograms to about 45 micrograms of DNA. In some preferred embodiments, the pharmaceutical compositions contain about 0.1 to about 500 micrograms of DNA. In some preferred embodiments, the pharmaceutical compositions contain about 1 to about 350 micrograms of DNA. In some preferred embodiments, the pharmaceutical compositions contain about 1 to about 250 micrograms of DNA. In some preferred embodiments, the pharmaceutical compositions contain about 2 to about 200 microgram DNA.

In some embodiments, pharmaceutical compositions according to the present disclosure comprise at least about 10, 15, 20, 25, 30, 35, 40, 45, 50, 55, 60, 65, 70, 75, 80, 85, 90, 95 or 100 nanograms of DNA of the vaccine. In some embodiments, the pharmaceutical compositions can comprise at least about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995 or 1000 micrograms of DNA of the vaccine. In some embodiments, the pharmaceutical composition can comprise at least 1.5, 2, 2.5, 3, 3.5, 4, 4.5, 5, 5.5, 6, 6.5, 7, 7.5, 8, 8.5, 9, 9.5 or 10 mg or more of DNA of the vaccine.

In other embodiments, the pharmaceutical composition can comprise up to and including about 10, 15, 20, 25, 30, 35, 40, 45, 50, 55, 60, 65, 70, 75, 80, 85, 90, 95 or 100 nanograms of DNA of the vaccine. In some embodiments, the pharmaceutical composition can comprise up to and including about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995, or 1000 micrograms of DNA of the vaccine. In some embodiments, the pharmaceutical composition can comprise up to and including about 1.5, 2, 2.5, 3, 3.5, 4, 4.5, 5, 5.5, 6, 6.5, 7, 7.5, 8, 8.5, 9, 9.5 or about 10 mg of DNA of the vaccine. The pharmaceutical composition can further comprise other agents for formulation purposes according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The vaccine can further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes

(ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or other known transfection facilitating agents. In some embodiments, the vaccine is a composition comprising a plasmid DNA molecule, RNA molecule or DNA/RNA hybrid molecule encoding an expressible nucleic acid sequence, the expressible nucleic acid sequence comprising a first nucleic acid encoding a self-assembling nanoparticle comprising a viral antigen, optionally encoding a leader sequence disclosed herein.

The transfection facilitating agent is a polyanion, polycation, including poly-L-glutamate (LGS), or lipid. The transfection facilitating agent is poly-L-glutamate, and more preferably, the poly-L-glutamate is present in the vaccine at a concentration less than 6 mg/ml. The transfection facilitating agent can also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid can also be used administered in conjunction with the genetic construct. In some embodiments, the DNA vector vaccines can also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in alternative plasmid or are deneurological systemed as proteins in combination with the plasmid above in the vaccine. The adjuvant can be selected from the group consisting of: α-interferon(IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80,CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof. In an exemplary embodiment, the adjuvant is IL-12.

Other genes which can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof or a combination thereof.

In some embodiments, adjuvant may be one or more proteins and/or nucleic acid molecules that encode proteins selected from the group consisting of: CCL-20, IL-2, IL-6, IL-7, IL-12, single-chain IL-12, IL-15, IL-27, IL-28, CTACK, TECK, MEC or RANTES. Examples of IL-12 constructs and sequences are disclosed in PCT application no. PCT/US1997/019502 and corresponding U.S. application Ser. No. 08/956,865, and U.S. Provisional Application Ser. No. 61/569,600 filed Dec. 12, 2011, which are each incorporated herein by reference in their entireties. Examples of IL-15 constructs and sequences are disclosed in PCT application no. PCT/US04/18962 and corresponding U.S. application Ser. No. 10/560,650, and in PCT application no. PCT/US07/00886 and corresponding U.S. application Ser. No. 12/160,766, and in PCT Application Serial No. PCT/US10/048827, which are each incorporated herein by reference in their entireties. Examples of IL-28 constructs and sequences are disclosed in PCT application no. PCT/US09/039648 and corresponding U.S. application Ser. No. 12/936,192, which are each incorporated herein by reference in their entireties. Examples of RANTES and other constructs and sequences are disclosed in PCT application no. PCT/US 1999/004332 and corresponding U.S. Application Serial No. and Ser. No. 09/622,452, which are each incorporated herein by reference in their entireties. Other examples of RANTES constructs and sequences are disclosed in PCT Application Serial No. PCT/US Ser. No. 11/024,098, which is incorporated herein by reference. Examples of RANTES and other constructs and sequences are disclosed in PCT Application Serial No. PCT/US 1999/004332 and corresponding U.S. application Ser. No. 09/622,452, which are each incorporated herein by reference. Other examples of RANTES constructs and sequences are disclosed in PCT application no. PCT/U.S. Ser. No. 11/024,098, which is incorporated herein by reference in its entirety. Examples of chemokines CTACK, TECK and MEC constructs and sequences are disclosed in PCT Application Serial No. PCT/US2005/042231 and corresponding U.S. application Ser. No. 11/719,646, which are each incorporated herein by reference in their entireties. Examples of OX40 and other immunomodulators are disclosed in U.S. application Ser. No. 10/560,653, which is incorporated herein by reference in its entirety. Examples of DR5 and other immunomodulators are disclosed in U.S. application Ser. No. 09/622,452, which is incorporated herein by reference in its entirety. In some embodiments, adjuvant may be a protein comprising at least 70%, 80%, 85%, 90%, 95%, 99% or more sequence identity to any proteins disclosed in any of the aforementioned patent applications incorporated by reference herein. In some embodiments, adjuvant may be a nucleic acid encoding a cytokine or chemokine comprising at least 70%, 80%, 85%, 90%, 95%, 99% or more sequence identity to any proteins disclosed in any of the aforementioned patent applications incorporated by reference herein.

The pharmaceutical composition may be formulated according to the mode of administration to be used. An injectable vaccine pharmaceutical composition may be sterile, pyrogen free and particulate free. An isotonic formulation or solution may be used. Additives for isotonicity may include sodium chloride, dextrose, mannitol, sorbitol, and lactose. The vaccine may comprise a vasoconstriction agent. The isotonic solutions may include phosphate buffered saline. Vaccine may further comprise stabilizers including gelatin and albumin. The stabilizing may allow the formulation to be stable at room or ambient temperature for extended periods of time such as LGS or polycations or polyanions to the vaccine formulation.

The vaccine can be a DNA or RNA vaccine. In some embodiments, the vaccine is a DNA vaccine. DNA vaccines are disclosed in U.S. Pat. Nos. 5,593,972, 5,739,118, 5,817,637, 5,830,876, 5,962,428, 5,981,505, 5,580,859, 5,703,055, and 5,676,594, which are incorporated herein fully by reference. The DNA vaccine can further comprise elements or reagents that inhibit it from integrating into the chromosome. Examples of attenuated live vaccines, those using recombinant vectors to foreign antigens, subunit vaccines and glycoprotein vaccines are described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference in their entireties.

In some embodiments, the vaccine is an LNP comprising one or a modified RNA molecule. In some embodiments, the vaccine comprises a modified mRNA. Modified polynucleotides (such as, but not limited to, primary constructs), formulations and compositions comprising modified polynucleotides, and methods of making, using and administering modified polynucleotides are described in co-pending U.S. Provisional Patent Application No. 61/618,862, filed Apr. 2, 2012, entitled Modified Polynucleotides for the Production of Biologics; U.S. Provisional Patent Application No. 61/681,645, filed Aug. 10, 2012, entitled Modified Polynucleotides for the Production of Biologics; U.S. Provisional Patent Application No. 61/737,130, filed Dec. 14, 2012, entitled Modified Polynucleotides for the Production of Biologics; U.S. Provisional Patent Application No. 61/618,866, filed Apr. 2, 2012, entitled Modified Polynucleotides for the Production of Antibodies; U.S. Provisional Patent Application No. 61/681,647, filed Aug. 10, 2012, entitled Modified Polynucleotides for the Production of Antibodies; U.S. Provisional Patent Application No. 61/737,134, filed Dec. 14, 2012, entitled Modified Polynucleotides for the Production of Antibodies; U.S. Provisional Patent Application No. 61/618,868, filed Apr. 2, 2012, entitled Modified Polynucleotides for the Production of Vaccines; U.S. Provisional Patent Application No. 61/681,648, filed Aug. 10, 2012, entitled Modified Polynucleotides for the Production of Vaccines; U.S. Provisional Patent Application No. 61/737,135, filed Dec. 14, 2012, entitled Modified Polynucleotides for the Production of Vaccines; U.S. Provisional Patent Application No. 61/618,873, filed Apr. 2, 2012, entitled Modified Polynucleotides for the Production of Secreted Proteins; U.S. Provisional Patent Application No. 61/681,650, filed Aug. 10, 2012, entitled Modified Polynucleotides for the Production of Secreted Proteins; U.S. Provisional Patent Application No. 61/737,147, filed Dec. 14, 2012, entitled Modified Polynucleotides for the Production of Secreted Proteins; U.S. Provisional Patent Application No. 61/618,878, filed Apr. 2, 2012, entitled Modified Polynucleotides for the Production of Plasma Membrane Proteins; U.S. Provisional Patent Application No. 61/681,654, filed Aug. 10, 2012, entitled Modified Polynucleotides for the Production of Plasma Membrane Proteins; U.S. Provisional Patent Application No. 61/737,152, filed Dec. 14, 2012, entitled Modified Polynucleotides for the Production of Plasma Membrane Proteins; U.S. Provisional Patent Application No. 61/618,885, filed Apr. 2, 2012, entitled Modified Polynucleotides for the Production of Cytoplasmic and Cytoskeletal Proteins; U.S. Provisional Patent Application No. 61/681,658, filed Aug. 10, 2012, entitled Modified Polynucleotides for the Production of Cytoplasmic and Cytoskeletal Proteins; U.S. Provisional Patent Application No. 61/737,155, filed Dec. 14, 2012, entitled Modified Polynucleotides for the Production of Cytoplasmic and Cytoskeletal Proteins; U.S. Provisional Patent Application No. 61/618,896, filed Apr. 2, 2012, entitled Modified Polynucleotides for the Production of Intracellular Membrane Bound Proteins; U.S. Provisional Patent Application No. 61/668,157, filed Jul. 5, 2012, entitled Modified Polynucleotides for the Production of Intracellular Membrane Bound Proteins; U.S. Provisional Patent Application No. 61/681,661, filed Aug. 10, 2012, entitled Modified Polynucleotides for the Production of Intracellular Membrane Bound Proteins; U.S. Provisional Patent Application No. 61/737,160, filed Dec. 14, 2012, entitled Modified Polynucleotides for the Production of Intracellular Membrane Bound Proteins; U.S. Provisional Patent Application No. 61/618,911, filed Apr. 2, 2012, entitled Modified Polynucleotides for the Production of Nuclear Proteins; U.S. Provisional Patent Application No. 61/681,667, filed Aug. 10, 2012, entitled Modified Polynucleotides for the Production of Nuclear Proteins; U.S. Provisional Patent Application No. 61/737,168, filed Dec. 14, 2012, entitled Modified Polynucleotides for the Production of Nuclear Proteins; U.S. Provisional Patent Application No. 61/618,922, filed Apr. 2, 2012, entitled Modified Polynucleotides for the Production of Proteins; U.S. Provisional Patent Application No. 61/681,675, filed Aug. 10, 2012, entitled Modified Polynucleotides for the Production of Proteins; U.S. Provisional Patent Application No. 61/737,174, filed Dec. 14, 2012, entitled Modified Polynucleotides for the Production of Proteins; U.S. Provisional Patent Application No. 61/618,935, filed Apr. 2, 2012, entitled Modified Polynucleotides for the Production of Proteins Associated with Human Disease; U.S. Provisional Patent Application No. 61/681,687, filed Aug. 10, 2012, entitled Modified Polynucleotides for the Production of Proteins Associated with Human Disease; U.S. Provisional Patent Application No. 61/737,184, filed Dec. 14, 2012, entitled Modified Polynucleotides for the Production of Proteins Associated with Human Disease; U.S. Provisional Patent Application No. 61/618,945, filed Apr. 2, 2012, entitled Modified Polynucleotides for the Production of Proteins Associated with Human Disease; U.S. Provisional Patent Application No. 61/681,696, filed Aug. 10, 2012, entitled Modified Polynucleotides for the Production of Proteins Associated with Human Disease; U.S. Provisional Patent Application No. 61/737,191, filed Dec. 14, 2012, entitled Modified Polynucleotides for the Production of Proteins Associated with Human Disease; U.S. Provisional Patent Application No. 61/618,953, filed Apr. 2, 2012, entitled Modified Polynucleotides for the Production of Proteins Associated with Human Disease; U.S. Provisional Patent Application No. 61/681,704, filed Aug. 10, 2012, entitled Modified Polynucleotides for the Production of Proteins Associated with Human Disease; U.S. Provisional Patent Application No. 61/737,203, filed Dec. 14, 2012, entitled Modified Polynucleotides for the Production of Proteins Associated with Human Disease; International Application No PCT/US2013/030062, filed Mar. 9, 2013, entitled Modified Polynucleotides for the Production of Biologics and Proteins Associated with Human Disease; International Application No. PCT/US2013/030064, entitled Modified Polynucleotides for the Production of Secreted Proteins; International Application No PCT/US2013/030059, filed Mar. 9, 2013, entitled Modified Polynucleotides for the Production of Membrane Proteins; International Application No. PCT/US2013/030066, filed Mar. 9, 2013, entitled Modified Polynucleotides for the Production of Cytoplasmic and Cytoskeletal Proteins; International Application No. PCT/US2013/030067, filed Mar. 9, 2013, entitled Modified Polynucleotides for the Production of Nuclear Proteins; International Application No. PCT/US2013/030060, filed Mar. 9, 2013, entitled Modified Polynucleotides for the Production of Proteins; International Application No. PCT/US2013/030061, filed Mar. 9, 2013, entitled Modified Polynucleotides for the Production of Proteins Associated with Human Disease; in co-pending U.S. Provisional Patent Application No. 61/681,720, filed Aug. 10, 2012, entitled Modified Polynucleotides for the Production of Cosmetic Proteins and Peptides; U.S. Provisional Patent Application No. 61/737,213, filed Dec. 14, 2012, entitled Modified Polynucleotides for the Production of Cosmetic Proteins and Peptides; U.S. Provisional Patent Application No. 61/681,742, filed Aug. 10, 2012, entitled Modified Polynucleotides for the Production of Oncology-Related Proteins and Peptides; International Application No. PCT/US2013/030070, filed Mar. 9, 2013, entitled Modified Polynucleotides for the Production of Oncology-Related Proteins and Peptides; in co-pending International Application No. PCT/US2013/030068, filed Mar. 9, 2013, entitled Modified Polynucleotides for the Production of Cosmetic Proteins and Peptides; in co-pending U.S. Provisional Patent Application No. 61/618,870, filed Apr. 2, 2012, entitled Modified Polynucleotides for the Production of Therapeutic Proteins and Peptides; in co-pending U.S. Provisional Patent Application No. 61/681,649, filed Aug. 10, 2012, entitled Modified Polynucleotides for the Production of Therapeutic Proteins and Peptides; in co-pending U.S. Provisional Patent Application No. 61/737,139, filed Dec. 14, 2012, Modified Polynucleotides for the Production of Therapeutic Proteins and Peptides; in co-pending International Application No PCT/US2013/030063, filed Mar. 9, 2013, entitled Modified Polynucleotides; and in co-pending International Application No PCT/US2013/031821, filed Mar. 15, 2013, entitled In Vivo Production of Proteins; the contents of each of which are herein incorporated by reference in their entireties. Any of the recited polypeptides of the modified polynucleotides of the foregoing are considered useful as a polypeptide of interest or antigen of the LNPs of the present invention.

The genetic construct can also be part of a genome of a recombinant viral vector, including recombinant adenovirus, recombinant adenovirus associated virus and recombinant vaccinia. The genetic construct can be part of the genetic material in attenuated live microorganisms or recombinant microbial vectors which live in cells.

The disclosure relates to a genetic construct or composition comprising any of the expressible nucleic acid molecules disclosed herein. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 1. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 2. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 3. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 4. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 5. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 6. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 7. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 8. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 9. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 10. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 11. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 12, In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 13. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 14. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 15. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 16. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 17. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 18. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 19. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 20. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 21. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 22. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 23. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 24. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 25. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 26. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 27. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 28. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 29. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 30. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 31. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 32. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 33. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 34. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 35. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 36. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 37. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 38. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 39. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 40. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 41. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 42. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 43. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 44. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 45. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 46. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 47. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 48. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 49. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 50. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 51. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 52. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 53. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 54. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 55. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 56. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 57. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 58. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 59. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 60. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 61. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 62. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 63. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 64. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 65. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 66. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 67. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 68. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 69. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 70. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 71. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 72. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 73. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 74. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 75. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 76. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 77. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 78. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 79. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 80. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 81. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 82. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 83. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 84. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 85. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 86. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 87. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 88. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 89. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 90. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 91. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 92. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 93. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 94. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 95. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 96. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 97. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 98. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 99. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 100. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 101. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 102. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 103. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 104. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 105. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 106. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 107. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 108. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 109. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 110. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 111. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 112. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 113. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 114. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 115. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 116. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 117. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 118. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 119. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 120. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 121. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 122. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 123. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 124. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 125. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 126. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 127. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 128. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 129. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 130. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 131. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 132. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 133. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 134. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 135. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 136. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 137. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 138. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 139. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 140. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 141. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 142. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 143. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 144. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 145. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 146. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 147. In some embodiments, the nucleic acid molecule comprises a nucleic acid sequence comprising (or encoding an amino acid sequence comprising) at least about 70%, 75%, 80%, 85%, 90%, 91%, 92%, 93%, 94%, 95%, 96%, 97%, 98%, 99% or 100% sequence identity to SEQ ID NO: 148.

In some embodiments, the disclosure relates to a composition comprising any of the self-assembling polypeptides disclosed herein, or the corresponding coding nucleic acid, in combination with any of the viral antigens disclosed herein, or the corresponding coding nucleic acid. In some embodiments, such composition further comprises any of the leader sequences disclosed herein, or the corresponding coding nucleic acid, and/or any of the linkers disclosed herein, or the corresponding coding nucleic acid. In some embodiments, when in form of a nucleic acid, such composition is present in any of the DNA backbonds disclosed herein.

In some embodiments, the disclosure relates to a composition comprising SEQ ID NO: 7 (or a variant thereof) in combination with SEQ ID NO: 9 (or a variant thereof), or the corresponding coding nucleic acid. In some embodiments, the disclosure relates to a composition comprising SEQ ID NO: 7 (or a variant thereof) in combination with SEQ ID NO: 45 (or a variant thereof), or the corresponding coding nucleic acid. In some embodiments, the disclosure relates to a composition comprising SEQ ID NO: 7 (or a variant thereof) in combination with SEQ ID NO: 47 (or a variant thereof), or the corresponding coding nucleic acid. In some embodiments, the disclosure relates to a composition comprising SEQ ID NO: 7 (or a variant thereof) in combination with SEQ ID NO: 49 (or a variant thereof), or the corresponding coding nucleic acid. In some embodiments, the disclosure relates to a composition comprising SEQ ID NO: 7 (or a variant thereof) in combination with SEQ ID NO: 51 (or a variant thereof), or the corresponding coding nucleic acid. In some embodiments, the disclosure relates to a composition comprising SEQ ID NO: 7 (or a variant thereof) in combination with SEQ ID NO: 53 (or a variant thereof), or the corresponding coding nucleic acid. In some embodiments, the disclosure relates to a composition comprising SEQ ID NO: 7 (or a variant thereof) in combination with SEQ ID NO: 55 (or a variant thereof), or the corresponding coding nucleic acid. In some embodiments, the disclosure relates to a composition comprising SEQ ID NO: 7 (or a variant thereof) in combination with SEQ ID NO: 65 (or a variant thereof), or the corresponding coding nucleic acid. In some embodiments, the disclosure relates to a composition comprising SEQ ID NO: 7 (or a variant thereof) in combination with SEQ ID NO: 66 (or a variant thereof), or the corresponding coding nucleic acid. In some embodiments, the disclosure relates to a composition comprising SEQ ID NO: 7 (or a variant thereof) in combination with SEQ ID NO: 67 (or a variant thereof), or the corresponding coding nucleic acid. In some embodiments, the disclosure relates to a composition comprising SEQ ID NO: 23 (or a variant thereof) in combination with SEQ ID NO: 9 (or a variant thereof), or the corresponding coding nucleic acid. In some embodiments, the disclosure relates to a composition comprising SEQ ID NO: 23 (or a variant thereof) in combination with SEQ ID NO: 45 (or a variant thereof), or the corresponding coding nucleic acid. In some embodiments, the disclosure relates to a composition comprising SEQ ID NO: 23 (or a variant thereof) in combination with SEQ ID NO: 47 (or a variant thereof), or the corresponding coding nucleic acid. In some embodiments, the disclosure relates to a composition comprising SEQ ID NO: 23 (or a variant thereof) in combination with SEQ ID NO: 49 (or a variant thereof), or the corresponding coding nucleic acid. In some embodiments, the disclosure relates to a composition comprising SEQ ID NO: 23 (or a variant thereof) in combination with SEQ ID NO: 51 (or a variant thereof), or the corresponding coding nucleic acid. In some embodiments, the disclosure relates to a composition comprising SEQ ID NO: 23 (or a variant thereof) in combination with SEQ ID NO: 53 (or a variant thereof), or the corresponding coding nucleic acid. In some embodiments, the disclosure relates to a composition comprising SEQ ID NO: 23 (or a variant thereof) in combination with SEQ ID NO: 55 (or a variant thereof), or the corresponding coding nucleic acid. In some embodiments, the disclosure relates to a composition comprising SEQ ID NO: 23 (or a variant thereof) in combination with SEQ ID NO: 65 (or a variant thereof), or the corresponding coding nucleic acid. In some embodiments, the disclosure relates to a composition comprising SEQ ID NO: 23 (or a variant thereof) in combination with SEQ ID NO: 66 (or a variant thereof), or the corresponding coding nucleic acid. In some embodiments, the disclosure relates to a composition comprising SEQ ID NO: 23 (or a variant thereof) in combination with SEQ ID NO: 67 (or a variant thereof), or the corresponding coding nucleic acid. In some embodiments, the disclosure relates to a composition comprising SEQ ID NO: 26 (or a variant thereof) in combination with SEQ ID NO: 9 (or a variant thereof), or the corresponding coding nucleic acid. In some embodiments, the disclosure relates to a composition comprising SEQ ID NO: 26 (or a variant thereof) in combination with SEQ ID NO: 45 (or a variant thereof), or the corresponding coding nucleic acid. In some embodiments, the disclosure relates to a composition comprising SEQ ID NO: 26 (or a variant thereof) in combination with SEQ ID NO: 47 (or a variant thereof), or the corresponding coding nucleic acid. In some embodiments, the disclosure relates to a composition comprising SEQ ID NO: 26 (or a variant thereof) in combination with SEQ ID NO: 49 (or a variant thereof), or the corresponding coding nucleic acid. In some embodiments, the disclosure relates to a composition comprising SEQ ID NO: 26 (or a variant thereof) in combination with SEQ ID NO: 51 (or a variant thereof), or the corresponding coding nucleic acid. In some embodiments, the disclosure relates to a composition comprising SEQ ID NO: 26 (or a variant thereof) in combination with SEQ ID NO: 53 (or a variant thereof), or the corresponding coding nucleic acid. In some embodiments, the disclosure relates to a composition comprising SEQ ID NO: 26 (or a variant thereof) in combination with SEQ ID NO: 55 (or a variant thereof), or the corresponding coding nucleic acid. In some embodiments, the disclosure relates to a composition comprising SEQ ID NO: 26 (or a variant thereof) in combination with SEQ ID NO: 65 (or a variant thereof), or the corresponding coding nucleic acid. In some embodiments, the disclosure relates to a composition comprising SEQ ID NO: 26 (or a variant thereof) in combination with SEQ ID NO: 66 (or a variant thereof), or the corresponding coding nucleic acid. In some embodiments, the disclosure relates to a composition comprising SEQ ID NO: 26 (or a variant thereof) in combination with SEQ ID NO: 67 (or a variant thereof), or the corresponding coding nucleic acid. In some embodiments, the disclosure relates to a composition comprising SEQ ID NO: 31 (or a variant thereof) in combination with SEQ ID NO: 9 (or a variant thereof), or the corresponding coding nucleic acid. In some embodiments, the disclosure relates to a composition comprising SEQ ID NO: 31 (or a variant thereof) in combination with SEQ ID NO: 45 (or a variant thereof), or the corresponding coding nucleic acid. In some embodiments, the disclosure relates to a composition comprising SEQ ID NO: 31 (or a variant thereof) in combination with SEQ ID NO: 47 (or a variant thereof), or the corresponding coding nucleic acid. In some embodiments, the disclosure relates to a composition comprising SEQ ID NO: 31 (or a variant thereof) in combination with SEQ ID NO: 49 (or a variant thereof), or the corresponding coding nucleic acid. In some embodiments, the disclosure relates to a composition comprising SEQ ID NO: 31 (or a variant thereof) in combination with SEQ ID NO: 51 (or a variant thereof), or the corresponding coding nucleic acid. In some embodiments, the disclosure relates to a composition comprising SEQ ID NO: 31 (or a variant thereof) in combination with SEQ ID NO: 53 (or a variant thereof), or the corresponding coding nucleic acid. In some embodiments, the disclosure relates to a composition comprising SEQ ID NO: 31 (or a variant thereof) in combination with SEQ ID NO: 55 (or a variant thereof), or the corresponding coding nucleic acid. In some embodiments, the disclosure relates to a composition comprising SEQ ID NO: 31 (or a variant thereof) in combination with SEQ ID NO: 65 (or a variant thereof), or the corresponding coding nucleic acid. In some embodiments, the disclosure relates to a composition comprising SEQ ID NO: 31 (or a variant thereof) in combination with SEQ ID NO: 66 (or a variant thereof), or the corresponding coding nucleic acid. In some embodiments, the disclosure relates to a composition comprising SEQ ID NO: 31 (or a variant thereof) in combination with SEQ ID NO: 67 (or a variant thereof), or the corresponding coding nucleic acid. In some embodiments, any of the aforementioned compositions further comprises SEQ ID NO: 6 (or a variant thereof), or the corresponding coding nucleic acid. In some embodiments, any of the aforementioned compositions further comprises SEQ ID NO: 40 (or a variant thereof), or the corresponding coding nucleic acid. In some embodiments, any of the aforementioned compositions further comprises SEQ ID NO: 101 (or a variant thereof), or the corresponding coding nucleic acid. In some embodiments, any of the aforementioned compositions further comprises SEQ ID NO: 8 (or a variant thereof), or the corresponding coding nucleic acid. In some embodiments, any of the aforementioned compositions further comprises SEQ ID NO: 18 (or a variant thereof), or the corresponding coding nucleic acid. In some embodiments, any of the aforementioned compositions further comprises SEQ ID NO: 22 (or a variant thereof), or the corresponding coding nucleic acid. In some embodiments, any of the aforementioned compositions further comprises SEQ ID NO: 27 (or a variant thereof), or the corresponding coding nucleic acid. In some embodiments, any of the aforementioned compositions further comprises SEQ ID NO: 32 (or a variant thereof), or the corresponding coding nucleic acid.

In some embodiments, the disclosure relates to a composition comprising SEQ ID NO: 7 (or a variant thereof) in combination with SEQ ID NO: 103 (or a variant thereof), or the corresponding coding nucleic acid. In some embodiments, the disclosure relates to a composition comprising SEQ ID NO: 7 (or a variant thereof) in combination with SEQ ID NO: 104 (or a variant thereof), or the corresponding coding nucleic acid. In some embodiments, the disclosure relates to a composition comprising SEQ ID NO: 7 (or a variant thereof) in combination with SEQ ID NO: 105 (or a variant thereof), or the corresponding coding nucleic acid. In some embodiments, the disclosure relates to a composition comprising SEQ ID NO: 7 (or a variant thereof) in combination with SEQ ID NO: 106 (or a variant thereof), or the corresponding coding nucleic acid. In some embodiments, the disclosure relates to a composition comprising SEQ ID NO: 7 (or a variant thereof) in combination with SEQ ID NO: 108 (or a variant thereof), or the corresponding coding nucleic acid. In some embodiments, the disclosure relates to a composition comprising SEQ ID NO: 7 (or a variant thereof) in combination with SEQ ID NO: 109 (or a variant thereof), or the corresponding coding nucleic acid. In some embodiments, the disclosure relates to a composition comprising SEQ ID NO: 7 (or a variant thereof) in combination with SEQ ID NO: 110 (or a variant thereof), or the corresponding coding nucleic acid. In some embodiments, the disclosure relates to a composition comprising SEQ ID NO: 7 (or a variant thereof) in combination with SEQ ID NO: 111 (or a variant thereof), or the corresponding coding nucleic acid. In some embodiments, the disclosure relates to a composition comprising SEQ ID NO: 23 (or a variant thereof) in combination with SEQ ID NO: 103 (or a variant thereof), or the corresponding coding nucleic acid. In some embodiments, the disclosure relates to a composition comprising SEQ ID NO: 23 (or a variant thereof) in combination with SEQ ID NO: 104 (or a variant thereof), or the corresponding coding nucleic acid. In some embodiments, the disclosure relates to a composition comprising SEQ ID NO: 23 (or a variant thereof) in combination with SEQ ID NO: 105 (or a variant thereof), or the corresponding coding nucleic acid. In some embodiments, the disclosure relates to a composition comprising SEQ ID NO: 23 (or a variant thereof) in combination with SEQ ID NO: 106 (or a variant thereof), or the corresponding coding nucleic acid. In some embodiments, the disclosure relates to a composition comprising SEQ ID NO: 23 (or a variant thereof) in combination with SEQ ID NO: 108 (or a variant thereof), or the corresponding coding nucleic acid. In some embodiments, the disclosure relates to a composition comprising SEQ ID NO: 23 (or a variant thereof) in combination with SEQ ID NO: 109 (or a variant thereof), or the corresponding coding nucleic acid. In some embodiments, the disclosure relates to a composition comprising SEQ ID NO: 23 (or a variant thereof) in combination with SEQ ID NO: 110 (or a variant thereof), or the corresponding coding nucleic acid. In some embodiments, the disclosure relates to a composition comprising SEQ ID NO: 23 (or a variant thereof) in combination with SEQ ID NO: 111 (or a variant thereof), or the corresponding coding nucleic acid. In some embodiments, the disclosure relates to a composition comprising SEQ ID NO: 26 (or a variant thereof) in combination with SEQ ID NO: 103 (or a variant thereof), or the corresponding coding nucleic acid. In some embodiments, the disclosure relates to a composition comprising SEQ ID NO: 26 (or a variant thereof) in combination with SEQ ID NO: 104 (or a variant thereof), or the corresponding coding nucleic acid. In some embodiments, the disclosure relates to a composition comprising SEQ ID NO: 26 (or a variant thereof) in combination with SEQ ID NO: 105 (or a variant thereof), or the corresponding coding nucleic acid. In some embodiments, the disclosure relates to a composition comprising SEQ ID NO: 26 (or a variant thereof) in combination with SEQ ID NO: 106 (or a variant thereof), or the corresponding coding nucleic acid. In some embodiments, the disclosure relates to a composition comprising SEQ ID NO: 26 (or a variant thereof) in combination with SEQ ID NO: 108 (or a variant thereof), or the corresponding coding nucleic acid. In some embodiments, the disclosure relates to a composition comprising SEQ ID NO: 26 (or a variant thereof) in combination with SEQ ID NO: 109 (or a variant thereof), or the corresponding coding nucleic acid. In some embodiments, the disclosure relates to a composition comprising SEQ ID NO: 26 (or a variant thereof) in combination with SEQ ID NO: 110 (or a variant thereof), or the corresponding coding nucleic acid. In some embodiments, the disclosure relates to a composition comprising SEQ ID NO: 26 (or a variant thereof) in combination with SEQ ID NO: 111 (or a variant thereof), or the corresponding coding nucleic acid. In some embodiments, the disclosure relates to a composition comprising SEQ ID NO: 31 (or a variant thereof) in combination with SEQ ID NO: 103 (or a variant thereof), or the corresponding coding nucleic acid. In some embodiments, the disclosure relates to a composition comprising SEQ ID NO: 31 (or a variant thereof) in combination with SEQ ID NO: 104 (or a variant thereof), or the corresponding coding nucleic acid. In some embodiments, the disclosure relates to a composition comprising SEQ ID NO: 31 (or a variant thereof) in combination with SEQ ID NO: 105 (or a variant thereof), or the corresponding coding nucleic acid. In some embodiments, the disclosure relates to a composition comprising SEQ ID NO: 31 (or a variant thereof) in combination with SEQ ID NO: 106 (or a variant thereof), or the corresponding coding nucleic acid. In some embodiments, the disclosure relates to a composition comprising SEQ ID NO: 31 (or a variant thereof) in combination with SEQ ID NO: 108 (or a variant thereof), or the corresponding coding nucleic acid. In some embodiments, the disclosure relates to a composition comprising SEQ ID NO: 31 (or a variant thereof) in combination with SEQ ID NO: 109 (or a variant thereof), or the corresponding coding nucleic acid. In some embodiments, the disclosure relates to a composition comprising SEQ ID NO: 31 (or a variant thereof) in combination with SEQ ID NO: 110 (or a variant thereof), or the corresponding coding nucleic acid. In some embodiments, the disclosure relates to a composition comprising SEQ ID NO: 31 (or a variant thereof) in combination with SEQ ID NO: 111 (or a variant thereof), or the corresponding coding nucleic acid. In some embodiments, any of the aforementioned compositions further comprises SEQ ID NO: 9 (or a variant thereof), or the corresponding coding nucleic acid. In some embodiments, any of the aforementioned compositions further comprises SEQ ID NO: 45 (or a variant thereof), or the corresponding coding nucleic acid. In some embodiments, any of the aforementioned compositions further comprises SEQ ID NO: 47 (or a variant thereof), or the corresponding coding nucleic acid. In some embodiments, any of the aforementioned compositions further comprises SEQ ID NO: 49 (or a variant thereof), or the corresponding coding nucleic acid. In some embodiments, any of the aforementioned compositions further comprises SEQ ID NO: 51 (or a variant thereof), or the corresponding coding nucleic acid. In some embodiments, any of the aforementioned compositions further comprises SEQ ID NO: 53 (or a variant thereof), or the corresponding coding nucleic acid. In some embodiments, any of the aforementioned compositions further comprises SEQ ID NO: 55 (or a variant thereof), or the corresponding coding nucleic acid. In some embodiments, any of the aforementioned compositions further comprises SEQ ID NO: 65 (or a variant thereof), or the corresponding coding nucleic acid. In some embodiments, any of the aforementioned compositions further comprises SEQ ID NO: 66 (or a variant thereof), or the corresponding coding nucleic acid. In some embodiments, any of the aforementioned compositions further comprises SEQ ID NO: 67 (or a variant thereof), or the corresponding coding nucleic acid. In some embodiments, any of the aforementioned compositions further comprises SEQ ID NO: 6 (or a variant thereof), or the corresponding coding nucleic acid. In some embodiments, any of the aforementioned compositions further comprises SEQ ID NO: 40 (or a variant thereof), or the corresponding coding nucleic acid. In some embodiments, any of the aforementioned compositions further comprises SEQ ID NO: 101 (or a variant thereof), or the corresponding coding nucleic acid. In some embodiments, any of the aforementioned compositions further comprises SEQ ID NO: 8 (or a variant thereof), or the corresponding coding nucleic acid. In some embodiments, any of the aforementioned compositions further comprises SEQ ID NO: 18 (or a variant thereof), or the corresponding coding nucleic acid. In some embodiments, any of the aforementioned compositions further comprises SEQ ID NO: 22 (or a variant thereof), or the corresponding coding nucleic acid. In some embodiments, any of the aforementioned compositions further comprises SEQ ID NO: 27 (or a variant thereof), or the corresponding coding nucleic acid. In some embodiments, any of the aforementioned compositions further comprises SEQ ID NO: 32 (or a variant thereof), or the corresponding coding nucleic acid.

In some embodiments, any of the aforementioned coding nucleic acids in the disclosed compositions further comprises SEQ ID NO: 56 (or a variant thereof). In some embodiments, any of the aforementioned coding nucleic acids in the disclosed compositions further comprises SEQ ID NO: 76 (or a variant thereof). In some embodiments, any of the aforementioned coding nucleic acids in the disclosed compositions further comprises SEQ ID NO: 77 (or a variant thereof). In some embodiments, any of the aforementioned coding nucleic acids in the disclosed compositions further comprises SEQ ID NO: 78 (or a variant thereof). In some embodiments, any of the aforementioned coding nucleic acids in the disclosed compositions further comprises SEQ ID NO: 79 (or a variant thereof). In some embodiments, any of the aforementioned coding nucleic acids in the disclosed compositions further comprises SEQ ID NO: 80 (or a variant thereof). In some embodiments, any of the aforementioned coding nucleic acids in the disclosed compositions further comprises SEQ ID NO: 81 (or a variant thereof). In some embodiments, any of the aforementioned coding nucleic acids in the disclosed compositions further comprises SEQ ID NO: 82 (or a variant thereof). As used herein, the term “variant” comprised in any of the disclosed compositions is a polypeptide or a nucleic acid that comprises at least about 70%, 75%, 80%, 85%, 90%, 91%, 92%, 93%, 94%, 95%, 96%, 97%, 98%, 99% or more sequence identity to the parent polypeptide or the parent nucleic acid as determined by sequence alignment programs and parameters described elsewhere herein.

In some embodiments, the disclosure relates to a DNA vector pVAX1 comprising any one or more of the expressible nucliec acid sequences disclosed herein or an RNA transcript thereof. In some embodiments, the disclosure relates to a pharmaceutical composition comprising a nucleic acid sequence that includes one or a plurality of expressible nucleic acid sequences disclosed herein or an RNA transcript thereof; and a pharmaceutically acceptable carrier.

### F. Methods

Disclosed are methods of vaccinating a subject comprising administering a therapeutically effective amount of any of the disclosed nucleic acid molecules, compositions, cells or pharmaceutical compositions to the subject. In some embodiments, the vaccination is against viral infection. In some embodiments, the viral infection is an infection of retroviridae. In some embodiments, the viral infection is an infection of a retrovirus. In some embodiments, the viral infection is an infection of a flavivirus. In some embodiments, the viral infection is an infection of Nipah virus. In some embodiments, the viral infection is an infection of West Nile virus. In some embodiments, the viral infection is an infection of human papillomavirus. In some embodiments, the viral infection is an infection of respiratory syncytial virus. In some embodiments, the viral infection is an infection of filovirus. In some embodiments, the viral infection is an infection of zaire ebolavirus. In some embodiments, the viral infection is an infection of sudan ebolavirus. In some embodiments, the viral infection is an infection of marburgvirus. In some embodiments, the viral infection is an infection of influenza virus. In some embodiments, the viral infection is an infection of HIV-1.

Disclosed are methods of inducing an immune response in a subject comprising administering to the subject any of the disclosed pharmaceutical compositions. In some embodiments, the methods are for inducing an immune response to a viral antigen in the subject. In some embodiments, the immune response induced by the disclosed methods is against a viral antigen from a retroviridae. In some embodiments, the immune response induced by the disclosed methods is against a viral antigen from a retrovirus. In some embodiments, the immune response induced by the disclosed methods is against a viral antigen from a flavivirus. In some embodiments, the immune response induced by the disclosed methods is against a viral antigen from a Nipah virus. In some embodiments, the immune response induced by the disclosed methods is against a viral antigen from a West Nile virus. In some embodiments, the immune response induced by the disclosed methods is against a viral antigen from a human papillomavirus. In some embodiments, the immune response induced by the disclosed methods is against a viral antigen from a respiratory syncytial virus. In some embodiments, the immune response induced by the disclosed methods is against a viral antigen from a filovirus. In some embodiments, the immune response induced by the disclosed methods is against a viral antigen from a zaire ebolavirus. In some embodiments, the immune response induced by the disclosed methods is against a viral antigen from a sudan ebolavirus. In some embodiments, the immune response induced by the disclosed methods is against a viral antigen from a marburgvirus. In some embodiments, the immune response induced by the disclosed methods is against a viral antigen from an influenza virus. In some embodiments, the immune response induced by the disclosed methods is against a viral antigen from HIV-1.

Disclosed are methods of neutralizing one or a plurality of viruses in a subject comprising administering to the subject any of the disclosed pharmaceutical compositions. In some embodiments, the virus being neutralized by the disclosed method is retroviridae. In some embodiments, the virus being neutralized by the disclosed method is a retrovirus. In some embodiments, the virus being neutralized by the disclosed method is flavivirus. In some embodiments, the virus being neutralized by the disclosed method is Nipah virus. In some embodiments, the virus being neutralized by the disclosed method is West Nile virus. In some embodiments, the virus being neutralized by the disclosed method is human papillomavirus. In some embodiments, the virus being neutralized by the disclosed method is respiratory syncytial virus. In some embodiments, the virus being neutralized by the disclosed method is filovirus. In some embodiments, the virus being neutralized by the disclosed method is zaire ebolavirus. In some embodiments, the virus being neutralized by the disclosed method is sudan ebolavirus. In some embodiments, the virus being neutralized by the disclosed method is marburgvirus. In some embodiments, the virus being neutralized by the disclosed method is influenza virus. In some embodiments, the virus being neutralized by the disclosed method is HIV-1.

Disclosed are methods of neutralizing infection of one or a plurality of viruses in a subject comprising administering to the subject any of the disclosed pharmaceutical compositions. In some embodiments, the viral infection being neutralized by the disclosed method is an infection of retroviridae. In some embodiments, the viral infection being neutralized by the disclosed method is an infection of a retrovirus. In some embodiments, the viral infection being neutralized by the disclosed method is an infection of flavivirus. In some embodiments, the viral infection being neutralized by the disclosed method is an infection of Nipah virus. In some embodiments, the viral infection being neutralized by the disclosed method is an infection of West Nile virus. In some embodiments, the viral infection being neutralized by the disclosed method is an infection of human papillomavirus. In some embodiments, the viral infection being neutralized by the disclosed method is an infection of respiratory syncytial virus. In some embodiments, the viral infection being neutralized by the disclosed method is an infection of filovirus. In some embodiments, the viral infection being neutralized by the disclosed method is an infection of zaire ebolavirus. In some embodiments, the viral infection being neutralized by the disclosed method is an infection of sudan ebolavirus. In some embodiments, the viral infection being neutralized by the disclosed method is an infection of marburgvirus. In some embodiments, the viral infection being neutralized by the disclosed method is an infection of influenza virus. In some embodiments, the viral infection being neutralized by the disclosed method is an infection of HIV-1.

Disclosed are methods of stimulating a therapeutically effective antigen-specific immune response against a virus in a mammal infected with the virus comprising administering any of the disclosed pharmaceutical compositions. In some embodiments, the disclosed method is a method of stimulating a therapeutically effective antigen-specific immune response against retroviridae. In some embodiments, the disclosed method is a method of stimulating a therapeutically effective antigen-specific immune response against a retrovirus. In some embodiments, the disclosed method is a method of stimulating a therapeutically effective antigen-specific immune response against flavivirus. In some embodiments, In some embodiments, the disclosed method is a method of stimulating a therapeutically effective antigen-specific immune response against Nipah virus. In some embodiments, the disclosed method is a method of stimulating a therapeutically effective antigen-specific immune response against West Nile virus. In some embodiments, the disclosed method is a method of stimulating a therapeutically effective antigen-specific immune response against human papillomavirus. In some embodiments, the disclosed method is a method of stimulating a therapeutically effective antigen-specific immune response against respiratory syncytial virus. In some embodiments, the disclosed method is a method of stimulating a therapeutically effective antigen-specific immune response against a filovirus. In some embodiments, the disclosed method is against zaire ebolavirus. In some embodiments, the disclosed method is a method of stimulating a therapeutically effective antigen-specific immune response against sudan ebolavirus. In some embodiments, the disclosed method is a method of stimulating a therapeutically effective antigen-specific immune responses against marburgvirus. In some embodiments, the disclosed method is a method of stimulating a therapeutically effective antigen-specific immune response against influenza virus. In some embodiments, the disclosed method is a method of stimulating a therapeutically effective antigen-specific immune response against HIV-1.

Disclosed are methods of inducing expression of a self-assembling vaccine in a subject comprising administering any of the disclosed pharmaceutical compositions. Also disclosed are methods of treating a subject having a viral infection or susceptible to becoming infected with a virus comprising administering to the subject a therapeutically effective amount of any of the disclosed pharmaceutical compositions. In some embodiments, the viral infection is an infection of retroviridae. In some embodiments, the viral infection is an infection of a retrovirus. In some embodiments, the viral infection is an infection of flavivirus. In some embodiments, the viral infection is an infection of Nipah virus. In some embodiments, the viral infection is an infection of West Nile virus. In some embodiments, the viral infection is an infection of human papillomavirus. In some embodiments, the viral infection is an infection of respiratory syncytial virus. In some embodiments, the viral infection is an infection of filovirus. In some embodiments, the viral infection is an infection of zaire ebolavirus. In some embodiments, the viral infection is an infection of sudan ebolavirus. In some embodiments, the viral infection is an infection of marburgvirus. In some embodiments, the viral infection is an infection of influenza virus. In some embodiments, the viral infection is an infection of HIV-1.

The disclosed pharmaceutical compositions may be administered by any route of administration. Accordingly, in some embodiments, the administering can be accomplished by oral administration. In some embodiments, the administering can be accomplished by parenteral administration. In some embodiments, the administering can be accomplished by sublingual administration. In some embodiments, the administering can be accomplished by transdermal administration. In some embodiments, the administering can be accomplished by rectal administration. In some embodiments, the administering can be accomplished by transmucosal administration. In some embodiments, the administering can be accomplished by topical administration. In some embodiments, the administering can be accomplished by inhalation. In some embodiments, the administering can be accomplished by buccal administration. In some embodiments, the administering can be accomplished by intrapleural administration. In some embodiments, the administering can be accomplished by intravenous administration. In some embodiments, the administering can be accomplished by intraarterial administration. In some embodiments, the administering can be accomplished by intraperitoneal administration. In some embodiments, the administering can be accomplished by subcutaneous administration. In some embodiments, the administering can be accomplished by intramuscular administration. In some embodiments, the administering can be accomplished by intranasal administration. In some embodiments, the administering can be accomplished by intrathecal administration. In some embodiments, the administering can be accomplished by intraarticular administration. In some embodiments, the administering can be accomplished by intradermal administration. In some embodiments, the above modes of action are accomplished by injection of the pharmaceutical compositions disclosed herein.

In some embodiments, the therapeutically effective dose can be from about 1 to about 30 micrograms of expressible nucleic acid sequence. In some embodiments, the therapeutically effective dose can be from about 0.001 micrograms of the composition per kilogram of subject to about 0.050 micrograms per kilogram of subject.

In some embodiments, any of the disclosed methods can be free of activating any mannose-binding lectin or complement process. In some embodiments, any of the disclosed methods may be performed without inducing the MBL-complement pathway.

In some embodiments, the subject can be a human. In some embodiments, the subject is diagnosed with or suspected of having a viral infection. For example, the subject can be diagnosed with or suspected of having an HIV-1 infection. In other embodiments, the subject can be diagnosed with or suspected of having a retrovirus infection. In some embodiments, the subject can be diagnosed with or suspected of having a flavivirus infection. In some embodiments, the subject can be diagnosed with or suspected of having a Nipah virus infection. In some embodiments, the subject can be diagnosed with or suspected of having a West Nile virus infection. In some embodiments, the subject can be diagnosed with or suspected of having a human papillomavirus infection. In some embodiments, the subject can be diagnosed with or suspected of having a respiratory syncytial virus infection. In some embodiments, the subject can be diagnosed with or suspected of having a filovirus infection. In some embodiments, the subject can be diagnosed with or suspected of having a zaire ebolavirus infection. In some embodiments, the subject can be diagnosed with or suspected of having a sudan ebolavirus infection. In some embodiments, the subject can be diagnosed with or suspected of having a marburgvirus infection. In some embodiments, the subject can be diagnosed with or suspected of having a influenza virus infection. In other embodiments, the subject can be diagnosed with or suspected of having an retrovirus infection. In some embodiments, the subject can be diagnosed with or suspected of having an flavivirus infection.

In some embodiments of the methods of inducing an immune response, the immune response can be an antigen-specific immune response. For example, the antigen-specific immune response can be an antigen-specific HIV-1 antigen immune response. In some embodiments, the antigen-specific immune response can be a therapeutically effective CD-4+ antigen-specific HIV-1 immune response. In some embodiments, the antigen-specific immune response can be a therapeutically effective CD-8+ antigen-specific HIV-1 immune response. In some embodiments, the antigen-specific immune response can be a therapeutically effective CD-4+ and CD-8+ antigen-specific HIV-1 immune response.

In some embodiments, the methods are free of administering any polypeptide directly to the subject.

In some embodiments, methods of inducing an immune response can include inducing a humoral or cellular immune response. A humoral immune response mainly refers to antibody production. A cellular immune response can include activation of CD4+ T-cells and activation CD8+ cells and associated cytotoxic activity. In one aspect, the present disclosure features a method of inducing an immune response in a subject, the method comprising administering to the subject in need thereof a pharmaceutically effective amount of any of the nucleic acid molecule comprising any one or plurality of the nucleic acid sequences or embodiments herein, or any one of the pharmaceutical compositions of any one of the aspects and embodiments herein. In one aspect, the present disclosure features a method of inducing a CD8+ T cell immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

In one aspect, the present disclosure features a method of enhancing an immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

In one aspect, the present disclosure features a method of enhancing a CD8+ T cell immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

In some embodiments, the subject has a viral infection and is in need of therapy for the viral infection. In some embodiments, the viral infection is an infection of retroviridae. In some embodiments, the viral infection is an infection of flavivirus. In some embodiments, the viral infection is an infection of Nipah Virus. In some embodiments, the viral infection is an infection of West Nile virus. In some embodiments, the viral infection is an infection of human papillomavirus. In some embodiments, the viral infection is an infection of respiratory syncytial virus. In some embodiments, the viral infection is an infection of filovirus. In some embodiments, the viral infection is an infection of zaire ebolavirus. In some embodiments, the viral infection is an infection of sudan ebolavirus. In some embodiments, the viral infection is an infection of marburgvirus. In some embodiments, the viral infection is an infection of influenza virus.

In some embodiments, the subject has previously been treated, and not responded to anti-viral therapy. In some embodiments, the nucleic acid molecule and/or expressible sequence is administered to the subject by electroporation.

The vaccine may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The vaccine may be administered by traditional syringes, needleless injection devices, “microprojectile bombardment gone guns”, or other physical methods such as electroporation (“EP”), “hydrodynamic method”, or ultrasound.

The plasmid of the vaccine may be delivered to the mammal by several well-known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The consensus antigen may be delivered via DNA injection and along with in vivo electroporation.

The vaccine or pharmaceutical composition can be administered by electroporation. Administration of the vaccine via electroporation of the plasmids of the vaccine may be accomplished using electroporation devices that can be configured to deliver to a desired tissue of a mammal a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation can be accomplished using an in vivo electroporation device, for example CELLECTRA® EP system (Inovio Pharmaceuticals, Inc., Blue Bell, PA) or Elgen electroporator (Inovio Pharmaceuticals, Inc.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the DNA vaccines of the present disclosure, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the DNA vaccines include those provided in co-pending and co-owned U.S. patent application Ser. No. 11/874,072, filed Oct. 17, 2007, which claims the benefit under 35 USC 119(c) to U.S. Provisional Application Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference in its entirety.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference in its entirety. The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge. Additionally, contemplated in some embodiments that incorporate

electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to a method of injecting DNA are contemplated herein. The above-patents are incorporated by reference in their entireties.

Methods of preparing the nucleic acid sequences are disclosed. In some embodiments, plasmid sequences with one or more multiple clining sites my be purchased from commercially available vendors and the expressible nucleic acid sequences disclosed herein may be ligated into the plasmids after a digestion with a known restriction enzyme needed to cute the plasmid DNA. In another alternative embodiment, membrane-based purification methods disclosed herein offer reduced cost, high binding capacity, and high flow rates, resulting in a superior purification process. The purification process is further demonstrated to produce plasmid products substantially free of genomic DNA, RNA, protein, and endotoxin.

In some embodiments, all of the described aspects of the present disclosure are advantageously combined to provide an integrated process for preparing substantially purified cellular components of interest from cells in bioreactors. Again, the cells are most preferably plasmid-containing cells, and the cellular components of interest are most preferably plasmids. The substantially purified plasmids are suitable for various uses, including, but not limited to, gene therapy, plasmid-mediated therapy, as DNA vaccines for human, veterinary, or agricultural use, or for any other application that requires large quantities of purified plasmid. In this aspect, all of the advantages described for individual aspects of the present disclosure accrue to the complete, integrated process, providing a highly advantageous method that is rapid, scalable, and inexpensive. Enzymes and other animal-derived or biologically sourced products are avoided, as are carcinogenic, mutagenic, or otherwise toxic substances. Potentially flammable, explosive, or toxic organic solvents are similarly avoided.

One aspect of the present disclosure is an apparatus for isolating plasmid DNA from a suspension of cells having both plasmid DNA and genomic DNA. An embodiment of the apparatus comprises a first tank and second tank in fluid communication with a mixer. The first tank is used for holding the suspension cells and the second tank is used for holding a lysis solution. The suspension of cells from the first tank and the lysis solution from the second tank are both allowed to flow into the mixer forming a lysate mixture or lysate fluid. The mixer comprises a high shear, low residence-time mixing device with a residence time of equal to or less than about 1 second. In a preferred embodiment, the mixing device comprises a flow through, rotor/stator mixer or emulsifier having linear flow rates from about 0.1 L/min to about 20 L/min. The lysate-mixture flows from the mixer into a holding coil for a period of time sufficient to lyse the cells and forming a cell lysate suspension, wherein the lysate-mixture has resident time in the holding coil in a range of about 2-8 minutes with a continuous linear flow rate.

The cell lysate suspension is then allowed to flow into a bubble-mixer chamber for precipitation of cellular components from the plasmid DNA. In the bubble mixer chamber, the cell lysate suspension and a precipitation solution or a neutralization solution from a third tank are mixed together using gas bubbles, which forms a mixed gas suspension comprising a precipitate and an unclarified lysate or plasmid containing fluid. The precipitate of the mixed gas suspension is less dense than the plasmid containing fluid, which facilitates the separation of the precipitate from the plasmid containing fluid. The precipitate is removed from the mixed gas suspension to give a clarified lysate having the plasmid DNA, and the precipitate having cellular debris and genomic DNA.

In some embodiments, the bubble mixer-chamber comprises a closed vertical column with a top, a bottom, a first, and a second side with a vent proximal to the top of the column. A first inlet port of the bubble mixer-chamber is on the first side proximal to the bottom of the column and in fluid communication with the holding coil. A second inlet port of the bubble mixer-chamber is proximal to the bottom on a second side opposite of the first inlet port and in fluid communication with a third tank, wherein the third tank is used for holding a precipitation or a neutralization solution. A third inlet port of the bubble mixer-chamber is proximal to the bottom of the column and about in the middle of the first and second inlets and is in fluid communication with a gas source the third inlet entering the bubble-mixer-chamber. A preferred embodiment utilizes a sintered sparger inside the closed vertical column of the third inlet port. The outlet port exiting the bubble mixing chamber is proximal to the top of the closed vertical column. The outlet port is in fluid communication with a fourth tank, wherein the mixed gas suspension containing the plasmid DNA is allowed to flow from the bubble-mixer-chamber into the fourth tank. The fourth tank is used for separating the precipitate of the mixed gas suspension having a plasmid containing fluid, and can also include an impeller mixer sufficient to provide uniform mixing of fluid without disturbing the precipitate. A fifth tank is used for a holding the clarified lysate or clarified plasmid containing fluid. The clarified lysate is then filtered at least once. A first filter has a particle size limit of about 5-10 μm and the second filter has a cut of about 0.2 μm. Although gravity, pressure, vacuum, or a mixture thereof can be used for transporting: suspension of cells; lysis solutions; precipitation solutions; neutralization solutions; or mixed gas suspensions from any of the tanks to mixers, holding coils or different tanks, pumps are utilized in a preferred embodiments. In a more preferred embodiment, at least one pump having a linear flow rate from about 0.1 to about 1 ft/second is used.

In another specific embodiment, a Y-connector having a having a first bifurcated branch, a second bifurcated branch and an exit branch is used to contact the cell suspension and the lysis solutions before they enter the high shear, low residence-time mixing device. The first tank holding the cell suspension is in fluid communication with the first bifurcated branch of the Y-connector through the first pump and the second tank holding the lysis solution is in fluid communication with the second bifurcated branch of the Y-connector through the second pump. The high shear, low residence-time mixing device is in fluid communication with an exit branch of the Y-connector, wherein the first and second pumps provide a linear flow rate of about 0.1 to about 2 ft/second for a contacted fluid exiting the Y-connector.

Another specific aspect of the present disclosure is a method of substantially separating plasmid DNA and genomic DNA from a bacterial cell lysate. The method comprises: delivering a cell lysate into a chamber; delivering a precipitation fluid or a neutralization fluid into the chamber; mixing the cell lysate and the precipitation fluid or a neutralization fluid in the chamber with gas bubbles forming a gas mixed suspension, wherein the gas mixed suspension comprises the plasmid DNA in a fluid portion (i.e. an unclarified lysate) and the genomic DNA is in a precipitate that is less dense than the fluid portion; floating the precipitate on top of the fluid portion; removing the fluid portion from the precipitate forming a clarified lysate, whereby the plasmid DNA in the clarified lysate is substantially separated from genomic DNA in the precipitate. In preferred embodiments: the chamber is the bubble mixing chamber as described above; the lysing solution comprises an alkali, an acid, a detergent, an organic solvent, an enzyme, a chaotrope, or a denaturant; the precipitation fluid or the neutralization fluid comprises potassium acetate, ammonium acetate, or a mixture thereof; and the gas bubbles comprise compressed air or an inert gas. Additionally, the decanted-fluid portion containing the plasmid DNA is preferably further purified with one or more purification steps selected from a group consisting of: ion exchange, hydrophobic interaction, size exclusion, reverse phase purification, endotoxin depletion, affinity purification, adsorption to silica, glass, or polymeric materials, expanded bed chromatography, mixed mode chromatography, displacement chromatography, hydroxyapatite purification, selective precipitation, aqueous two-phase purification, DNA condensation, thiophilic purification, ion-pair purification, metal chelate purification, filtration through nitrocellulose, or ultrafiltration.

In some embodiments, a method for isolating a plasmid DNA from cells comprising: mixing a suspension of cells having the plasmid DNA and genomic DNA with a lysis solution in a high-shear-low-residence-time-mixing-device for a first period of time forming a cell lysate fluid; incubating the cell lysate fluid for a second period of time in a holding coil forming a cell lysate suspension; delivering the cell lysate suspension into a chamber; delivering a precipitation/neutralization fluid into the chamber; mixing the cell lysate suspension and the a precipitation/neutralization fluid in the chamber with gas bubbles forming a gas mixed suspension, wherein the gas mixed suspension comprises an unclarified lysate containing the plasmid DNA and a precipitate containing the genomic DNA, wherein the precipitate is less dense than the unclarified lysate; floating the precipitate on top of the unclarified lysate; removing the precipitate from the unclarified lysate forming a clarified lysate, whereby the plasmid DNA is substantially separated from genomic DNA; precipitating the plasmid DNA from the clarified lysate forming a precipitated plasmid DNA; and resuspending the precipitated plasmid DNA in an aqueous solution.

The disclosure also relates to a method of treating and/or preventing viral infection in a subject comprising administering to the subject a therapeutically and/or prophylactically effective amount (as applicable) of a pharmaceutical composition comprising at least one expressible nucleic acid sequence, the expressible nucleic acid sequence comprising in 5′ to 3′ orientation a first, second and third nucleic acid sequence; wherein the first nucleic acid sequence encodes a leader sequence, the second nucleic acid sequence encodes a self-assembling polypeptide, and the third nucleic acid sequence encodes a viral antigen. In some embodiments, the first, second and third nucleic acid sequences are contiguous. In some embodiments, the first, second, third nucleic acid sequence are non-contiguous and are separated by one or a plurality of other independently selectable nucleic acids encoding the same or different viral antigens. In some embodiments, the first, second, third nucleic acid sequence are non-contiguous and are separated by one or a plurality of other independently selectable nucleic acids encoding the same or different self-assembling peptides.

### G. Vaccines

Disclosed are vaccines comprising at least about 70%, 75%, 80%, 85%, 90%, 91%, 92%, 93%, 94%, 95%, 96%, 97%, 98%, 99% or 100% sequence identity to SEQ ID NO: 69, or a functional fragment thereof. In some embodiment, the vaccines disclosed herein comprises the amino acid sequence of SEQ ID NO: 69, or a functional fragment thereof.

Also disclosed are DNA vaccines comprising an expressible nucleic acid sequence encoding a polypeptide comprising at least about 70%, 75%, 80%, 85%, 90%, 91%, 92%, 93%, 94%, 95%, 96%, 97%, 98%, 99% or 100% sequence identity to SEQ ID NO: 69, or a functional fragment thereof. In some embodiment, the DNA vaccines disclosed herein encodes a polypeptide comprising the amino acid sequence of SEQ ID NO: 69, or a functional fragment thereof. In some embodiment, the DNA vaccines disclosed herein comprises at least about 70%, 75%, 80%, 85%, 90%, 91%, 92%, 93%, 94%, 95%, 96%, 97%, 98%, 99% or 100% sequence identity to SEQ ID NO: 68, or a functional fragment thereof. In some embodiment, the DNA vaccines disclosed herein comprises the nucleic acid sequence of SEQ ID NO: 68, or a functional fragment thereof. In some embodiments, the disclosed DNA vaccine further comprises a pharmaceutically acceptable excipient. In some embodiments, the pharmaceutically acceptable excipient is an adjuvant.

Also disclosed are vaccines comprising at least 70%, 75%, 80%, 85%, 90%, 91%, 92%, 93%, 94%, 95%, 96%, 97%, 98%, 99% or 100% sequence identity to any of the sequences disclosed in Examples 8-11.

In some embodiments, the vaccine comprises a lipid nanoparticle (LNP) comprising one or a plurality of nucleic acid molecules disclosed herein. In some embodiments, delivery to a target cell is enhanced in vitro, while in other aspects, delivery to a target cell is enhanced in vivo. When administered in vivo, in one embodiment, target cell target cell delivery LNPs demonstrate enhanced delivery of agents to the liver and spleen when compared to reference LNPs. In some aspects, the target cell, e.g., a liver cell (e.g., a hepatocyte) or splenic cell, is contacted with the LNP in vitro. In some aspects, the target cell is contacted with the LNP in vivo by administering the LNP to a subject, e.g., a human subject. In one embodiment, the subject is one that would benefit from modulation of protein expression of a target protein, e.g., in a target cell. In some aspects, the LNP is administered intravenously. In some aspects, the LNP is administered intramuscularly. In some aspects, the LNP is administered by a route selected from the group consisting of subcutaneously, intranodally and intratumorally.

In some embodiments, the agent may comprise or consist of a nucleic acid molecule. In some aspects, the nucleic acid molecule is selected from the group consisting of RNA, mRNA, RNAi, dsRNA, siRNA, antisense RNA, ribozyme, CRISPR/Cas9, ssDNA and DNA. In some aspects, the nucleic acid molecule is RNA selected from the group consisting of a shortmer, an antagomir, an antisense, a ribozyme, a small interfering RNA (siRNA), an asymmetrical interfering RNA (aiRNA), a microRNA (miRNA or miR), a Dicer-substrate RNA (dsRNA), a small hairpin RNA (shRNA), a messenger RNA (mRNA), and mixtures thereof. In some embodiments, the nucleic acid molecule is an siRNA molecule. In some embodiments, the nucleic acid molecule is a miR. In some embodiments, the nucleic acid molecule is an antagomir. In some aspects, the nucleic acid molecule is DNA. In some aspects, the nucleic acid molecule is mRNA.

Accordingly, in one aspect the disclosure relates to a target cell delivery lipid nanoparticle (LNP) comprising:
(i) an ionizable lipid, e.g., an amino lipid;
(ii) a sterol or other structural lipid;
(iii) a non-cationic helper lipid or phospholipid;
(iv) a nucleic acids sequence disclosed herein; and
(v) optionally, a PEG-lipid,
wherein the target cell delivery LNP results in one, two, three or all of:
(a) enhanced payload level (e.g., expression) in a target cell, organ, cellular compartment, or fluid compartment e.g., liver or plasma (e.g., increased distribution, delivery, and/or expression of payload), e.g., relative to a different target cell, organ or cellular compartment, or relative to a reference LNP;
(b) enhanced lipid level in a target cell, organ, cellular compartment or fluid compartment, e.g., in the liver or plasma (e.g., increased distribution, delivery, or exposure of lipid), e.g., relative to a different target cell, organ or cellular compartment, or relative to a reference LNP;
(c) expression and/or activity of the nucleic acid sequence in greater than 30%, 40%, 50%, 60%, 65%, 70%, 75% or more total liver cells, e.g., in about 60% of total liver cells; or
(d) enhanced payload level (e.g., expression) and/or lipid level, e.g., about 1.5-fold, 2-fold, 3-fold, 4-fold, 5-fold, 6-fold (e.g., about 3-fold), in liver cell expression, e.g., hepatocyte expression, relative to a reference LNP.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising any of the elements of the disclosed nucleic acid compositions. For example, disclosed are kits comprising nucleic acid sequences comprising a leader sequence, a linker sequence, a nucleic acid sequence encoding a self-assembling polypeptide, and/or a nucleic acid sequence encoding a viral antigen. In some embodiments, the kits can further comprise a plasmid backbone.

### EXAMPLES

### Example 1. Production of and Experimentation with Plasmids Expressing Self-Assembling Nanoparticles

### Study Design

We were informed by our prior findings that synthetic DNA and electroporation (DNA/EP) can be used to deliver in vivo biologics such as antibodies and enzymes, and sought to determine in this study whether more complex structures such as macromolecular nanoparticle assemblies can also be delivered by DNA/EP. Nano-vaccines have historically been shown to induce more potent humoral responses than their monomeric counterparts but may be challenging to produce on a large scale. Therefore, a method to simplify the process by producing these nano-vaccines in the hosts may be relevant. Sample sizes in the study were pre-determined by power analyses with results from another set of the pilot studies. All the collected datapoints were included in the final analyses except for a single guinea pig in the DLmono_GT8 group which showed pre-existing antibody titers to GT8. All data were collected in at least technical duplicates, and except for the guinea pig and the challenge experiment, all findings were replicated successfully at least once in the study. Animals were randomly allocated to cages at the initiation of the study and were not further randomized. Data collection and analyses were not blinded. Detailed sample sizes are provided in the brief description of the drawings.

### Structure Modeling and Design of 3BVE, Ferritin, LS, PfV and Flu Nanoparticles

The nanoparticle structures for ferritin (PDB ID: 3BVE), lumazine synthase (PDB ID: 1HQK) and PfV (2E0Z) were used to seed the modeling simulations. The structure of eOD-GT8 (PDB ID: 5IDL) and HAI (PDB ID: 3GBN) were used to decorate the nanoparticles. N-linked glycans with missing density were added using glycan modeling modules of Rosetta [66]. Next, we wrote a new algorithm (simpleNanoparticleModeling) in the Molecular Software Library [67]. Briefly, we aligned the appropriate number of immunogens at the nanoparticle surface using coordinate frames constructed by 3 C-α atoms of the terminal positions of each protein. Immunogens were then tilted by random rotations around the x- and y-axes up to 30 degrees for the first ¾ of the simulation and up to 75 degrees for the last ¼ of the simulation, with a 120-degree rotation allowed for the z-axis. The immunogens were also translated by 10 Å to 200 Å along an axis projected away from the nanoparticle surface. Clashes were detected at each iteration and the models with the lowest number of clashes at each translation was written out as a potential structural model. The models were manually inspected and utilized to construct linkers as glycine-serine repeats using 30 Å per 9 linker residues as a guide. The sequence of the HA isolate H1 NC99 (A/New Caledonia/30/1999 (H1N1)) from residues 65-276 was used to construct the flu nanoparticle.

### DNA Design and Plasmid Synthesis

Protein sequences for IgE Leader Sequence and eOD-GT8-60mer were as previously reported[34, 68] and disclosed herein as SEQ ID NO: 6 and 9, respectively. Protein sequences for 3BVE-ferritin, PfV and HA_CA09 were obtained from UniProt (accession numbers: Q9ZLI1, 16U7J4, and C3W5X2). Protein sequence for HAI_NC99 was obtained from GenBank (accession number AY289929.1). DNA encoding protein sequences were codon and RNA optimized as previously described [34]. The optimized transgenes were synthesized de novo (GenScript, Piscataway, NJ) and cloned into a modified pVAX-1 backbone under the control of the human CMV promoter and bovine growth hormone poly-adenylation signal. All the plasmid maxi-preps were produced commercially (GenScript, Piscataway, NJ; Aldevron, Fargo, ND), with low endotoxin level (<0.005EU/μg). Production of His-tagged GT8-monomer and recombinant protein DLnanos

Expi293F cells were transfected with pVAX plasmid vector carrying the DLnano or His-tagged GT8-monomer transgene with PEI/OPTI-MEM and harvested 6 days post-transfection. Transfection supernatant was first purified with affinity chromatography using the AKTA pure 25 system and an IMAC Nickel column (for His-tagged GT8) and gravity flow columns filled with GNL Lectin beads (for DLnanos). The eluate fractions from the affinity purification were pooled, concentrated and dialyzed into 1×PBS buffer before being loaded onto the SEC column and then purified with size exclusion chromatography, for which the Superdex 75 10/300 GL column was used to purify His-tagged GT8-monomer and the Superose 6 Increase 10/300 GL column was used for DLnanos (run at 0.5 mL/min). Identified eluate fractions were then collected and concentrated to 1 mg/mL in PBS.

### Immunization

All mouse experiments were carried out in accordance with animal protocols 1127760 and 112782 approved by the Wistar Institute Institutional Animal Care and Use Committee (IACUC) (Philadelphia, PA). For DNA-based immunization, 6-8 week old female C57BL/6, BALB/c and CDI mice or 6-8 week old male BALB/c mice purchased from Jackson Laboratory or Charles River Laboratories were immunized one to three times (three-weeks apart) with DLmono_GT8, DLmono_HA_NC99, DLmono_HA_CA09, DNA-encoded LS_HA_CA09, DL_GT8_IMX313P or DLnano_LS_GT8, DLnano_CD4MutLS_GT8, DLnano_3 BVE_GT8, DLnano_PfV_GT8, DLnano_LS_HA_NC99 and DLnano_3 BVE_HA_CA09 via intramuscular injections into the tibialis anterior muscles (over two sites), followed by intramuscular electroporation with the CELLECTRA 3P device (Inovio Pharmaceuticals). For electroporation, 2 sets of 2 pulses (at 0.1 Amps) were delivered. Each set of 2 pulses lasts 52 milliseconds with a 1 second delay. For all DNA-encoded GT8-based immunizations (except for dosing studies), 25 μg of plasmid DNA was used, a standard DNA dose as in prior study [69]. For the control experiment to assess the importance of antigen decoration on nanoparticle, balb/c mice were immunized with 1:1 co-formulated (25 μg each) DLmono_GT8 with pVAX, DLmono_GT8 with DLnano_LS (core), and DLnano_LS_GT8 with pVAX and followed for seven d.p.i for sero-conversion. For all DNA-encoded HA-based immunizations, doses of 1 μg were used for each immunization for studies of humoral responses and 10 μg for studies of cellular responses. MBL knockout mice (B6.129S4-Mbl1tm1Kata Mbl2tm1Kata/J) and CR2 knockout mice (B6.129S7(NOD)-Cr2tm1Hmo/J) purchased from Jackson Laboratory were immunized in the same fashion.

For protein-based immunization, 6-8 week old female C57BL/6, MBL knockout and CR2 knockout mice were immunized subcutaneously over two sites with a high dose of 10 μg of recombinant eOD-GT8-60mer protein in 50 μL co-formulated with 50 μL Sigma adjuvant system (SigmaAldrich); the protein dose was 2.7-times higher than a prior study [26].

Female Hartley guinea pigs (8-10 weeks old) purchased from Charles River Laboratories (Wilmington, MA) were group housed and handled at AccuLab (San Diego, CA) with ad libitum access to food and water according to the standards of the Institutional Animal Care and Use Committee (IACUC) protocol CalMI2-043. Following acclimation, each guinea pig was given a single immunization of 50 μg of DLnano_LS_GT8 or DLmono_GT8 over 2 sites on the flank followed by intradermal EP with CELLECTRA 3P device. The animals were then bled at the indicated time points for humoral analyses.

### GT8-Binding ELISA

Corning 96-well half area plates were coated at room temperature for 6 hours with 1 μg/mL MonoRab anti-His antibody (GenScript), followed by overnight blocking with solution containing 1×PBS, 5% skim milk, 10% goat serum, 1% BSA, 1% FBS, and 0.2% Tween-20. The plates were then incubated with 2 μg/mL of his-tagged GT8-monomer at room temperature for 2 hours, followed by addition of mice sera serially diluted with PBS with 1% FBS and 0.1% Tween and incubation at 37° C. for 2 hours. The plates were then incubated at room temperature for 1 hour with Peroxidase AffiniPure Goat Anti-Mouse IgG, Fcγ fragment specific at 1:5,000 dilution (Jackson Immunoresearch) or AffiniPure Goat Anti-Mouse IgM, u chain specific, (Jackson Immunoresearch) at 1:5000 dilution followed by addition of TMB substrates (ThermoFisher) and then quenched with IM H2SO4. Absorbance at 450 nm and 570 nm were recorded with BioTEK plate reader. Endpoint titer is defined as the highest dilution at which the OD of the post-immune sera exceeds the cut-off (mean OD of naïve animals plus standard deviations of the OD in the naïve sera multiplied with standard deviation multiplier f at the 99% confidence level).

### VRC01 Competition ELISA

The plates were coated, and blocked, followed by addition with GT8-his as described in the last section. Serially diluted mice sera were then incubated with the plates at 37° C. for 1 hour, followed by addition of purified VRC01 antibody (NIH AIDS Reagent) for an additional 1 hour at room temperature. The plates were then incubated with anti-human Fc (cross-adsorbed against rabbits and mice) (Jackson Immunoresearch) at 1:10,000 dilution for 1 hour, followed by addition of TMB substrate for detection. Absorbance at 450 nm and 570 nm were recorded with BioTEK plate reader.

### MBL Binding ELISA

The plates were coated with 5 μg/mL recombinant mouse MBL protein (R&D system) in 0.1 M CaCl2 at room temperature for 6 hours, followed by blocking with 1% BSA in 0.1 M CaCl2 in PBS overnight at 4° ° C. Transfection supernatant or muscle homogenates containing DLmono_GT8 or DLnano_LS_GT8 were then added to the plates for 2 hour incubation at 37° C., followed by Week 5 sera of BALB/c mice previously immunized twice with 25 μg DLnano_LS_GT8. The plates were then incubated with anti-mouse IgG H+L (cross-adsorbed against human) HRP (Jackson Immunoresearch) at 1:10,000 dilution, followed by addition of TMB substrates. Absorbance at 450 nm and 570 nm were recorded with BioTEK plate reader.

### VRC01 Binding ELISA

ELISA format as described in the MBL binding ELISA section except that the recombinant MBL used in the coating step is replaced by 5 μg/mL of VRC01 (NIH AIDS Reagent). Absorbance at 450 nm and 570 nm were recorded with BioTEK plate reader. Antigenic profile characterization of designed GT8-nano-vaccines

Corning half-area 96-well plates were coated with 2 μg/mL of GT8-monomer, or 3BVE_GT8-24mer, eOD-GT8-60mer, CD4Mut_LS_GT8-60mer and PfV_GT8-180mer at 4° C. overnight. The plates were then blocked with the buffer as described in the GT8-binding ELISA section for 2 hours at room temperature, followed by incubation with serially diluted VRC01 at room temperature for 2 hours. The plates were then incubated with anti-human Fc (cross-adsorbed against rabbits and mice) (Jackson Immunoresearch) at 1:10,000 dilution for 1 hour, followed by addition of TMB substrate for detection. Absorbance at 450 nm and 570 nm were recorded with BioTEK plate reader.

### HA-Binding ELISA

Corning 96-well half area plates were coated at 4° C. overnight with 2 μg/mL of recombinant HA(ΔTM)(H1N1/A/New Caledonia/20/1999) or HA(ΔTM)(A/California/04/2009)(H1N1) (Immune Technology), and blocked at room temperature for 2 hour with the buffer as described in the GT8-binding ELISA section. The plates were subsequently incubated with serially diluted mouse sera in PBS with 1% FBS and 0.1% Tween at 37° C. for 2 hours, followed by 1-hour incubation with anti-mouse IgG H+L HRP (Bethyl) at 1:20,000 dilution at room temperature and development with the use of TMB substrate. Absorbance at 450 nm and 570 nm were recorded with BioTEK plate reader. HAI assay

Mice sera were treated with receptor-destroying enzyme (RDE, 1:3 ratio) at 37° C. overnight for 18-20 hours followed by complement and enzyme inactivation at 56° C. for 45 minutes. RDE-treated sera were subsequently cross-adsorbed with 10% rooster red blood cells (Lampire Biologicals) in PBS at 4° ° C. for 1 hour. The cross-adsorbed sera were then serially diluted with PBS in a 96-well V-bottom microtiter plates (Corning). Four hemagglutinating doses (HAD) of A/Solomon Islands/03/06 virus, A/New Caledonia/20/99, or A/California/07/2009 (BEI) were added to each well and the serum-virus mixture was incubated at room temperature for 1 hour and then incubated with 50 μl 0.5% v/v rooster red blood cells in 0.9% saline for 30 minutes at room temperature. The HAI antibody titer was scored with the dot method, and the reciprocal of the highest dilution that did not exhibit agglutination of the rooster red blood cells was recorded.

### Immunofluorescence

For lymph node staining, 7 days after BALB/c mice were immunized with 80 μg DNA co-formulated with 12U Hyaluronidase (Sigma) encoding GT8-monomer or DLnano_LS_GT8, tibialis anterior muscles of the mice were injected with 5 μg of anti-mouse CD35 BV421 (BD-Bioscience) for in situ labelling of follicular dendritic cells 16 hours prior to harvest. Ipsilateral iliac lymph nodes from the mice were harvested the next day and preserved in O.C.T medium (Fisher) for cryo-sectioning. The sections were fixed with 4% paraformaldehyde, then blocked in 3% BSA/PBS for 1 hour at room temperature, followed by overnight staining with 6 μg/mL VRC01. The sections were then washed and stained with anti-human Alexa Fluor 488 antibody and imaged with Leica SP5 confocal microscopes.

For muscle staining, four days after BALB/c mice were immunized with 80 μg DNA encoding GT8-monomer, DLnano_LS_GT8, DLnano_3 BVE_GT8 or DLnano_PfV_GT8 co-formulated with 12U Hyaluronidase in the tibialis anterior muscles of the mice were harvested and preserved in 4% PFA/PBS for 2 hours at room temperature and then stored overnight in 70% EtOH/H2O at 4° C. The tissues were then serially dehydrated and blocked in 3% BSA/PBS for 1 hour at room temperature, followed by overnight staining with 6 μg/mL VRC01. The sections were then washed, and stained with anti-human Alexa Fluor 488 antibody, counterstained with 0.5 μg/mL DAPI and imaged with Leica SP5 confocal microscopes.

For transfected cells, HEK293T cells were cultured in poly-lysine coated glass chambers overnight, and then transfected with DNA encoding GT8-monomer or eOD-GT8-60mer with GeneJammer (Agilent). The cells were harvested 48 hours post transfection, fixed with 4% paraformaldehyde, permeabilized with 0.5% Triton X-100/PBS, blocked and stained as in the section describing muscle immunofluorescence staining.

### Immunohistochemistry

For immunohistochemistry staining of muscle sections, BALB/C mice were immunized with 80 μg DLmono_GT8 or DLnano_LS_GT8 co-formulated with 12U hyaluronidase (Sigma). Transfected muscles were harvested seven days post immunization, cryo-sectioned, fixed, permeabilized, and blocked as described in the Immunofluorescence section. The muscle sections were then stained with goat anti-mouse MBL at 1:200 dilution in 1% BSA/PBS (R&D system) overnight, and then with secondary Rabbit anti-goat (H+L) HRP conjugated at 1:500 dilution (BioRad) and DAB substrates for development.

### Transmission EM of Muscles

Tibialis anterior muscles from BALB/c mice immunized with 80 μg DLmono_GT8 or DLnano_LS_GT8 co-formulated with 12U hyaluronidase were collected seven d.p.i. The muscles were then fixed in 2.5% glutaraldehyde, serially dehydrated in acetone/ethanol solvents, and then embedded in epoxy and LR white resin. The resin was then sectioned to a thickness of 70 nm and deposited onto a metal grid, blocked overnight in 3% BSA/PBS, followed by staining with 60 μg/mL VRC01 (diluted in 3% BSA/PBS) overnight, and with 1:200 anti-human 6 nm gold nanoparticles (Jackson Immunoresearch) for 1 hour. The sections were then washed with 0.1% Tween in PBS, and water, followed by post-staining fixation with 2.5% glutaraldehyde in PBS for 5 minutes at room temperature followed by staining with 2% Uranyl acetate for 1 hour. The grids were subsequently imaged with JEOL JEM 1010 transmission electron microscope. For quantitative analyses, total number of gold-labeled clusters, and order of each cluster were manually counted. Frequency of a cluster of a particular order in a field of view was normalized relative to the total number of clusters observed.

### Negative Stain EM of Purified Nanoparticles

The nanoparticles were produced in Expi293 cells, purified using Agarose bound lectin beads (Agarose Galanthus Nivalis Lectin, Vector Laboratories) followed by size exclusion chromatography (GE Healthcare) using the Superose 6 Increase 10/300 GL column. The proteins were further dialyzed into Tris-buffered saline (TBS). A total of 3 μL of purified proteins was adsorbed onto glow discharged carbon-coated Cu400 EM grids. The grids were then stained with 3 μL of 2% uranyl acetate, blotted, and stained again with 3 μL of the stain followed by a final blot. Image collection and data processing was performed on a FEI Tecnai T12 microscope equipped with an Oneview Gatan camera at 90,450× magnification at the camera and a pixel size of 1.66 Å.

### ELISpot Assay

Spleens from immunized mice were collected 5 weeks post the first immunization (2 weeks post the second) and homogenized into single cell suspension with a tissue stomacher in 10% FBS/1% Penicillin-streptomycin in RPMI 1640. Red blood cells were subsequently lysed with ACK lysing buffer (ThermoFisher) and percentage of viable cells were determined with Trypan Blue exclusion. 200,000 cells were then plated in each well in the mouse IFNγ ELISpot plates (MabTech), followed by addition of peptide pools that span both the lumazine synthase, GT8 or HA domains at 5 μg/mL of final concentration for each peptide (GenScript). The cells were then stimulated at 37° C. for 16-18 hours, followed by development according to the manufacturer's instructions. Spots for each well were then imaged and counted with ImmunoSpot Macro Analyzer.

### Intracellular Cytokine Staining

Single cell suspension from spleens of immunized animals were prepared as described in the previous section and stimulated with 5 μg/mL of peptides spanning both the lumazine synthase, GT8 or HA domains(GenScript) for 5 hours at 37° C. in the presence of 1:500 protein transport inhibitor (ThermoFisher) and anti-mouse CD107a-FITC(ThermoFisher). The cells were then incubated with live/dead for 10 minutes at room temperature, surface stains (anti-mouse CD4 BV510, anti-mouse CD8 APC-Cy7, anti-mouse CD62L BV711 and anti-mouse CD44 AF700) (BD-Biosciences) at room temperature for 30 minutes. The cells were then fixed and permeabilized according to manufacturer's instructions for BD Cytoperm Cytofix kit and stained with intracellular stains anti-mouse IL-2 PE-Cy7, anti-mouse IFN-γ APC, anti-mouse CD3c PE-Cy5 and anti-mouse TNFα BV605 (BioLegend) at 4° C. for 1 hour. The cells were subsequently analyzed with LSR II 18-color flow cytometer.

### Immunoblotting

Tibialis anterior muscles of immunized animals were harvested and homogenized in T-PER extraction buffer (ThermoFisher) and protease inhibitor (Roche). Muscle homogenates were subsequently concentrated 20× with Amicon Ultra 0.5 mL Centrifugation kits with 3 kDA cutoffs (Milipore Sigma) and protein concentrations were quantified with BCA assays (ThermoFisher). For electrophoresis, 8 μL supernatants of Expi293F cells transfected with pVAX, DLmono_GT8, eOD-GT8-60mer or 50 μg muscle homogenates from mice immunized with the 80 μg aforementioned constructs co-formulated with 12U hyaluronidase were loaded onto 4-12% SDS Bis-Tris Gel (SDS-PAGE) or 3-8% Tris-Acetate Gel (pseudo-Native PAGE) for electrophoresis. For SDS-PAGE, all samples were reduced with heating of the samples in the presence of a reducing agent and LDS sample buffer (ThermoFisher) at 70° C. for 10 minutes. For Pseudo-Native PAGE, samples were only incubated with the LDS buffer at room temperature and loaded directly onto the 3-8% TA gel without boiling. Proteins were subsequently transferred to PVDF membrane from the gels, and stained with 3 μg/mL of VRC01 and 1 μg/mL anti-human GAPDH (for SDS-PAGE only, Clone D4C6R, Cell Signaling) in Odyssey Blocking Buffer/PBS/0.1% Tween (LI-COR Biosciences) overnight at 4° C., and 1:10,000 IRDye 800CW goat anti-human IgG (LI-COR Biosciences) in Odyssey Blocking Buffer/0.1% Tween/0.1% SDS at room temperature for 1 hour, and then scanned with LI-COR Odyssey CLx.

### Determination of the Antigen-Specific B-Cells in Spleen

Recombinant 3BVE-GT8 was labelled with FITC with the lightning link kits according to manufacturer's instructions (Expedon). Spleens were harvested five weeks post the second immunization of 25 μg of DLnaono_LS_GT8, DLmono_GT8 or from Naïve mice. Single cells were then labelled with Live/Dead dye ultraviolet reactive (ThermoFisher) at room temperature for 10 minutes and incubated with mouse Fc-Block (Clone 93, ThermoFisher) at 1:200 dilution. Avi-Tagged GT8 was biotinylated and tetramerized with an excess of APC-streptavidin (ThermoFisher) as previously described [27]. The cells were washed with PBS and incubated with 1:200 A488-3BVE-GT8 and 1:200 APC-GT8-tetramer at 4° C. for 30 minutes. Without being washed, the cells were incubated with 1:200 anti-mIgD-APC/Cy7 (BioLegend), anti-mIgM-BV711 (Fisher Scientific), anti-mCD19-PECy7 (Biolegend), anti-mIgG-BV510 (Biolegend) in 1% FBS/PBS solution. The cells were then resuspended in 1× BDFix buffer and analysed with LSR II 18-color flow cytometer.

### Lethal H1/A/California/07/09 Influenza Challenge

6-8 week old female BALB/c mice (Jackon Laboratory) were immunized with 1 μg of pVAX vector, DLmono_HA_CA09 or DLnano_3 BVE_HA_CA09 twice three weeks apart. The mice were subsequently transferred to BioQual Inc for challenge experiment. 35 days post the second immunization, the mice were intranasally inoculated with 10LD50 H1/A/California/07/09 in PBS. Weights of the mice were pre-recorded prior to the challenge and daily after the challenge until 7 days post inoculation, at which lungs from the mice were harvested and snap-frozen for viral load assay by RT-qPCR and histo-pathology by H&E staining. At any point, mice exhibiting more than 20% of weight loss as compared to baseline were euthanized (humane endpoint).

### RT-qPCR Assay for Viral Load Determination

The amounts of RNA copies per gram lung tissue was determined using a real-time quantitative PCR (qPCR) assay. This assay utilized primers and a probe specifically designed to amplify and bind to a conserved region of the NP gene of influenza virus. The signal was compared to a known standard curve and calculated to give copies per gram tissue. Viral RNA was extracted from lung homogenates using MiniElute Virus Spin Kit (Qiagen). TAQMAN RT-PCR kit (Applied Biosystems, Inc., Carlsbad, CA) was used for amplification of viral RNA in the presence of 600 nM primers (CAL-1-U: ATGGCGTCTCAAGGCACCAA and CAL-1-D: GCACATTTGGATGTAGAATCTC) and 140 nM probe (CAL-1-P: 6FAM-CAGAGCATCTGTCGGAAGAATGATTG-TAMRA) with the following Thermocycler setting: 48° C. for 30 minutes, 95° C. for 10 minutes followed by 40 cycles of 95° C. for 15 seconds, and 1 minute at 60° C.

### Statistics

We have performed power analysis with R based on our preliminary data to determine the smallest sample size that would allow us to achieve a power of 0.9 with a pre-set a-value of 0.05. All statistical analyses were performed with PRISM V8.0 and R V3.5.1. Each individual data point was sampled independently. Two-tailed Mann Whitney Rank Tests were used to compare differences between groups. Bonferroni corrections were used when multiple comparisons were made.

### Example 2. DNA-Launched GT8 Nanoparticles Expressed and Assembled In Vitro and In Vivo

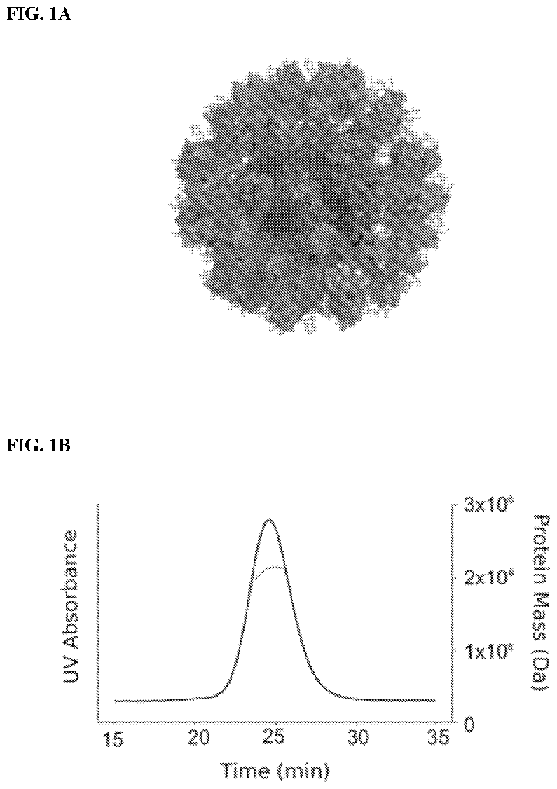

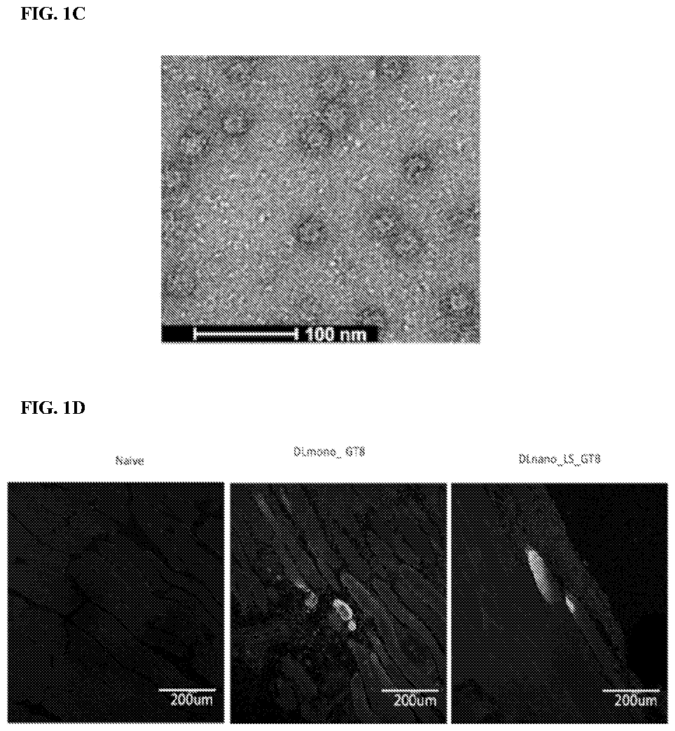

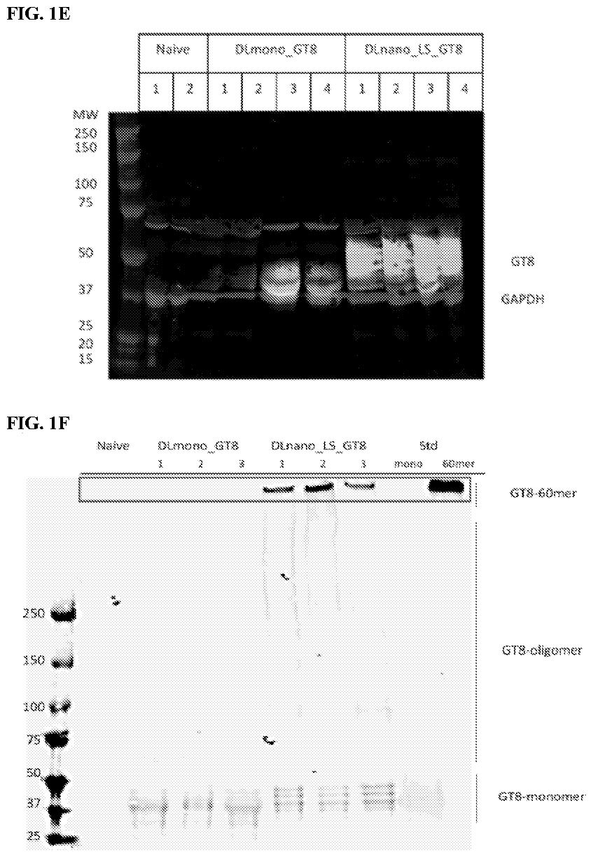

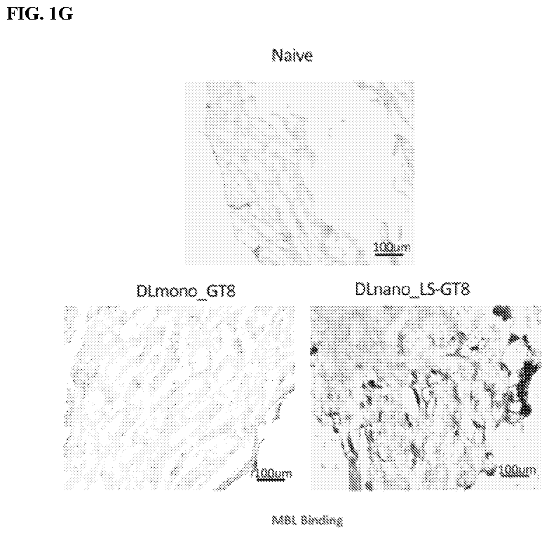

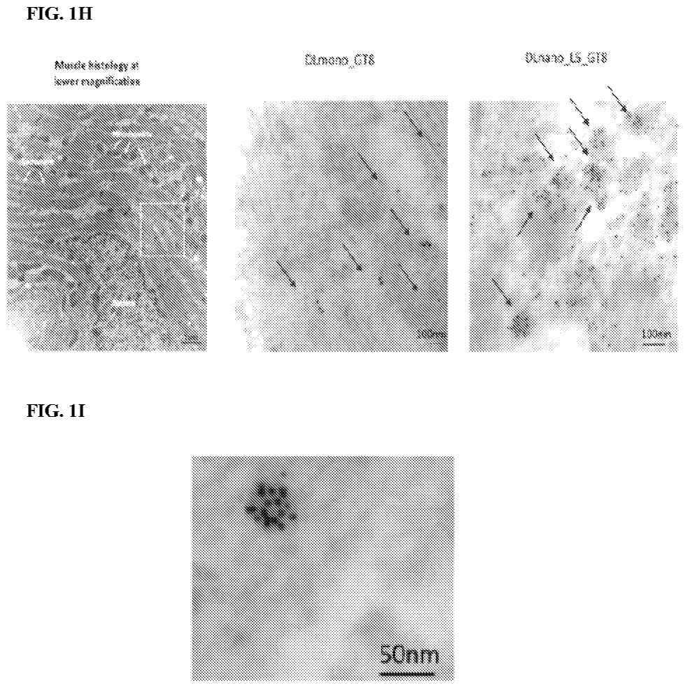

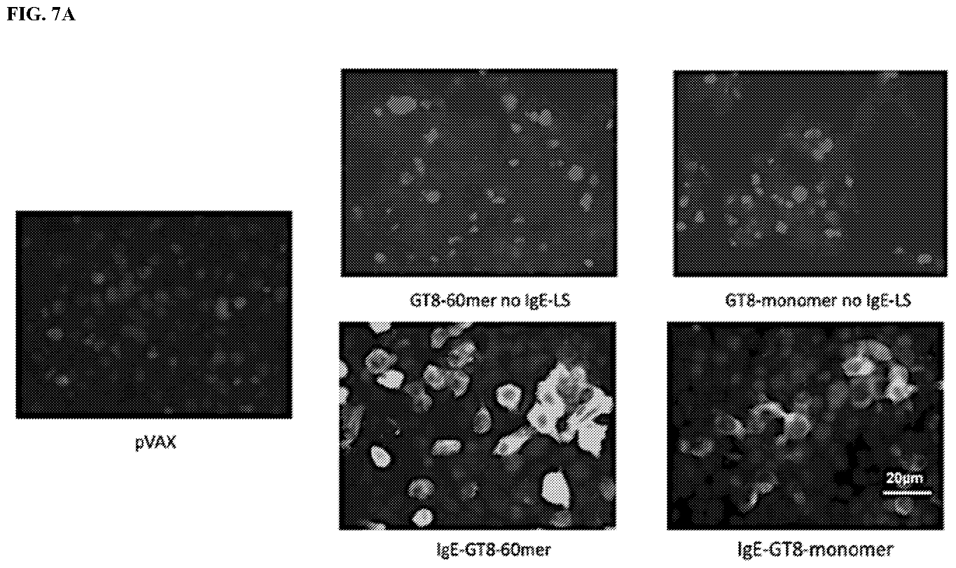

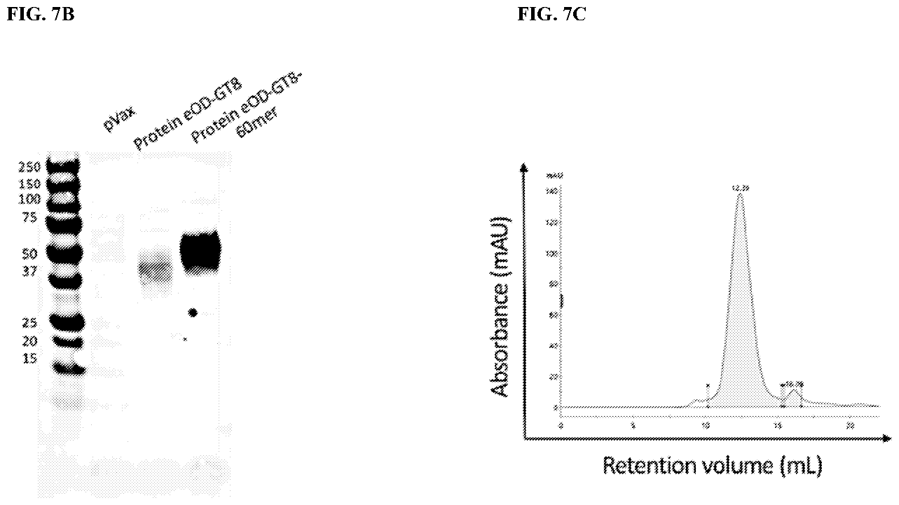

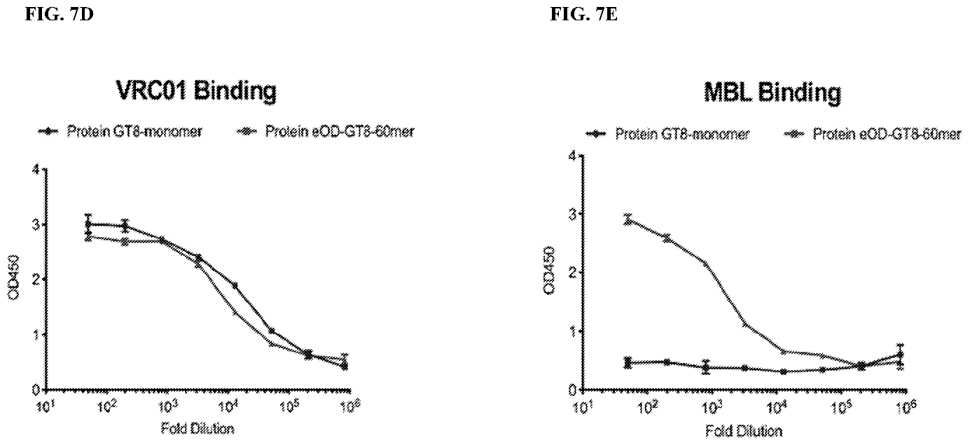

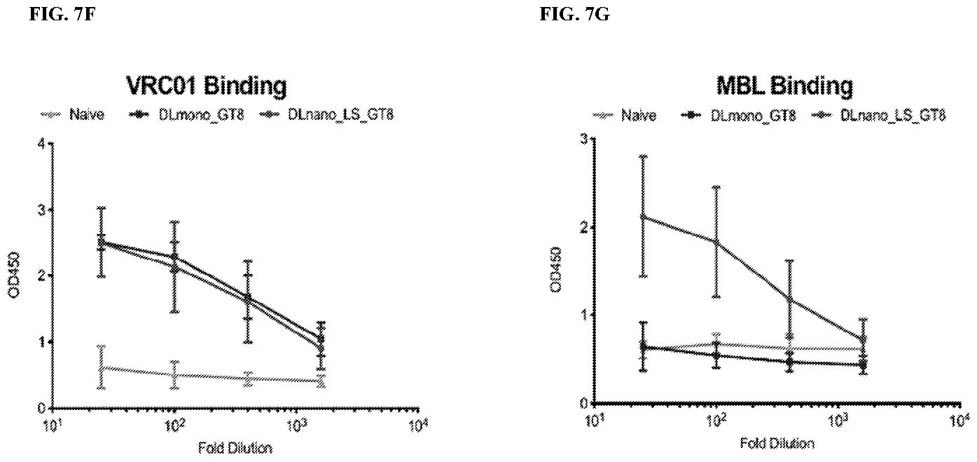

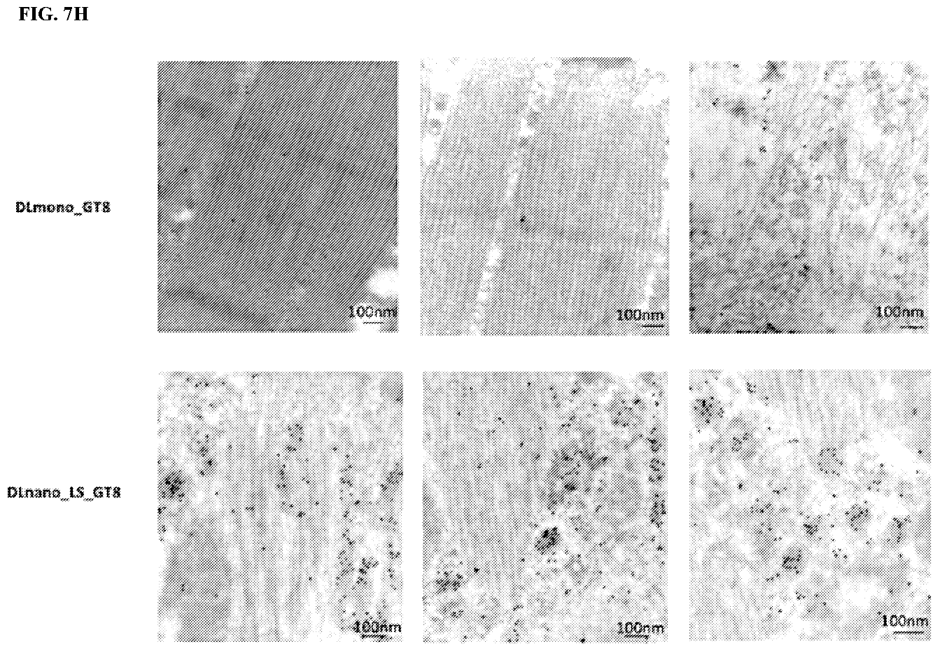

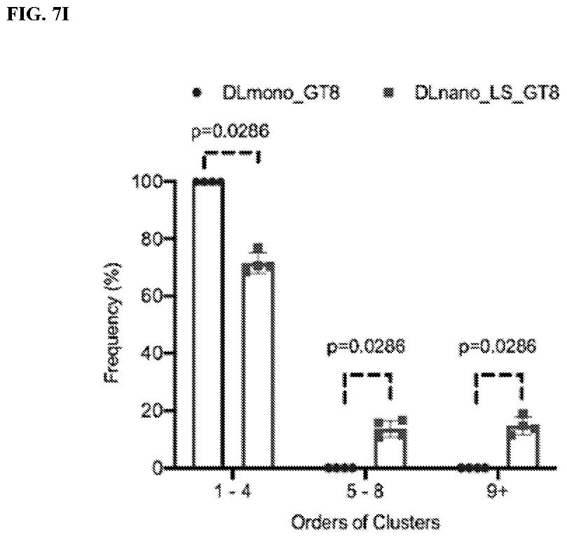

To determine whether DNA/EP could be used to launch structurally designed, self-assembling protein nanoparticles in vivo, we encoded the transgene eOD-GT8-60mer in the pVAX1 vector and optimized the DNA cassette for in vivo nanoparticle expression (FIG. 1A). We first evaluated expression, secretion and assembly of plasmid encoded GT8 constructs in vitro. We engineered the GT8 constructs to incorporate an optimized human IgE-leader sequence and found the in vitro intracellular expression of this construct to be strongly enhanced as compared to GT8 constructs without any leader sequence (FIG. 7A). We therefore used the IgE-constructs for subsequent experiments. In addition, reducing SDS-PAGE analysis of transfection supernatants supported that both plasmid-encoded GT8-monomer and eOD-GT8-60mer could be secreted (FIG. 7B). Lectin-purified protein eOD-GT8-60mer eluted as a homogenous fraction by size-exclusion chromatography (SEC) (FIG. 7C). The assembled protein was observed to be approximately 2 MDa as determined by protein conjugate analysis with size-exclusion multiangle light scattering, SEC-MALS (FIG. 1B). Negative stain electron microscopy (nsEM) also supported correct assembly of protein COD-GT8-60mer with a diameter of around 32 nm (FIG. 1C).

Next, we examined the in vivo expression of both DNA-encoded GT8 monomer and nanoparticle constructs. Immunofluorescent staining of mouse muscles transfected with DNA/EP four days post injection (d.p.i) with VRC01 (a human broadly neutralizing antibody with high-affinity for GT8) showed that both DNA-encoded GT8 constructs expressed in vivo (FIG. 1D). Reducing SDS-PAGE western analyses of muscle homogenates four d.p.i with VRC01 (in green) also confirmed in vivo expression of GT8 antigens, even though in vivo expression of DLnano_LS_GT8 was stronger and more consistent than DLmono_GT8 (FIG. 1E). The assembly states of in vivo produced DLnano_LS_GT8 as compared to DLmono_GT8 in mouse muscle homogenates was examined with pseudo-native PAGE. Well-formed 60mer GT8-nanoparticles, as defined by the migration pattern of SEC-purified recombinant protein eOD-GT8-60mer standard, was observed only in DLnano_LS_GT8 treated but not in DLmono_GT8 treated mice (FIG. 1F). Bands that corresponded to monomeric and oligomeric GT8 band were also observed in DLnano_LS_GT8 muscle homogenates but were significantly less intense than the 60-mer band and may represent newly synthesized GT8-subunits or partially assembled GT8-oligomer transiting through cellular secretory networks.

Next, we used a mannose binding lectin (MBL) labelling experiment to assess for in vivo antigen multimerization and nanoparticle assembly. MBL is a protein that can form hexamer and preferentially bind to highly repeated glycan structures on a pathogen/antigen surface [44]. A recent study by Tokatilian et al. demonstrated that only highly multimerized glycan structures (eOD-GT8-60mer but not eOD-GT8-monomer) could bind to MBL [26]. In our study, we similarly showed using ELISA that while VRC01 could bind to both protein GT8-60mer and GT8-monomer, murine MBL could only bind to protein GT8-60mer but not protein GT8-monomer (FIG. 7D and FIG. 7E). Using this assay as a multimerization readout, we demonstrated that in vivo produced DLnano_LS_GT8, but not DLmono_GT8 could bind to MBL (FIG. 7F and FIG. G). Further, we observed that DLnano_LS_GT8 could be strongly labelled by endogenous murine MBL via an immune-histochemistry experiment (FIG. 1G).

As an additional way to assess in vivo nanoparticle formation, we employed a transmission electron microscopy-based technique, where thin sections of transfected muscles were stained with VRC01 and gold-conjugated anti-human IgG. Clusters of gold-labelled macromolecules suggestive of in vivo launched nanoparticles decorated with multiple copies of GT8 were only observed in mice injected with DLnano_LS_GT8 but not with DLmono_GT8 (FIG. 1H and FIG. 7H). In DLnano_LS_GT8 immunized mice, these clusters often had a valency greater than 10 (FIG. 1I). We expected some reduction in labelling valency due to both steric hindrance in binding of VRC01 to individual GT8 subunits and limited solvent exposure on nanoparticle surfaces with thin sample sectioning. Quantitative measurements of the orders of clusters in different fields of interests demonstrated that partially formed (orders between 5 and 8) and well-formed (orders no less than 9) nanoparticles were significantly more frequent in mice treated with DLnano_LS_GT8 than with DLmono_GT8, confirming in vivo assembly of these complex nano-vaccines (FIG. 7I).

### Example 3. DLnano_LS_GT8 Elicited More Rapid Seroconversion and Higher Setpoint Antibody Titers than DLmono_GT8 and Similar Titers to Protein eOD-GT8-60mer

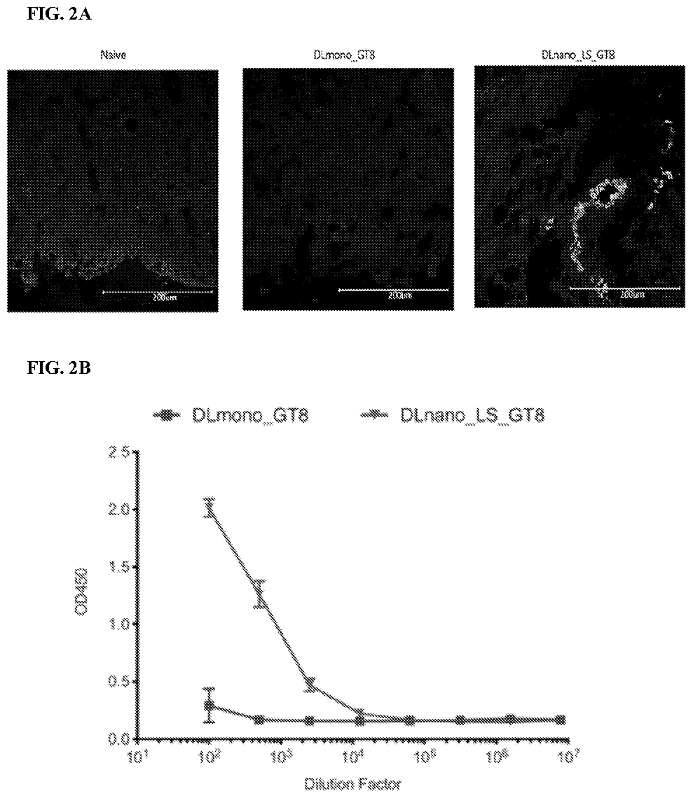

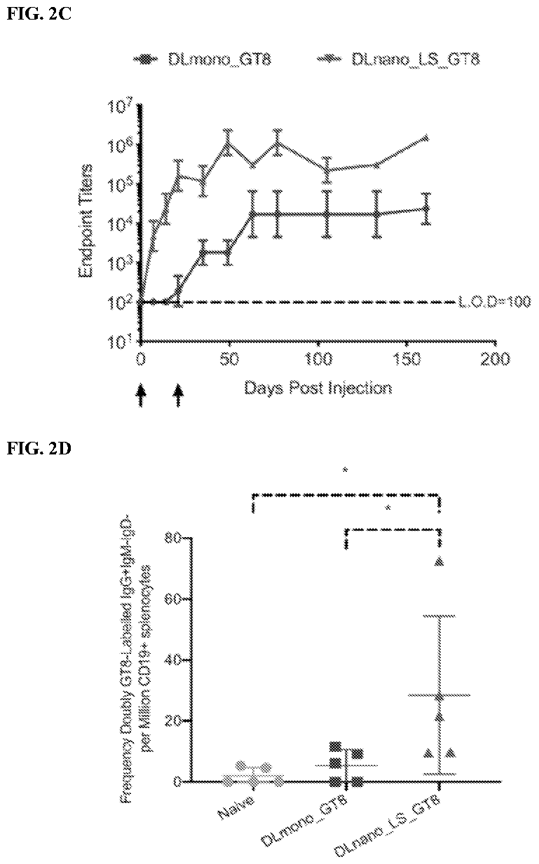

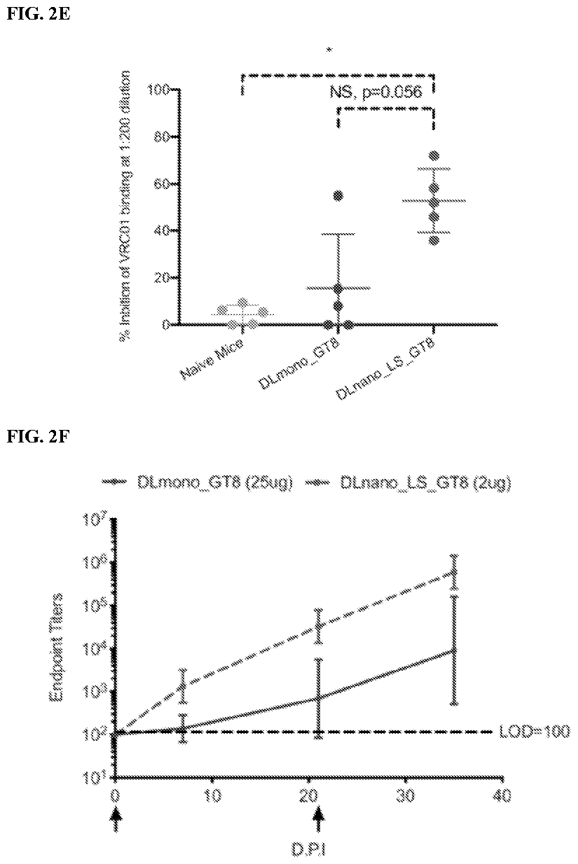

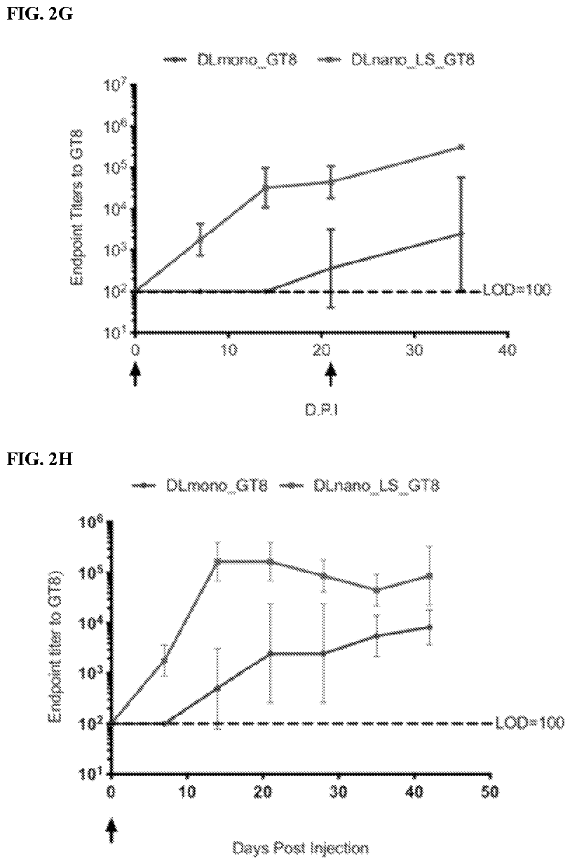

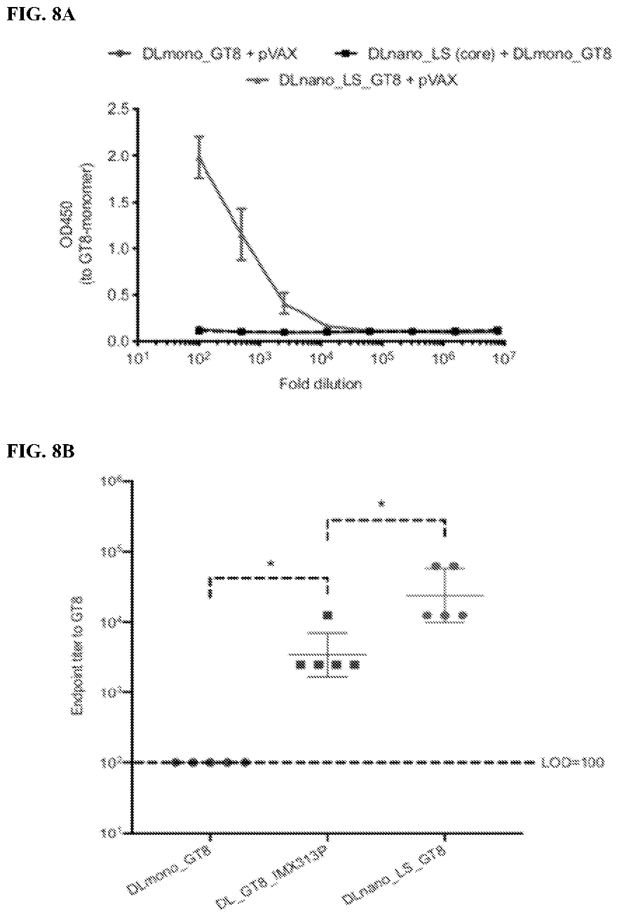

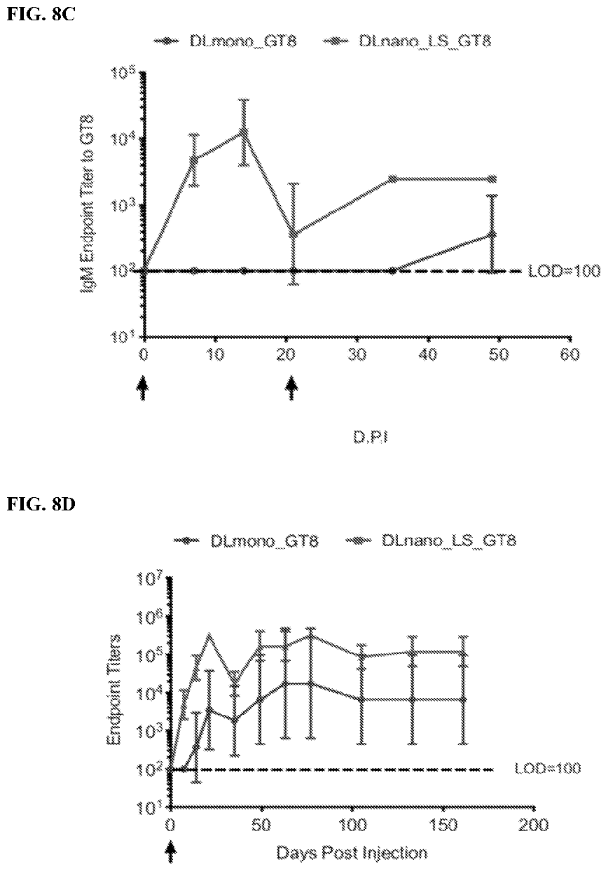

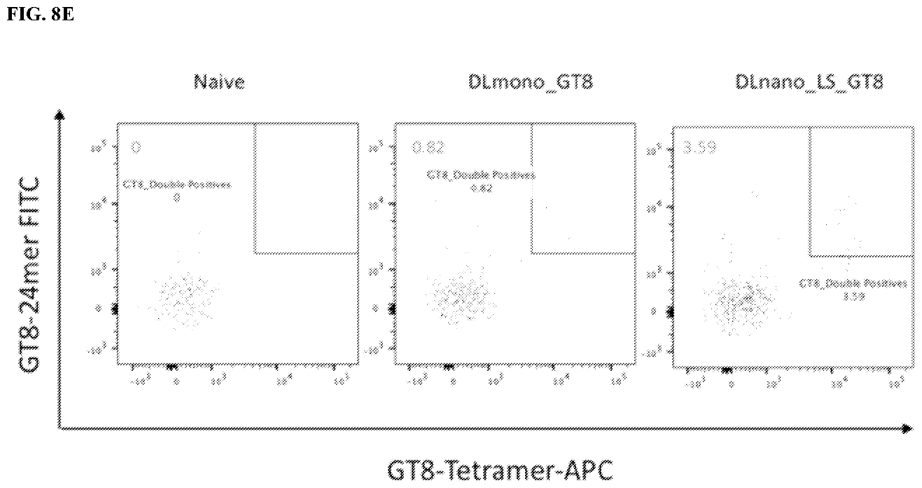

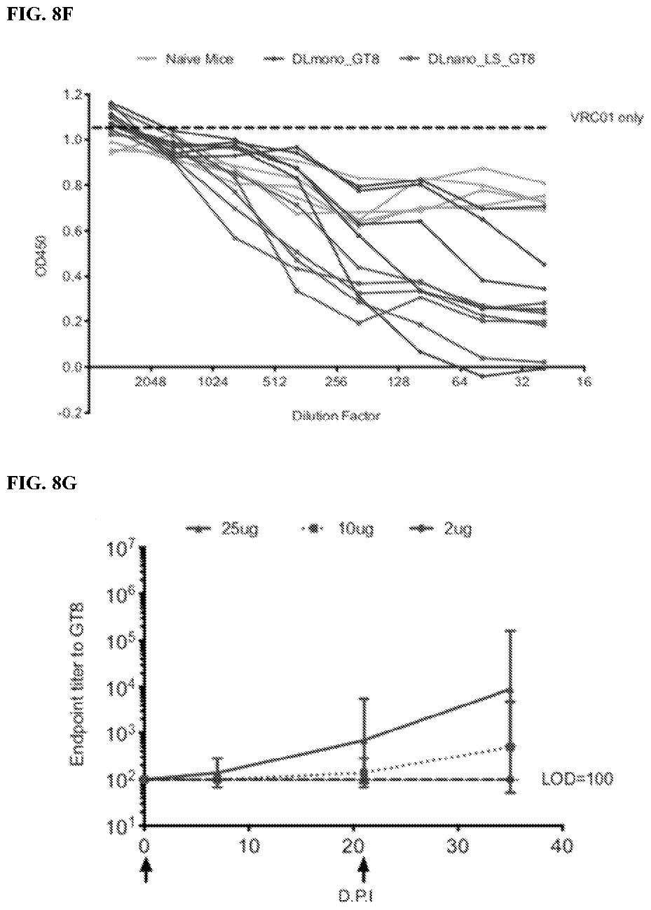

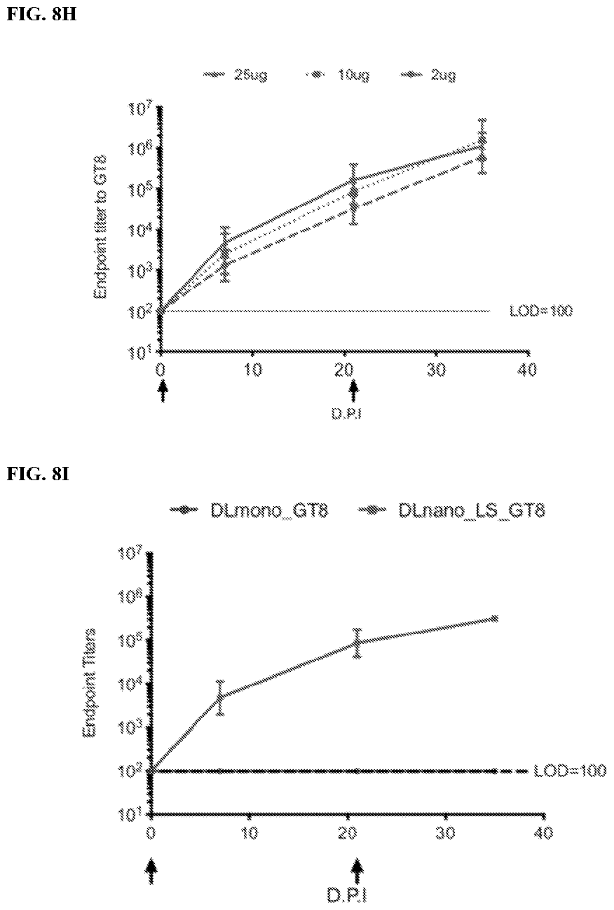

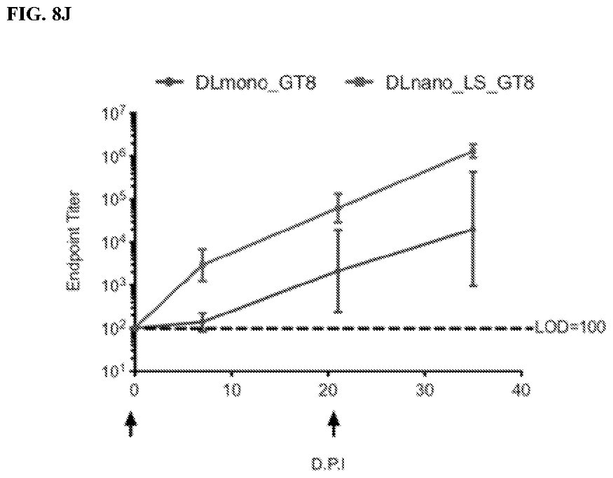

Using immunofluorescence staining with VRC01 (green), we determined that DLnano_LS_GT8 trafficked more efficiently to the draining lymph node and co-localized with the CD35+ follicular dendritic cells (in blue) in contrast with the DLmono_GT8 seven d.p.i. (FIG. 2A). This observation is consistent with recent findings on trafficking of recombinant protein nanoparticle vaccines [26]. To determine whether improved immunogen trafficking correlated with enhanced adaptive immunity, we followed humoral responses in immunized BALB/c mice. After seven d.p.i, we found that DLnano_LS_GT8 induced more rapid GT8-directed seroconversion than DLmono_GT8 (FIG. 2B). Decoration of the GT8-antigens on the LS nanoparticle core is essential for the observed early response as co-transfection of mice muscles with 1:1 ratio of DLmono_GT8 and DNA-encoded lumazine synthase core (DLnano_LS_core) did not lead to seroconversion at this timepoint (FIG. 8A). We next examined if GT8 scaffolded with a simpler multimerization domain, IMX313P, would perform similarly. Heptameric DNA-encoded GT8-IMX313P (DL_GT8_IMX313P) led to limited seroconversion at seven d.p.i, but the induced antibody titer was 6.9-fold lower than that of DLnano_LS_GT8 (FIG. 8B). Antigen-specific circulating IgMs can play a role in protection from challenge [45]. Here, we measured induced IgM responses and found that DLnano_LS_GT8 induced stronger IgM responses than DLmono_GT8 with two immunizations (FIG. 8C). Further, the IgG titers were 1.3-log and 1.8-log higher for DLnano_LS_GT8 with single immunization (FIG. 8D) or two immunizations (FIG. 2C) respectively. Consistent with this observation, we found the frequency of CD19+IgD-IgM-IgG+GT8 antigen-specific B cells in the spleens of mice immunized with DLnano_LS_GT8 to be 5.3-fold higher relative to mice immunized with DLmono_GT8 (FIG. 2D), even though relatively few CD19+IgD-IgM-IgG+GT8-24mer+GT8-tetramer+B cells have been recovered per million splenocytes analyzed (FIG. 8E). DLnano_LS_GT8 retained folding and presentation of a key conformational epitope in vivo, as elicited murine antibodies could outcompete VRC01 binding to GT8 in competition ELISA (FIG. 8F and FIG. 2E). A striking dose-sparing effect was observed for DLnano_LS_GT8. While humoral responses were remarkably attenuated for DLmono_GT8 at 2 and 10 μg doses (FIG. 8G), DLnano_LS_GT8 given at 2, 10 or 25 μg doses all induced similar levels of antibody responses (FIG. 8H). Importantly, differences in antibody responses induced by DLnano_LS_GT8 and DLmono_GT8 were probably not solely due to increased antigen expression for DLnano_LS_GT8 (FIG. 1E), as DLnano_LS_GT8 still outperformed DLmono_GT8 at less than one tenth of the monomer dose (FIG. 2F).

The ability of DLnano_LS_GT8 to improve humoral responses was observed in other animal models. Strikingly, two immunizations in C57BL/6 mice of DLmono_GT8 failed to induce seroconversion, while DLnano_LS_GT8 induced strong humoral responses (FIG. 8I). In genetically diverse CDI mice, we also observed more rapid seroconversion and more robust responses for DLnano_LS_GT8 (FIG. 8J). Additionally, we observed DLnano_LS_GT8 significantly improved humoral responses in both female (FIG. 2C) and male (FIG. 2G) BALB/c mice relative to DLmono_GT8. Finally, in guinea pigs, a single 50 μg intradermal vaccination of DLnano_LS_GT8 remarkably induced seroconversion seven d.p.i and 1.2-log higher antibody titers than DLmono_GT8 over time (FIG. 2H). We proceeded with studies of intradermal (ID) vaccination in guinea pigs as ID delivery has additional advantages of simplicity, improved tolerability, and being dose sparing [38, 40].

We next compared the antibody responses induced by protein eOD-GT8-60mer and DLnano_LS_GT8. Protein eOD-GT8-60mer was subcutaneously administered in mice to be consistent with prior studies involving administration of this immunogen to mice [27, 28]; further, a relative high protein dose of 10 μg was used in this study as compared to prior study for protein versus DNA comparison [26]. We observed that two sequential immunizations of protein eOD-GT8-60mer co-formulated with Sigma Adjuvant System or DLnano_LS_GT8 in C57BL/6 mice induced similar humoral responses (FIG. 2I). It has been recently reported that uptake and trafficking of protein-based nanoparticles are dependent on the mannose binding lectin (MBL) complement pathway [26, 46]. We explored if DNA-launched nanoparticles depended on a similar mechanism. Similar to previous reports [26], humoral responses elicited by protein-based GT8 nanoparticles in transgenic MBL and CR2 knockout mice were attenuated as compared to in the wildtype C57BL/6 mice seven d.p.i (FIG. 2J). Strikingly, similar humoral responses were induced in the MBL or CR2 knockout mice as compared to the wildtype C57BL/6 mice by DLnano_LS_GT8 (FIG. 2J), highlighting DLnano immunogens may act independently of MBL-complement pathway, potentially through redundant mechanisms of antigen presentation.

### Example 4. DLnano_LS_GT8 Elicited Superior Cellular Responses than DLmono_GT8 and Uniquely Induced CD8+ T-Cell Responses Relative to Protein eOD-GT8-60mer

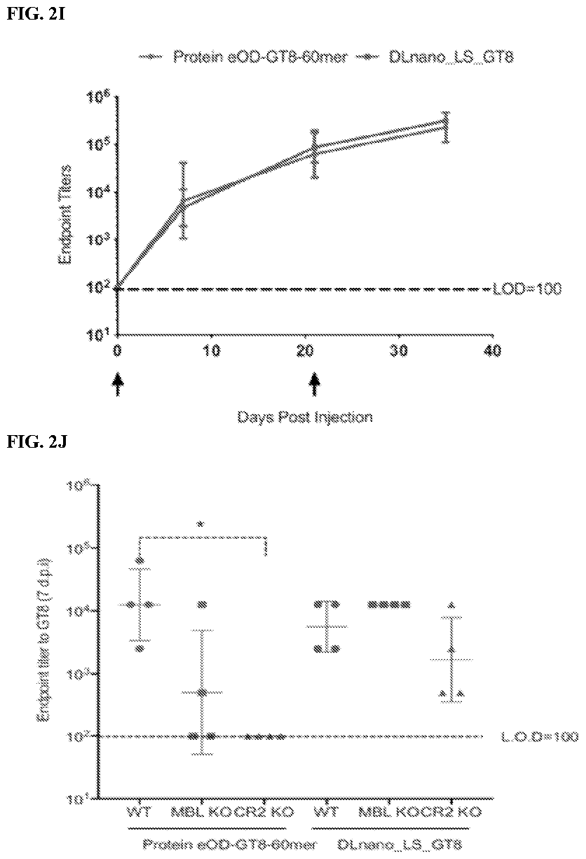

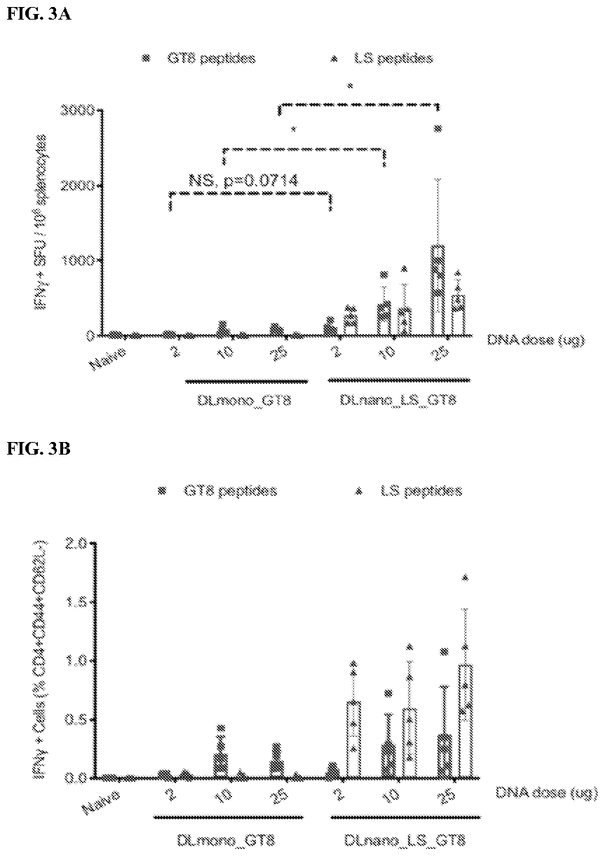

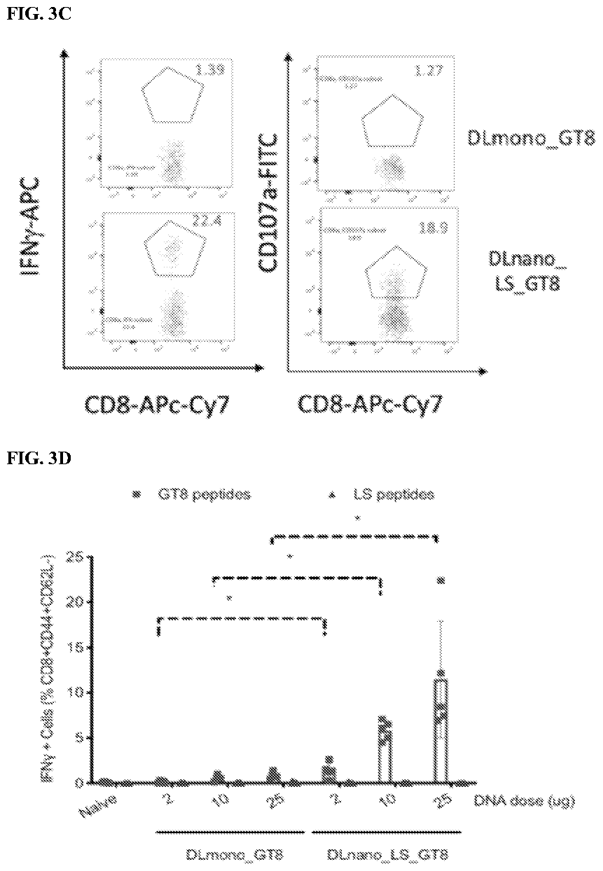

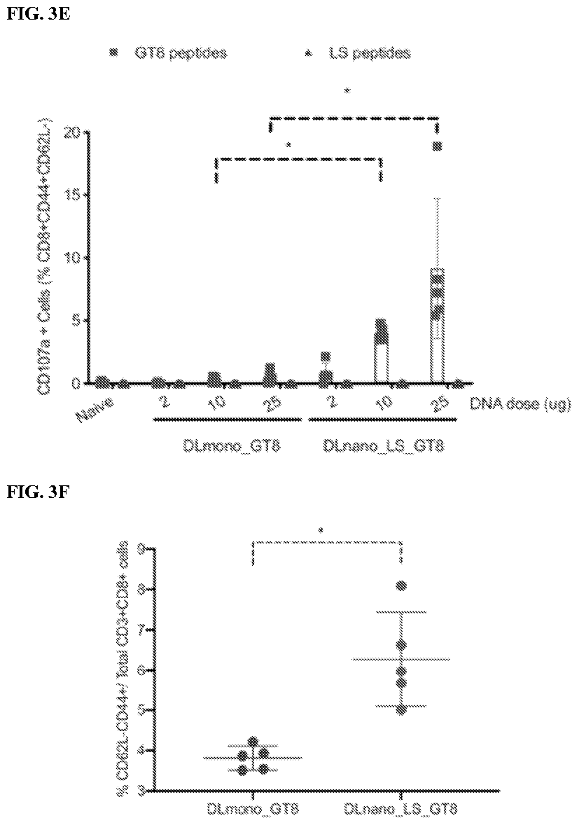

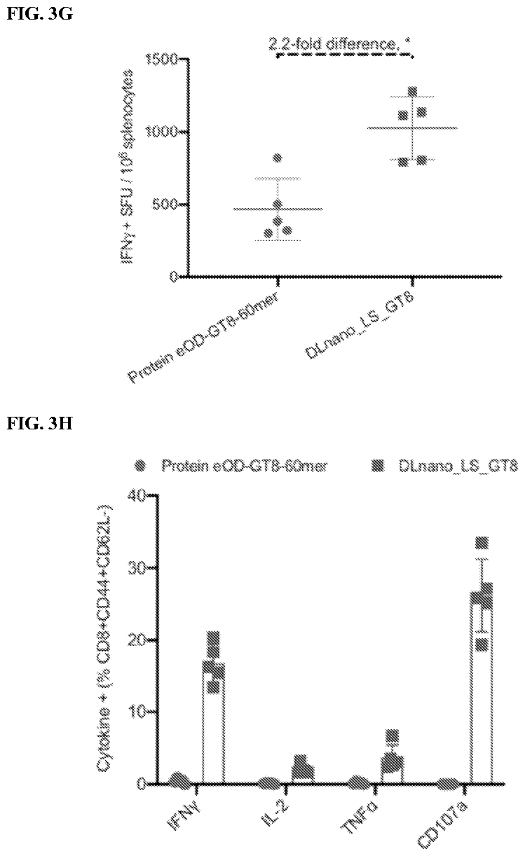

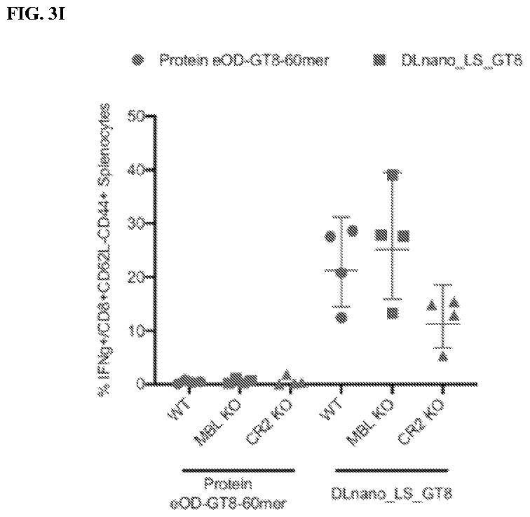

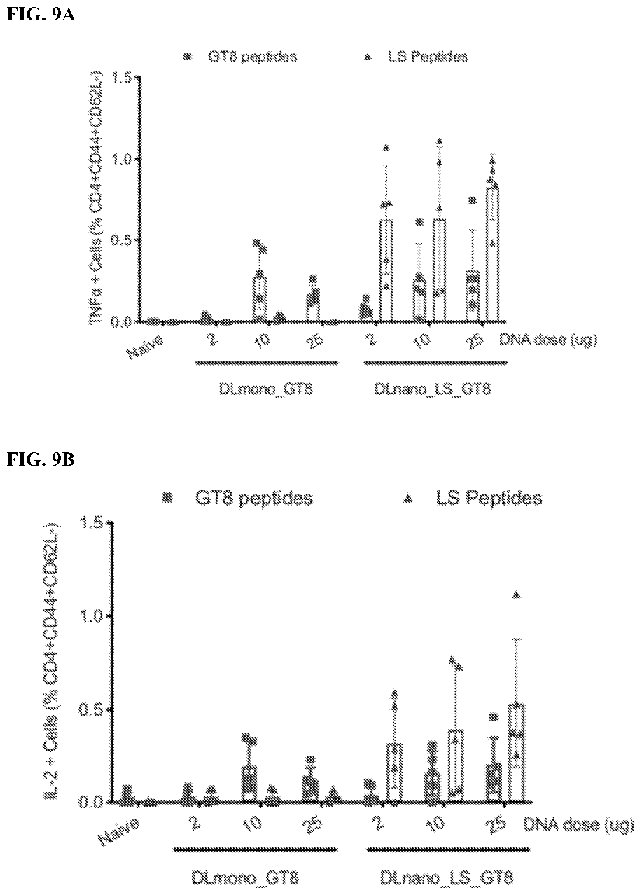

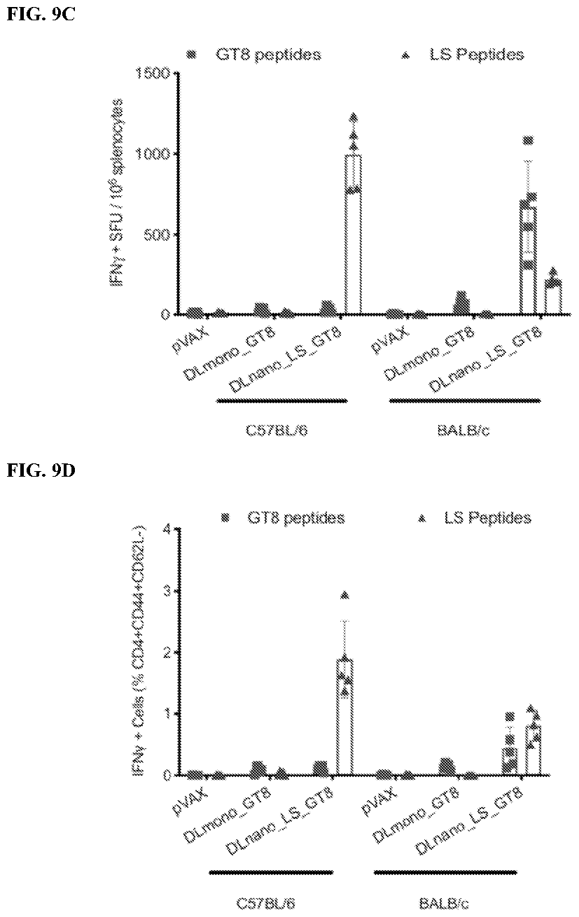

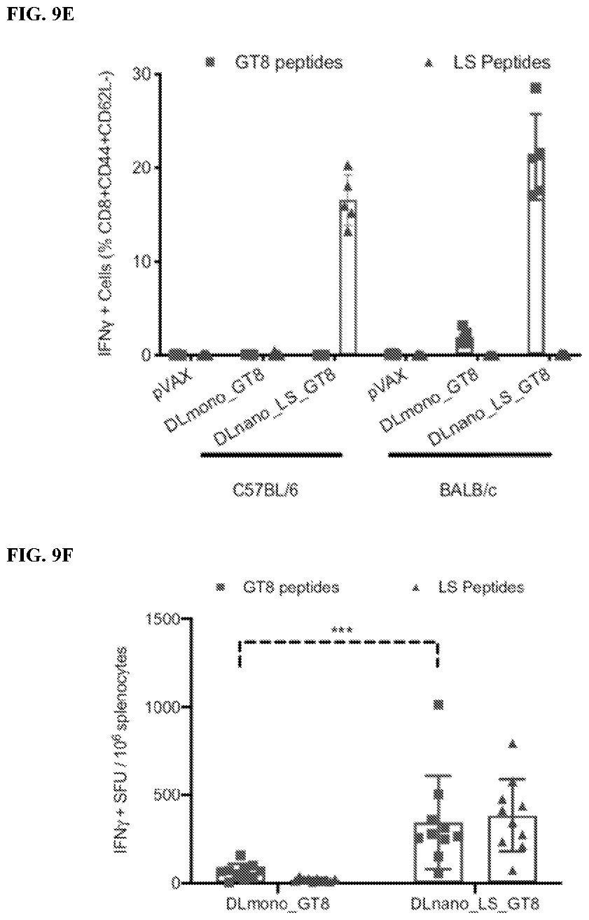

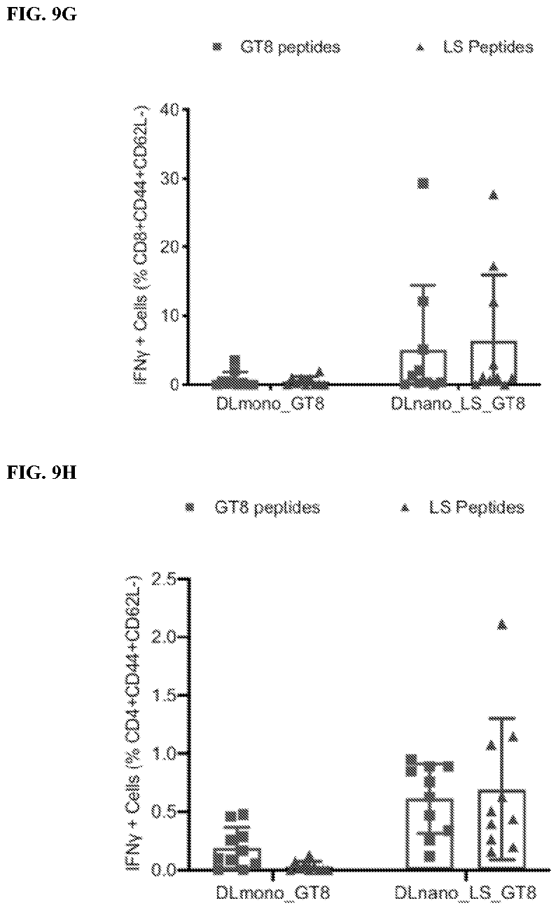

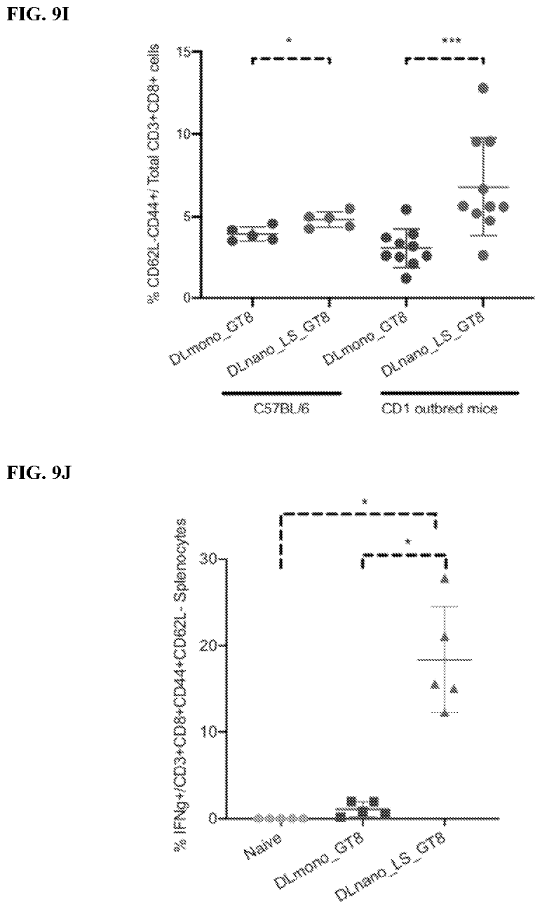

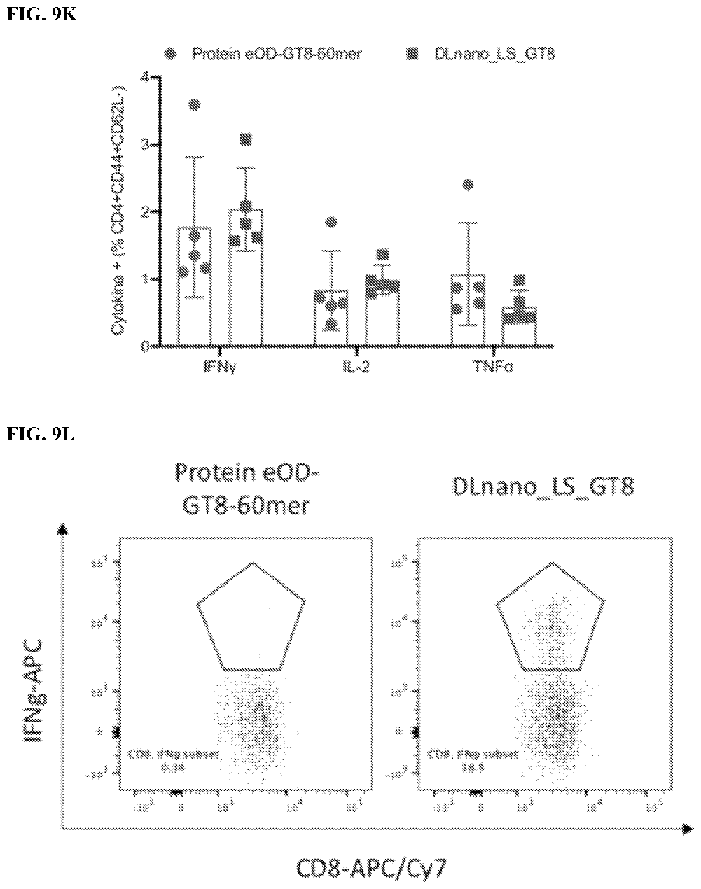

We next examined the induction of antigen-specific cellular responses by DNA nano-vaccines. DLnano_LS_GT8 elicited significantly stronger antigen (GT8)-specific cellular responses than DLmono_GT8 in BALB/c mice as determined by IFNγ-ELIspot assays (FIG. 3A). Intracellular cytokine staining (ICS) revealed that the scaffolding LS domain drove predominantly CD4+ responses, since a higher proportion of effector memory CD3+CD4+CD44+CD62L− T-cells produced IFNγ, TNFα and IL-2 when stimulated by the LS peptides than by GT8 peptides (FIG. 3B and FIG. 9A-9B). In contrast, we found that effector memory CD3+CD8+CD44+CD62L− T cells induced by DLnano LS GT8 were more reactive to the GT8 domain than to the LS domain. DLnano_LS_GT8 induced more antigen-specific effector memory CD8+ T-cells that expressed activation cytokines IFNγ and exhibited effector phenotypes (CD107a+) than DLmono_GT8 in BALB/c mice (FIG. 3C-3E).

In C57BL/6 mice, we also found that DLnano_LS_GT8 elicited strong T-cell responses to the full immunogen. Both CD4+ and CD8+ responses were predominantly to the LS domain, possibly due to the lack of CD8+ T-cell epitope in the GT8 domain for this inbred strain (FIG. 9C-9E). To determine the ability of DLnano_LS_GT8 to elicit T-cell responses to the antigenic domain in a model with more diverse HLA haplotypes, we used the outbred CDI mice and found that DLnano_LS_GT8 induced stronger CD4+ and CD8+ effector memory T-cell responses to the GT8 domain than DLmono_GT8 (FIG. 9F-9H). In all mice strains studied, we observed DLnano_LS_GT8 elicited significantly higher frequencies of effector memory CD8+ T-cells than could DLmono_GT8 (FIG. 3F and FIG. 9I). Additionally, DLnano_LS_GT8 was observed to induce stronger CD8+ T-cell responses to the GT8 domain in both female (FIG. 3D) and male (FIG. 9J) BALB/c mice.

In comparison to protein eOD-GT8-60mer, we observed two immunizations of DLnano_LS_GT8 induced 2.2-fold higher T-cell responses by IFNγ-ELIspot assay (FIG. 3G). In addition, ICS revealed that while both protein and DNA-encoded GT8-nanoparticles induced CD4+ responses (FIG. 9K), only DNA-launched but not protein-based nanoparticles elicited potent CD8+ T-cell responses (FIG. 3H and FIG. 9L). Recombinant protein nanoparticle failed to induce CD8+ T-cell responses in both WT and transgenic MBL and CR2 knockout mice; whereas DLnano_LS_GT8 induced robust CD8+ T-cell responses in these strains (FIG. 3I), confirming our prior observations that DLnano-vaccines may act independently of the MBL-complement pathway.

### Example 5. Designed DNA-Launched GT8-Nanoparticles with Alternative Scaffolds Analogously Induced Improved Adaptive Immune Responses

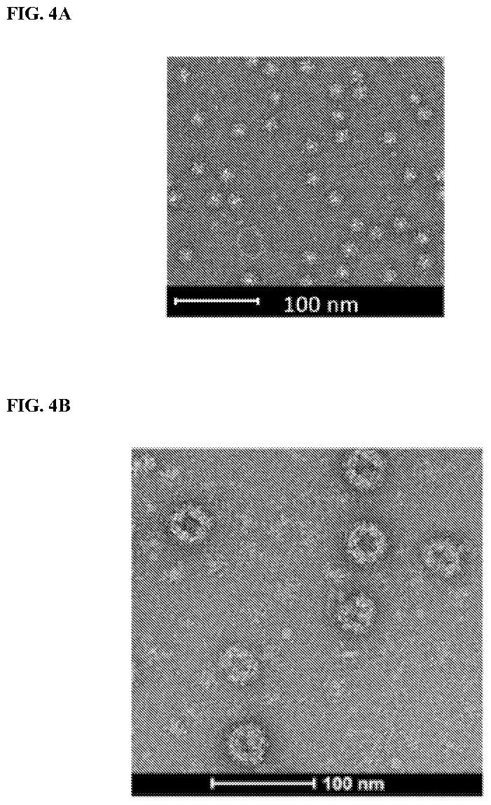

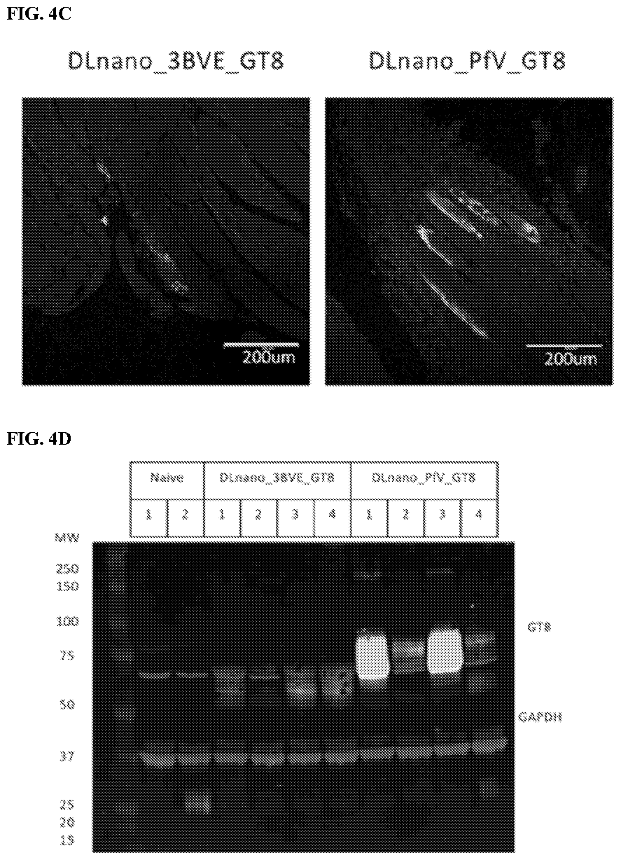

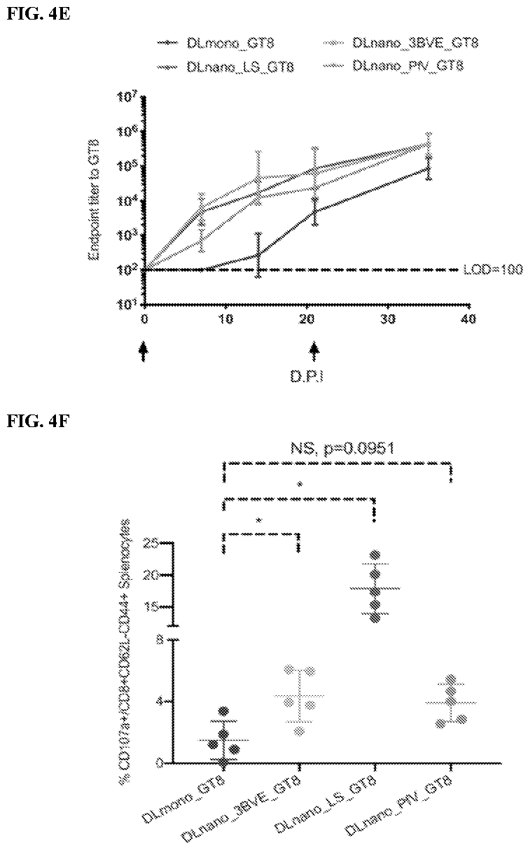

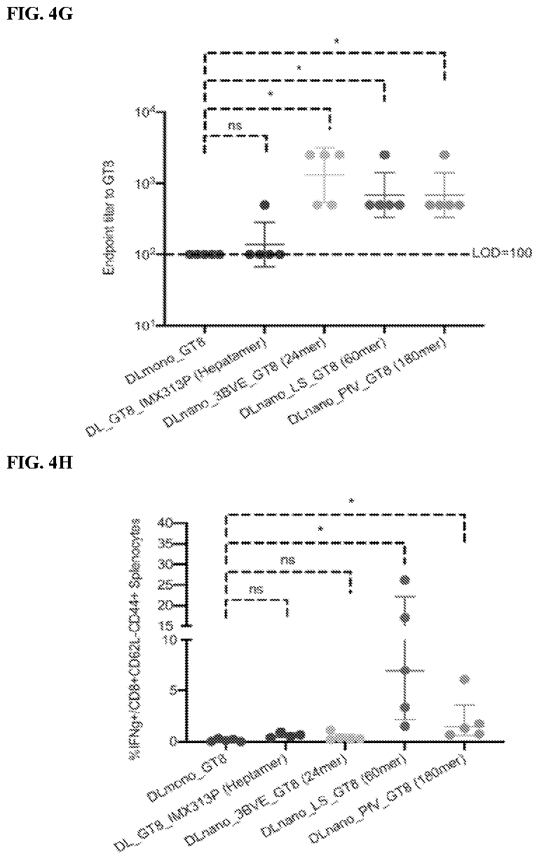

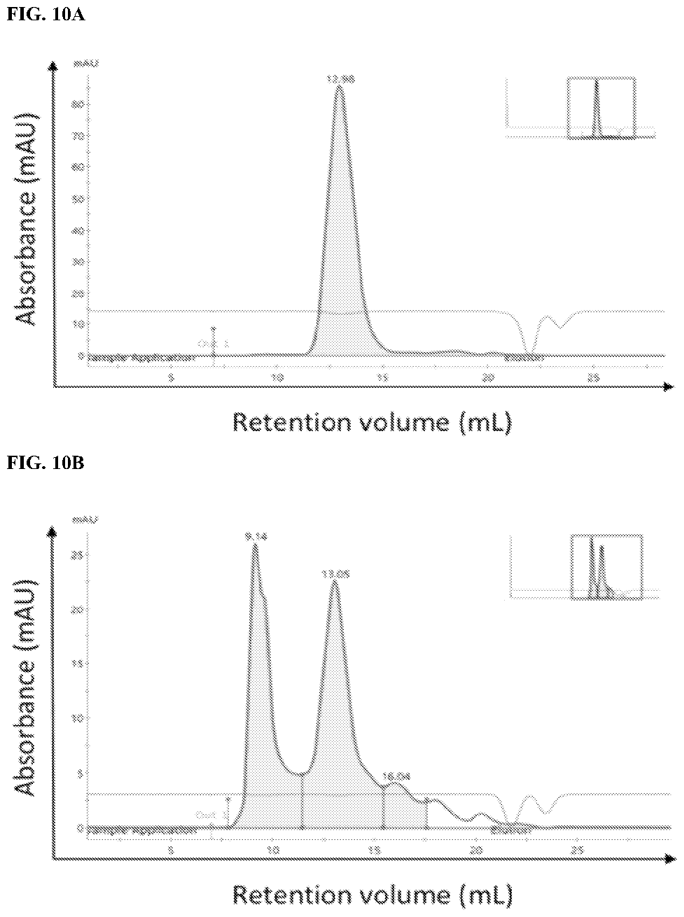

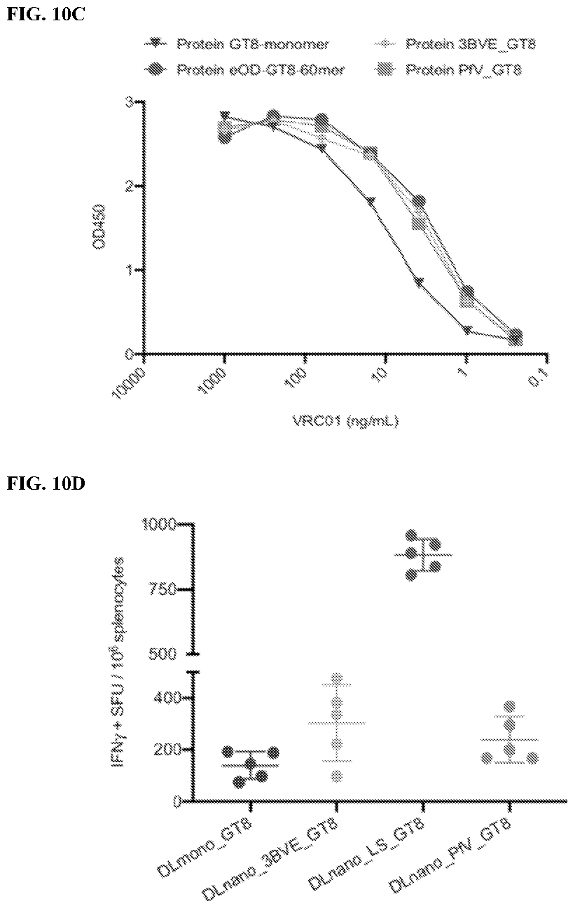

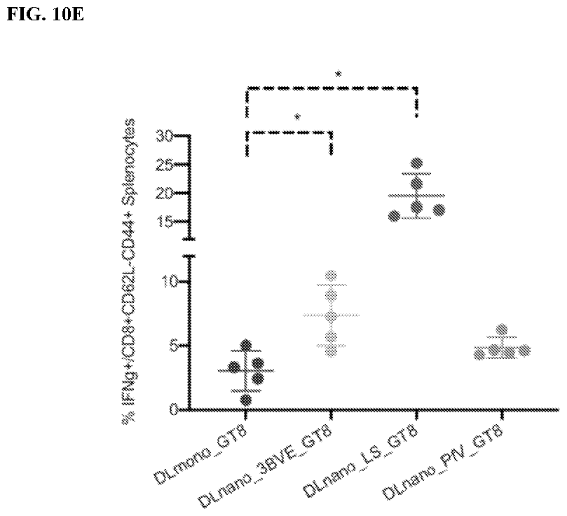

To ensure that the observed phenomena were not limited to lumazine synthase scaffolded nanoparticles, we computationally designed additional GT8 nanoparticles. Using the crystal structures of ferritin from Helicobacter pylori (3BVE, a 24-mer), and PfV viral cage from Pyrococcus furiosus(2e0z, a 180-mer), we modeled GT8 at various geometries relative to the particle surface and designed appropriate flexible linkers. 3BVE-GT8 homogenously assembled into spherical nanoparticles by nsEM (FIG. 10A and FIG. 4A). For PfV_GT8, we observed mixed species, but the predominant peak at 9.14 mL retention time, which accounted for approximately 60% of overall intensity, corresponded to torus shaped nanoparticle by nsEM (FIG. 10B and FIG. 4B). To demonstrate decoration of the designed nanoparticles with GT8, recombinantly produced protein 3BVE_GT8, eOD-GT8-60mer and PfV_GT8 were all tested and observed to bind to VRC01 (FIG. 10C). Immunofluorescence demonstrated that both DLnano_3 BVE_GT8 and DLnano_PfV_GT8 expressed in vivo four d.p.i (FIG. 4C), even though in vivo expression of DLnano_PfV_GT8 was found to be stronger on average than DLnano_3 BVE_GT8 by SDS-PAGE analysis (FIG. 4D). Functionally, BALB/c mice immunized with DLnano_3 BVE_GT8, DLnano_LS_GT8 and DLnano_PIV_GT8 all rapidly sero-converted seven d.p.i and mounted stronger antibody responses over the five-week period than mice immunized with DLmono_GT8 (FIG. 4E). In addition, BALB/c mice immunized with two doses of DLnano_3 BVE_GT8, DLnano_LS_GT8 and DLnano_PfV_GT8 all developed stronger CD8+ effector memory T-cell responses to the antigenic GT8 domain than those immunized with DLmono_GT8 by IFNγ ELIspot and ICS assays (FIG. 4F, and FIG. 10D-10E).

Valency of nanoparticles was found to be relevant to the dose-sparing phenomenon observed (FIG. 2F). At low DNA dose of 2 μg, we found that 24, 60 and 180-meric DNA-launched GT8 nanoparticle vaccines but not heptameric DL_GT8_IMX313P was capable of inducing seroconversion in BALB/c mice at 7 d.p.i (FIG. 4G). In terms of cellular immunity at this dose, we found that only 60- and 180-meric but not hepta- and 24-meric DNA-launched GT8 nano-vaccines were capable of inducing improvement in CD8+ T-cell immunity relative to DLmono_GT8 (FIG. 4H). Overall, we observed that the aforementioned nanoparticle domains can be designed to display antigens like GT8 to elicit rapid and strong adaptive immune responses.

### Example 6. Designed DNA-Launched Hemagglutinin Nano-Vaccine Induced Improved Functional Antibody Responses and Stronger CD8+ T-Cell Immunity

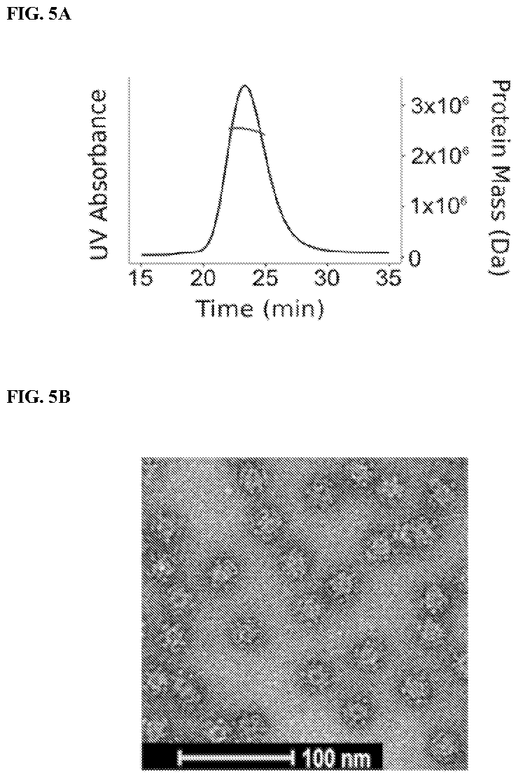

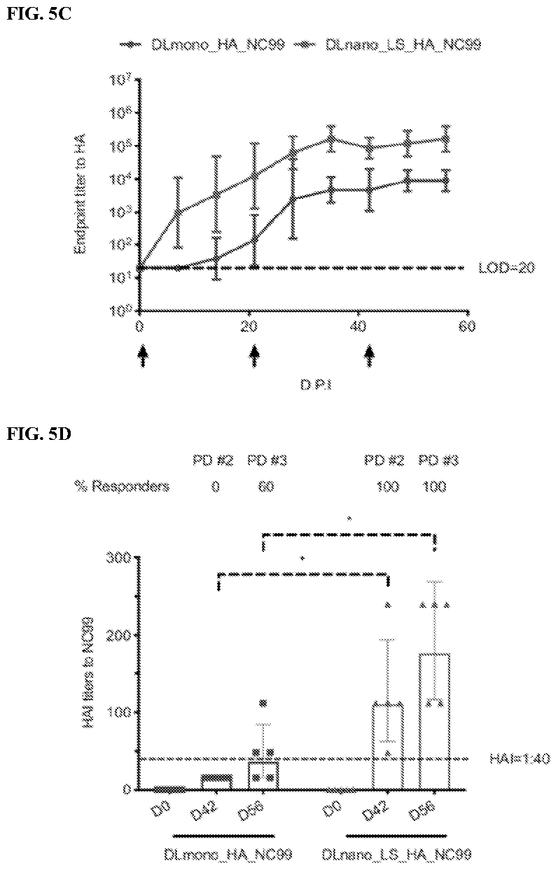

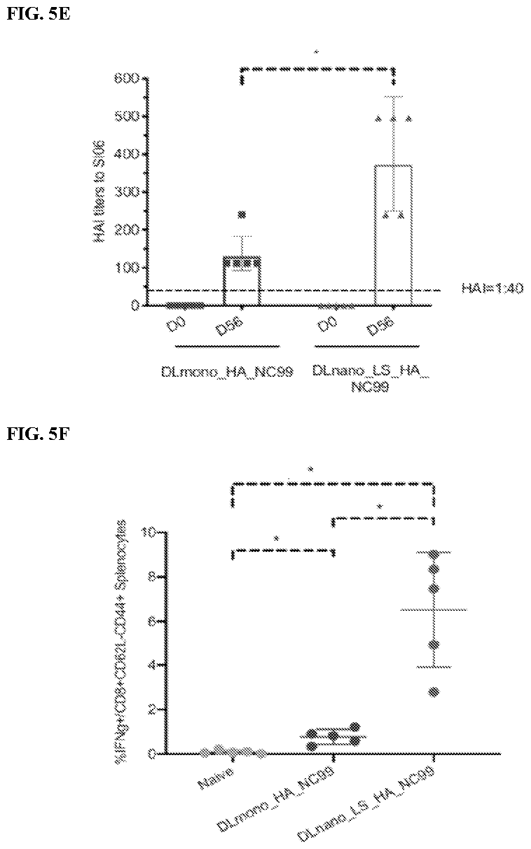

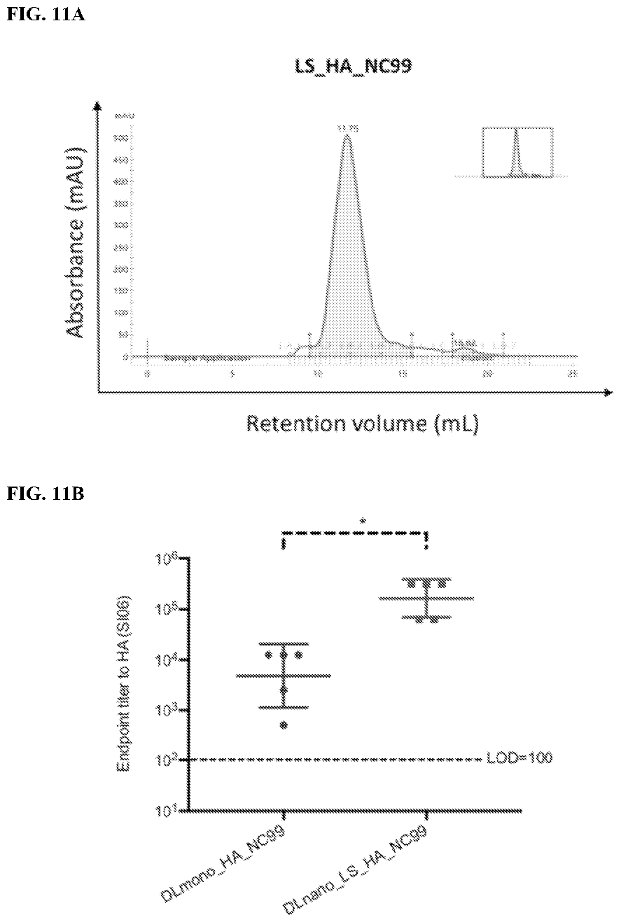

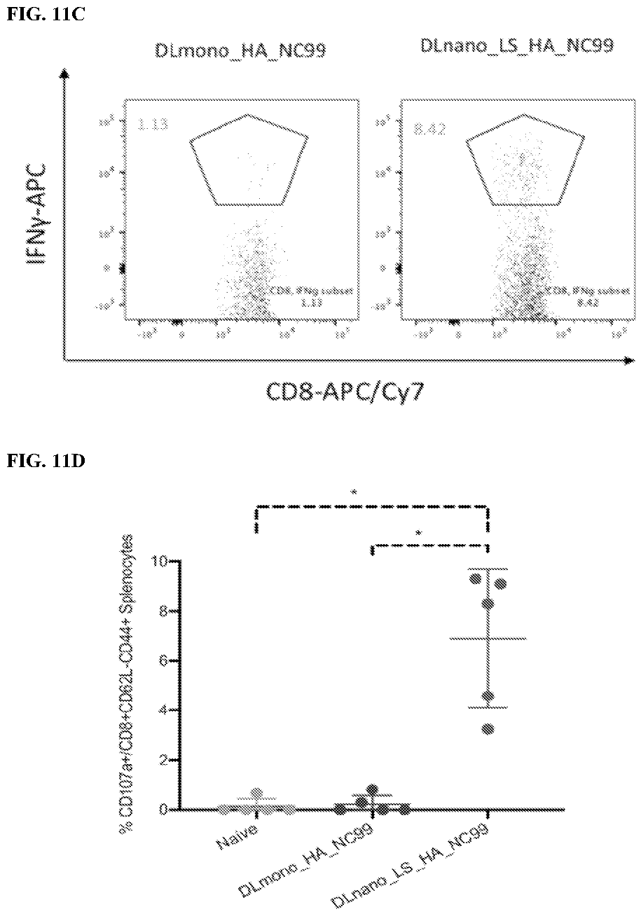

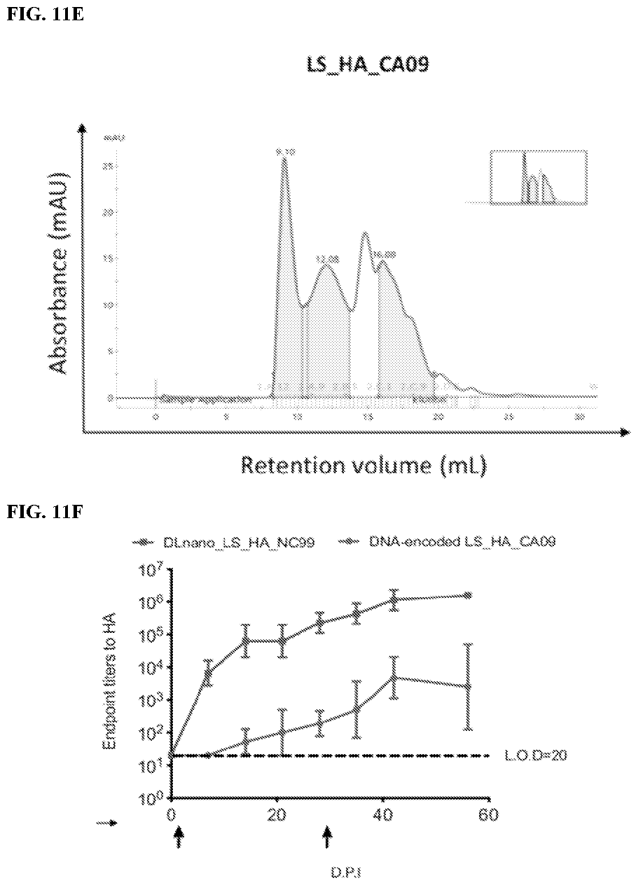

To determine if these findings could be applied to an immunogen relevant to a different infectious disease, we computationally designed a LS nanoparticle to display the receptor binding domain of the head of influenza hemagglutinin (LS_HA_NC99) based on the H1N1 strain A/New Caledonia/20/1999 and confirmed its assembly into homogenous 60-mer by both SEC, SEC-MAL and nsEM (FIG. 11A and FIG. 5A-5B). A dose-sparing phenomenon was observed for DLnano_LS_HA_NC99, as at a remarkably low plasmid vaccine dose of 1 μg, DLnano_LS_HA_NC99 induced significantly stronger humoral responses in BALB/c mice than DLmono_HA_NC99 (FIG. 5C). Hemagglutinin inhibition titers (HAI) against the autologous NC99 strain were found to be higher than 1:40 (which correlated with 50% reduction in the risk of infections in humans [47]) in 100% of mice immunized with two doses of DLnano_LS_GT8 and 0% in mice immunized with two doses of DLmono_HA_NC99 (FIG. 5D). At the final timepoint (56 d.p.i) after three immunizations, both the DLmono_HA_NC99 and DLnano_LS_HA_NC99 groups developed binding and HAI antibodies to the heterologous H1N1 influenza A/Solomon Island/3/06 strain (FIG. 11B and FIG. 5E), and both the binding and HAI titers were still significantly higher for the DLnano_LS_HA_NC99 group. HAI of a more distant H1 strain, A/California/07/2009, was not detected in either group.

Additionally, in terms of elicited cellular responses, two immunizations of DLnano_LS_HA_NC99 induced 8.4-fold higher effector memory CD8+ T-cell responses than DLmono_HA_NC99 at 10 μg dose in terms of CD107a and IFNγ expression, similar to our prior findings (FIG. 5F and FIG. 11C-11D).

Finally, we examined if homogenous in vitro assembly of the designed DLnano-vaccine was a pre-requisite to its enhanced potency. To this end, we studied the in vivo properties of a poorly folded nanoparticle. We used an alternative lumazine synthase scaffolded influenza construct, DNA-encoded LS_HA_CA09, based on the A/California/07/2009 strain which did not pass our biophysical filters, as in vitro expression of the construct showed 3+ peaks with the two largest peaks consisting of aggregates or smaller unassembled protein by SEC (FIG. 11E). We found DNA-encoded LS_HA_CA09 could not induce the characteristic early sero-conversion in BALB/c mice (FIG. 11F). Even when the immunized mice were followed over time, the antibody responses induced by DNA-encoded LS_HA_CA09 still lagged behind those by DLnano_LS_HA_NC99, highlighting downstream success of DLnano-vaccine predicated upon preliminary computational design and biophysical characterization.

### Example 7. DNA-Launched Hemagglutinin Nano-Vaccine Conferred Improved Protection to Lethal Pandemic Influenza H1 A/California/07/09 Challenge in Mice

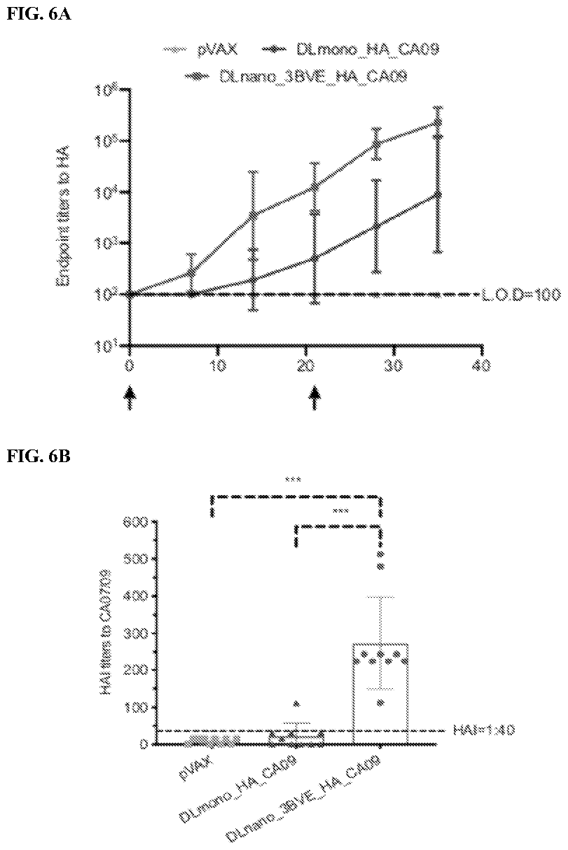

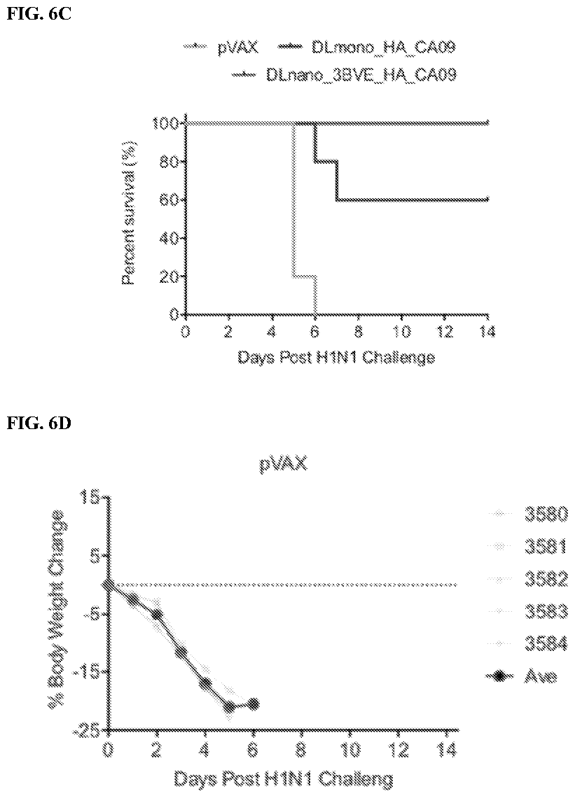

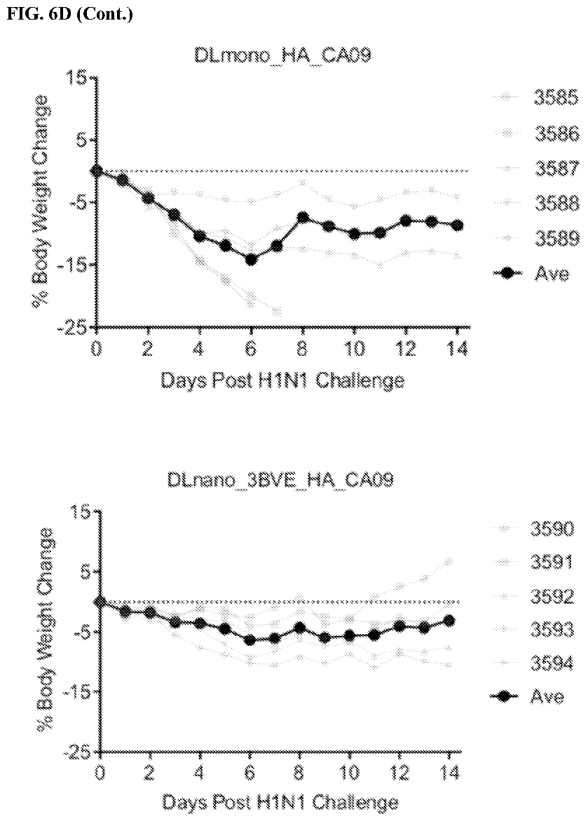

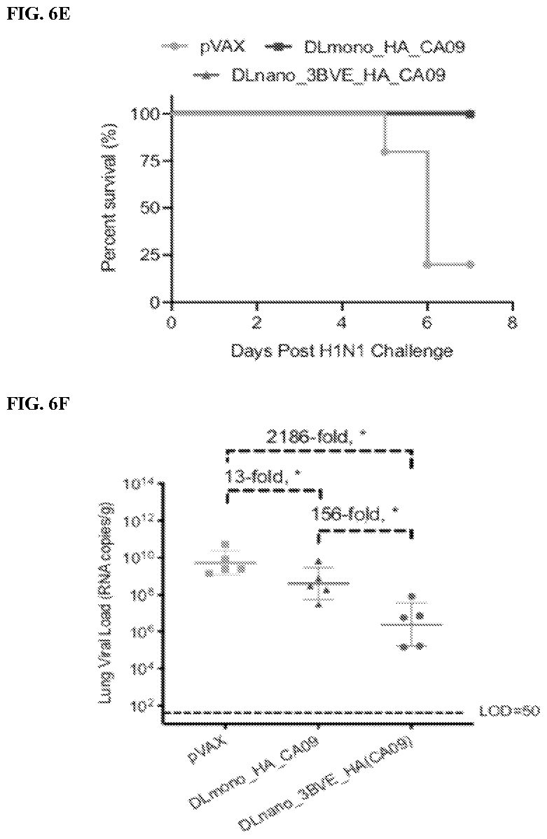

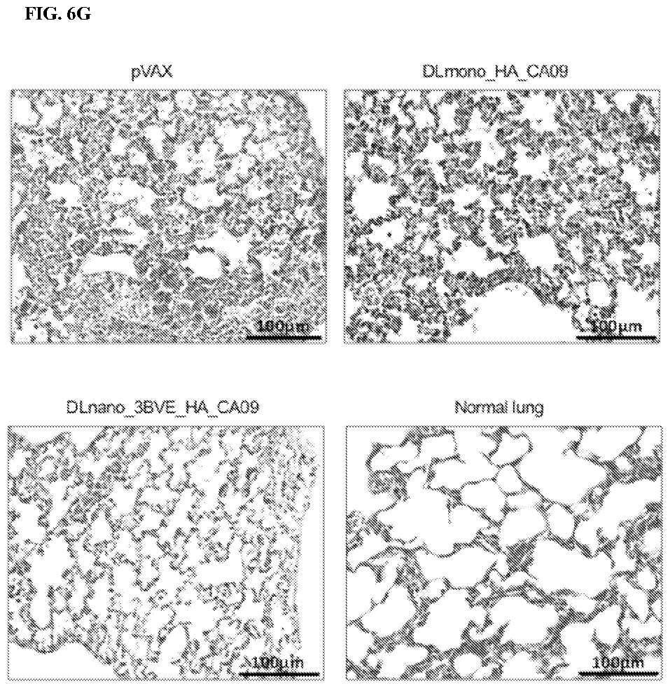

To further evaluate the induction of functional immune responses by DLnano-vaccines, we utilized a lethal influenza challenge model in mice. We constructed a ferritin-scaffolded receptor binding domain of hemagglutinin from H1/California/07/09 strain, DLnano_3 BVE_HA_CA09, that was leader sequence, codon and mRNA-optimized as compared to a previously reported construct [48]. We first confirmed its in vitro assembly into nanoparticles by SEC and nsEM (FIG. 6A-6B). We then immunized three groups of mice twice with minimal doses (1 μg) of DNA encoding either DLmono_HA_CA09, DLnano_3 BVE_HA_CA09 or control backbone pVAX vector three weeks apart. We observed improved induction of binding antibody responses in mice immunized with DLnano_3 BVE_HA_CA09 than those with DLmono_HA_CA09 (FIG. 6A). Five weeks post the first immunization, we observed significant 8-fold improvement in HAI titers in mice immunized with DLnano_3 BVE_HA_CA09 than those with DLmono_HA_CA09 (FIG. 6B). We then set up two lethal influenza challenge experiments in these three groups of mice, five weeks post the final immunization. Each mouse was intranasally inoculated with 10LD50 homologous H1/California/07/09 virus and was followed for two weeks for weight loss. Any mouse losing more than 20% of baseline body weight would have met the humane endpoint for euthanasia. In this experiment, we observed only mice immunized with DLnano_3 BVE_HA_CA09 fully survived the lethal challenge (FIG. 6C), whereas 40% (2/5) of mice immunized with DLmono_HA_CA09 or 100% (5/5) of mice immunized with control pVAX backbone succumbed to infections. Additionally, amongst mice that survived the challenge, substantially lower weight loss was observed in mice immunized with DLnano_3 BVE_HA_CA09 than DLmono_HA_CA09 (FIG. 6D).

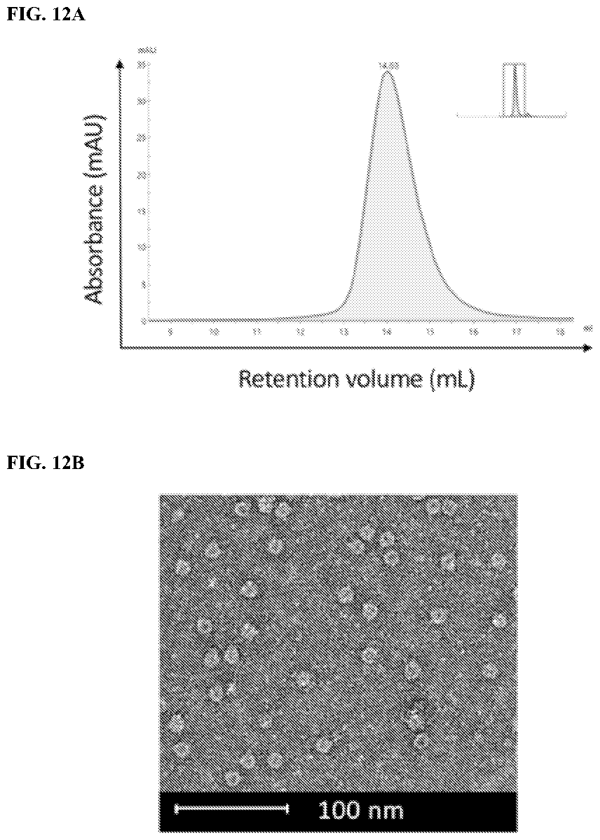

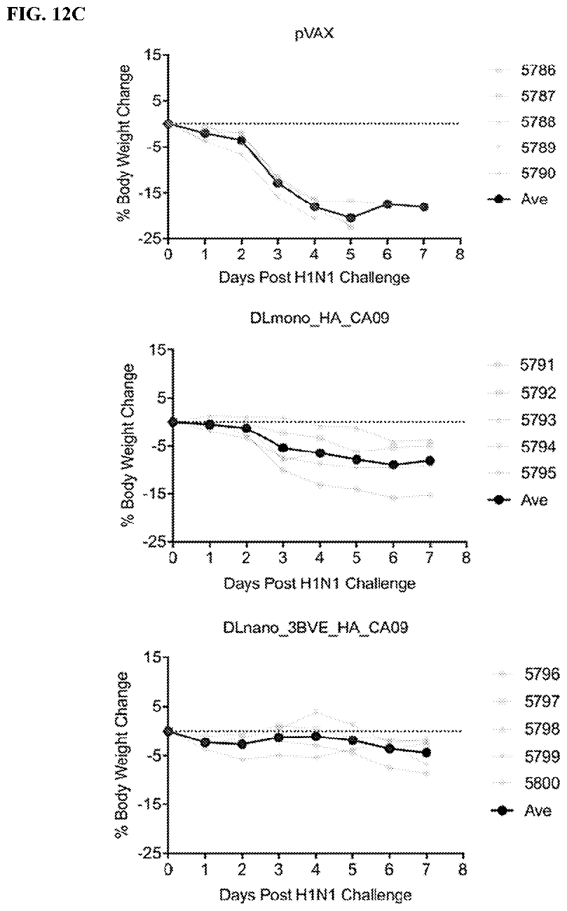

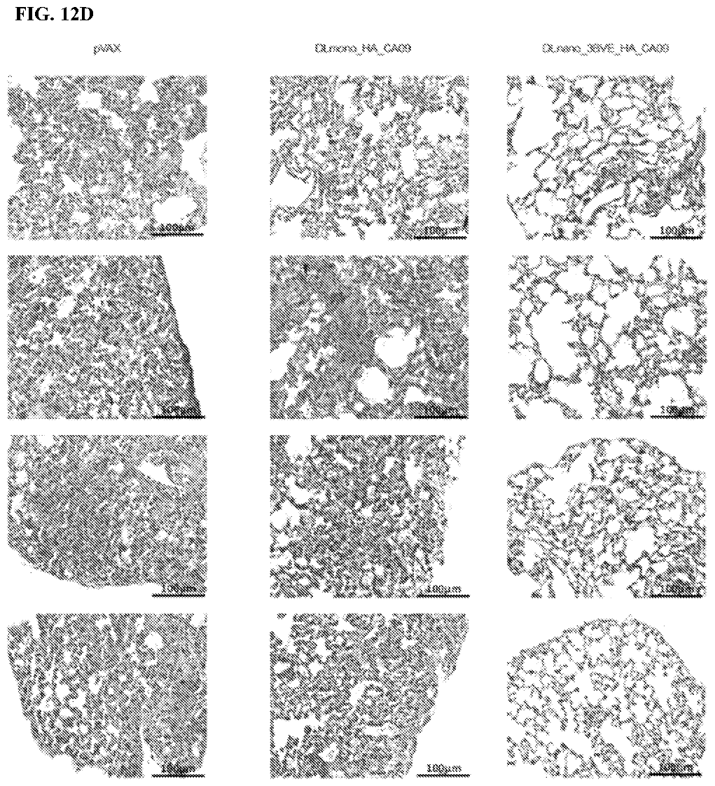

In a separate set of experiments, we followed these three groups of immunized mice seven days post H1/CA09 challenge to determine lung viral load and pathology. It was observed, in this challenge study, that within the first seven days, 80% (4/5) of mice immunized with control pVAX vector succumbed to infection, but mice immunized with either DLmono_HA_CA09 and DLnano_3 BVE_HA_CA09 survived the first seven days (FIG. 6E), even though mice immunized with DLmono_HA_CA09 still lost substantially more weight than those immunized with DLnano_3 BVE_HA_CA09 (FIG. 12C). Additionally, we observed significant reduction in viral load of mice immunized with DLnano_3 BVE_HA_CA09 as compared to mice immunized with pVAX (2186-fold reduction) or with DLmono_HA_CA09 (156-fold reduction) (FIG. 6F). Finally, H&E staining of lung specimens at seven days post challenge or at the time of euthanasia revealed that mice immunized with DLnano_3 BVE_HA_CA09 but not with DLmono_HA_CA09 were protected from lung pathology, including the observations of eosinophilic necrotic deposits within the alveolar spaces and or thickening of alveolar septa, associated with influenza infection (FIG. 6G and FIG. 12D). The lethal challenge study illustrated that the DLnano-vaccine could confer significant functional advantages in an infectious disease model.

### DISCUSSION AND CONCLUSION

Development of vaccines can be a challenging endeavor due to poor immunogenicity of certain vaccine antigens, which results in the need to increase the number of required vaccinations, dose per vaccination, and the required interval for patients to complete the vaccine regime. Particulate vaccine formulations can help boost immunogenicity but can be slow to develop on a large scale due to manufacturing complexities. Synthetic nucleic-acid based methods for the delivery of vaccine antigens have shown great promises, as they are often produced at significantly lower costs than their protein counterparts, can be manufactured to scale and bypass complex processes of assembly [49], offer superior safety profile and demonstrate remarkable thermo-stability to allow for extended shelf-lives [51].

In this study, through the use of computational modeling and biophysical characterization, we engineered multimeric forms of HIV and influenza antigens which folded properly in vitro and displayed the desired antigenic profiles. We showed that these designer nano-vaccines can assemble in vivo when delivered using synthetic DNA and adaptive electroporation, through direct evidence from pseudo-Native PAGE analysis (FIG. 1F) and transmission electron microscopy (FIG. 1H-1I), and indirect evidence of binding of murine MBL to in vivo produced DLnano_LS_GT8 but not to DLmono_GT8 (FIG. 1G). The in vivo nanoparticle assembly resulted in improved antigen trafficking and induction of potent adaptive immune responses, including rapid sero-conversion, higher binding and functional HAI antibody titers yet with significant dose sparing. Enhanced antibody responses were also induced when DLnano-vaccine was administered via intradermal DNA vaccination, a newer and clinically important route of DNA vaccination [37, 40]. Importantly, enhanced immune responses induced by DLnano-vaccines also conferred functional advantages. The DLnano-vaccines were more efficient at driving HAI, CD8+ T-cell responses, and ultimately generating protection to animals from intranasal influenza challenge. DNA vaccine approach can effectively synergize with structure-guided protein engineering to quickly produce in vivo designer nano-vaccine constructs for rapid evaluation.

This work interrogated factors which might contribute to the enhanced adaptive immune responses of DLnano-vaccines. Homogeneous in vitro assembly of these computationally designed DLnano-vaccines is important for their downstream success, as poorly assembled DNA-encoded LS_HA_CA09 did not elicit similarly potent immune responses (FIG. 11F). Homogeneous in vitro assembly will likely help increase the fraction of more fully assembled nanoparticles in vivo, contributing to the overall immunogenicity of the vaccine. It is in theory possible that the improved immunogenicity described here can be attributed to differences in levels of antigen expression. However, two observations suggest that antigen expression is not solely responsible for improved responses. First, we showed that DLnano_LS_GT8 induced stronger humoral responses than DLmono_GT8 in BALB/c mice at less than one tenth of the dose (FIG. 2F). Second, while DLnano_PfV_GT8 expressed at higher levels in vivo than DLnano_3 BVE_GT8 (FIG. 4D DLnano_PfV_GT8 and DLnano_3 BVE_GT8 induced similar antibody titers and T cell responses at 25 μg dose (FIG. 4E-4F). The exact contribution of nanoparticle assembly, expression and valency for the induction of optimal immune responses will require further investigation.

When DNA-launched nano-vaccines were compared to recombinant protein nano-vaccines, widely considered as an extremely potent vaccine formulation in terms of induction of antibody responses [52], we observed DLnano-vaccines induced comparable humoral responses to recombinant protein nanovaccines, but uniquely induced potent CD8+ T-cell responses in an MBL-complement independent manner. The observation that DLnano-vaccines function independently of MBL-complement pathway is likely of interest for clinical translation of such vaccines, as approximately 5-20% of human populations have MBL deficiency (plasma MBL <100 ng/mL) [53, 54]. The role of T cells in immune surveillance to mediate protection provides a strong rationale for exploring this unique property of DLnano-vaccine [55], especially for such diseases as liver-stage malaria [56], influenza for the elderlies [57, 58], and cancer [59]. The unique ability for DLnano vaccination to induce CD8+ T-cell responses may be related to its distinct mechanism of antigen uptake and presentation. Antigen presenting cells, such as macrophages, are known to migrate into the site of electroporation to scavenge antigens expressed through DNA cassettes associated with apoptotic cells [49]. Prior studies observed that co-delivery of DNA vaccines with pro-apoptotic mutated Caspase 2 or Fas significantly increased both CD4+ and CD8+ T-cell responses to the vaccine antigens [60, 61]. Such distinct mechanism of antigen processing might lead to more efficient cross-presentation to the MHC Class I pathway. Additionally, APCs including DCs and macrophages may also be directly transfected with the inoculated DNA cassettes in vivo [62, 63], and the two mechanisms may be synergistic in the induction of CD8+ T-cell immunity. Our findings also demonstrated that DLnano-vaccine could improve induced CD8+ T-cell responses by eight to ten-fold relative to their monomeric counterparts. Given that DNA-vaccines can already induce CD8+ T-cell responses in patients to cause histopathological regression of HPV-driven cervical dysplasia [4], the finding is relevant and whether DLnano-vaccines can further improve the clinical response rates should be explored.

Importantly, significant dose sparing can be realized with DLnano-vaccines. A dose of 1 μg of plasmid DNA, a dose at which we historically would not expect to observe robust sero-conversion [64], was able to induce clear functional HAI titers in mice. Fewer immunizations of DLnano-vaccine could induce the same, if not higher, titers of antibodies. Given recent advances in the electroporation technology has improved the potency and consistency of immune responses induced by DNA vaccines in patients [4, 37-39, 63, 65], it will be important to determine whether DLnano-vaccines can also help to reduce doses used in the clinic and lower the number of clinical visits required for vaccination. These advances may have important implications for outbreak control, and for global deployment including of vaccinations in more resource limited settings.

It will be important to build on these initial studies to improve DLnano-vaccines. For example, while it is known that cross-linking of B-cell receptors through multivalent antigen display can improve B-cell responses [15], studies to examine the mechanisms for the improved CD8+ T-cell responses for DLnano-vaccines relative to their monomeric counterparts are also important. Due to the unique ability of DLnano-vaccine to elicit strong CD8+ T-cell immunity, new DLnano-vaccines should be designed and evaluated to target diseases like cancer and T-cell dependent infectious diseases. The combined advantages of a simplified cost-effective temperature-stable platform, with the ability to retain in vivo structural integrity may be of value for the development of additional vaccines for HIV, influenza as well as other infectious diseases.

This work demonstrates that that advances in synthetic DNA and adaptive electroporation technologies have allowed for in vivo assembly of complex computationally designed particulate nano-vaccines to induce improved humoral and cellular responses, and to confer functional protective benefits in a survival study. As DNA can be rapidly manufactured to scale with low costs, it can be envisioned that computationally designed nano-vaccines can be rapidly evaluated to expedite clinical translational and global deployment of various promising vaccine candidates.

### Example 8. Self-Assembling Nanoparticle Vaccine Platform Developed for Infectious Diseases

Compositions comprising self-assembling vaccines and methods of using the same have been previously described in International Application No. PCT/US2019/068444 filed on Dec. 23, 2018, and U.S. provisional application No. 62/982,038 filed on Feb. 26, 2020, each of which is incorporated by reference in its entirety herewith. In those disclosures, it was demonstrated that nanoparticles can be engineered by rational methods and assemble in vitro and in vivo. Using two different antigens, HIV antigen and influenza antigen, it was shown that DNA-Launched nanoparticles (DL-nano) can make superior vaccines.

### Example 9. DNA-Launched Nanoparticle Vaccine Elicits CD8+ T-Cell Immunity Introduction

Cytolytic T cells (CTL) is an extremely important branch of adaptive immune system, which can selectively kill target cells through release of cytokines, granzyme and perforin, as well as mediating target cell apoptosis through Fas and Fas-Ligand interaction [70]. T cells engineered with artificial receptors for anti-tumor activity is an exciting area of research but may come with limitations in throughput and costs. Therefore, induction of broad endogenous antigen-specific CTL through vaccination may, therefore, be an important approach to treat cancer [71].

However, induction of CTL through vaccination can be challenging as it requires antigen presenting cells (APCs) to present HLA-restricted epitopes to the MHC I pathway and provide costimulatory signal to prime CD8+ T cells [72]. Using viral vectors modified to encode target antigens is an approach to drive CTL responses, potentially through direct transduction of target cells [73]. Additionally, special adjuvants can be used to facilitate cross-presentation of target antigens, which to varying degrees promote phago-lysosomal escape and retro-translocation of target antigens into the cytosol [74]. Alternatively, dendritic cells (DCs) can be loaded ex vivo with saturating concentrations of peptides and adoptively transferred back into the host to prime CD8+ T cells [75]. Each approach has certain drawback: relatively few adjuvants have been demonstrated to help prime CD8+ T-cell responses in clinic [76], viral-vectored vaccine can be limited by pre-existing immunity against the viral vector [77], and manufacturing issues with large-scale production of DC vaccines.

DNA vaccination has also been observed to elicit CD8+ T-cell responses both in preclinical animal models and in clinical trials [78], plausibly through a combination of direct transfection of target cells along with cross presentation of DNA-encoded antigens [80]. We previously reported a new strategy to use DNA to launched in vivo produced nanoparticle vaccines, which resulted in significant improvements in the rate of seroconversion, magnitude of humoral response, dose sparing, and protection from viral challenge [81]. Additionally, we have also observed DNA vaccination of antigens scaffolded by in vivo assembled nanoparticles induced significantly improved CD8+ T-cell responses compared to DNA-encoding non-scaffolded antigens [81]. In this work, we compared the ability of DNA and protein vaccinations of identical nanoparticle antigens to induce CTL responses in mice. We discovered that intramuscular DNA vaccination, but not RIBI, CpG, or poly (I:C) adjuvanted protein vaccination, was capable of inducing robust CTL responses against influenza and HIV antigens. Mechanistic studies revealed that in comparison to protein vaccination, DNA launched nanoparticle vaccines uniquely induced apoptosis of transfected muscle tissues, which was followed by infiltration of antigen presenting cells (APCs), including pro-inflammatory M1 macrophages, to scavenge antigens. CTL responses were significantly attenuated when phagocytic macrophages were depleted or in cDC deficient BATFK3 KO mice. Finally, DNA launched nanoparticle vaccines induced more potent and consistent CTL responses against immunodominant epitopes on tumor-associated antigens Trp2 and Gp100 than conventional monomeric DNA vaccines or CpG adjuvanted peptide vaccines and facilitated tumor rejection in 80% of mice in the prophylactic B16-F10 melanoma model. This work therefore provides a demonstration that induction of transient physical stress through DNA-launched nanoparticle vaccination may be a safe, easy and robust way to facilitate antigen cross presentation and CTL priming to target cancer.

### 1. Materials and Methods

i. Design of DNA-Launched Nanoparticles

The self-assembling nanoparticles scaffolding Trp2 and Gp100 peptides were engineered using structure-guided design, as described in the results section. DNnano_LS_GT8 and DLnano_LS_HA(NC99) were developed in prior work and disclosed in International Application No. PCT/US2019/068444 filed on Dec. 23, 2018, and U.S. provisional application No. 62/982,038 filed on Feb. 26, 2020, each of which is incorporated by reference in its entirety herewith.

ii. DNA Design and Plasmid Synthesis

Protein sequences for IgE Leader Sequence and eOD-GT8-60mer were as previously reported [82, 83]. Protein sequence for HAI_NC99 was obtained from GenBank (accession number AY289929.1). DNA encoding protein sequences were codon and RNA optimized as previously described [82]. The optimized transgenes were synthesized de novo (GenScript) and cloned into a modified pVAX-1 backbone under the control of the human CMV promoter and bovine growth hormone poly-adenylation signal.

iii. Animals

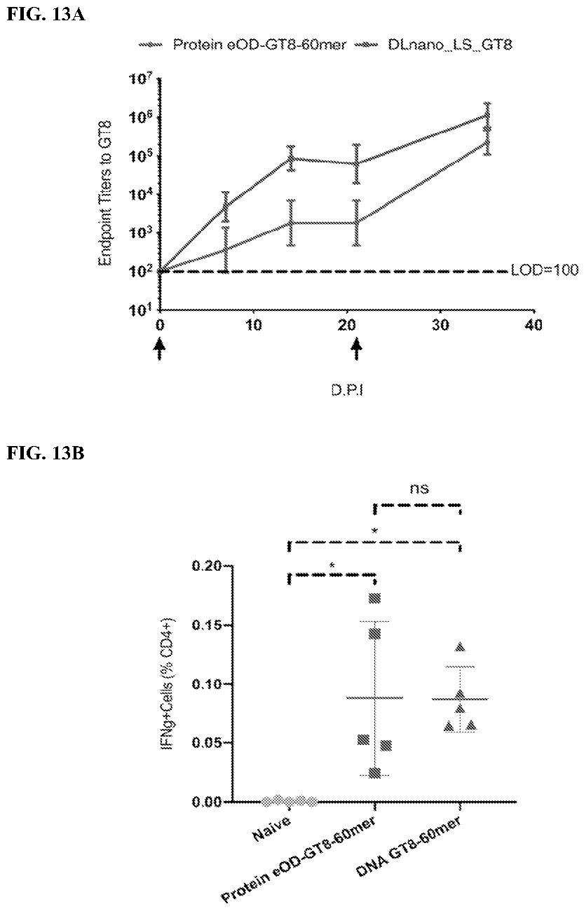

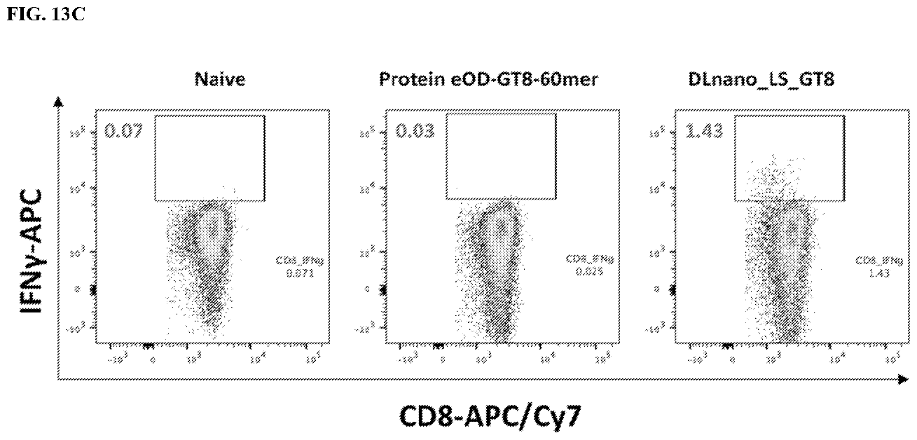

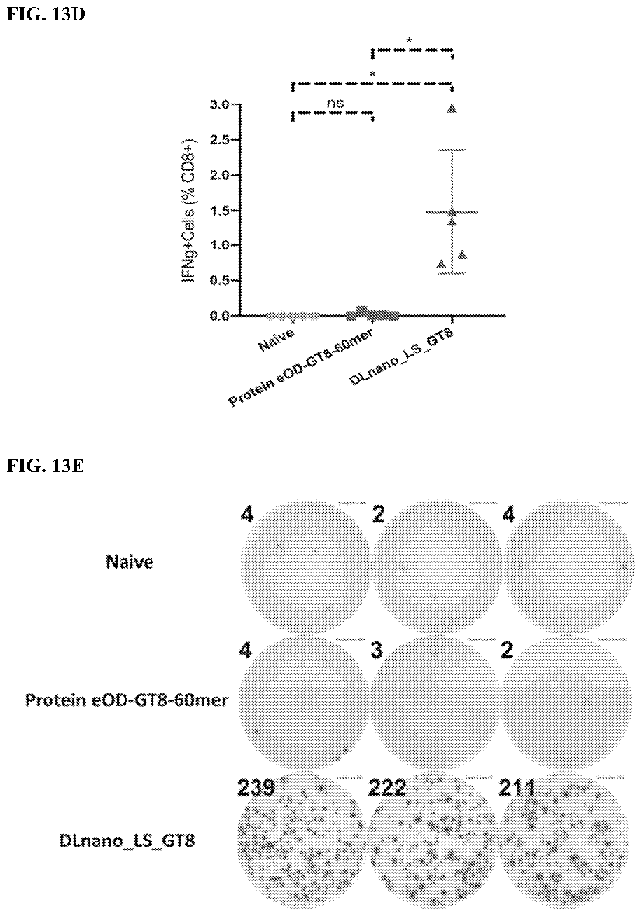

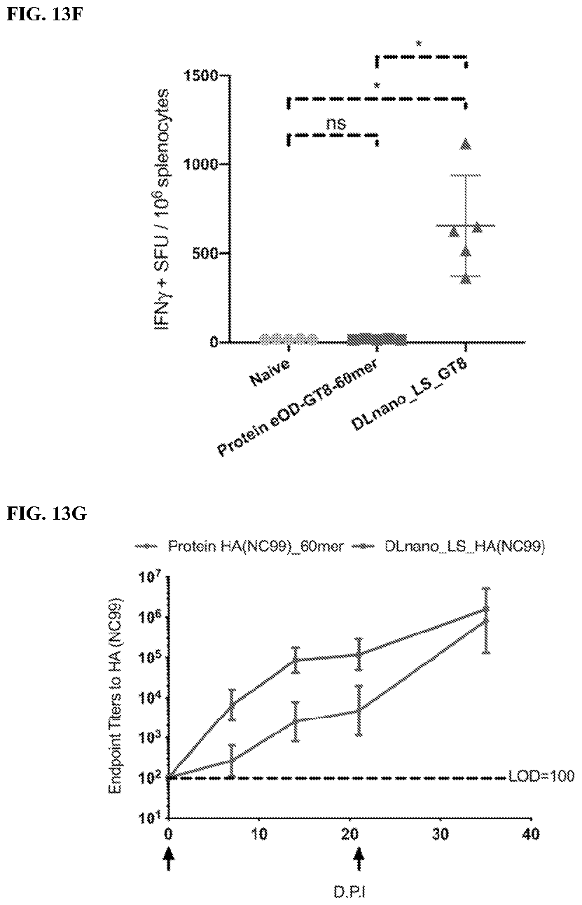

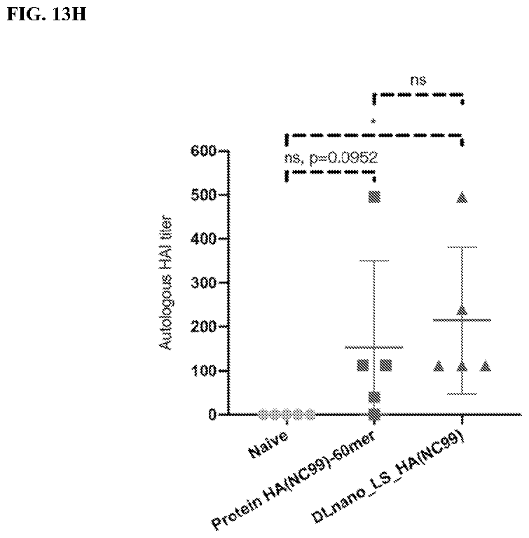

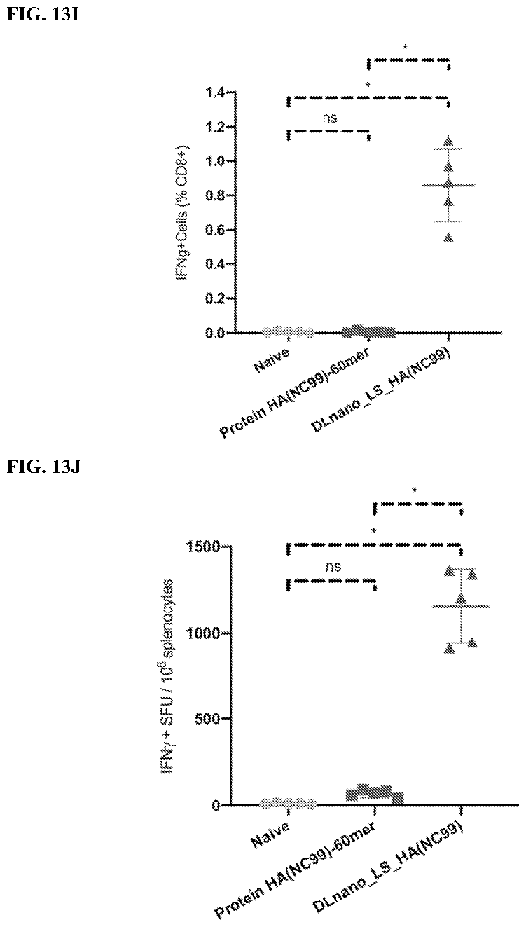

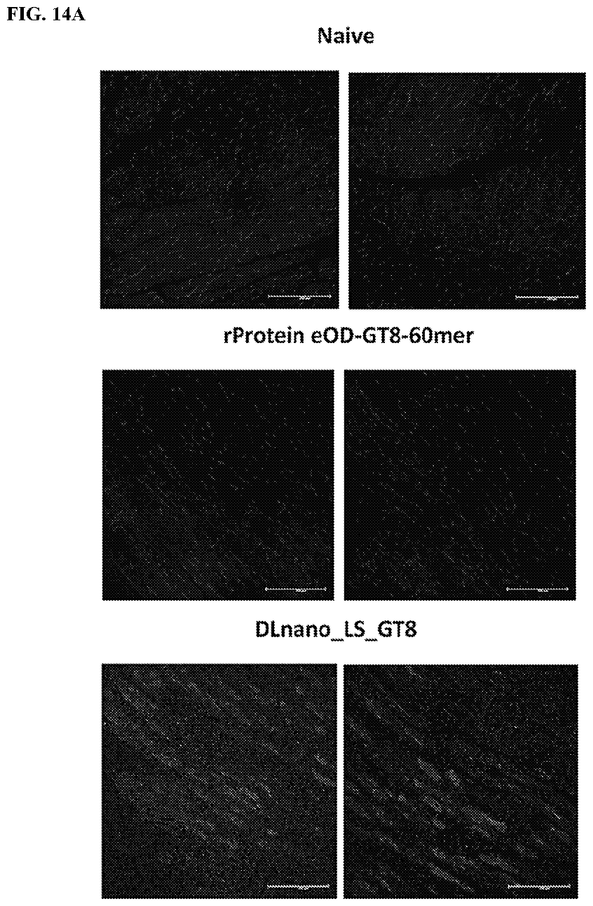

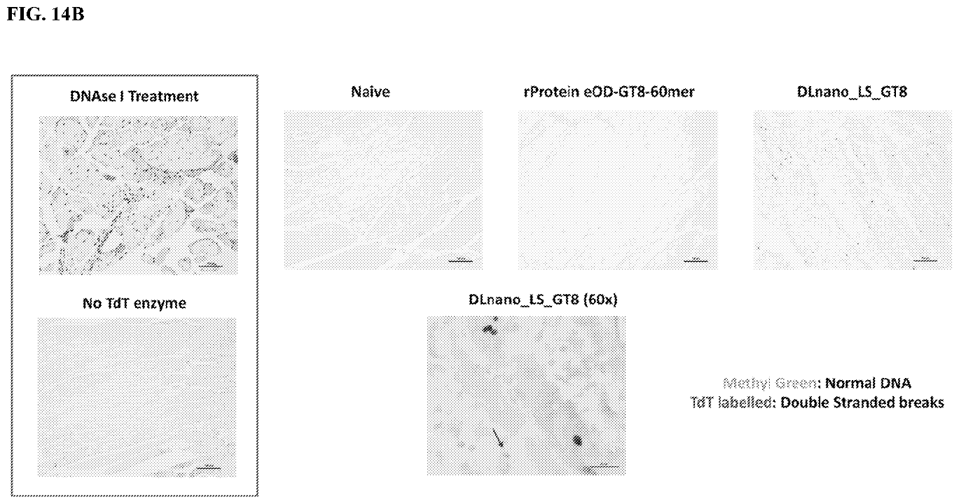

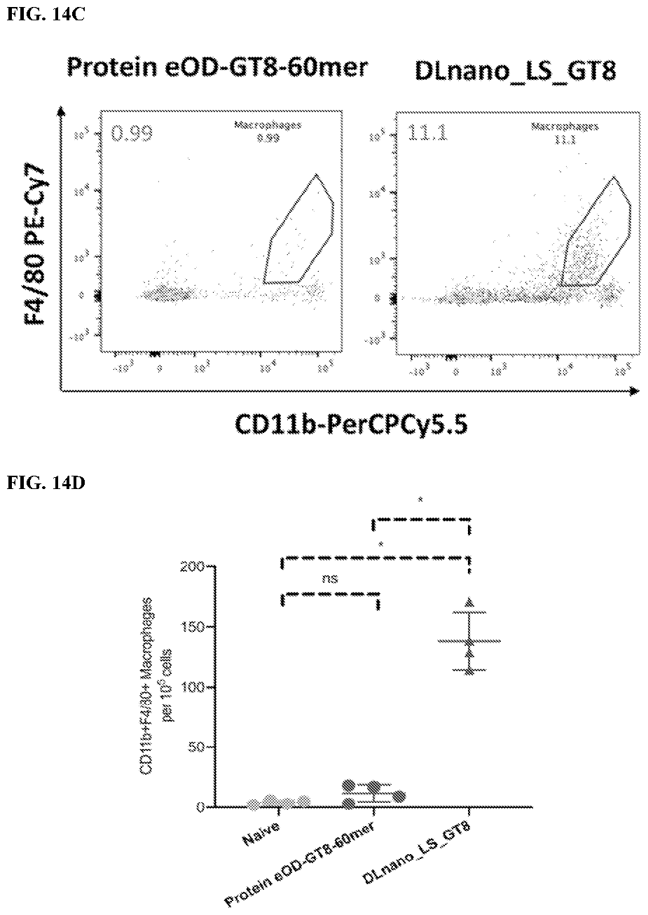

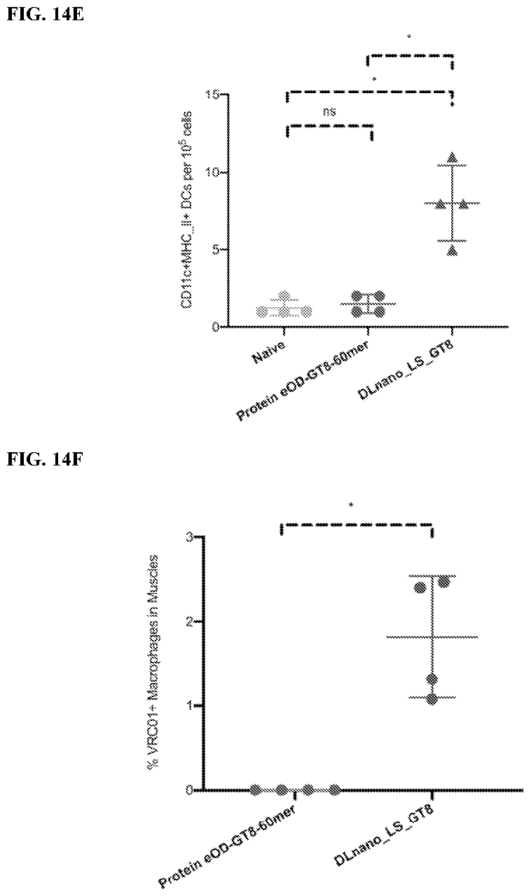

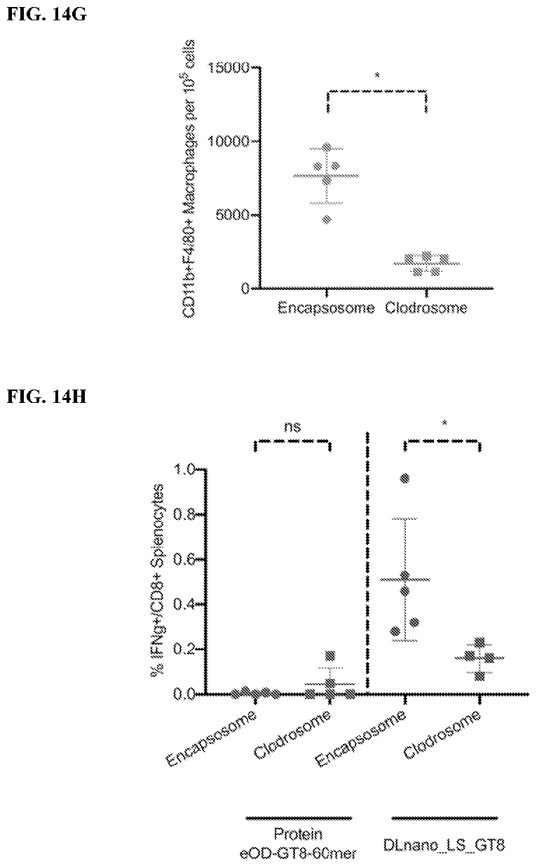

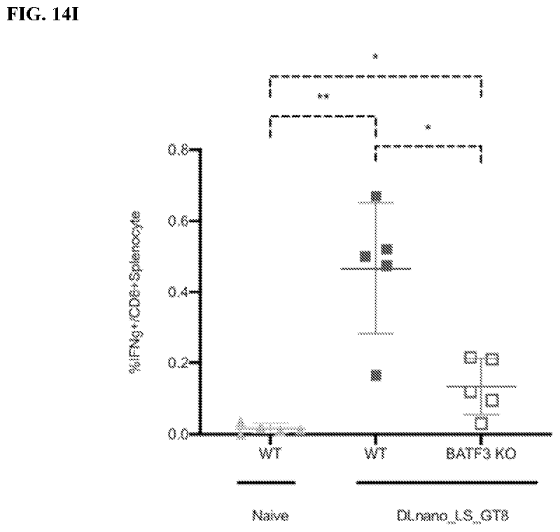

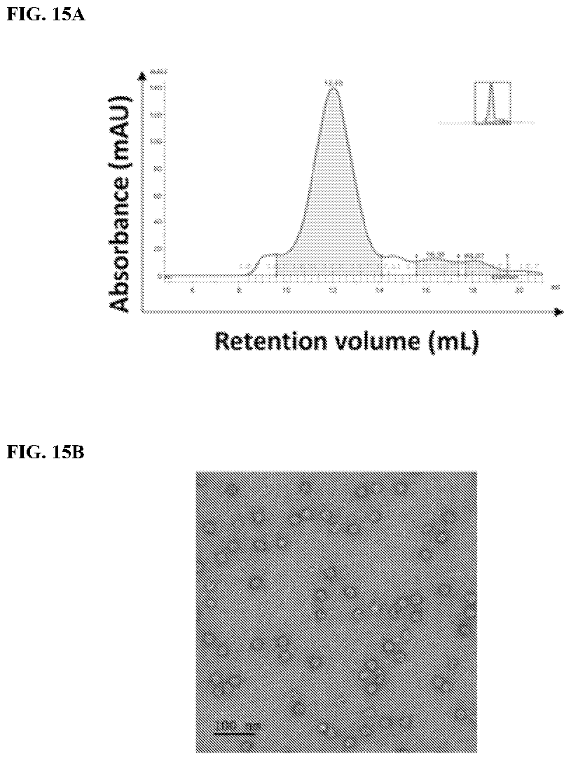

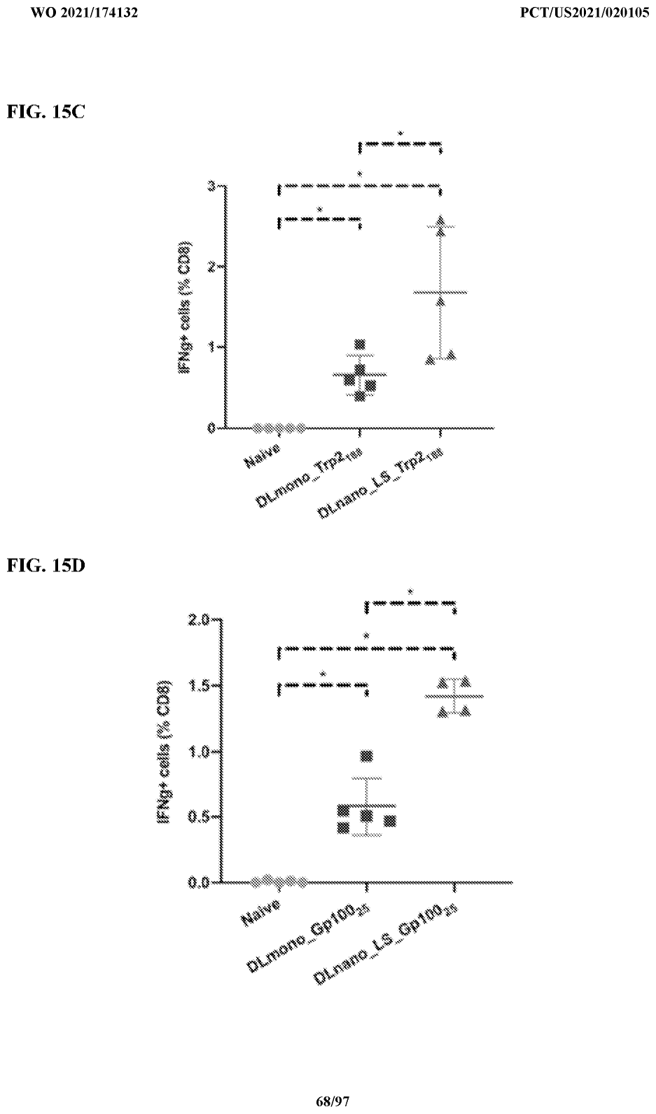

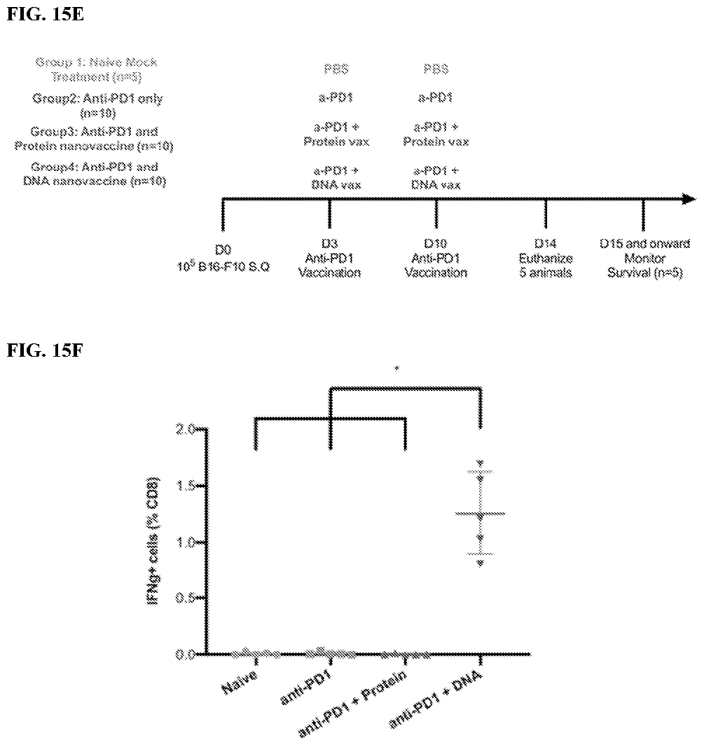

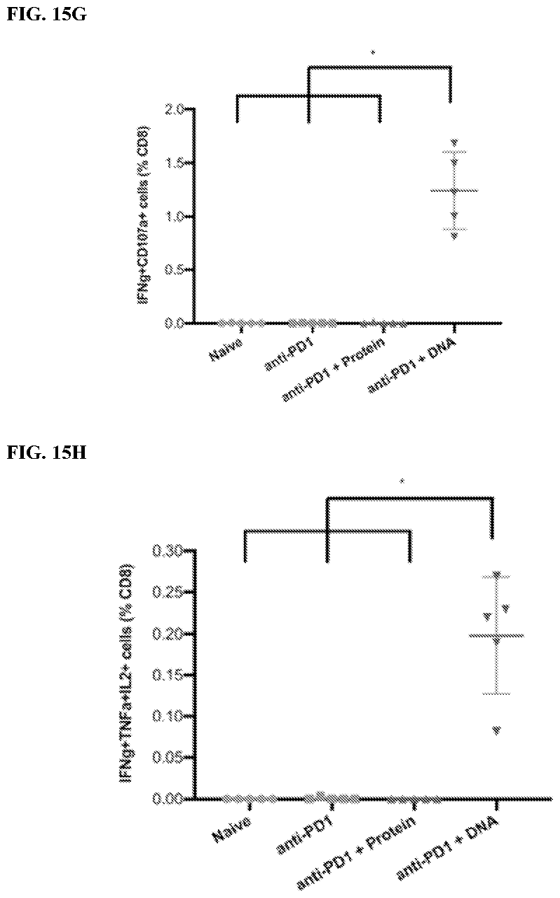

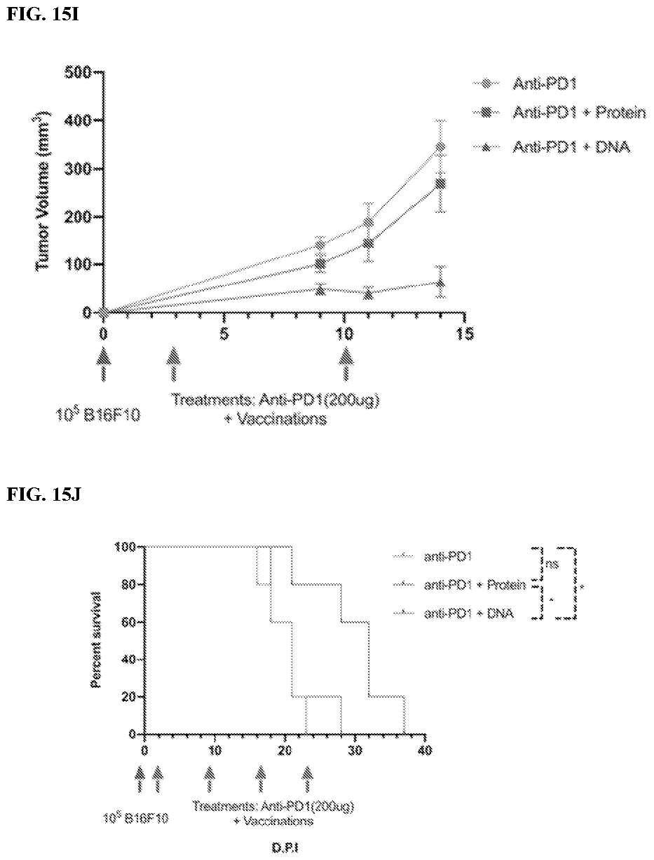

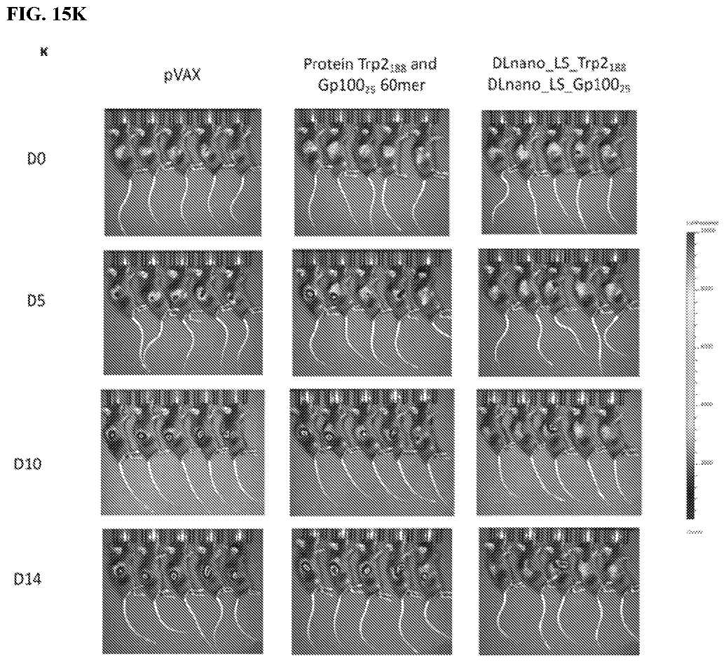

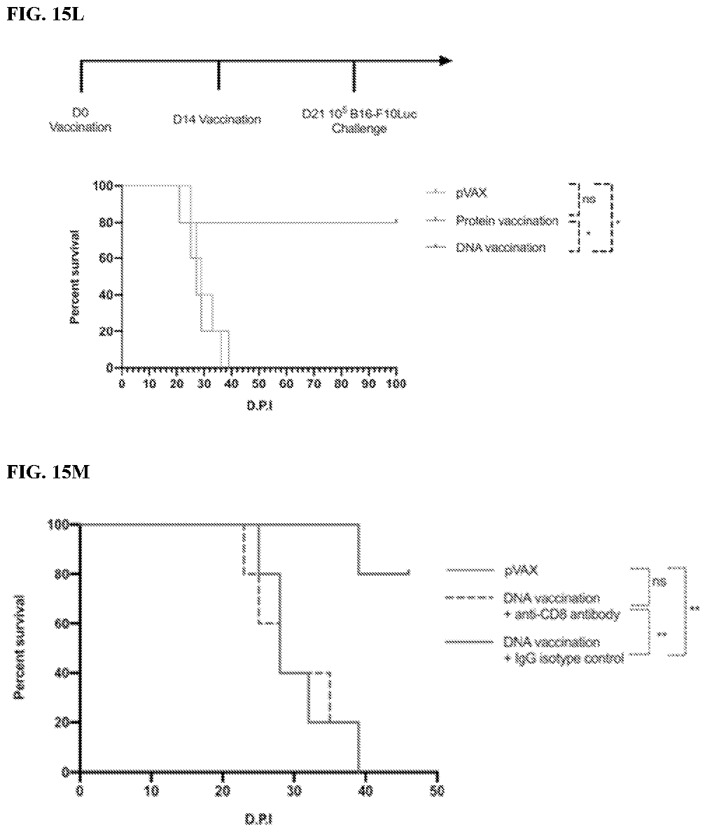

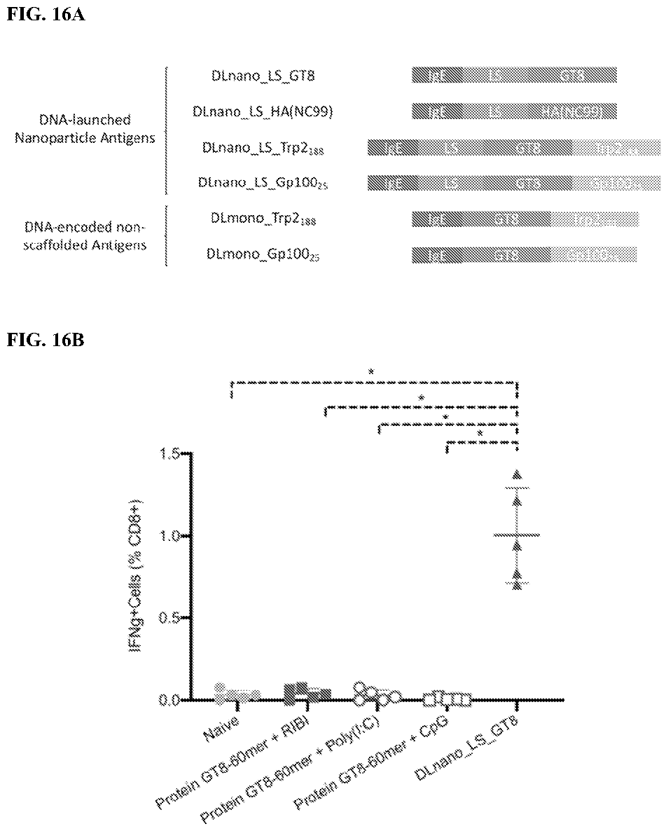

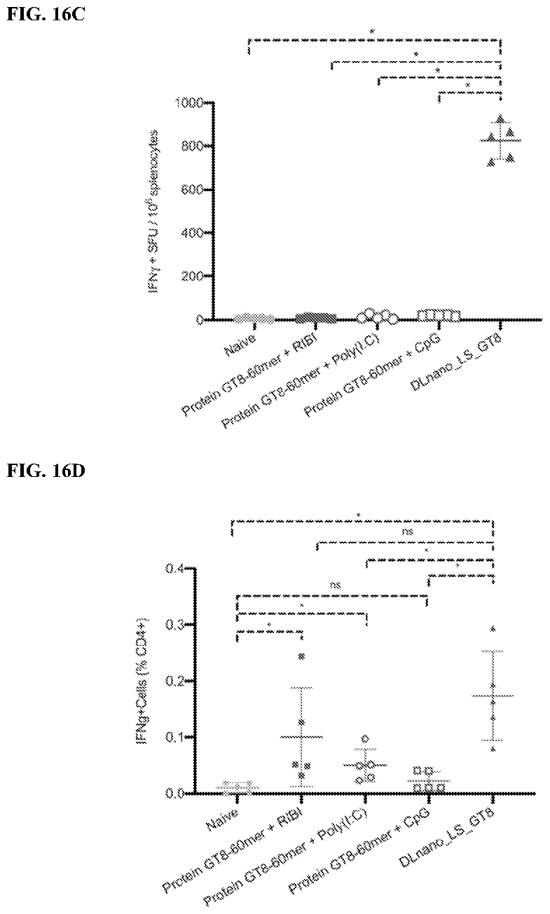

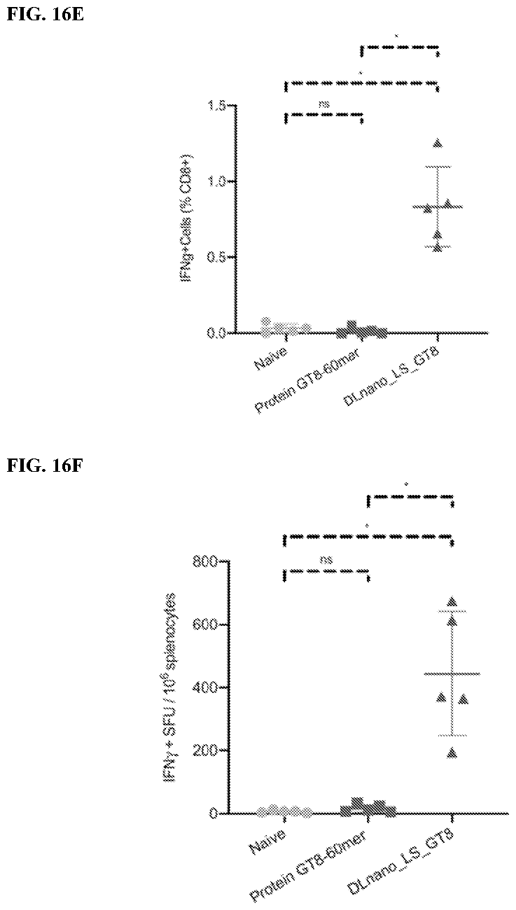

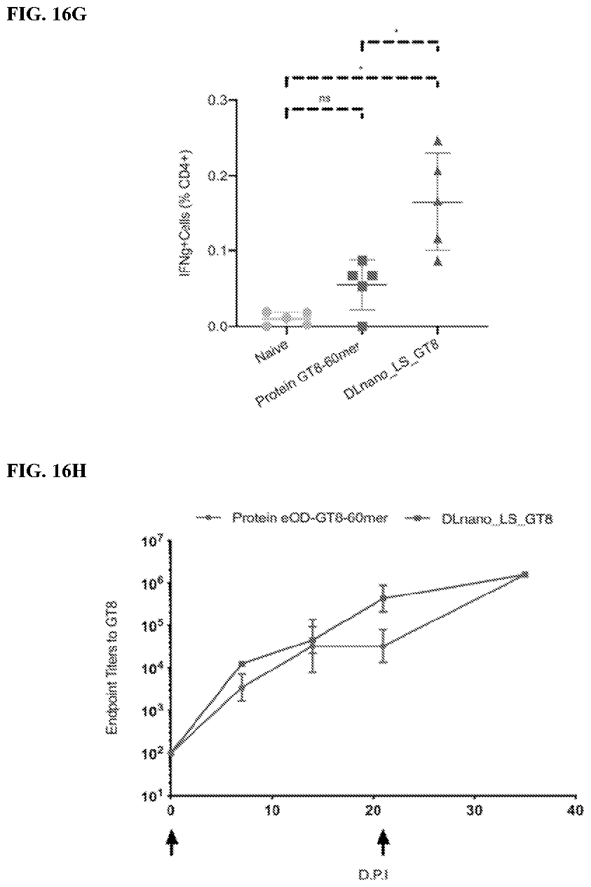

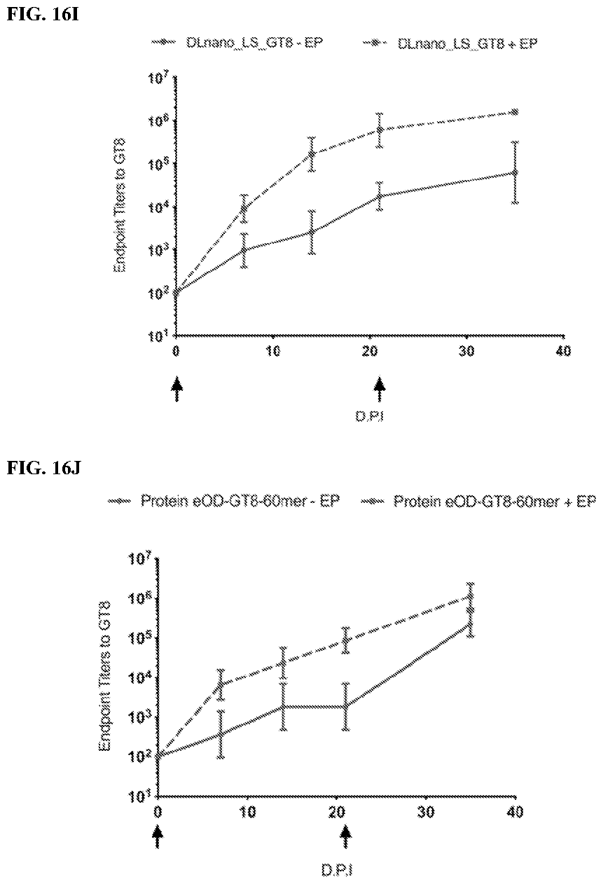

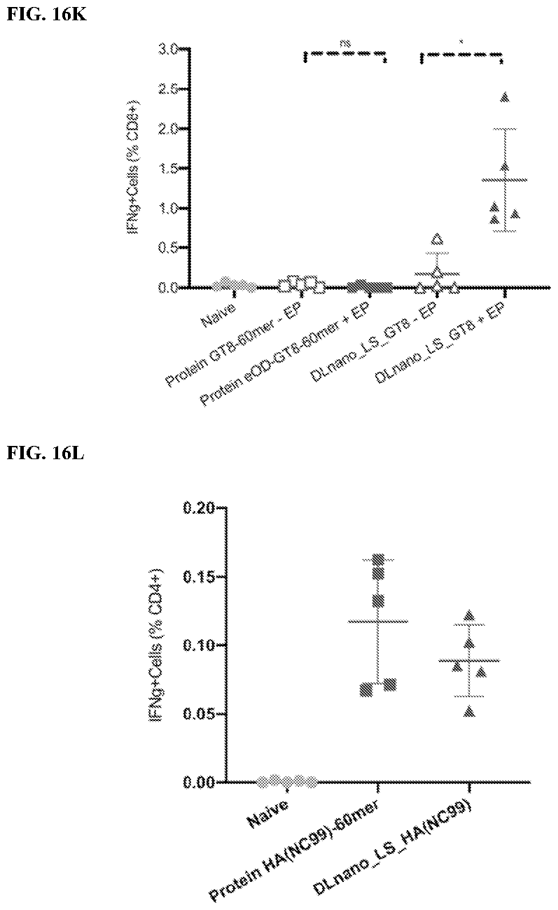

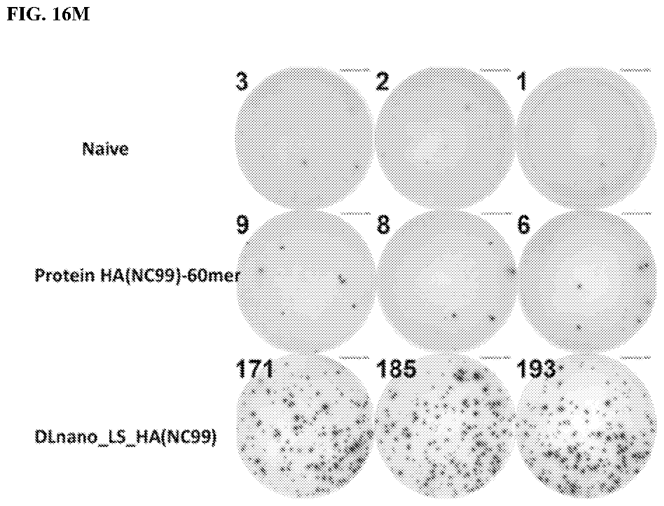

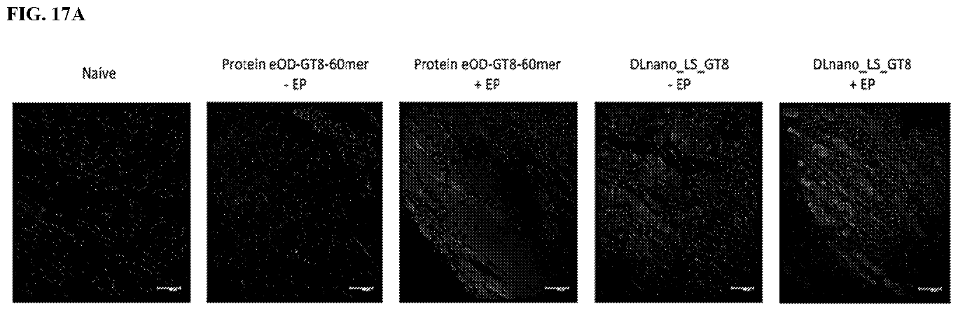

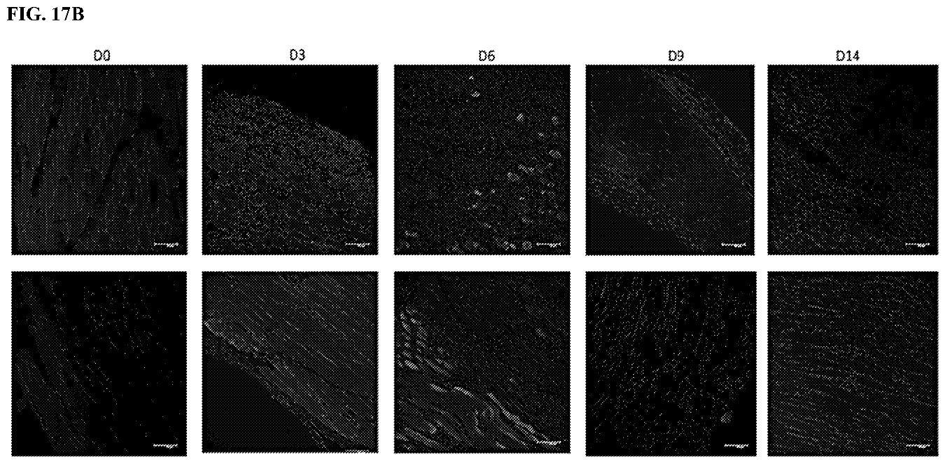

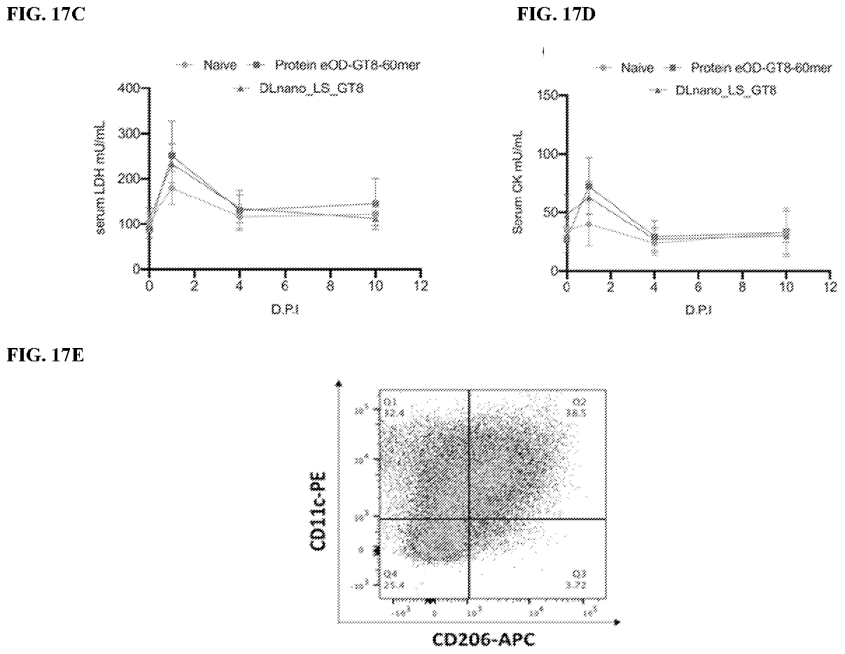

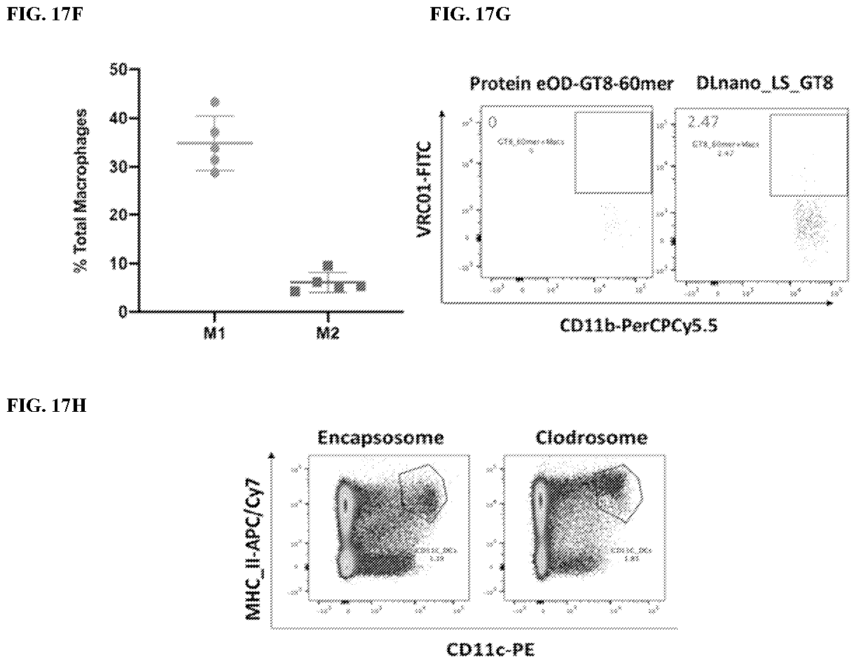

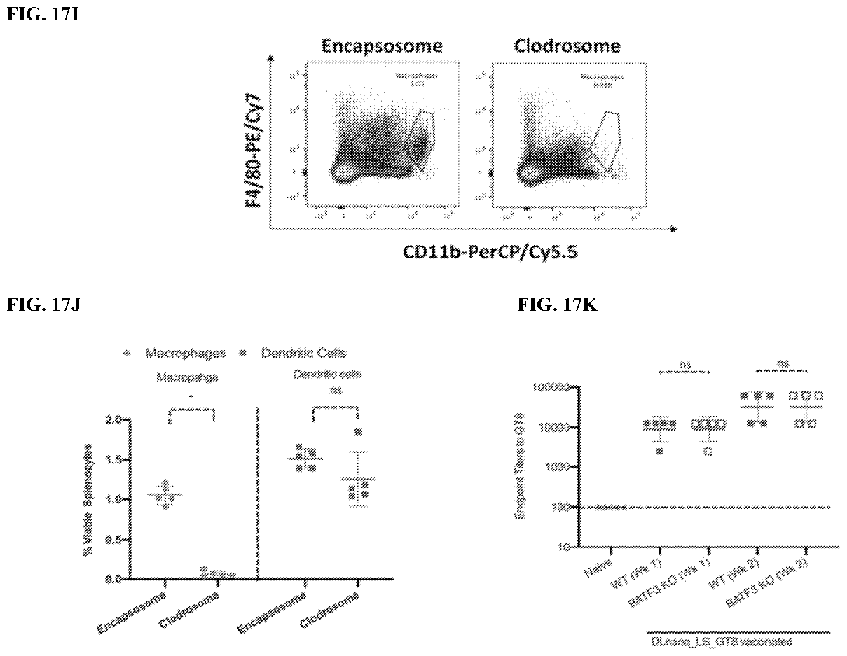

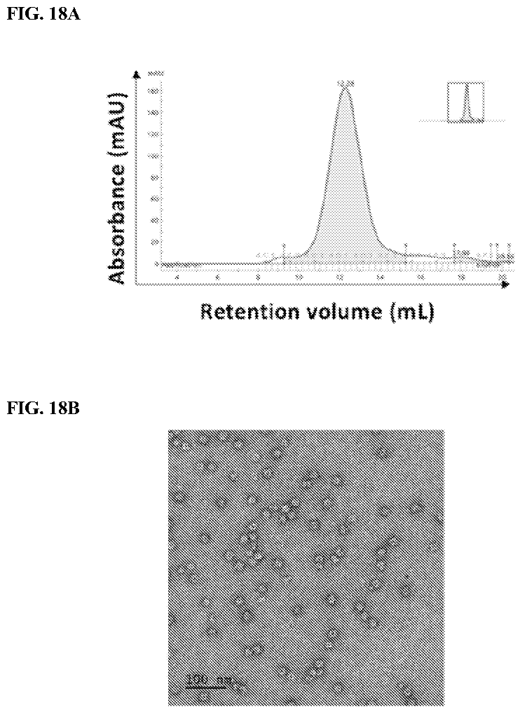

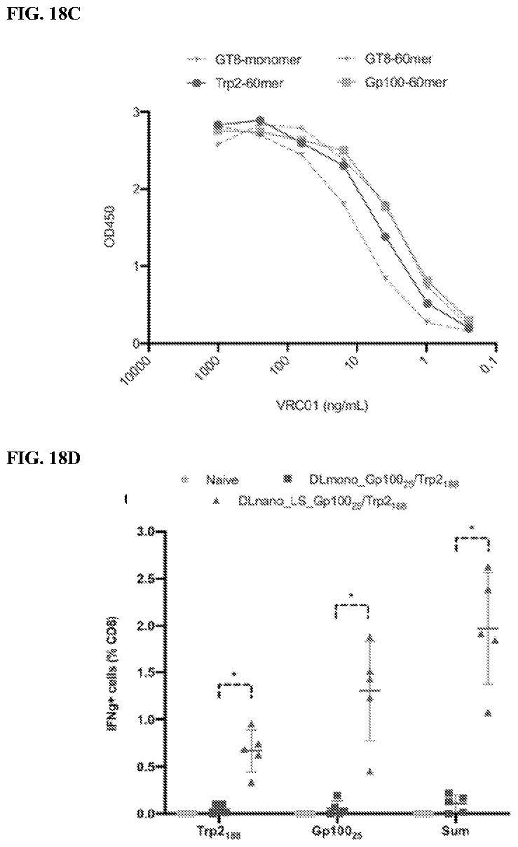

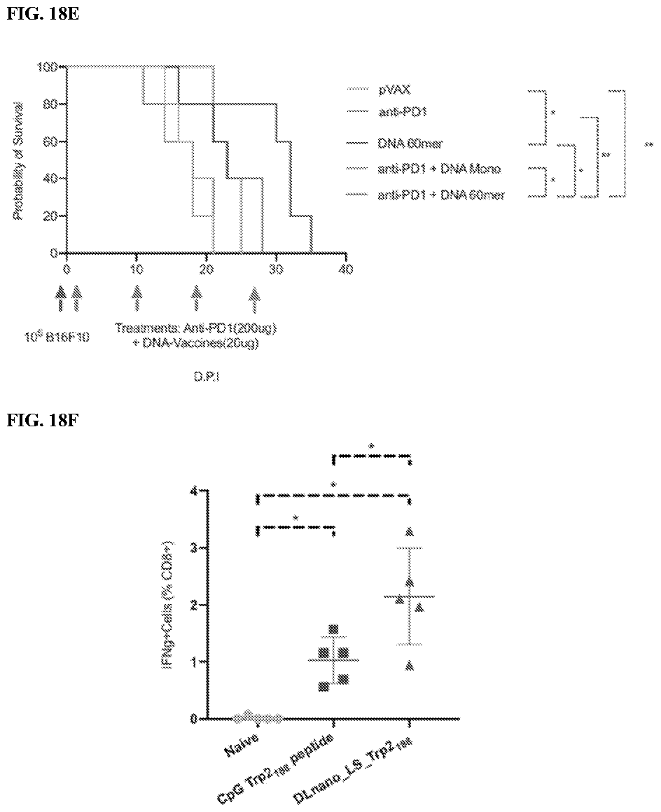

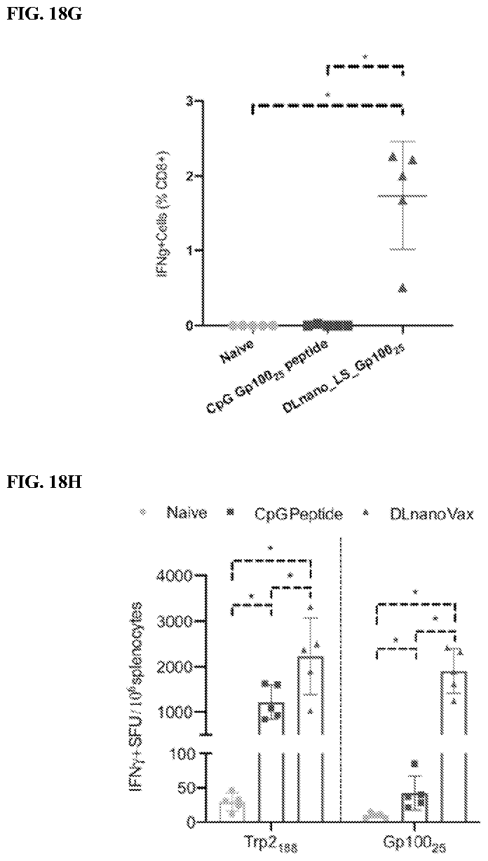

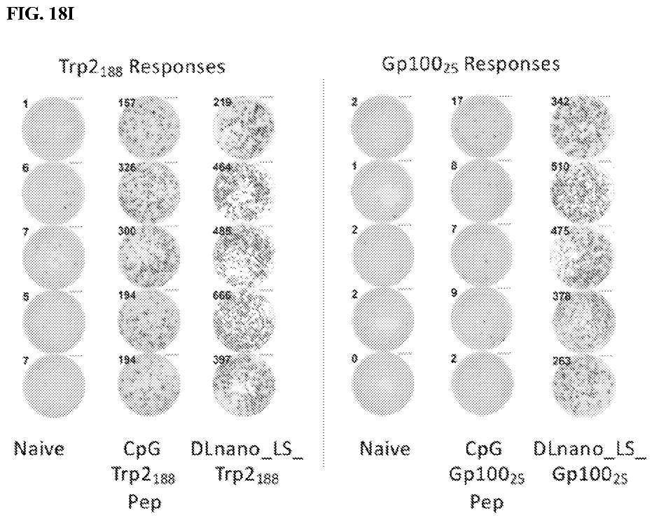

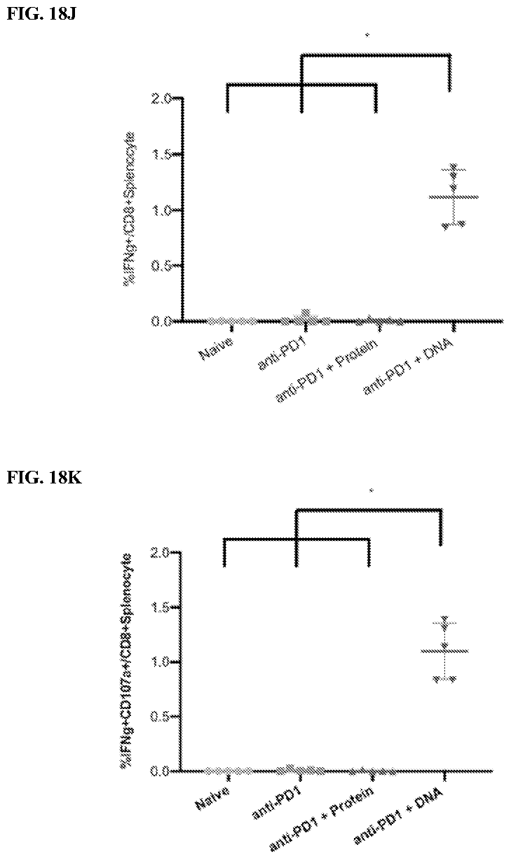

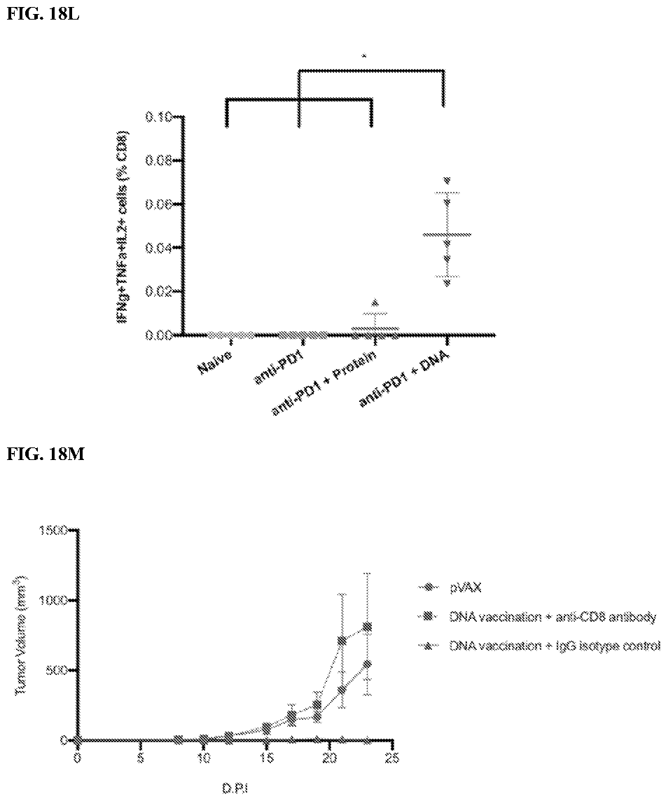

All animal experiments were carried out in accordance with animal protocols 201214, 201115, and 201221 approved by the Wistar Institute Institutional Animal Care and Use Committee (IACUC). For DNA-based immunization, 6 to 8 week old female C57BL/6, BALB/c or B6.129S(C)-Batf3tm1Kmm/J mice (Jackson Laboratory) were immunized with DNA vaccines via intramuscular injections into the tibialis anterior muscles, coupled with or without intramuscular EP with the CELLECTRA 3P device (Inovio Pharmaceuticals). For DNA immunizations using DLnano_LS_GT8 and DLnano_LS_HA(NC99) as antigens (except in dose comparison study, FIG. 16E-16H), 25 μg of plasmid DNA was used, a standard DNA dose that has been utilized in prior study [81, 84]. For DNA immunizations involving DLnano_LS_Trp2₁₈₈, DLnano_LS_Gp100₂₅, DLmono_Trp2₁₈₈, and DLmono_Gp100₂₅, 10 μg of individual plasmid DNA was used either alone or in combination. For vaccinations involving recombinant protein, a high protein dose was used in this study as compared to prior studies [85]. 6 to 8-week-old female BALB/c were immunized subcutaneously with 10 μg of recombinant eOD-GT8-60mer protein co-formulated with either 50 μL Sigma Adjuvant System (SigmaAldrich), 50 μg poly (I:C) (InvivoGen), or 20 μg CpG ODN 1826 VacciGrade (InvivoGen), with or without EP. Alternatively, BALB/c or C57BL/6 mice were immunized with 10 μg protein HA(NC99)-60mer, and 4 μg dose for LS_Trp2₁₈₈-60mer and LS_Gp100₂₅-60mer in 50 μL PBS co-formulated with 50 μL Sigma Adjuvant System (SigmaAldrich). For the high dose protein versus DNA comparison study reported in FIG. 16E-16H, 50 μg DLnano_LS_GT8 was administered with EP, or 50 μg protein eOD-GT8-60mer coformulated with RIBI was administered without EP. For peptide vaccinations, C57BL/6 mice received intramuscular vaccinations of 10 μg Trp2₁₈₈ or 10 μg Gp100₂₅ peptides (GenScript) co-formulated with 20 μg CpG ODN 1826 VacciGrade in sterile saline. For all immunizations reported in FIG. 13 or FIG. 16, the mice were immunized twice three weeks apart and euthanized two weeks post the second vaccination for cellular analysis. For immunizations reported in FIG. 14H, FIG. 14I, or FIG. 17K, the mice were immunized once and euthanized two weeks post the vaccination. For all immunogenicity studies in FIG. 15C-15D, FIG. 18D, and FIG. 18F-18I, the mice were immunized twice two weeks apart and euthanized two weeks post the second vaccination.

For vaccinations in mechanistic study (FIG. 14 and FIG. 17), C57BL/6 mice received intramuscular injections of 80 μg of DLnano_LS_GT8 co-formulated with 12U Hyaluronidase (SigmaAldrich) with or without intramuscular EP or 20 μg of protein eOD-GT8-60mer co-formulated 1:1 with Sigma Adjuvant System with or without intramuscular EP. For in vivo macrophage depletion, 6 to 8 week old female C57BL/6 mice received one or three intravenous injection of 750 μg clodronate disodium formulated in clodrosome (150 μL injection, Encapsula Nanoscience) via retro-orbital injections; control mice each received 150 μL of encapsosome (Encapsula Nanoscience) IV at the same timepoints.

For tumor challenge, B16-F10 (ATCC) were maintained in 10% FBS/DMEM under low passage (less than 10) and B16-F10-Luc2 cells (ATCC) were maintained in 10% FBS/DMEM enriched with 10 μg/mL blasticidin (Gibco) with routine testing for mycoplasma contaminations and other mouse pathogens. Cells were trypsinized and strained through 70 μm strainer to generate single cell suspensions. They were then administered sub-cutaneously to mice (10⁵ cells to each mouse in 100 μL PBS). The tumor size was measured every two days with a digital caliper, and tumor volume was determined with the formula V=0.5W²L (V=tumor volume, W=tumor width, L=tumor length). Mice with tumor volume greater than 2000 mm³ or with any dimension exceeding 2 cm were euthanized for humane purposes. For recombinant anti-PD1 (RMP1-14, Bio X Cell) administration in melanoma therapeutic treatment model, 200 μg of antibody was injected intraperitoneally in 100 u L PBS to each mouse weekly. For mechanistic study to determine the role of CD8+ T-cells in mediating in vivo control of tumor growth (FIG. 15M and FIG. 18I), prophylactically vaccinated mice received 200 μg anti-mouse CD8α antibody (2.43, BioXcell) or 200 μg Rat IgG2b isotype control (LTF-2, BioXcell) in 100 μL sterile PBS.

### 2. Supplemental Methods

i. Production of Recombinant Protein eOD-GT8-60Mer, HA(NC99)-60Mer, LS_Trp2188-60Mer and LS_Gp10025-60Mer

Expi293F cells (Invitrogen) were transfected with pVAX plasmid vector encoding the eOD-GT8-60mer, HA(NC99)-60mer, LS_Trp2₁₈₈-60mer or LS_Gp100₂₅-60mer with PEI (Sigma Aldrich)/OPTI-MEM (Invitrogen) and harvested 6 days post-transfection. Transfection supernatant was first purified with affinity chromatography using the AKTA pure 25 system and gravity flow columns filled with GNL Lectin beads (GE Healthcare). The eluate fractions from the affinity purification were pooled, concentrated and dialyzed into 1×PBS buffer before being loaded onto the SEC column and then purified with size exclusion chromatography using the Superose 6 Increase 10/300 GL column. (GE healthcare) Identified eluate fractions were then collected and concentrated to 1 mg/mL in PBS as previously described (81).

ii. ELISA—GT8-Binding ELISA

96-well half area plates (Corning) were coated at room temperature for 8 hours with 1 μg/mL MonoRab anti-His antibody (GenScript), followed by overnight blocking with blocking buffer containing 1×PBS, 5% skim milk, 10% goat serum, 1% BSA, 1% FBS, and 0.2% Tween-20. The plates were then incubated with 2 μg/mL of his-tagged GT8-monomer at room temperature for 2 hours, followed by addition of mice sera serially diluted with PBS with 1% FBS and 0.1% Tween and incubation at 37° C. for 2 hours. The plates were incubated at room temperature for 1 hour with anti-mouse IgG H+L HRP (Bethyl) at 1:20,000 dilution, followed by addition of TMB substrates (ThermoFisher) and then quenched with 1M H2SO4. Absorbance at 450 nm and 570 nm were recorded with BioTEK plate reader. Endpoint titer is defined as the highest dilution at which the OD of the post-immune sera exceeds the cut-off (mean OD of naïve animals plus standard deviations of the OD in the naïve sera multiplied with standard deviation multiplier f at the 99% confidence level).

iii. ELISA—HA-Binding ELISA

96-well half area plates were coated at 4° C. overnight with 2 μg/mL of recombinant HA(ΔTM)(H1N1/A/New Caledonia/20/1999) (Immune Technology), and blocked at room temperature for 2 hour with the buffer as described above. The plates were subsequently incubated with serially diluted mouse sera at 37° C. for 2 hours, followed by 1-hour incubation with anti-mouse IgG H+L HRP (Bethyl) at 1:20,000 dilution at room temperature and developed with TMB substrate. Absorbance at 450 nm and 570 nm were recorded with BioTEK plate reader.

iv. Antigenic Profile Characterization of Designed GT8, Tpr2 and Gp100-Nano-Vaccines

Corning half-area 96-well plates were coated with 2 μg/mL of GT8-monomer, COD-GT8-60mer, Trp2-60mer and Gp100-60mer at 4° ° C. overnight. The plates were then blocked with the buffer as described in the GT8-binding ELISA section for 2 hours at room temperature, followed by incubation with serially diluted VRC01 at room temperature for 2 hours. The plates were then incubated with anti-human Fc (cross-adsorbed against rabbits and mice) (Jackson Immunoresearch) at 1:10,000 dilution for 1 hour, followed by addition of TMB substrate for detection. Absorbance at 450 nm and 570 nm were recorded with BioTEK plate reader.

v. HAI Assay

Mice sera were treated with receptor-destroying enzyme (RDE, 1:3 ratio; SEIKEN) at 37° ° C. overnight for 18-20 hours followed by complement and enzyme inactivation at 56° C. for 45 minutes. RDE-treated sera were subsequently cross-adsorbed with 10% rooster red blood cells (Lampire Biologicals) in PBS at 4° C. for 1 hour. The cross-adsorbed sera were then serially diluted with PBS in a 96-well V-bottom microtiter plates (Corning). Four hemagglutinating doses (HAD) of A/New Caledonia/20/99 (BEI) were added to each well and the serum-virus mixture was incubated at room temperature for 1 hour. The mixture was then incubated with 50 μl 0.5% v/v rooster red blood cells in 0.9% saline for 30 minutes at room temperature. The HAI antibody titer was scored with the dot method, and the reciprocal of the highest dilution that did not cause agglutination of the rooster red blood cells was recorded.

vi. ELISpot Assay

Spleens from immunized mice were collected and homogenized into single cell suspension with a tissue stomacher in 10% FBS/1% Penicillin-streptomycin in RPMI 1640. Red blood cells were subsequently lysed with ACK lysing buffer (ThermoFisher) and percentage of viable cells were determined with Trypan Blue exclusion using Vi-CELL XR (Beckman Coulter). 200,000 cells were then plated in each well in the mouse IFNγ ELISpot plates (MabTech), followed by addition of peptide pools that span both the lumazine synthase, GT8 or HA domains, or individual Trp2 (SVYDFFVWL) and Gp100₂₅ peptide (EGPRNQDWL) at 5 μg/mL of final concentration for each peptide (GenScript). The cells were then stimulated at 37° C. for 16-18 hours, followed by development according to the manufacturer's instructions. Spots for each well were then imaged and counted with ImmunoSpot Macro Analyzer.

vii. Intracellular Cytokine Staining

Single cell suspension from spleens of immunized animals were prepared as described before and stimulated with 5 μg/mL of peptides (GenScript) for 5 hours at 37° C. in the presence of 1:500 protein transport inhibitor (ThermoFisher) and anti-mouse CD107a-FITC(ThermoFisher). The cells were then incubated with live/dead for 10 minutes at room temperature, surface stains (anti-mouse CD4 BV510, anti-mouse CD8 APC-Cy7) (BD-Biosciences) at room temperature for 30 minutes. The cells were then fixed and permeabilized according to manufacturer's instructions for BD Cytoperm Cytofix kit and stained with intracellular stains anti-mouse IL-2 PE-Cy7, anti-mouse IFN-γ APC, anti-mouse CD3c PE-Cy5 and anti-mouse TNFα BV605 (BioLegend) at 4° C. for 1 hour. The cells were subsequently analyzed with LSR II 18-color flow cytometer.

viii. Immunofluorescence

To detect presence of cleaved caspase-3 using immunofluorescence, four days after C57BL/6 mice were intramuscularly immunized with DLnano_LS_GT8 or protein COD-GT8-60mer, tibialis anterior muscles of the mice were harvested and preserved in 4% PFA/PBS for 4 hours at room temperature and then stored overnight in 70% EtOH/H2O at 4° C. The tissues were then serially dehydrated for paraffin embedding, and blocked in 3% BSA/PBS for 1 hour at room temperature, permeabilized with 0.5% Triton X-100/PBS, followed by overnight staining with rabbit anti-cleaved caspase 3 (1:200, Cell Signaling). The sections were then washed and stained with anti-Rabbit Alexa Fluor 594 antibody (1:200, Invitrogen) for 1 hour at room temperature, then counterstained with 0.5 μg/mL DAPI and imaged with Leica SP5 confocal microscopes.

ix. Serum CK and LDH Assay

Mouse serum CK and LDH enzymatic activity was measured 0, 1, 4, 7 and 10 days following intramuscular administrations of 25 μg DLnano_LS_GT8 or 10 μg RIBI-adjuvanted protein eOD-GT8-60mer or from untreated control mice using colorimetric assays according to manufacturer's protocol (Abcam Ab 155901 and Ab 65393). Changes in absorbance at 450 nm was measured at 37° C. under kinetics mode over time using the BioTEK plate reader.

x. TUNEL Assay

TUNEL assay was performed on fixed muscle specimen according to manufacturer's protocol (Abcam). Briefly, four days post treatments with DNA or protein vaccine, tibialis anterior muscles were harvested and fixed in 4% paraformaldehyde/PBS for 3 hours at 4° C. The tissues were then dehydrated and paraffin-embedded, the sample sections were then rehydrated by serial transfer of the tissues from 100% Ethanol to 70% Ethanol/water. The specimen was then permeabilized by treatment with Proteinase K at room temperature for 20 minutes. Endogenous peroxidase was then quenched by treatment of tissue sections with 3% H2O2 in methanol. The sections were then incubated with TdT enzyme in the equilibration buffer at room temperature for 1.5 hours in a humidified chamber, and the reactions were stopped with the stop solution provided in the kit. The sections were then blocked with provided blocking buffer at room temperature for 10 minutes and labelled with provided detection conjugate diluted 1:25 in blocking buffer at room temperature for 30 minutes. The sections were then developed with DAB substrate at room temperature for 15 minutes and then counter stained with methyl green before they were imaged with Nikon Eclipse 80i under 20× and 60× magnification.

xi. Flow Analyses of Muscle Tissues Post DNA or Protein Vaccination

Following intramuscular protein or DNA vaccinations in C57BL/6 mice, at each specified timepoint, transfected muscles were harvested. Cells were extracted from muscles through 30 minutes incubation digestion buffer-Hanks' Balanced Salt Solution [Gibco], supplemented with 0.5% collagenase (Life Technologies), 0.2% BSA, and 0.025% trypsin at 37° C. The cells were then stained with Ultra-Violet reactive (Live/Dead dye, 1:333 in PBS), and then with 1:200 dilution of anti-CD11b-PerCPCy5.5, anti-CD11c-PE, anti-MHC_Class II APC_Cy7, anti-F4/80_PE Cy 7 and anti-CD206-APC (Biolegend) in 2% FBS/PBS at room temperature for 1 hour before they were permeabilized with BD CytoPerm/CytoFix for 20 minutes at 4° C. and subsequently labelled with FITC-lightning link labelled VRC01 (Expedon) at 4° C. for 1 hour. The cells were then resuspended in 1× BD Permeabilization Wash buffer for analyses on LSR II 18-color flow cytometer.

xii. Negative Stain EM of Purified Nanoparticles

The nanoparticles were produced in Expi293 cells, purified using Agarose bound lectin beads (Agarose Galanthus Nivalis Lectin, Vector Laboratories) followed by size exclusion chromatography (GE Healthcare) using the Superose 6 Increase 10/300 GL column. The proteins were further dialyzed into Tris-buffered saline (TBS). A total of 3 μL of purified proteins was adsorbed onto glow discharged carbon-coated Cu400 EM grids. The grids were then stained with 3 μL of 2% uranyl acetate, blotted, and stained again with 3 μL of the stain followed by a final blot. Image collection and data processing was performed on a FEI Tecnai T12 microscope equipped with a Oneview Gatan camera at 90,450× magnification at the camera and a pixel size of 1.66 Å.

xiii. In Vivo Imaging

For B16-F10-Luc2 challenge study, tumor challenged mice received 150 mg/kg administration of VivoGlo™ Luciferin (Promega) at each timepoint formulated in sterile PBS, and then imaged with IVIS Spectrum CT for Bioluminescence with the auto-exposure settings (or for 60s, whichever is shorter) 10 minutes post injection.

xiv. Statistics

Power analysis was performed with R based on our preliminary data to determine the smallest sample size that would allow us to achieve a power of 0.9 with a pre-set a-value of 0.05. All statistical analyses were performed with PRISM V8.2.1 and R V3.5.1. Each individual data point was sampled independently. Two-tailed Mann Whitney Rank Tests were used to compare differences between groups. Log Rank test was used to compare survival between two groups in challenge survival studies. Bonferroni corrections were used to adjust for multiple comparisons.

### 3. Results

i. DNA-Launched but not Recombinant Protein Nanoparticle Vaccines Induced CD8+ T-Cell Responses to HIV and Influenza Antigens

First, we compared adaptive immune responses induced by DNA vaccination and protein vaccination adjuvanted by Sigma Adjuvant System (or RIBI). We utilized two model antigens: a priming HIV antigen eOD-GT8-60mer and an influenza hemagglutinin (H1 A/NewCaledonia/20/1999) 60mer antigen scaffolded by lumazine synthase [86, 87]. DNA-Launched nanoparticle Lumazine Synthase decorated with an anti-HIV-1 immunogen eOD-GT8 (DLnano_LS_GT8, FIG. 16A) and protein eOD-GT8-60mer induced similar antibody titers in BALB/c mice after two immunizations, even though antibody titer induced by DLnano_LS_GT8 was slightly higher after a single immunization (FIG. 13A). Epitope mapping conducted in prior studies identified that CD4+ T-cell responses elicited by LS-scaffolded DLnano-vaccines were predominantly specific for the LS domain. In this study, CD4+ T-cell responses to the LS domain after two vaccinations, as measured by intracellular cytokine staining (ICS) of splenocytes following peptide stimulations, were similar between DLnano_LS_GT8 and protein eOD-GT8-60mer (FIG. 13B). Strikingly, we observed robust induction of CD8+ T-cell responses to the GT8 domain in mice immunized with DLnano_LS_GT8 but not in those vaccinated with protein eOD-GT8-60mer as determined by ICS (FIG. 13C-13D) and IFNγ ELISpot assay (FIG. 13E-13F). We further determined if this observation was limited by our choice of RIBI as the protein adjuvant and performed a head to head comparison with 25 μg DLnano_LS_GT8 versus 10 μg protein eOD-GT8-60mer adjuvanted with either RIBI, 50 μg Poly(I:C) or 20 μg CpG ODN. We chose to evaluate Poly(I:C) and CpG ODN, as they are well-established in adjuvanting CD8+ T-cell responses when administered along with peptide vaccines [88, 89]. By ICS and IFNγ ELISpot (FIG. 16B-16C), we observed only DLnano_LS_GT8, but not protein eOD-GT8-60mer administered with any form of adjuvant, was capable of inducing CD8+ T-cell responses in our assays, even though both DLnano_LS_GT8 and protein eOD-GT8-60mer induced CD4+ T-cell responses to varying degrees, as previously observed (FID. 16C). We next determined if this observation might apply when higher vaccine doses were administered, and compared immune responses induced by DLnano_LS_GT8 and RIBI-adjuvanted protein eOD-GT8-60mer both at 50 μg dose. By ICS and IFNγ ELISpot (FIG. 16E-16F), only DLnano_LS_GT8 but not protein eOD-GT8-60mer was observed to induce CD8+ T-cell responses at this higher dose, even though CD4+ T-cell responses and humoral responses were both induced these routes of vaccination (FIG. 16G-16H). In addition, we determined the role of EP in mediating CD8+ T-cell responses. DLnano_LS_GT8 and RIBI-adjuvanted protein eOD-GT8-60mer were administered with or without EP. While EP significantly adjuvanted humoral responses for DLnano_LS_GT8 (FIG. 16I) and slightly improved humoral responses for protein eOD-GT8-60mer (FIG. 16J), EP could only adjuvant CD8+ T-cell responses in the DLnano_LS_GT8 groups, with no responses observed in the protein eOD-GT8-60mer groups with or without EP (FIG. 16K). Antigens supplied by protein versus DNA-launched eOD-GT8-60mer existed in different forms. Protein was given exogenously as a depot and existed as soluble factors. DNA-launched nano-vaccines, however, were expressed by with hosts' own myocytes and cell-associated. This finding suggested that induction of CD8+ T-cell immunity, in this context, required cell-associated antigens expressed in the host cells from DNA-cassettes.

We determined if the observation could be replicated when an alternative nanoparticle-scaffolded influenza antigen was evaluated. Indeed, two vaccinations of DLnano_LS_HA(NC99) and RIBI-adjuvanted protein HA(NC99)_60 mer induced similar binding antibody titers to recombinant NC99 hemagglutinin (FIG. 13G) as well as hemagglutination inhibition titers against the autologous A/NewCaledonia/20/1999 virus (FIG. 13H). CD4+ T-cell responses induced by DLnano_LS_HA(NC99) and protein HA(NC99)_60 mer were also similar (FIG. 16L). However, similar to what was observed for the HIV antigen, only DLnano_LS_HA(NC99) but not protein HA(NC99)_60 mer induced CD8+ T-cell response to the HA domain as measured by ICS (FIG. 13I) and by IFNγ ELIspot (FIG. 13J and FIG. 16M).

ii. Induction of Tissue Apoptosis and APC Infiltration by DNA-Launched Nanoparticle Vaccines were Important for CD8+ T-Cell Priming

We hypothesized distinct modality of antigen uptake and presentation for DNA-launched nanoparticle vaccine as compared to protein nanoparticle vaccine may attribute to unique induction of CTL responses by DLnano-vaccines. Previous studies observed that co-administration of DNA-vaccines with DNA-cassettes encoding pro-apoptotic genes led to significantly improved induced CTL responses [90]. We, therefore, hypothesized that DNA/EP vaccinations could create a pro-inflammatory environment at the site of transfection through the induction of tissue apoptosis, thereby promoting infiltration of APCs and antigen uptake from apoptotic myocytes expressing DNA-cassette encoded antigens.

We first assessed the extent of muscle tissue damage upon vaccine administration with an immunofluorescence assay to stain for cleaved caspase 3, which would be present in cellular cytosol following apoptotic signaling events [91]. In C57BL/6 mice, four days post injection (d.p.i), hypercellularity and expression of cleaved caspase 3 were only observed in the muscles of mice immunized with DLnano_LS_GT8 combined with EP but not in those vaccinated with protein eOD-GT8-60mer co-formulated in RIBI without EP or in naïve mice (FIG. 14A). Alternatively, we used an additional Terminal deoxynucleotidyl transferase (TdT) dUTP nick end labeling (TUNEL) assay to assess for double stranded DNA breaks, a surrogate marker for apoptosis [92], in the vaccinated muscle tissues (FIG. 14B). To ensure robustness of staining, we generated a positive control specimen by incubating muscle specimen with DNAse I, which generated double-stranded DNA breaks throughout the specimen, resulting in brown staining of all nuclei in the specimen. The negative control sample was generated by staining of the specimen in the absence of TdT, resulting in green appearance of all nuclei because of methyl green counterstain (FIG. 14B). Four d.p.i, brown nuclei suggestive of cellular apoptosis were only observed in muscle tissues treated with DLnano_LS_GT8 and electroporation but not in muscle sections from naïve mice or those treated with protein eOD-GT8-60mer formulated in RIBI without EP. At a higher level of magnification, nuclei fragmentation was also observed in the DNA group (FIG. 14B). EP was determined to be instrumental to the induction of myocyte apoptosis as cleaved caspase 3 expression was observed four d.p.i when either protein eOD-GT8-60mer or DLnano_LS_GT8 was delivered with EP. Without EP, low level cleaved caspase 3 expression was observed only for DLnano_LS_GT8 but not for protein eOD-GT8-60mer (FIG. 17A).

We performed a time course experiment and observed that DLnano_LS_GT8 induced tissue apoptosis was transient, peaking at 6 d.p.i and was fully resolved by 14 d.p.i (FIG. 17B). Additionally, tissue apoptosis should be limited locally to the injection sites. Indeed, we did not observe indicators of tissue damage such as significant elevations of serum lactose dehydrogenase (LDH) and creatine kinase (CK) levels in mice immunized with DLnano_LS_GT8 or protein eOD-GT8-60mer relative to untreated control mice post injections (FIG. 17C-17D).

We next examined the consequences of induced tissue apoptosis in terms of tissue APC infiltration. Indeed, seven d.p.i, we observed increased influx of CD11b+F4/80+ macrophages into muscles of mice treated with DLnano_LS_GT8 and EP compared to protein eOD-GT8-60mer formulated in RIBI without EP (FIG. 14C-14D). Furthermore, infiltrating macrophages were observed to be predominantly pro-inflammatory CD11c+CD206− M1 macrophages rather than anti-inflammatory CD11c−CD206+M2 macrophages (FIG. 17E-17F). Increased influx of CD11c+MHC Class II+ dendritic cells (DCs) was also observed for mice treated with DLnano_LS_GT8 compared to those treated with protein eOD-GT8-60mer; however, infiltrating DCs were significantly less abundant than macrophages by approximately 20-fold (FIG. 14E). Finally, staining of antigen-presenting cells with VRC01 (broadly neutralizing antibody with high affinity for GT8)-FITC demonstrated that infiltrating macrophages induced by DLnano_LS_GT8 but not those induced by protein eOD-GT8-60mer had taken up the GT8 antigen (FIG. 14F and FIG. 17G).

To determine the functional roles played by macrophages in priming of CD8+ T-cell responses by DNA-launched nanoparticle vaccines, we systemically depleted the macrophage populations in C57BL/6 mice prior to DNA vaccinations. Depletion was carried out through intravenous (IV) injection of clodrosome, which specifically depleted phagocytic macrophages [93]. Single IV infusion of clodrosome specifically depleted CD11b+F4/80+ macrophages but not CD11c+MHC Class II+ DCs in the spleens of animals one d.p.i (FIG. 17H-17J). Depletion of infiltrating macrophages in the muscles post vaccination required increased doses of clodrosome on −3, 0, and 3 d.p.i (with respect to DNA vaccination), which significantly reduced macrophage infiltration into the muscles by approximately four-fold (FIG. 14G). Importantly, this depletion scheme significantly attenuated induced CD8+ T-cell responses in vaccinated C57BL/6 mice following one vaccination of DLnano_LS_GT8 14 d.p.i by approximately three-fold (FIG. 14H). It has previously been reported that monocytic populations could transfer sequestered peptides through gap junctions to cDCs, which can more efficiently prime CD8+ T cells [94]. We investigated the role of CD11c+CD8α+ conventional dendritic cells (cDCs) in cross-presentation of DLnano-immunogens, using BATF3KO transgenic mice which lacks splenic development of CD8α+ cDCs as a model [95]. Single immunization of DLnano_LS_GT8 induced similar humoral responses to GT8 in BATF3KO and wildtype C57BL/6 mice (FIG. 17K), even though induced CD8+ T-cell responses were significantly attenuated by approximately four-fold in BATF3KO mice (FIG. 14I). Taken together, the data highlights the importance of infiltrating macrophage populations in scavenging in vivo produced DLnano-immunogens, and the role of both phagocytic macrophages and CD8α+ cDCs in cross-presentation to and priming of CD8+ T-cells.

iii. DNA-Launched Nanoparticle Vaccines Scaffolding Trp2188 and Gp10025 Peptides Mediated Protection Against Melanoma Challenge in Mice

To demonstrate the functional relevance of CD8+ T-cell priming by DNA-launched nanoparticle vaccines in the treatment of cancer, we designed novel nanoparticle vaccines displaying immunodominant CD8+ T-cell epitopes. The self-assembling nanoparticles were engineered using a structure-guided process. In our preliminary experiments, Lumazine Synthase on its own does not express well in mammalian cell lines and we hypothesized that it could not be used on its own to scaffold anti-tumor peptide antigens. We engineered a new version of DLnano_LS_GT8 capable of displaying peptide antigens. In this construction, the heavily glycosylated GT8 domain facilitates solubilization and secretion of designed nanoparticles and could potentially be replaced by other heavily glycosylated domains. To ensure epitope accessibility and homogenous nanoparticle assembly, N-linked glycans in proximity to the C-terminus of GT8 were removed by mutations in the PNGS sequence; additionally, a 15 amino acid linker was also incorporated to the C-terminus of GT8 upstream of the scaffolded anti-tumor peptide(s). We designed nanoparticles presenting 60 copies of Trp2₁₈₈ peptide (DLnano_LS_Trp2₁₈₈) and Gp100₂₅ peptide (DLnano_LS_Gp100₂₅). Homogenous in vitro assemblies of DLnano_LS_Trp2₁₈₈ and DLnano_LS_Gp100₂₅ were observed by Size Exclusion Chromatography, SEC, (FIG. 15A and FIG. 18A) and by negative stain Electron Microscopy, nsEM (FIG. 15B and FIG. 18B), respectively. Additionally, we demonstrated the multivalent nature of designed DLnano_LS_Trp2₁₈₈ and DLnano_LS_Gp100₂₅ nanoparticles, which bind to VRC01 with affinity similar to that of eOD-GT8-60mer, and higher than that of GT8-monomer (FIG. 18C). Groups of C57BL/6 mice were immunized with designed DNA-launched nanoparticle Trp2₁₈₈ or Gp100₂₅ vaccines individually, and were observed to induce significantly improved CD8+ T-cell responses to the Trp2₁₈₈ and Gp100₂₅ peptides compared to DNA vaccines encoding monomeric GT8-scaffolded Trp2₁₈₈ and Gp100₂₅ respectively (DLmono_Trp2₁₈₈ and DLmono_Gp100₂₅, FIG. 15C-15D). To determine if we could deliver both epitopes at once, we performed a follow-up experiment where we co-administered DLnano_LS_Trp2₁₈₈ and DLnano_LS_Gp100₂₅ in separate sites in a single animal. The co-administration resulted in improved elicitation of CD8+ T-cell responses to both Trp2 and Gp100 peptides as compared to DLmono_Trp2₁₈₈ and DLmono_Gp100₂₅ (FIG. 18D). We evaluated potency of designed DLmono_Trp2₁₈₈, DLmono_Gp100₂₅, DLnano_LS_Trp2₁₈₈ and DLnano_LS_Gp100₂₅ in therapeutic B16-F10 melanoma challenge survival study (FIG. 18E). It was observed DLnano_LS_Trp2₁₈₈ and DLnano_LS_Gp100₂₅ treatments, in the absence of anti-PD1 treatment, significantly extended median survival in the mice as compared to the pVAX group. Potency of DLnano_LS_Trp2₁₈₈ and DLnano_LS_Gp100₂₅ treatments could be further enhanced when anti-PD1 antibody was included in the treatment cocktail. Most importantly, when anti-PD1 antibody was co-administered, DLnano_LS_Trp2₁₈₈ and DLnano_LS_Gp100₂₅ significantly improved the survival outcome as compared to DLmono_Trp2₁₈₈ and DLmono_Gp100₂₅, extending the median survival by 9 days.

We next compared CTL responses induced by DLnano-vaccines to CpG adjuvanted peptide vaccines head-to-head. We observed that both DLnano_LS_Trp2₁₈₈ and CpG adjuvanted Trp2₁₈₈ peptide vaccination induced peptide-specific CD8+ T-cell responses, though responses induced by DLnano_LS_Trp2₁₈₈ vaccination were significantly higher (FIG. 18F). For Gp100₂₅ peptide, prior studies did not observe CpG adjuvanted Gp100₂₅ vaccination to induce strong CD8+ T-cell responses [96]. Consistent with this prior observation, our study indicated that vaccination with only DLnano_LS_Gp100₂₅, but not CpG adjuvanted Gp100₂₅ peptide, induced robust peptide-specific CD8+ T-cell responses by ICS (FIG. 18G), or by the more sensitive IFNg ELIspot assay (FIG. 18H and FIG. 18I).

We next compared induction of CD8+ T-cell responses to Trp2 (SVYDFFVWL) and Gp100 (EGPRNQDWL) epitopes in B16-F10 melanoma bearing mice, which received cither control PBS treatment or protein versus DNA vaccinations of nanoparticles scaffolding 60 copies of Trp2 and Gp100 peptides. Anti-PD1 treatments were administered to three groups of mice (Groups 2-4) in the study three days post tumor inoculation and weekly thereafter (FIG. 15E) [99]. We observed that 14 days post subcutaneous inoculation of 105 B16-F10 cells and following two combined vaccines/anti-PD1 treatments, CD8+ T-cell responses to Trp2 was only observed in mice treated with DNA vaccinations but not those receiving anti-PD1 treatment alone or anti-PD1 with protein vaccinations (FIG. 15F). Induced CD8+ T-cells specific for Trp2 were observed to exhibit effector phenotypes, IFNγ+CD107a+(FIG. 15G), and were also poly-functional as determined by co-expression of IFNγ, TNFα and IL2 (FIG. 15H). Similar observations were also made in terms of CD8+ T-cell responses to Gp100, even though lower frequency of epitope-specific poly-functional CD8+ T-cell was observed upon DNA vaccination (FIG. 18J-18L). In this therapeutic model, DNA but not protein vaccination of Trp2₁₈₈ and Gp100₂₅-60mer was observed to suppress B16-F10 tumor growth in mice (FIG. 15I). Additionally, DNA but not protein vaccination was observed to significantly prolong median survival of mice in the therapeutic model by 11 days (p-value=0.0177 by Log-Rank test) (FIG. 15J). The effect was even more pronounced when mice were vaccinated prior to tumor inoculation. Two vaccinations of DLnano_LS_Trp2₁₈₈ and DLnano_LS_Gp100₂₅ prior to 105 B16-F10-Luc challenge completely prevented tumor growth in 80% (4/5) mice; whereas tumor growth was observed in all pVAX control mice or mice treated with protein Trp2₁₈₈ and Gp100₂₅-60mer (FIG. 15K). Additionally, while all mice in the pVAX and protein groups died, 80% of mice in the DNA group had tumor-free survival until the end of the study (FIG. 15L), highlighting therapeutic utility of CTL priming by DLnano-vaccines. Finally, we determined whether DLnano-mediated protection in the prophylactic challenge model was CD8+ T cell mediated (FIG. 15M and FIG. 18M). Mice received cither pVAX or DLnano_LS_Trp2₁₈₈ and DLnano_LS_Gp100₂₅ vaccinations twice before they were challenged with 105 B16-F10 Luc cells a week after. In the DLnano-vaccinated groups, a group of mice received 200 μg anti-CD8 depletion antibody, whereas the other group of mice received 200 μg Rat IgG2b isotype control antibody. Rapid tumor growths were observed in all mice in the pVAX and CD8-depletion groups, but not in the isotype control group (FIG. 18M); upon completion of the study, all mice in the pVAX and CD8-depletion groups had died, whereas 80% (4/5) mice in the isotype control group had tumor-free survival (FIG. 15M). Taken together, the data suggests DLnano vaccination can uniquely elicit CD8+ T-cell responses to confer protection against melanoma both in prophylactic and therapeutic B16-F10 challenge model.

### 4. Discussion

CD8+ T-cells can play an extremely important role in surveillance against intracellular pathogens and tumors. In the cancer space, presence of tumor infiltrating CD8+ T-cell is correlated with improved prognosis in cancer patients [100, 101]. Induction of CD8+ T-cell responses also corresponds to improved tumor control and survival in various preclinical animal studies [102]. Such observations have led to the use of T-cell based therapies, such as DC vaccines or in vitro expansion and adoptive transfer of tumor infiltrating lymphocytes (TIL), in cancer patients, achieving varying degrees of success [103, 104]. Alternatively, several reports also describe the anti-tumor activity of antigen-specific CD4 T-cells. T_{H}1 cells, for instance, are responsible for immune responses against tumors by either enhancing CD8 T-cell response or activating macrophages to phagocytose cancerous cells [105]. CD4 CTL is another sub-set of CD4 T cells that have acquired cytolytic activity and demonstrated clear anti-tumor activity. Transfer of these tumor-reactive CD4 CTL cells into lymphopenic hosts, followed by tumor irradiation and anti-CTLA-4 antibody treatment, results in T-cell expansion and the expression of IFN-g and granzyme-B, as well as regression of established tumors [106]. Our work in this paper mainly focuses on designing DNA-launched nanoparticle vaccines scaffolding CD8+ T-cell epitopes, but nano-vaccines scaffolding CD4+ T-cell epitopes should also be closely examined in future work.

Induction of CTL responses by vaccination can be challenging and not readily achieved by vaccination with protein or inactivated virus. For example, the most advanced malaria vaccines under development, RTS,S, which contains repeat and T-cell epitopes of CircumSporozoite Protein fused to hepatitis B surface antigen to form protein particles, could induce antibody but not CD8+ T-cell response to the vaccine antigens [107].

DNA vaccines have been shown to elicit CD8+ T-cell responses both in preclinical animal models and in clinical trials [78, 84]. Prior mechanistic studies highlighted plausible contributions by direct transfection of DCs and macrophages with DNA cassettes [79]. Additionally, cross presentation of DNA-cassettes encoded antigens was also believed to be important for priming CTL through more indirect evidence [80]. In this work, we observed DNA/EP mediated tissue apoptosis to trigger macrophage infiltration and uptake of antigen expressed by and associated with the host cells, which may subsequently transfer sequestered peptides to cDCs for priming of CD8+ T cells. It should be emphasized that while macrophages were found to be essential to the induction of CD8+ T-cell responses induced by DLnano-vaccines, the clodrosome treatments described in this manuscript systemically depleted macrophages, including—but were not limited to—the electroporation-induced muscle-infiltrating macrophages. Future study to further characterize the biology of these muscle-infiltrating macrophages, such as through the use of single-cell transcriptomics, is likely important, as the insights may be harnessed to promote the induction of more robust anti-tumor CD8+ T-cell immunity by DLnano- and other types of cancer vaccines. Induced tissue apoptosis was observed to be transient and local, as we would expect given strong safety profile of DNA vaccines in clinic [78].

We also demonstrated the importance of CD8+ T-cell priming by DNA-launched nanoparticle vaccines in a melanoma challenge model. We designed a novel and generalizable nanoparticle platform for displaying CD8+ T cell epitopes to various tumor antigens. Several mutations were introduced near the C-terminus of the eOD-GT8-60mer scaffolding domain to knock out the glycosylation sites to reduce steric hinderance, allowing for more efficient display of CTL epitopes at the C-terminus and homogeneous assembly of the designed nano-vaccines into 60-mer. As a proof-of-principle, we engineered two nanoparticle vaccines scaffolding 60 copies of CD8+ epitopes from Trp2 and Gp100. DNA-cassettes encoding anti-melanoma LS-nanoparticles scaffolding Trp2 and Gp100 epitopes could elicit significantly improved CTL responses to both targets as compared to conventional monomeric DNA vaccines or CpG adjuvanted peptide vaccines. DNA but not protein vaccinations of Trp2₁₈₈ and Gp100₂₅ 60mer elicited CTL responses in mice, suppressed tumor growth and prolonged overall survival in the therapeutic tumor challenge model, and conferred 80% protection when the vaccines were prophylactically administered. The work provided a demonstration that robust CTL responses can be generated with great simplicity by harnessing physical injury to create a pro-inflammatory environment that favored APC infiltration, antigen uptake and cross-presentation. Induced CTL responses can be directed to whole antigens, such as to GT8 or to influenza (FIG. 13), or to selected peptides (FIG. 15), creating possibility of developing whole antigen or neoantigen peptide-oriented DNA-launched nanoparticle vaccines against various cancer targets, and creating a viable and attractive strategy in the exciting era of cancer immunotherapy.

The sequences used in this Example or the ones that can be used in the method described herein are provided in Table X below.

TABLE X
Sequences for Example 9 (Leader sequence: underlined; Scaffolding domain:
italic; CTL epitope: bold and italic; Linker: bold)
1. gp100_25_GT60 mer (DLnano_LS_Gp100₂₅)
MDWTWILFLVAAATRVHSMQIYEGKLTAEGLRFGIVASRANHALVDRLVEGAIDAIVRH
GGREEDITLVRVCGSWEIPVAAGELARKEDIDAVIAIGVLCRGATPSFDYIASEVSKGLADLS
LELRKPITFGVITADTLEQAIEAAGTCHGNKGWEAALCAIEMANLFKSLRGGSGGSGGSG
GSGGGDTITLPCRPAPPPHCSSNITGLILTRQGGYSNDNTVIFRPSGGDWRDIARCQIAGTV
VSTQLFLNGSLAEEEVVIRSEDWRDNAKSICVQLNTSVEINCTGAGHCNISRAKWDNTLKQI
ASKLREQYGNKTIIFKPSSGGDPEFVNHSFNCGGEFFYCDSTQLFDSTWFDSTGGSGSGG
GSEGPRNQDWL (SEQ ID NO: 112)
DNA sequence
Atggactggacctggatcctgttcctggtggccgccgccaccagggtgcacagcatgcagatctacgagggcaagctgaccgccg
agggcctgaggttcggcatcgtggccagcagggccaaccacgccctggtggacaggctggtggagggcgccatcgacgccatcgt
gaggcacggcggcagggaggaggacatcaccctggtgagggtgtgcggcagctgggagatccccgtggccgccggcgagctgg
ccaggaaggaggacatcgacgccgtgatcgccatcggcgtgctgtgcaggggcgccacccccagcttcgactacatcgccagcga
ggtgagcaagggcctggccgacctgagcctggagctgaggaagcccatcaccttcggcgtgatcaccgccgacaccctggagcag
gccatcgaggccgccggcacctgccacggcaacaagggctgggaggccgccctgtgcgccatcgagatggccaacctgttcaag
agcctgaggggcggcagcggcggcagcggcggcagcggcggcagcggcggcggcgacaccatcaccctgccctgcaggccc
gccccccccccccactgcagcagcaacatcaccggcctgatcctgaccaggcagggggctacagcaacgacaacaccgtgatctt
caggcccagcggcggcgactggagggacatcgccaggtgccagatcgccggcaccgtggtgagcacccagctgttcctgaacgg
cagcctggccgaggaggaggtggtgatcaggagcgaggactggagggacaacgccaagagcatctgcgtgcagctgaacacca
gcgtggagatcaactgcaccggcgccggccactgcaacatcagcagggccaagtgggacaacaccctgaagcagatcgccagca
agctgagggagcagtacggcaacaagaccatcatcttcaagcccagcagcggcggcgaccccgagttcgtgaaccacagcttcaa
ctgcggcggcgagttcttctactgcgacagcacccagctgttcgacagcacctggttcgacagcaccggcggcagcggcagcggc
ggcggcagcgagggccccaggaaccaggactggctg (SEQ ID NO: 113)
2. TRP2_188_GT60 mer (DLnano_LS_Trp2₁₈₈)
MDWTWILFLVAAATRVHSMQIYEGKLTAEGLRFGIVASRANHALVDRLVEGAIDAIVRH
GGREEDITLVRVCGSWEIPVAAGELARKEDIDAVIAIGVLCRGATPSFDYIASEVSKGLADLS
LELRKPITFGVITADTLEQAIEAAGTCHGNKGWEAALCAIEMANLFKSLRGGSGGSGGSG
GSGGGDTITLPCRPAPPPHCSSNITGLILTRQGGYSNDNTVIFRPSGGDWRDIARCQIAGTV
VSTQLFLNGSLAEEEVVIRSEDWRDNAKSICVQLNTSVEINCTGAGHCNISRAKWDNTLKQI
ASKLREQYGNKTIIFKPSSGGDPEFVNHSFNCGGEFFYCDSTQLFDSTWFDSTGGSGSGG
GSSVYDFFVWL (SEQ ID NO: 114)
DNA sequence
Atggactggacctggatcctgttcctggtggccgccgccaccagggtgcacagcatgcagatctacgagggcaagctgaccgccg
agggcctgaggttcggcatcgtggccagcagggccaaccacgccctggtggacaggctggtggagggcgccatcgacgccatcgt
gaggcacggcggcagggaggaggacatcaccctggtgagggtgtgcggcagctgggagatccccgtggccgccggcgagctgg
ccaggaaggaggacatcgacgccgtgatcgccatcggcgtgctgtgcaggggcgccacccccagcttcgactacatcgccagcga
ggtgagcaagggcctggccgacctgagcctggagctgaggaagcccatcaccttcggcgtgatcaccgccgacaccctggagcag
gccatcgaggccgccggcacctgccacggcaacaagggctgggaggccgccctgtgcgccatcgagatggccaacctgttcaag
agcctgaggggcggcagcggcggcagcggcggcagcggcggcagcggcggcggcgacaccatcaccctgccctgcaggccc
gccccccccccccactgcagcagcaacatcaccggcctgatcctgaccaggcagggggctacagcaacgacaacaccgtgatctt
caggcccagcggcggcgactggagggacatcgccaggtgccagatcgccggcaccgtggtgagcacccagctgttcctgaacgg
cagcctggccgaggaggaggtggtgatcaggagcgaggactggagggacaacgccaagagcatctgcgtgcagctgaacacca
gcgtggagatcaactgcaccggcgccggccactgcaacatcagcagggccaagtgggacaacaccctgaagcagatcgccagca
agctgagggagcagtacggcaacaagaccatcatcttcaagcccagcagcggcggcgaccccgagttcgtgaaccacagcttcaa
ctgcggcggcgagttcttctactgcgacagcacccagctgttcgacagcacctggttcgacagcaccggcggcagcggcagcggc
ggcggcagcagcgtgtacgacttcttcgtgtggctg (SEQ ID NO: 115)
3. TC1_HPV_E743_GT60 mer
MDWTWILFLVAAATRVHSMQIYEGKLTAEGLRFGIVASRANHALVDRLVEGAIDAIVRH
GGREEDITLVRVCGSWEIPVAAGELARKEDIDAVIAIGVLCRGATPSFDYIASEVSKGLADLS
LELRKPITFGVITADTLEQAIEAAGTCHGNKGWEAALCAIEMANLFKSLRGGSGGSGGSG
GSGGGDTITLPCRPAPPPHCSSNITGLILTRQGGYSNDNTVIFRPSGGDWRDIARCQIAGTV
VSTQLFLNGSLAEEEVVIRSEDWRDNAKSICVQLNTSVEINCTGAGHCNISRAKWDNTLKQI
ASKLREQYGNKTIIFKPSSGGDPEFVNHSFNCGGEFFYCDSTQLFDSTWFDSTGGSGSGG
GSRAHYNMQDIFIEDF (SEQ ID NO: 116)
DNA sequence
Atggactggacctggatcctgttcctggtggccgccgccaccagggtgcacagcatgcagatctacgagggcaagctgaccgccg
agggcctgaggttcggcatcgtggccagcagggccaaccacgccctggtggacaggctggtggagggcgccatcgacgccatcgt
gaggcacggcggcagggaggaggacatcaccctggtgagggtgtgcggcagctgggagatccccgtggccgccggcgagctgg
ccaggaaggaggacatcgacgccgtgatcgccatcggcgtgctgtgcaggggcgccacccccagcttcgactacatcgccagcga
ggtgagcaagggcctggccgacctgagcctggagctgaggaagcccatcaccttcggcgtgatcaccgccgacaccctggagcag
gccatcgaggccgccggcacctgccacggcaacaagggctgggaggccgccctgtgcgccatcgagatggccaacctgttcaag
agcctgaggggcggcagcggcggcagcggcggcagcggcggcagcggcggcggcgacaccatcaccctgccctgcaggccc
gccccccccccccactgcagcagcaacatcaccggcctgatcctgaccaggcagggcggctacagcaacgacaacaccgtgatctt
caggcccagcggcggcgactggagggacatcgccaggtgccagatcgccggcaccgtggtgagcacccagctgttcctgaacgg
cagcctggccgaggaggaggtggtgatcaggagcgaggactggagggacaacgccaagagcatctgcgtgcagctgaacacca
gcgtggagatcaactgcaccggcgccggccactgcaacatcagcagggccaagtgggacaacaccctgaagcagatcgccagca
agctgagggagcagtacggcaacaagaccatcatcttcaagcccagcagcggcggcgaccccgagttcgtgaaccacagcttcaa
ctgcggcggcgagttcttctactgcgacagcacccagctgttcgacagcacctggttcgacagcaccggcggcagcggcagcggc
ggcggcagcagggcccactacaacatcgtgaccttc (SEQ ID NO: 117)
4. gp100_3pep_link6_GT60 mer_pVax
MDWTWILFLVAAATRVHSMQIYEGKLTAEGLRFGIVASRANHALVDRLVEGAIDAIVRH
GGREEDITLVRVCGSWEIPVAAGELARKEDIDAVIAIGVLCRGATPSFDYIASEVSKGLADLS
LELRKPITFGVITADTLEQAIEAAGTCHGNKGWEAALCAIEMANLFKSLRGGSGGSGGSG
GSGGGDTITLPCRPAPPPHCSSNITGLILTRQGGYSNDNTVIFRPSGGDWRDIARCQIAGTV
VSTQLFLNGSLAEEEVVIRSEDWRDNAKSICVQLNTSVEINCTGAGHCNISRAKWDNTLKQI
ASKLREQYGNKTIIFKPSSGGDPEFVNHSFNCGGEFFYCDSTQLFDSTWFDSTGGSGSGG
GSTGRAMLGTHTMEVTVYHGGIMDQVPFSVGGEGPRNQDWL (SEQ ID NO: 118)
DNA sequence
atggactggacctggatcctgttcctggtggccgccgccaccagggtgcacagcatgcagatctacgagggcaagctgaccgccga
gggcctgaggttcggcatcgtggccagcagggccaaccacgccctggtggacaggctggtggagggcgccatcgacgccatcgt
gaggcacggcggcagggaggaggacatcaccctggtgagggtgtgcggcagctgggagatccccgtggccgccggcgagctgg
ccaggaaggaggacatcgacgccgtgatcgccatcggcgtgctgtgcagggggccacccccagcttcgactacatcgccagcga
ggtgagcaagggcctggccgacctgagcctggagctgaggaagcccatcaccttcggcgtgatcaccgccgacaccctggagcag
gccatcgaggccgccggcacctgccacggcaacaagggctgggaggccgccctgtgcgccatcgagatggccaacctgttcaag
agcctgaggggcggcagcggcggcagcggcggcagcggcggcagcggcggcggcgacaccatcaccctgccctgcaggccc
gccccccccccccactgcagcagcaacatcaccggcctgatcctgaccaggcagggcggctacagcaacgacaacaccgtgatctt
caggcccagcggcggcgactggagggacatcgccaggtgccagatcgccggcaccgtggtgagcacccagctgttcctgaacgg
cagcctggccgaggaggaggtggtgatcaggagcgaggactggagggacaacgccaagagcatctgcgtgcagctgaacacca
gcgtggagatcaactgcaccggcgccggccactgcaacatcagcagggccaagtgggacaacaccctgaagcagatcgccagca
agctgagggagcagtacggcaacaagaccatcatcttcaagcccagcagcggcggcgaccccgagttcgtgaaccacagcttcaa
ctgcggcggcgagttcttctactgcgacagcacccagctgttcgacagcacctggttcgacagcaccggcggcagcggcagcggc
ggcggcagcaccggcagggccatgctgggcacccacaccatggaggtgaccgtgtaccacggcggcatcatggaccaggtgccc
ttcagcgtgggcggcgagggccccaggaaccaggactggctg (SEQ ID NO: 119)
5. P53_link6_GT60mer_pVax
MDWTWILFLVAAATRVHSMQIYEGKLTAEGLRFGIVASRANHALVDRLVEGAIDAIVRH
GGREEDITLVRVCGSWEIPVAAGELARKEDIDAVIAIGVLCRGATPSFDYIASEVSKGLADLS
LELRKPITFGVITADTLEQAIEAAGTCHGNKGWEAALCAIEMANLFKSLRGGSGGSGGSG
GSGGGDTITLPCRPAPPPHCSSNITGLILTRQGGYSNDNTVIFRPSGGDWRDIARCQIAGTV
VSTQLFLNGSLAEEEVVIRSEDWRDNAKSICVQLNTSVEINCTGAGHCNISRAKWDNTLKQI
ASKLREQYGNKTIIFKPSSGGDPEFVNHSFNCGGEFFYCDSTQLFDSTWFDSTGGSGSGG
GSKYICNSSCM (SEQ ID NO: 120)
DNA sequence
Atggactggacctggatcctgttcctggtggccgccgccaccagggtgcacagcatgcagatctacgagggcaagctgaccgccg
agggcctgaggttcggcatcgtggccagcagggccaaccacgccctggtggacaggctggtggagggcgccatcgacgccatcgt
gaggcacggcggcagggaggaggacatcaccctggtgagggtgtgcggcagctgggagatccccgtggccgccggcgagctgg
ccaggaaggaggacatcgacgccgtgatcgccatcggcgtgctgtgcaggggcgccacccccagcttcgactacatcgccagcga
ggtgagcaagggcctggccgacctgagcctggagctgaggaagcccatcaccttcggcgtgatcaccgccgacaccctggagcag
gccatcgaggccgccggcacctgccacggcaacaagggctgggaggccgccctgtgcgccatcgagatggccaacctgttcaag
agcctgaggggcggcagcggcggcagcggcggcagcggcggcagcggcggcggcgacaccatcaccctgccctgcaggccc
gccccccccccccactgcagcagcaacatcaccggcctgatcctgaccaggcagggcggctacagcaacgacaacaccgtgatctt
caggcccagcggcggcgactggagggacatcgccaggtgccagatcgccggcaccgtggtgagcacccagctgttcctgaacgg
cagcctggccgaggaggaggtggtgatcaggagcgaggactggagggacaacgccaagagcatctgcgtgcagctgaacacca
gcgtggagatcaactgcaccggcgccggccactgcaacatcagcagggccaagtgggacaacaccctgaagcagatcgccagca
agctgagggagcagtacggcaacaagaccatcatcttcaagcccagcagcggcggcgaccccgagttcgtgaaccacagcttcaa
ctgcggcggcgagttcttctactgcgacagcacccagctgttcgacagcacctggttcgacagcaccggcggcagcggcagcggc
ggcggcagcaagtacatctgcaacagcagctgcatg (SEQ ID NO: 121)
6. gp100_pep174_L6GT60_pVax
MDWTWILFLVAAATRVHSMQIYEGKLTAEGLRFGIVASRANHALVDRLVEGAIDAIVRH
GGREEDITLVRVCGSWEIPVAAGELARKEDIDAVIAIGVLCRGATPSFDYIASEVSKGLADLS
LELRKPITFGVITADTLEQAIEAAGTCHGNKGWEAALCAIEMANLFKSLRGGSGGSGGSG
GSGGGDTITLPCRPAPPPHCSSNITGLILTRQGGYSNDNTVIFRPSGGDWRDIARCQIAGTV
VSTQLFLNGSLAEEEVVIRSEDWRDNAKSICVQLNTSVEINCTGAGHCNISRAKWDNTLKQI
ASKLREQYGNKTIIFKPSSGGDPEFVNHSFNCGGEFFYCDSTQLFDSTWFDSTGGSGSGG
GSTGRAMLGTHTMEVTVYH (SEQ ID NO: 122)
DNA sequence
Atggactggacctggatcctgttcctggtggccgccgccaccagggtgcacagcatgcagatctacgagggcaagctgaccgccg
agggcctgaggttcggcatcgtggccagcagggccaaccacgccctggtggacaggctggtggagggcgccatcgacgccatcgt
gaggcacggcggcagggaggaggacatcaccctggtgagggtgtgcggcagctgggagatccccgtggccgccggcgagctgg
ccaggaaggaggacatcgacgccgtgatcgccatcggcgtgctgtgcaggggcgccacccccagcttcgactacatcgccagcga
ggtgagcaagggcctggccgacctgagcctggagctgaggaagcccatcaccttcggcgtgatcaccgccgacaccctggagcag
gccatcgaggccgccggcacctgccacggcaacaagggctgggaggccgccctgtgcgccatcgagatggccaacctgttcaag
agcctgaggggcggcagcggcggcagcggcggcagcggcggcagcggcggggcgacaccatcaccctgccctgcaggccc
gccccccccccccactgcagcagcaacatcaccggcctgatcctgaccaggcagggggctacagcaacgacaacaccgtgatctt
caggcccagcggcggcgactggagggacatcgccaggtgccagatcgccggcaccgtggtgagcacccagctgttcctgaacgg
cagcctggccgaggaggaggtggtgatcaggagcgaggactggagggacaacgccaagagcatctgcgtgcagctgaacacca
gcgtggagatcaactgcaccggcgccggccactgcaacatcagcagggccaagtgggacaacaccctgaagcagatcgccagca
agctgagggagcagtacggcaacaagaccatcatcttcaagcccagcagcggcggcgaccccgagttcgtgaaccacagcttcaa
ctgcggcggcgagttcttctactgcgacagcacccagctgttcgacagcacctggttcgacagcaccggcggcagcggcagcggc
ggcggcagcaccggcagggccatgctgggcacccacaccatggaggtgaccgtgtaccac (SEQ ID NO: 123)
7. gp100_pep209_L6GT60_pVax
MDWTWILFLVAAATRVHSMQIYEGKLTAEGLRFGIVASRANHALVDRLVEGAIDAIVRH
GGREEDITLVRVCGSWEIPVAAGELARKEDIDAVIAIGVLCRGATPSFDYIASEVSKGLADLS
LELRKPITFGVITADTLEQAIEAAGTCHGNKGWEAALCAIEMANLFKSLRGGSGGSGGSG
GSGGGDTITLPCRPAPPPHCSSNITGLILTRQGGYSNDNTVIFRPSGGDWRDIARCQIAGTV
VSTQLFLNGSLAEEEVVIRSEDWRDNAKSICVQLNTSVEINCTGAGHCNISRAKWDNTLKQI
ASKLREQYGNKTIIFKPSSGGDPEFVNHSFNCGGEFFYCDSTQLFDSTWFDSTGGSGSGG
GSIMDQVPFSV (SEQ ID NO: 124)
DNA sequence
Atggactggacctggatcctgttcctggtggccgccgccaccagggtgcacagcatgcagatctacgagggcaagctgaccgccg
agggcctgaggttcggcatcgtggccagcagggccaaccacgccctggtggacaggctggtggagggcgccatcgacgccatcgt
gaggcacggcggcagggaggaggacatcaccctggtgagggtgtgcggcagctgggagatccccgtggccgccggcgagctgg
ccaggaaggaggacatcgacgccgtgatcgccatcggcgtgctgtgcaggggcgccacccccagcttcgactacatcgccagcga
ggtgagcaagggcctggccgacctgagcctggagctgaggaagcccatcaccttcggcgtgatcaccgccgacaccctggagcag
gccatcgaggccgccggcacctgccacggcaacaagggctgggaggccgccctgtgcgccatcgagatggccaacctgttcaag
agcctgaggggcggcagcggcggcagcggcggcagcggcggcagcggcggcggcgacaccatcaccctgccctgcaggccc
gccccccccccccactgcagcagcaacatcaccggcctgatcctgaccaggcagggggctacagcaacgacaacaccgtgatctt
caggcccagcggcggcgactggagggacatcgccaggtgccagatcgccggcaccgtggtgagcacccagctgttcctgaacgg
cagcctggccgaggaggaggtggtgatcaggagcgaggactggagggacaacgccaagagcatctgcgtgcagctgaacacca
gcgtggagatcaactgcaccggcgccggccactgcaacatcagcagggccaagtgggacaacaccctgaagcagatcgccagca
agctgagggagcagtacggcaacaagaccatcatcttcaagcccagcagcggcggcgaccccgagttcgtgaaccacagcttcaa
ctgcggcggcgagttcttctactgcgacagcacccagctgttcgacagcacctggttcgacagcaccggcggcagcggcagcggc
ggcggcagcatcatggaccaggtgcccttcagcgtg (SEQ ID NO: 125)
8. NYESO1_A2_L6GT60_pVax
MDWTWILFLVAAATRVHSMQIYEGKLTAEGLRFGIVASRANHALVDRLVEGAIDAIVRH
GGREEDITLVRVCGSWEIPVAAGELARKEDIDAVIAIGVLCRGATPSFDYIASEVSKGLADLS
LELRKPITFGVITADTLEQAIEAAGTCHGNKGWEAALCAIEMANLFKSLRGGSGGSGGSG
GSGGGDTITLPCRPAPPPHCSSNITGLILTRQGGYSNDNTVIFRPSGGDWRDIARCQIAGTV
VSTQLFLNGSLAEEEVVIRSEDWRDNAKSICVQLNTSVEINCTGAGHCNISRAKWDNTLKQI
ASKLREQYGNKTIIFKPSSGGDPEFVNHSFNCGGEFFYCDSTQLFDSTWFDSTGGSGSGG
GSSLLMWITQC (SEQ ID NO: 126)
DNA sequence
Atggactggacctggatcctgttcctggtggccgccgccaccagggtgcacagcatgcagatctacgagggcaagctgaccgccg
agggcctgaggttcggcatcgtggccagcagggccaaccacgccctggtggacaggctggtggagggcgccatcgacgccatcgt
gaggcacggcggcagggaggaggacatcaccctggtgagggtgtgcggcagctgggagatccccgtggccgccggcgagctgg
ccaggaaggaggacatcgacgccgtgatcgccatcggcgtgctgtgcaggggcgccacccccagcttcgactacatcgccagcga
ggtgagcaagggcctggccgacctgagcctggagctgaggaagcccatcaccttcggcgtgatcaccgccgacaccctggagcag
gccatcgaggccgccggcacctgccacggcaacaagggctgggaggccgccctgtgcgccatcgagatggccaacctgttcaag
agcctgaggggcggcagcggcggcagcggcggcagcggcggcagcggcggcggcgacaccatcaccctgccctgcaggccc
gccccccccccccactgcagcagcaacatcaccggcctgatcctgaccaggcagggcggctacagcaacgacaacaccgtgatctt
caggcccagcggcggcgactggagggacatcgccaggtgccagatcgccggcaccgtggtgagcacccagctgttcctgaacgg
cagcctggccgaggaggaggtggtgatcaggagcgaggactggagggacaacgccaagagcatctgcgtgcagctgaacacca
gcgtggagatcaactgcaccggcgccggccactgcaacatcagcagggccaagtgggacaacaccctgaagcagatcgccagca
agctgagggagcagtacggcaacaagaccatcatcttcaagcccagcagcggggcgaccccgagttcgtgaaccacagcttcaa
ctgcggcggcgagttcttctactgcgacagcacccagctgttcgacagcacctggttcgacagcaccggcggcagcggcagcggc
ggcggcagcagcctgctgatgtggatcacccagtgc (SEQ ID NO: 127)
9. NYESO1_B18C3_L6GT60_pVax
MDWTWILFLVAAATRVHSMQIYEGKLTAEGLRFGIVASRANHALVDRLVEGAIDAIVRH
GGREEDITLVRVCGSWEIPVAAGELARKEDIDAVIAIGVLCRGATPSFDYIASEVSKGLADLS
LELRKPITFGVITADTLEQAIEAAGTCHGNKGWEAALCAIEMANLFKSLRGGSGGSGGSG
GSGGGDTITLPCRPAPPPHCSSNITGLILTRQGGYSNDNTVIFRPSGGDWRDIARCQIAGTV
VSTQLFLNGSLAEEEVVIRSEDWRDNAKSICVQLNTSVEINCTGAGHCNISRAKWDNTLKQI
ASKLREQYGNKTIIFKPSSGGDPEFVNHSFNCGGEFFYCDSTQLFDSTWFDSTGGSGSGG
GSLEFYLAMPFATPMEAEL (SEQ ID NO: 128)
DNA sequence
Atggactggacctggatcctgttcctggtggccgccgccaccagggtgcacagcatgcagatctacgagggcaagctgaccgccg
agggcctgaggttcggcatcgtggccagcagggccaaccacgccctggtggacaggctggtggagggcgccatcgacgccatcgt
gaggcacggcggcagggaggaggacatcaccctggtgagggtgtgcggcagctgggagatccccgtggccgccggcgagctgg
ccaggaaggaggacatcgacgccgtgatcgccatcggcgtgctgtgcaggggcgccacccccagcttcgactacatcgccagcga
ggtgagcaagggcctggccgacctgagcctggagctgaggaagcccatcaccttcggcgtgatcaccgccgacaccctggagcag
gccatcgaggccgccggcacctgccacggcaacaagggctgggaggccgccctgtgcgccatcgagatggccaacctgttcaag
agcctgaggggcggcagcggcggcagcggcggcagcggcggcagcggcggggcgacaccatcaccctgccctgcaggccc
gccccccccccccactgcagcagcaacatcaccggcctgatcctgaccaggcagggggctacagcaacgacaacaccgtgatctt
caggcccagcggcggcgactggagggacatcgccaggtgccagatcgccggcaccgtggtgagcacccagctgttcctgaacgg
cagcctggccgaggaggaggtggtgatcaggagcgaggactggagggacaacgccaagagcatctgcgtgcagctgaacacca
gcgtggagatcaactgcaccggcgccggccactgcaacatcagcagggccaagtgggacaacaccctgaagcagatcgccagca
agctgagggagcagtacggcaacaagaccatcatcttcaagcccagcagcggcggcgaccccgagttcgtgaaccacagcttcaa
ctgcggcggcgagttcttctactgcgacagcacccagctgttcgacagcacctggttcgacagcaccggcggcagcggcagcggc
ggcggcagcctggagttctacctggccatgcccttcgccacccccatggaggccgagctg (SEQ ID NO: 129)

### Example 10. Development of CD40L and CD40L/Anti-PD1 Antibody Nanoparticles In Vitro

Optimal CTL activity may require the antigen be robustly presented on APCs, one molecular adjuvant employed in cancer vaccine models involved in DC activation is CD40 ligand (CD40L). It is possible that molecular adjuvant, such as CD40L, could also be synergistic with anti-PD1 antibodies. We thus examined whether CD40L can be formulated as a nanoparticle since simple genetic fusions do not work. The next generation designs were used to form nanoparticles. We then examined whether CD40L nanoparticles can express in vivo and if so, whether CD40L nanoparticles can function as an adjuvant in vivo.

We first designed three constructs by formulating CD40L as a trimer or 60mers. These three constructs encode the following proteins.
1. Trimer 1—CD40L-FoldOn (CD40L: underlined; linker: bold)

(SEQ ID NO: 130)
DKVEEEVNLHEDFVFIKKLKRCNKGEGSLSLLNCEEMRRQFEDLVKDIT
LNKEEKKENSFEMQRGDEDPQIAAHVVSEANSNAASVLQWAKKGYYTMK
SNLVMLENGKQLTVKREGLYYVYTQVTFCSNREPSSQRPFIVGLWLKPS
SGSERILLKAANTHSSSQLCEQQSVHLGGVFELQAGASVFVNVTEASQV
IHRVGFSSFGLLKLGGSGSGGYIPEAPRDGQAYVRKDGEWVLLSTFLG

2. Trimer 2—CD40L-GCN4 (CD40L: underlined; linker: bold; a trimeric leucine zipper GCN4pII heptad repeat derived from the wildtype dimeric GCN4 repeat found in Saccharomyces cerevisiae: italic)

(SEQ ID NO: 131)
DKVEEEVNLHEDFVFIKKLKRCNKGEGSLSLLNCEEMRRQFEDLVKDIT
LNKEEKKENSFEMQRGDEDPQIAAHVVSEANSNAASVLQWAKKGYYTMK
SNLVMLENGKQLTVKREGLYYVYTQVTFCSNREPSSQRPFIVGLWLKPS
SGSERILLKAANTHSSSQLCEQQSVHLGGVFELQAGASVFVNVTEASQV
IHRVGFSSFGLLKLGGSGSGRMKQIEDKIEEILSKIYHIENEIARIKKL
IGER

3. 60mer—CD40L-1HQK (CD40L: underlined; linker: bold; self-assembling polypeptide: italic)

(SEQ ID NO: 132)
MQIYEGKLTAEGLRFGIVASRFNHALVDRLVEGAIDAIVRHGGREEDIT
LVRVPGSWEIPVAAGELARKEDIDAVIAIGVLIRGATPHFDYIASEVSK
GLADLSLELRKPITFGVITADTLEQAIERAGTKHGNKGWEAALSAIEMA
NLFKSLRGGSGSGGGSDKVEEEVNLHEDFVFIKKLKRCNKGEGSLSLLN
CEEMRRQFEDLVKDITLNKEEKKENSFEMQRGDEDPQIAAHVVSEANSN
AASVLQWAKKGYYTMKSNLVMLENGKQLTVKREGLYYVYTQVTFCSNRE
PSSQRPFIVGLWLKPSSGSERILLKAANTHSSSQLCEQQSVHLGGVFEL
QAGASVFVNVTEASQVIHRVGESSFGLLKL

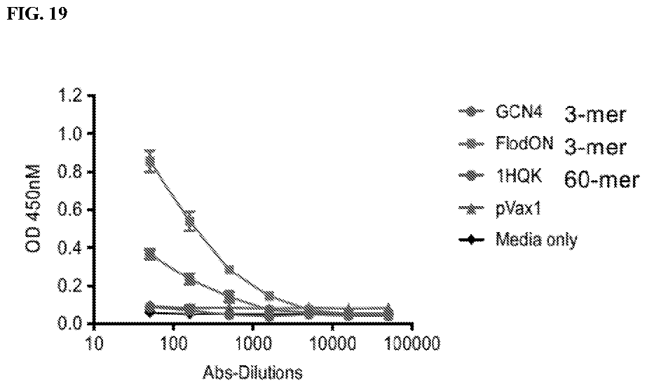

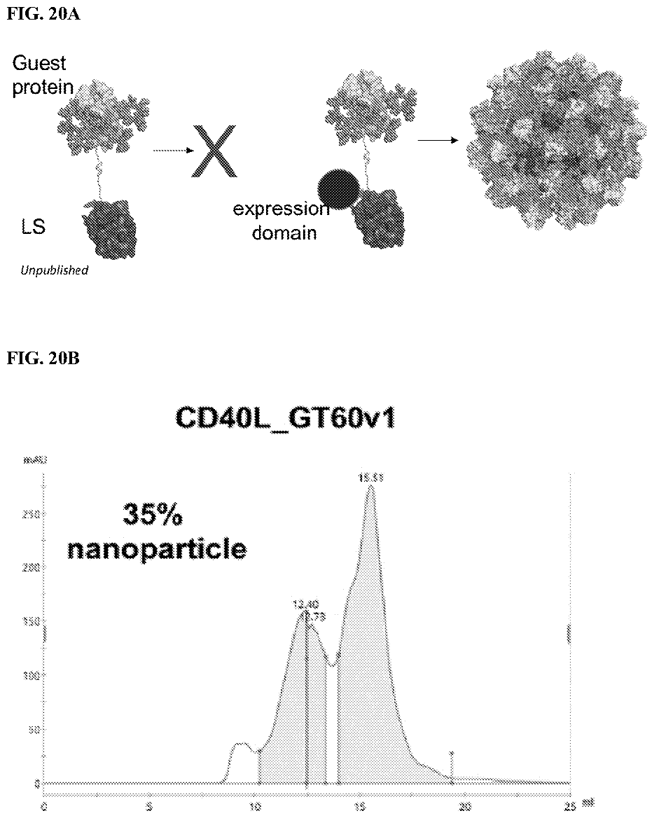

As shown in FIG. 19, these constructs did not express. When an expression domain was included into the self-assembling polypeptide (FIG. 20A), it enabled CD40L nanoparticle expression and assembly (FIG. 20B).

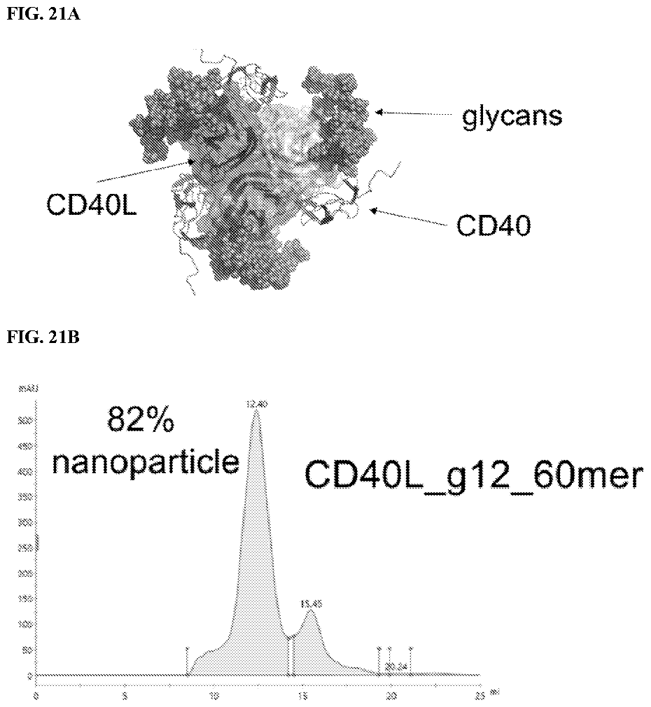

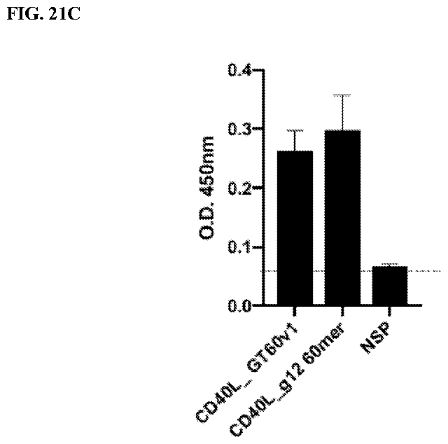

We then examined whether modification to CD40L can facilitate the nanoparticle's expression and assembly. We found that a structural design of 3 new glycans to CD40L (FIG. 21A) or 12 total N-linked glycans (CD40L_g12) resulted in an expression and assembly without the need to add the expression domain (FIG. 21B-21C). We thus demonstrated that CD40L can be engineered to facilitate nanoparticle expression and assembly. The CD40L_g12 construct encodes the following sequence (CD41L: underlined; linker: bold; self-assembling polypeptide: italic):

(SEQ ID NO: 133)
MQIYEGKLTAEGLRFGIVASRANHALVDRLVEGAIDAIVRHGGREEDIT
LVRVCGSWEIPVAAGELARKEDIDAVIAIGVLCRGATPSFDYIASEVSK
GLADLSLELRKPITFGVITADTLEQAIEAAGTCHGNKGWEAALCAIEMA
NLFKSLRGGSGGSGGSGGSGGGIAAHVVSEANSNATSVLQWAKKGYYTM
KSNLVMLENGSQLTVNRSGLYYVYTQVTFCSNREPSSQRPFIVGLWLKP
SSGSERILLKAANTHSSSQLCEQQSVHLGGVFELQAGASVFVNVTEASQ
VIHRVGFSSFGLLKLG**

Other constructs involved in the development of this work include constructs encoding the following polypeptides:

4KVU 6-stranded coiled-coil (12-mer or 4 trimers)
1. CD40L_4 mer_mC
(SEQ ID NO: 134)
DKVEEEVNLHEDFVFIKKLKRCNKGEGSLSLLNCEEMRRQFEDLVKDITLNKEEK
KENSFEMQRGDEDPQIAAHVVSEANSNAASVLQWAKKGYYTMKSNLVMLENG
KQLTVKREGLYYVYTQVTFCSNREPSSQRPFIVGLWLKPSSGSERILLKAANTHS
SSQLCEQQSVHLGGVFELQAGASVFVNVTEASQVIHRVGFSSFGLLKLGGSGGS
GGSGGSGGGGELKAIAQELKAIAKECKAIAYELKAIAQGAGGGSGGSGGSGGS
GGGDKVEEEVNLHEDFVFIKKLKRCNKGEGSLSLLNCEEMRRQFEDLVKDITLN
KEEKKENSFEMQRGDEDPQIAAHVVSEANSNAASVLQWAKKGYYTMKSNLVM
LENGKQLTVKREGLYYVYTQVTFCSNREPSSQRPFIVGLWLKPSSGSERILLKAA
NTHSSSQLCEQQSVHLGGVFELQAGASVFVNVTEASQVIHRVGFSSFGLLKLGG
GHHHHHH**
2. CD40L_SC_mC
(SEQ ID NO: 135)
GIAAHVVSEANSNAASVLQWAKKGYYTMKSNLVMLENGKQLTVKREGLYYVY
TQVTFCSNREPSSQRPFIVGLWLKPSSGSERILLKAANTHSSSQLCEQQSVHLGGV
FELQAGASVFVNVTEASQVIHRVGFSSFGLLKLGGGGIAAHVVSEANSNAASVL
QWAKKGYYTMKSNLVMLENGKQLTVKREGLYYVYTQVTFCSNREPSSQRPFIV
GLWLKPSSGSERILLKAANTHSSSQLCEQQSVHLGGVFELQAGASVFVNVTEAS
QVIHRVGFSSFGLLKLGGGGIAAHVVSEANSNAASVLQWAKKGYYTMKSNLV
MLENGKQLTVKREGLYYVYTQVTFCSNREPSSQRPFIVGLWLKPSSGSERILLKA
ANTHSSSQLCEQQSVHLGGVFELQAGASVFVNVTEASQVIHRVGFSSFGLLKLG
GGHHHHHH**
7-stranded coiled-coil with SC version gives 14 trimer copies (or 42-mer)
3. CD40L_SC_14 mer_mC
(SEQ ID NO: 136)
GIAAHVVSEANSNAASVLQWAKKGYYTMKSNLVMLENGKQLTVKREGLYYVY
TQVTFCSNREPSSQRPFIVGLWLKPSSGSERILLKAANTHSSSQLCEQQSVHLGGV
FELQAGASVFVNVTEASQVIHRVGFSSFGLLKLGGGGIAAHVVSEANSNAASVL
QWAKKGYYTMKSNLVMLENGKQLTVKREGLYYVYTQVTFCSNREPSSQRPFIV
GLWLKPSSGSERILLKAANTHSSSQLCEQQSVHLGGVFELQAGASVFVNVTEAS
QVIHRVGFSSFGLLKLGGGGIAAHVVSEANSNAASVLQWAKKGYYTMKSNLV
MLENGKQLTVKREGLYYVYTQVTFCSNREPSSQRPFIVGLWLKPSSGSERILLKA
ANTHSSSQLCEQQSVHLGGVFELQAGASVFVNVTEASQVIHRVGFSSFGLLKLG
GSGGSGGSGGSGGGMKVKqLADAVEELASANYHLANAVARLAKAVGERGGS
GGSGGSGGSGGGGIAAHVVSEANSNAASVLQWAKKGYYTMKSNLVMLENGK
QLTVKREGLYYVYTQVTFCSNREPSSQRPFIVGLWLKPSSGSERILLKAANTHSSS
QLCEQQSVHLGGVFELQAGASVFVNVTEASQVIHRVGFSSFGLLKLGGGGIAAH
VVSEANSNAASVLQWAKKGYYTMKSNLVMLENGKQLTVKREGLYYVYTQVTF
CSNREPSSQRPFIVGLWLKPSSGSERILLKAANTHSSSQLCEQQSVHLGGVFELQA
GASVFVNVTEASQVIHRVGFSSFGLLKLGGGGIAAHVVSEANSNAASVLQWAK
KGYYTMKSNLVMLENGKQLTVKREGLYYVYTQVTFCSNREPSSQRPFIVGLWL
KPSSGSERILLKAANTHSSSQLCEQQSVHLGGVFELQAGASVFVNVTEASQVIHR
VGFSSFGLLKLGGGHHHHHH**
GT8-60 mer, with CD40L on end of GT8
4. CD40L-GT60v1_m
(SEQ ID NO: 137)
MQIYEGKLTAEGLRFGIVASRANHALVDRLVEGAIDAIVRHGGREEDITLVRVCG
SWEIPVAAGELARKEDIDAVIAIGVLCRGATPSFDYIASEVSKGLADLSLELRKPIT
FGVITADTLEQAIEAAGTCHGNKGWEAALCAIEMANLFKSLRGGSGGSGGSGGS
GGGDTITLPCRPAPPPHCSSNITGLILTRQGGYSNDNTVIFRPSGGDWRDIARCQIA
GTVVSTQLFLNGSLAEEEVVIRSEDWRDNAKSICVQLNTSVEINCTGAGHCNISR
AKWNNTLKQIASKLREQYGNKTIIFKPSSGGDPEFVNHSFNCGGEFFYCDSTQLF
NSTWFNSTGGSGSGGGSDKVEEEVNLHEDFVFIKKLKRCNKGEGSLSLLNCEEM
RRQFEDLVKDITLNKEEKKENSFEMQRGDEDPQIAAHVVSEANSNAASVLQWA
KKGYYTMKSNLVMLENGKQLTVKREGLYYVYTQVTFCSNREPSSQRPFIVGLW
LKPSSGSERILLKAANTHSSSQLCEQQSVHLGGVFELQAGASVFVNVTEASQVIH
RVGFSSFGLLKL**
GT8-60 mer, with CD40L on end of GT8
5. CD40L-dGly_GT60v1_m
(SEQ ID NO: 138)
MQIYEGKLTAEGLRFGIVASRANHALVDRLVEGAIDAIVRHGGREEDITLVRVCG
SWEIPVAAGELARKEDIDAVIAIGVLCRGATPSFDYIASEVSKGLADLSLELRKPIT
FGVITADTLEQAIEAAGTCHGNKGWEAALCAIEMANLFKSLRGGSGGSGGSGGS
GGGDTITLPCRPAPPPHCSSNITGLILTRQGGYSNDNTVIFRPSGGDWRDIARCQIA
GTVVSTQLFLNGSLAEEEVVIRSEDWRDNAKSICVQLNTSVEINCTGAGHCNISR
AKWDNTLKQIASKLREQYGNKTIIFKPSSGGDPEFVNHSFNCGGEFFYCDSTQLF
NASTWFASTGGSGSGGGSDKVEEEVNLHEDFVFIKKLKRCNKGEGSLSLLNCEE
MRRQFEDLVKDITLNKEEKKENSFEMQRGDEDPQIAAHVVSEANSNAASVLQW
AKKGYYTMKSNLVMLENGKQLTVKREGLYYVYTQVTFCSNREPSSQRPFIVGL
WLKPSSGSERILLKAANTHSSSQLCEQQSVHLGGVFELQAGASVFVNVTEASQVI
HRVGFSSFGLLKL**
GT8-60 mer, with CD40L at C-term (3-fold axis) and GT8 on N-term of 60 mer
6. CD40L-GT60v2_m
(SEQ ID NO: 139)
DTITLPCRPAPPPHCSSNITGLILTRQGGYSNDNTVIFRPSGGDWRDIARCQIAGTV
VSTQLFLNGSLAEEEVVIRSEDWRDNAKSICVQLNTSVEINCTGAGHCNISRAKW
NNTLKQIASKLREQYGNKTIIFKPSSGGDPEFVNHSFNCGGEFFYCDSTQLFNSTW
FNSTGGSGGSGGSGGSGGGMQIYEGKLTAEGLRFGIVASRANHALVDRLVEGAI
DAIVRHGGREEDITLVRVCGSWEIPVAAGELARKEDIDAVIAIGVLCRGATPSFDY
IASEVSKGLADLSLELRKPITFGVITADTLEQAIEAAGTCHGNKGWEAALCAIEM
ANLFKSLRGGSGGSGGSGGSGGGDKVEEEVNLHEDFVFIKKLKRCNKGEGSLSL
LNCEEMRRQFEDLVKDITLNKEEKKENSFEMQRGDEDPQIAAHVVSEANSNAAS
VLQWAKKGYYTMKSNLVMLENGKQLTVKREGLYYVYTQVTFCSNREPSSQRPF
IVGLWLKPSSGSERILLKAANTHSSSQLCEQQSVHLGGVFELQAGASVFVNVTEA
SQVIHRVGFSSFGLLKL**
GT8-60 mer with CD40L-sc trimer on end of GT8
60 trimer copies (or 180-mer)
7. CD40L_SC_GT60_m
(SEQ ID NO: 140)
MQIYEGKLTAEGLRFGIVASRANHALVDRLVEGAIDAIVRHGGREEDITLVRVCG
SWEIPVAAGELARKEDIDAVIAIGVLCRGATPSFDYIASEVSKGLADLSLELRKPIT
FGVITADTLEQAIEAAGTCHGNKGWEAALCAIEMANLFKSLRGGSGGSGGSGGS
GGGDTITLPCRPAPPPHCSSNITGLILTRQGGYSNDNTVIFRPSGGDWRDIARCQIA
GTVVSTQLFLNGSLAEEEVVIRSEDWRDNAKSICVQLNTSVEINCTGAGHCNISR
AKWNNTLKQIASKLREQYGNKTIIFKPSSGGDPEFVNHSFNCGGEFFYCDSTQLF
NSTWFNSTGGSGSGGGSGIAAHVVSEANSNAASVLQWAKKGYYTMKSNLVML
ENGKQLTVKREGLYYVYTQVTFCSNREPSSQRPFIVGLWLKPSSGSERILLKAAN
THSSSQLCEQQSVHLGGVFELQAGASVFVNVTEASQVIHRVGFSSFGLLKLGGG
GIAAHVVSEANSNAASVLQWAKKGYYTMKSNLVMLENGKQLTVKREGLYYVY
TQVTFCSNREPSSQRPFIVGLWLKPSSGSERILLKAANTHSSSQLCEQQSVHLGGV
FELQAGASVFVNVTEASQVIHRVGFSSFGLLKLGGGGIAAHVVSEANSNAASVL
QWAKKGYYTMKSNLVMLENGKQLTVKREGLYYVYTQVTFCSNREPSSQRPFIV
GLWLKPSSGSERILLKAANTHSSSQLCEQQSVHLGGVFELQAGASVFVNVTEAS
QVIHRVGFSSFGLLKLG**
CD40L without GT8
8. CD40L_SC_g9_m
(SEQ ID NO: 141)
MQIYEGKLTAEGLRFGIVASRANHALVDRLVEGAIDAIVRHGGREEDITLVRVCG
SWEIPVAAGELARKEDIDAVIAIGVLCRGATPSFDYIASEVSKGLADLSLELRKPIT
FGVITADTLEQAIEAAGTCHGNKGWEAALCAIEMANLFKSLRGGSGGSGGSGGS
GGGIAAHVVSEANSNAASVLQWAKKGYYTMKSNLVMLENGSQLTVNRSGLYY
VYTQVTFCSNREPSSQRPFIVGLWLKPSSGSERILLKAANTHSSSQLCEQQSVHLG
GVFELQAGASVFVNVTEASQVIHRVGFSSFGLLKLGGGGIAAHVVSEANSNAAS
VLQWAKKGYYTMKSNLVMLENGSQLTVNRSGLYYVYTQVTFCSNREPSSQRPF
IVGLWLKPSSGSERILLKAANTHSSSQLCEQQSVHLGGVFELQAGASVFVNVTEA
SQVIHRVGFSSFGLLKLGGGGIAAHVVSEANSNAASVLQWAKKGYYTMKSNLV
MLENGSQLTVNRSGLYYVYTQVTFCSNREPSSQRPFIVGLWLKPSSGSERILLKA
ANTHSSSQLCEQQSVHLGGVFELQAGASVFVNVTEASQVIHRVGFSSFGLLKLG**
9. CD40L_SC_g12_m
(SEQ ID NO: 142)
MQIYEGKLTAEGLRFGIVASRANHALVDRLVEGAIDAIVRHGGREEDITLVRVCG
SWEIPVAAGELARKEDIDAVIAIGVLCRGATPSFDYIASEVSKGLADLSLELRKPIT
FGVITADTLEQAIEAAGTCHGNKGWEAALCAIEMANLFKSLRGGSGGSGGSGGS
GGGIAAHVVSEANSNATSVLQWAKKGYYTMKSNLVMLENGSQLTVNRSGLYY
VYTQVTFCSNREPSSQRPFIVGLWLKPSSGSERILLKAANTHSSSQLCEQQSVHLG
GVFELQAGASVFVNVTEASQVIHRVGFSSFGLLKLGGGGIAAHVVSEANSNATS
VLQWAKKGYYTMKSNLVMLENGSQLTVNRSGLYYVYTQVTFCSNREPSSQRPF
IVGLWLKPSSGSERILLKAANTHSSSQLCEQQSVHLGGVFELQAGASVFVNVTEA
SQVIHRVGFSSFGLLKLGGGGIAAHVVSEANSNATSVLQWAKKGYYTMKSNLV
MLENGSQLTVNRSGLYYVYTQVTFCSNREPSSQRPFIVGLWLKPSSGSERILLKA
ANTHSSSQLCEQQSVHLGGVFELQAGASVFVNVTEASQVIHRVGFSSFGLLKLG**
10. CD40L_g12_m
(SEQ ID NO: 143)
MQIYEGKLTAEGLRFGIVASRANHALVDRLVEGAIDAIVRHGGREEDITLVRVCG
SWEIPVAAGELARKEDIDAVIAIGVLCRGATPSFDYIASEVSKGLADLSLELRKPIT
FGVITADTLEQAIEAAGTCHGNKGWEAALCAIEMANLFKSLRGGSGGSGGSGGS
GGGIAAHVVSEANSNATSVLQWAKKGYYTMKSNLVMLENGSQLTVNRSGLYY
VYTQVTFCSNREPSSQRPFIVGLWLKPSSGSERILLKAANTHSSSQLCEQQSVHLG
GVFELQAGASVFVNVTEASQVIHRVGFSSFGLLKLG**

### Example 11. Co-Immunization of DNA Cancer Vaccine (hTERT) with CD40L Nanoparticles and Anti-PD1 Will Promote Higher Induction of Antigen-Specific T Cells In Vivo

We next examined whether it is possible to formulate CD40L nanoparticle with anti-PD1 antibodies. To this end, we created a plasmid DNA that encodes the engineered CD40L and a PD1 scFv-CD40L fusion protein. The PD1 scFv used in this experiment contains the following sequences:

Variable heavy chain
(SEQ ID NO: 147)
EVQLQESGPGLVKPSQSLSLTCSVTGYSITSSYRWNWIRKFPGNRLEWM
GYINSAGISNYNPSLKRRISITRDTSKNQFFLQVNSVTTEDAATYYCAR
SDNMGTTPFTYWGQGTLVTVSS,
and
Variable light chain
(SEQ ID NO: 148)
DIVMTQGTLPNPVPSGESVSITCRSSKSLLYSDGKTYLNWYLQRPGQSP
QLLIYWMSTRASGVSDRFSGSGSGTDFTLKISGVEAEDVGIYYCQQGLE
FPTFGGGTKLELK.

This fusion protein (CD40L-(Gly₃Ser₁)₄linker-pembroPD1-VH-Linker(C₄S₁)₃-PembroPD1-VL-Hig Tag) has the following sequence (CD40L: underlined; pembroPD1-VH: italic and underlined; linker: bold; PembroPD1-VL: bold and underlined; self-assembling polypeptide: italic):

(SEQ ID NO: 144)
MQIYEGKLTAEGLRFGIVASRANHALVDRLVEGAIDAIVRHGGREEDIT
LVRVCGSWEIPVAAGELARKEDIDAVIAIGVLCRGATPSFDYIASEVSK
GLADLSLELRKPITFGVITADTLEQAIEAAGTCHGNKGWEAALCAIEMA
NLFKSLRGGSGGSGGSGGSGGGIAAHVVSEANSNATSVLQWAKKGYYTM
KSNLVMLENGSQLTVNRSGLYYVYTQVTFCSNREPSSQRPFIVGLWLKP
SSGSERILLKAANTHSSSQLCEQQSVHLGGVFELQAGASVFVNVTEASQ
VIHRVGFSSFGLLKLGGGGSGGGSGGGSGGGSQVQLVQSGVEVKKPGAS
VKVSCKASGYTFTNYYMYWVRQAPGQGLEWMGGINPSNGGTNFNEKFKN
RVTLTTDSSTTTAYMELKSLQFDDTAVYYCARRDYRFDMGFDYWGQGTT
VTVSSGGGGSGGGGSGGGGSEIVLTQSPATLSLSPGERATLSCRASKGV
STSGYSYLHWYQQKPGQAPRLLIYLASYLESGVPARFSGSGSGTDFTLT
ISSLEPEDFAVYYCQHSRDLPLTFGGGTKVEIKR.

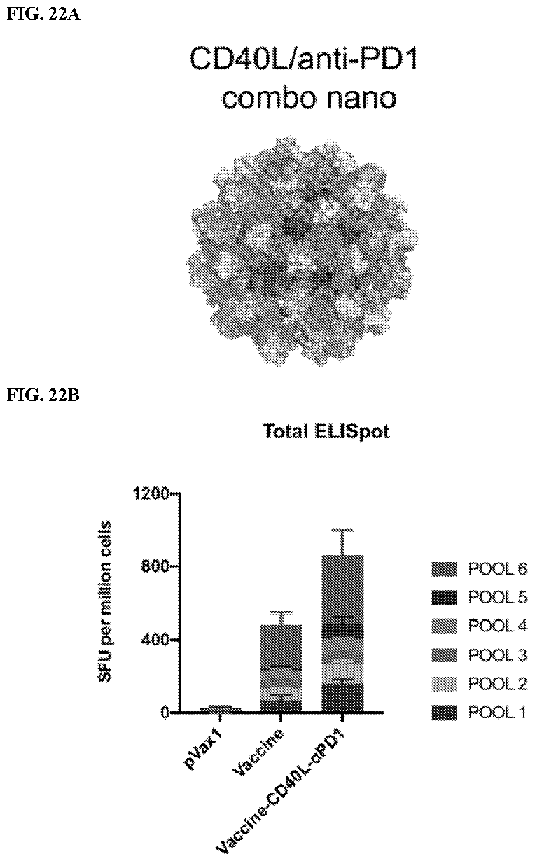

A schematic of the resulted CD40L/anti-PD1 combo nanoparticle is shown in FIG. 22A. To test whether the resulted CD40L/anti-PD1 combo nanoparticles can enhance vaccine-induced T cell responses, one group of mice were injected with 50 μg hTERT DNA vaccine plus 50 μg of the resulted CD40L/anti-PD1 combo nanoparticles. As controls, one group of mice were injected with control DNA plasmid and one group of mice were injected with 50 μg hTERT DNA vaccine alone. As shown in FIG. 22B, mice injected with the DNA vaccine and the CD40L/anti-PD1 combo nanoparticles exhibited better T-cell responses as compared to the mice injected with the DNA vaccine alone.

Other constructs involved in the development of this work include CD40L-Tri cloning and CD40L-Tri cloning.

The CD40L-Tri coding sequence is composed of an optimized IL-2 signal (MRRMQLLLLIALSLALVTNS), an octahistidine, a trimeric leucine zipper GCN4pII heptad repeat derived from the wildtype dimeric GCN4 repeat found in Saccharomyces cerevisiae (GDRMKQIEDKIEEILSKIYHIENEIARIKKLIGER), a flexible 17 amino acid linker (TSGGSGGTGGSGGTGGS), and the extracellular domain of human CD40L (amino acids 51-261). The coding sequence was codon-optimized for CHO cell expression and synthesized by Genscript (Piscataway, NJ). The calculated molecular weight after signal cleavage is 30,093 daltons with a pI of 7.63. There is one predicted N-glycosylation site, which is expected to increase the molecular weight to the observed 35 kDa. The amino acid sequence of the resulted protein has the following sequence (IL-2 signal: bold and underlined; trimeric leucine zipper GCN4pII heptad repeat: italic; linker: bold; CD41L: underlined):

(SEQ ID NO: 145)
MRRMQLLLLIALSLALVTNSGDRMKQIEDKIEEILSKIYHIENEIARIK
KLIGERTSGGSGGTGGSGGTGGSDKVEEEVNLHEDFVFIKKLKRCNKGE
GSLSLLNCEEMRRQFEDLVKDITLNKEEKKENSFEMQRGDEDPQIAAHV
VSEANSNAASVLQWAKKGYYTMKSNLVMLENGKQLTVKREGLYYVYTQV
TFCSNREPSSQRPFIVGLWLKPSSGSERILLKAANTHSSSQLCEQQSVH
LGGVFELQAGASVFVNVTEASQVIHRVGFSSFGLLKL

The mCD40L-Tri-PD-1ScFv cloning resulted in a construct encoding the following amino acid sequence (IL-2 signal: bold and underlined; trimeric leucine zipper GCN4pII heptad repeat: italic; linker: bold; CD41L: underlined; pembroPD1-VH: italic and underlined; linker: bold; PembroPD1-VL: bold and underlined; His tag: bold, italic and underlined):

(SEQ ID NO: 146)
MRRMQLLLLIALSLALVTNSGDRMKQIEDKIEEILSKIYHIENEIARIK
KLIGERTSGGSGGTGGSGGTGGSDKVEEEVNLHEDFVFIKKLKRCNKGE
GSLSLLNCEEMRRQFEDLVKDITLNKEEKKENSFEMQRGDEDPQIAAHV
VSEANSNAASVLQWAKKGYYTMKSNLVMLENGKQLTVKREGLYYVYTQV
TFCSNREPSSQRPFIVGLWLKPSSGSERILLKAANTHSSSQLCEQQSVH
LGGVFELQAGASVFVNVTEASQVIHRVGFSSFGLLKLGGGSGGGSGGGS
GGGSEVQLQESGPGLVKPSQSLSLTCSVTGYSITSSYRWNWIRKFPGNR
LEWMGYINSAGISNYNPSLKRRISITRDTSKNQFFLQVNSVTTEDAATY
YCARSDNMGTTPFTYWGQGTLVTVSSGGGGSGGGGSGGGGSDIVMTQGT
LPNPVPSGESVSITCRSSKSLLYSDGKTYLNWYLQRPGQSPQLLIYWMS
TRASGVSDRFSGSGSGTDFTLKISGVEAEDVGIYYCQQGLEFPTFGGGT
KLELKHHHHHH

### REFERENCES

[1] L. C. Lambert, A. S. Fauci, N Engl J Med 2010, 363, 2036.
[2] Y. Gao, P. F. Mckay, J. F. S. Mann, Viruses 2018, 10.
[3] S. Plotkin, Proc Natl Acad Sci USA 2014, 111, 12283.
[4] C. L. Trimble, et al., Lancet 2015, 386, 2078.
[5] A. E. Gregory, et al., Front Cell Infect Microbiol 2013, 3, 13.
[6] L. Zhao, et al., Vaccine 2014, 32, 327.
[7] S. Al-Halifa, et al., Front Immunol 2019, 10, 22.
[8] W. Szmuness, et al., N Engl J Med 1980, 303, 833.
[9] D. M. Harper, L. R. DeMars, Gynecol Oncol 2017, 146, 196.
[10] J. W. Stone, et al., Nanotechnology 2013, 24, 295102.
[11] K. Cheng, et al., ACS Appl Mater Interfaces 2012, 4, 235.
[12] A. K. Giddam, et al., Nanomedicine (Lond) 2012, 7, 1877.
[13] J. Fuenmayor, F. Godia, L. Cervera, N Biotechnol 2017, 39, 174.
[14] S. A. Kaba, et al., Vaccine 2018, 36, 906.
[15] Z. Xu, D. W. Kulp, Curr Opin Immunol 2019, 59, 49.
[16] J. Marcandalli, et al., Cell 2019, 176, 1420.
[17] K. Sliepen, et al., Retrovirology 2015, 12, 82.
[18] S. Chattopadhyay, J. Y. Chen, H. W. Chen, C. J. Hu, Nanotheranostics 2017, 1, 244.
[19] N. Desai, AAPS J 2012, 14, 282.
[20] J. Feng, C et al., Journal of Translational Medicine 2019, 17, 200.
[21] L. H. Lua, et al., Biotechnol Bioeng 2014, 111, 425.
[22] A. Urakami, et al., J Virol 2017, 91.
[23] Z. Jiang, G. Tong, B. Cai, Y. Xu, J. Lou, Protein Expr Purif 2011, 80, 203.
[24] A. C. Eifler, C. S. Thaxton, Methods Mol Biol 2011, 726, 325.
[25] B. S. Graham, M. S. A. Gilman, J. S. Mclellan, Annu Rev Med 2019, 70, 91.
[26] T. Tokatlian, et al., Science 2019, 363, 649.
[27] J. G. Jardine, et al., Science 2015, 349, 156.
[28] D. Sok, et al., Science 2016, 353, 1557.
[29] J. G. Jardine, et al., Science 2016, 351, 1458.
[30] K. A. Dowd, S et al., Science 2016, 354, 237.
[31] B. S. Davis, et al., J Virol 2001, 75, 4040.
[32] B. Wang, et al., Proc Natl Acad Sci USA 1993, 90, 4156.
[33] N. Y. Sardesai, D. B. Weiner, Curr Opin Immunol 2011, 23, 421.
[34] Z. Xu, et al., EBioMedicine 2018, 35, 97.
[35] M. Khoshnejad, et al., Mol Ther 2019, 27, 188.
[36] M. C. Wise, et al., J Clin Invest 2019.
[37] P. Tebas, et al., N Engl J Med 2017.
[38] P. Tebas, et al., J Infect Dis 2019.
[39] S. A. Kalams, et al., J Infect Dis 2013, 208, 818.
[40] K. Modjarrad, et al., Lancet Infect Dis 2019.
[41] F. Brod, et al., Front Immunol 2018, 9, 2780.
[42] K. Tomusange, et al., Sci Rep 2016, 6, 29131.
[43] [None]
[44] P. Abbink, et al., Science 2016, 353, 1129.
[45] C. Bohannon, et al., Nat Commun 2016, 7, 11826.
[46] B. Selander, et al., J Clin Invest 2006, 116, 1425.
[47] A. Benoit, J. et al., Open Forum Infect Dis 2015, 2, ofv067.
[48] M. Kanekiyo, et al., Nat Immunol 2019, 20, 362.
[49] M. A. Kutzler, D. B. Weiner, Nat Rev Genet 2008, 9, 776.
[50] S. Lu, S. Wang, J. M. Grimes-Serrano, Expert Rev Vaccines 2008, 7, 175.
[51] D. Hobernik, M. Bros, Int J Mol Sci 2018, 19.
[52] M. Kanekiyo, et al., Nature 2013, 499, 102.
[53] R. B. Zinyama-Gutsire, et al., PLOS One 2015, 10, e0122659.
[54] M. Dahl, et al., J Exp Med 2004, 199, 1391.
[55] N. Zhang, M. J. Bevan, Immunity 2011, 35, 161.
[56] N. Van Braeckel-Budimir, J. T. Harty, Front Microbiol 2014, 5, 272.
[57] E. J. Grant, et al., Curr Opin Virol 2016, 16, 132.
[58] M. Koutsakos, et al., Nat Immunol 2019, 20, 613.
[59] A. Durgeau, Y. Virk, S. Corgnac, F. Mami-Chouaib, Front Immunol 2018, 9, 14.
[60] S. Sasaki, et al., Nat Biotechnol 2001, 19, 543.
[61] M. A. Chattergoon, et al., Nat Biotechnol 2000, 18, 974.
[62] M. A. Chattergoon, et al., J Immunol 1998, 160, 5707.
[63] O. Akbari, et al., J Exp Med 1999, 189, 169.
[64] J. Yan, et al., Vaccine 2018, 36, 3079.
[65] P. M. Patel, et al., Oncoimmunology 2018, 7, e1433516.
[66] J. W. Labonte, et al., J Comput Chem 2017, 38, 276.
[67] D. W. Kulp, S et al., J Comput Chem 2012, 33, 1645.
[68] B. Briney, et al., Cell 2016, 166, 1459.
[69] E. K. Duperret, et al., Mol Ther 2018, 26, 435.
[70] Halle S, et al., Trends Immunol 2017; 38:432-43.
[71] Farhood B, et al., J Cell Physiol 2019; 234:8509-21.
[72] Chen L, et al., Nat Rev Immunol 2013; 13:227-42.
[73] Tan W G, et al., J Virol 2013; 87:1359-72.
[74] Gros M, et al., Front Immunol 2019; 10:41.
[75] Saxena M, et al., Trends Cancer 2018; 4:119-37.
[76] Cheng K, et al., ACS Appl Mater Interfaces 2012; 4:235-43.
[77] Fausther-Bovendo H, et al., Hum Vaccin Immunother 2014; 10:2875-84.
[78] Trimble C L, et al., Lancet 2015; 386:2078-88.
[79] Akbari O, et al., J Exp Med 1999; 189:169-78.
[80] Fu T M, et al., Mol Med 1997; 3:362-71.
[81] Xu Z, et al., Adv Sci (Weinh) 2020; 7:1902802.
[82] Xu Z, et al., EBioMedicine 2018; 35:97-105.
[83] Briney B, et al., Cell 2016; 166:1459-70 e11.
[84] Duperret E K, et al., Mol Ther 2018; 26:435-45.
[85] Tokatlian T, et al., Science 2019; 363:649-54.
[86] Jardine J G, et al., Science 2016; 351:1458-63.
[87] Xu Z, et al., Curr Opin Immunol 2019; 59:49-56.
[88] Salem M L, et al., Vaccine 2009; 27:549-57.
[89] Speiser D E, et al., J Clin Invest 2005; 115:739-46.
[90] Chattergoon M A, et al., Nat Biotechnol 2000; 18:974-9.
[91] Porter A G, et al., Cell Death Differ 1999; 6:99-104.
[92] Kyrylkova K, et al., Methods Mol Biol 2012; 887:41-7.
[93] Weisser S B, v et al., J Vis Exp 2012:4105.
[94] Huang M-N, et al., The Journal of Clinical Investigation 2019.
[95] Hildner K, et al., Science 2008; 322:1097-100.
[96] Moynihan K D, et al., Nat Med 2016; 22:1402-10.
[97] Shimizu S, et al., Cancer Med 2019; 8:80-93.
[98] Gooden M J, et al., Br J Cancer 2011; 105: 93-103.
[99] Beyranvand Nejad E, et al., J Immunother Cancer 2019; 7:25.
[100] Rohaan M W, et al., J Immunother Cancer 2018; 6:102.
[101] Wu R. et al., Cancer J 2012; 18:160-75.
[102] Kim H J. et al., Cancer Immunol Res 2014; 2:91-8.
[103] Quezada S A. et al., J Exp Med 2010; 207:637-50.
[104] Moorthy V S. et al., Malar J 2009; 8:312.

1. A composition comprising an expressible nucleic acid sequence comprising:
a) a first nucleic acid sequence encoding a scaffold domain comprising a self-assembling polypeptide; and
b) a second nucleic acid sequence encoding a viral antigen,
wherein the self-assembling polypeptide is from Aquifex aeolicus, Helicobacter pylori, Pyrococcus furiosus or Thermotoga maritima.

2. (canceled)

3. The composition of claim 1, wherein the self-assembling polypeptide comprises at least 70% sequence identity to SEQ ID NO: 7, SEQ ID NO: 23, SEQ ID NO: 31 or SEQ ID NO: 26.

4. The composition of claim 1, wherein the viral antigen is an antigen from a retrovirus, flavivirus, Nipah virus, West Nile virus, human papillomavirus, respiratory syncytial virus, filovirus, zaire ebolavirus, sudan ebolavirus, marburgvirus or influenza virus.

5. (canceled)

6. The composition of claim 1, wherein the viral antigen comprises at least 70% sequence identity to SEQ ID NO: 9, SEQ ID NO: 45, SEQ ID NO: 47, SEQ ID NO: 49, SEQ ID NO: 51, SEQ ID NO: 53, SEQ ID NO: 55, SEQ ID NO: 65, SEQ ID NO: 66 or SEQ ID NO: 67.

7. The composition of claim 1, wherein the expressible nucleic acid sequence further comprises a third nucleic acid sequence encoding a linker domain comprising a linker peptide, said third nucleic acid sequence positioned between the first nucleic acid sequence and the second nucleic acid sequence in the 5′ to 3′ orientation.

8.-12. (canceled)

13. A pharmaceutical composition comprising (i) a therapeutically effective amount of the composition of claim 1, and (ii) a pharmaceutically acceptable carrier.

14.-15. (canceled)

16. A method of vaccinating a subject in need thereof comprising administering to the subject a therapeutically effective amount of the pharmaceutical composition of claim 13.

17. The method of claim 16, wherein the administering is accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or a combination thereof.

18.-19. (canceled)

20. The method of claim 16, wherein the therapeutically effective dose is from about 0.3 micrograms of the composition per kilogram of subject to about 30 micrograms per kilogram of subject.

21.-28. (canceled)

29. A method of neutralizing one or plurality of viruses in a subject comprising administering to the subject the pharmaceutical composition of claim 13.

30. (canceled)

31. The method of claim 29, wherein the administering comprises administering from about 1 to about 30 micrograms of the expressible nucleic acid sequence to the subject; and wherein the subject is human.

32.-33. (canceled)

34. A method of stimulating a therapeutically effective, antigen-specific immune response against a virus in a mammal infected with the virus comprising administering a therapeutically effective amount of the pharmaceutical composition of claim 13 to the mammal.

35. The method of claim 34, wherein the mammal is infected with a HIV virus.

36. (canceled)

37. A method of inducing expression of a self-assembling vaccine in a subject comprising administering the pharmaceutical composition of claim 13.

38. The method of claim 37, wherein the method is free of administering any polypeptide directly to the subject.

39.-90. (canceled)

91. A nanoparticle comprising:
(i) from about 7 to about 120 monomers, each monomer comprising at least about 70% sequence identity to SEQ ID NO: 7, SEQ ID NO: 23, SEQ ID NO: 26, or SEQ ID NO: 31, or a functional fragment thereof; and
(ii) a CD40L polypeptide.

92. The nanoparticle of claim 91 further comprising a polypeptide that is a viral antigen.

93. The nanoparticle of claim 91, wherein the CD40L polypeptide comprises at least 70% sequence identity to SEQ ID NO: 103, SEQ ID NO: 104, SEQ ID NO: 105, SEQ ID NO: 106, SEQ ID NO: 108, SEQ ID NO: 109, SEQ ID NO: 110, or SEQ ID NO: 111, or a functional fragment thereof.

94.-118. (canceled)

119. The composition of claim 91, wherein the CD40L polypeptide is encoded by a nucleic acid sequence comprising at least 70% sequence identity to SEQ ID NO: 102 or SEQ ID NO: 107, or a functional fragment thereof that comprises at least about 70%, sequence identity to SEQ ID NO: 102 or SEQ ID NO: 107.

120. The composition of claim 1, wherein the scaffold domain is encoded by a nucleic acid sequence comprising at least 70% sequence identity to SEQ ID NO: 2, SEQ ID NO: 13, SEQ ID NO: 14, or SEQ ID NO: 15, or a functional fragment thereof that comprises at least about 70% sequence identity to SEQ ID NO: 2, SEQ ID NO: 13, SEQ ID NO: 14, or SEQ ID NO: 15.

121. A method of treating viral infection in a subject in need thereof comprising administering to the subject a pharmaceutical composition of claim 13.

122. The method of claim 121, wherein the viral infection is HIV-1 infection.


SEQUENCE LISTING (148 sequences)

SEQ ID NO: 1 - DNA, 54 bp, Artificial Sequence
  Leader sequence
atggactggacctggattctgttcctggtggccgccgccacaagggtgcacagc

SEQ ID NO: 2 - DNA, 462 bp, Artificial Sequence
  Self-assembling polypeptide from lumazine synthase of Aquifex aeolicus
atgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcga

SEQ ID NO: 3 - DNA, 45 bp, Artificial Sequence
  Linker
ggaggctccggaggatctggagggagtggaggctcaggaggaggc

SEQ ID NO: 4 - DNA, 516 bp, Artificial Sequence
  Fragment of gp120
gacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccacc

SEQ ID NO: 5 - DNA, 1083 bp, Artificial Sequence
  Expressible nucleic acid sequence
atggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccacctgataa

SEQ ID NO: 6 - protein, 18 aa, Artificial Sequence
  IgE leader sequence
MDWTWILFLVAAATRVHS

SEQ ID NO: 7 - protein, 154 aa, Artificial Sequence
  Self-assembling polypeptide
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLR

SEQ ID NO: 8 - protein, 15 aa, Artificial Sequence
  Linker
GGSGGSGGSGGSGGG

SEQ ID NO: 9 - protein, 172 aa, Artificial Sequence
  Viral antigen fragment of gp120
DTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNST

SEQ ID NO: 10 - protein, 359 aa, Artificial Sequence
  Expressed self assembly viral antigen
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNST

SEQ ID NO: 11 - DNA, 594 bp, Artificial Sequence
  IgE-GLT1
ggatccgccaccatggactggacctggattctgttcctggtggccgccgccacaagggtgcacagcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccacctgataactcgag

SEQ ID NO: 12 - protein, 190 aa, Artificial Sequence
  IgE-gp120
MDWTWILFLVAAATRVHSDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNST

SEQ ID NO: 13 - DNA, 501 bp, Artificial Sequence
  Self-assembling polypeptide 3BVE
gggctgagtaaggacattatcaagctgctgaacgaacaggtgaacaaagagatgcagtctagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaatgagaaccacgggctgtacctggcagatcagtatgtcaagggcatcgctaagtcaaggaaaagc

SEQ ID NO: 14 - DNA, 1032 bp, Artificial Sequence
  Self-assembling polypeptide RBE
ctgagcattgcccccacactgattaaccgggacaaaccctacaccaaagaggaactgatggagattctgagactggctattatcgctgagctggacgccatcaacctgtacgagcagatggcccggtattctgaggacgagaatgtgcgcaagatcctgctggatgtggccagggaggagaaggcacacgtgggagagttcatggccctgctgctgaacctggaccccgagcaggtgaccgagctgaagggcggctttgaggaggtgaaggagctgacaggcatcgaggcccacatcaacgacaataagaaggaggagagcaacgtggagtatttcgagaagctgagatccgccctgctggatggcgtgaataagggcaggagcctgctgaagcacctgcctgtgaccaggatcgagggccagagcttcagagtggacatcatcaagtttgaggatggcgtgcgcgtggtgaagcaggagtacaagcccatccctctgctgaagaagaagttctacgtgggcatcagggagctgaacgacggcacctacgatgtgagcatcgccacaaaggccggcgagctgctggtgaaggacgaggagtccctggtcatccgcgagatcctgtctacagagggcatcaagaagatgaagctgagctcctgggacaatccagaggaggccctgaacgatctgatgaatgccctgcaggaggcatctaacgcaagcgccggaccattcggcctgatcatcaatcccaagagatacgccaagctgctgaagatctatgagaagtccggcaagatgctggtggaggtgctgaaggagatcttccggggcggcatcatcgtgaccctgaacatcgatgagaacaaagtgatcatctttgccaacacccctgccgtgctggacgtggtggtgggacaggatgtgacactgcaggagctgggaccagagggcgacgatgtggcctttctggtgtccgaggccatcggcatcaggatcaagaatccagaggcaatcgtggtgctggag

SEQ ID NO: 15 - DNA, 645 bp, Artificial Sequence
  Self-assembling polypeptide I3
gagaaagcagccaaagcagaggaagcagcacggaagatggaagaactgttcaagaagcacaagatcgtggccgtgctgagggccaactccgtggaggaggccaagaagaaggccctggccgtgttcctgggcggcgtgcacctgatcgagatcacctttacagtgcccgacgccgataccgtgatcaaggagctgtctttcctgaaggagatgggagcaatcatcggagcaggaaccgtgacaagcgtggagcagtgcagaaaggccgtggagagcggcgccgagtttatcgtgtcccctcacctggacgaggagatctctcagttctgtaaggagaagggcgtgttttacatgccaggcgtgatgacccccacagagctggtgaaggccatgaagctgggccacacaatcctgaagctgttccctggcgaggtggtgggcccacagtttgtgaaggccatgaagggccccttccctaatgtgaagtttgtgcccaccggcggcgtgaacctggataacgtgtgcgagtggttcaaggcaggcgtgctggcagtgggcgtgggcagcgccctggtgaagggcacacccgtggaagtcgctgagaaggcaaaggcattcgtggaaaagattagggggtgtactgag

SEQ ID NO: 16 - DNA, 72 bp, Artificial Sequence
  Linker
ggcggctctggcggaagtggcggaagtgggggaagtggaggcggcggaagcgggggaggcagcgggggaggg

SEQ ID NO: 17 - DNA, 27 bp, Artificial Sequence
  Linker
ggcggaagcggcggaagcggcgggtct

SEQ ID NO: 18 - protein, 241 aa, Artificial Sequence
  Rigid linker 4QTL-115 Angstroms, single chain 3-helix bundle
NEDDMKKLYKQMVQELEKARDRMEKLYKEMVELIQKAIELMRKIFQEVKQEVEKAIEEMKKLYDEAKKKIEQMIQQIKQGGDKQKMEELLKRAKEEMKKVKDKMEKLLEKLKQIMQEAKQKMEKLLKQLKEEMKKMKEKMEKLLKEMKQRMEEVKKKMDGDDELLEKIKKNIDDLKKIAEDLIKKAEENIKEAKKIAEQLVKRAKQLIEKAKQVAEELIKKILQLIEKAKEIAEKVLKGLE

SEQ ID NO: 19 - DNA, 36 bp, Artificial Sequence
  Linker
ggcggcagcggcggcagcggcgggagcggaggaagt

SEQ ID NO: 20 - DNA, 1104 bp, Artificial Sequence
  IgE-GLT1-3BVE
atggactggacctggattctgttcctggtggccgccgccacaagggtgcacagcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccaccggcggaagcggcggaagcggcgggtctgggctgagtaaggacattatcaagctgctgaacgaacaggtgaacaaagagatgcagtctagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaatgagaaccacgggctgtacctggcagatcagtatgtcaagggcatcgctaagtcaaggaaaagctgataa

SEQ ID NO: 21 - protein, 366 aa, Artificial Sequence
  IgE-GLT1-3BVE
MDWTWILFLVAAATRVHSDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNSTGGSGGSGGSGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 22 - protein, 9 aa, Artificial Sequence
  Linker
GGSGGSGGS

SEQ ID NO: 23 - protein, 167 aa, Artificial Sequence
  3BVE scaffold
GLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 24 - DNA, 1257 bp, Artificial Sequence
  IgE-GLT1-I3
atggactggacctggattctgttcctggtggccgccgccacaagggtgcacagcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccaccggcggcagcggcggcagcggcgggagcggaggaagtgagaaagcagccaaagcagaggaagcagcacggaagatggaagaactgttcaagaagcacaagatcgtggccgtgctgagggccaactccgtggaggaggccaagaagaaggccctggccgtgttcctgggcggcgtgcacctgatcgagatcacctttacagtgcccgacgccgataccgtgatcaaggagctgtctttcctgaaggagatgggagcaatcatcggagcaggaaccgtgacaagcgtggagcagtgcagaaaggccgtggagagcggcgccgagtttatcgtgtcccctcacctggacgaggagatctctcagttctgtaaggagaagggcgtgttttacatgccaggcgtgatgacccccacagagctggtgaaggccatgaagctgggccacacaatcctgaagctgttccctggcgaggtggtgggcccacagtttgtgaaggccatgaagggccccttccctaatgtgaagtttgtgcccaccggcggcgtgaacctggataacgtgtgcgagtggttcaaggcaggcgtgctggcagtgggcgtgggcagcgccctggtgaagggcacacccgtggaagtcgctgagaaggcaaaggcattcgtggaaaagattagggggtgtactgagtgataa

SEQ ID NO: 25 - protein, 417 aa, Artificial Sequence
  IgE-GLT1-I3
MDWTWILFLVAAATRVHSDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNSTGGSGGSGGSGGSEKAAKAEEAARKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 26 - protein, 215 aa, Artificial Sequence
  I3 scaffold
EKAAKAEEAARKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 27 - protein, 12 aa, Artificial Sequence
  Linker
GGSGGSGGSGGS

SEQ ID NO: 28 - DNA, 1674 bp, Artificial Sequence
  IgE-GLT1-RBE
atggactggacctggattctgttcctggtggccgccgccacaagggtgcacagcctgagcattgcccccacactgattaaccgggacaaaccctacaccaaagaggaactgatggagattctgagactggctattatcgctgagctggacgccatcaacctgtacgagcagatggcccggtattctgaggacgagaatgtgcgcaagatcctgctggatgtggccagggaggagaaggcacacgtgggagagttcatggccctgctgctgaacctggaccccgagcaggtgaccgagctgaagggcggctttgaggaggtgaaggagctgacaggcatcgaggcccacatcaacgacaataagaaggaggagagcaacgtggagtatttcgagaagctgagatccgccctgctggatggcgtgaataagggcaggagcctgctgaagcacctgcctgtgaccaggatcgagggccagagcttcagagtggacatcatcaagtttgaggatggcgtgcgcgtggtgaagcaggagtacaagcccatccctctgctgaagaagaagttctacgtgggcatcagggagctgaacgacggcacctacgatgtgagcatcgccacaaaggccggcgagctgctggtgaaggacgaggagtccctggtcatccgcgagatcctgtctacagagggcatcaagaagatgaagctgagctcctgggacaatccagaggaggccctgaacgatctgatgaatgccctgcaggaggcatctaacgcaagcgccggaccattcggcctgatcatcaatcccaagagatacgccaagctgctgaagatctatgagaagtccggcaagatgctggtggaggtgctgaaggagatcttccggggcggcatcatcgtgaccctgaacatcgatgagaacaaagtgatcatctttgccaacacccctgccgtgctggacgtggtggtgggacaggatgtgacactgcaggagctgggaccagagggcgacgatgtggcctttctggtgtccgaggccatcggcatcaggatcaagaatccagaggcaatcgtggtgctggagggcggctctggcggaagtggcggaagtgggggaagtggaggcggcggaagcgggggaggcagcgggggaggggacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccacc

SEQ ID NO: 29 - DNA, 72 bp, Artificial Sequence; identical to SEQ ID NO: 16
  Linker

SEQ ID NO: 30 - protein, 558 aa, Artificial Sequence
  IgE leader-RBE-linker-HIV antigen
MDWTWILFLVAAATRVHSLSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGGSGGSGGSGGSGGGGSGGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNST

SEQ ID NO: 31 - protein, 344 aa, Artificial Sequence
  RBE scaffold
LSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLE

SEQ ID NO: 32 - protein, 24 aa, Artificial Sequence
  Linker
GGSGGSGGSGGSGGGGSGGGSGGG

SEQ ID NO: 33 - DNA, 1254 bp, Artificial Sequence
  NivFtop_stab2_gMax_Nt_60mer
atggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcggggtcacttgtgccggacgagccatcggaaatgctaccgccgcccagattactgccggagtcgccctgtatgaagccatgaagaatgccgacaacatcaataagctgaagagctccatcgagagcaccaacgaggccgtggtgaagctgcaggagacagccgagaagacagtgtacgtgctgacagccctgcaggactatatcaacaccaatctggtgcccacaatcgataagatcagctgcaagcagaccgaggcatccctggacgccgccctgtccaagtacctgtctgatctgctgtacgtgttcggccccaacctgagcgaccccgtgagcaattctatgcctatccaggccatctctcaggccttcggcggcaactacagcaccctgctgaggacactgggctatgccccagaggactttgacgatctgctggagagcgattccatcacaggccagatcatctacgtggacctgtctagctactatatcatcgtgagagtgtattttccaaatggctccggccccctgaccaaggatatcgtgatcaagatgatccccaacgtgtctaatatgagccagtgtacaggctctgtgatggagaactacaagaccaggctgaatggcatcctgacacctatcaagggcgccctggagatctataagaataactgtcacgatggatgataa

SEQ ID NO: 34 - DNA, 693 bp, Artificial Sequence
  NivFtop_stab2_gMax
ggggtcacttgtgccggacgagccatcggaaatgctaccgccgcccagattactgccggagtcgccctgtatgaagccatgaagaatgccgacaacatcaataagctgaagagctccatcgagagcaccaacgaggccgtggtgaagctgcaggagacagccgagaagacagtgtacgtgctgacagccctgcaggactatatcaacaccaatctggtgcccacaatcgataagatcagctgcaagcagaccgaggcatccctggacgccgccctgtccaagtacctgtctgatctgctgtacgtgttcggccccaacctgagcgaccccgtgagcaattctatgcctatccaggccatctctcaggccttcggcggcaactacagcaccctgctgaggacactgggctatgccccagaggactttgacgatctgctggagagcgattccatcacaggccagatcatctacgtggacctgtctagctactatatcatcgtgagagtgtattttccaaatggctccggccccctgaccaaggatatcgtgatcaagatgatccccaacgtgtctaatatgagccagtgtacaggctctgtgatggagaactacaagaccaggctgaatggcatcctgacacctatcaagggcgccctggagatctataagaataactgtcacgatggatgataa

SEQ ID NO: 35 - protein, 416 aa, Artificial Sequence
  Expressed self assembly viral antigen
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGGVTCAGRAIGNATAAQITAGVALYEAMKNADNINKLKSSIESTNEAVVKLQETAEKTVYVLTALQDYINTNLVPTIDKISCKQTEASLDAALSKYLSDLLYVFGPNLSDPVSNSMPIQAISQAFGGNYSTLLRTLGYAPEDFDDLLESDSITGQIIYVDLSSYYIIVRVYFPNGSGPLTKDIVIKMIPNVSNMSQCTGSVMENYKTRLNGILTPIKGALEIYKNNCHDG

SEQ ID NO: 36 - protein, 229 aa, Artificial Sequence
  Expressed NivFtop_stab2_gMax
GVTCAGRAIGNATAAQITAGVALYEAMKNADNINKLKSSIESTNEAVVKLQETAEKTVYVLTALQDYINTNLVPTIDKISCKQTEASLDAALSKYLSDLLYVFGPNLSDPVSNSMPIQAISQAFGGNYSTLLRTLGYAPEDFDDLLESDSITGQIIYVDLSSYYIIVRVYFPNGSGPLTKDIVIKMIPNVSNMSQCTGSVMENYKTRLNGILTPIKGALEIYKNNCHDG

SEQ ID NO: 37 - DNA, 1254 bp, Artificial Sequence
  NivFtop_stab2_gMax_Ct_60mer
atggactggacctggattctgttcctggtggccgccgccacaagggtgcacagcggggtcacttgtgccggacgagccatcggaaatgctaccgccgcccagattactgccggagtcgccctgtatgaagccatgaagaatgccgacaacatcaataagctgaagagctccatcgagagcaccaacgaggccgtggtgaagctgcaggagacagccgagaagacagtgtacgtgctgacagccctgcaggactatatcaacaccaatctggtgcccacaatcgataagatcagctgcaagcagaccgaggcatccctggacgccgccctgtccaagtacctgtctgatctgctgtacgtgttcggccccaacctgagcgaccccgtgagcaattctatgcctatccaggccatctctcaggccttcggcggcaactacagcaccctgctgaggacactgggctatgccccagaggactttgacgatctgctggagagcgattccatcacaggccagatcatctacgtggacctgtctagctactatatcatcgtgagagtgtattttccaaatggctccggccccctgaccaaggatatcgtgatcaagatgatccccaacgtgtctaatatgagccagtgtacaggctctgtgatggagaactacaagaccaggctgaatggcatcctgacacctatcaagggcgccctggagatctataagaataactgtcacgatggaggaggctccggaggatctggagggagtggaggctcaggaggaggcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgatgataa

SEQ ID NO: 38 - protein, 416 aa, Artificial Sequence
  Expressed NivFtop_stab2_gMax_Ct_60mer
MDWTWILFLVAAATRVHSGVTCAGRAIGNATAAQITAGVALYEAMKNADNINKLKSSIESTNEAVVKLQETAEKTVYVLTALQDYINTNLVPTIDKISCKQTEASLDAALSKYLSDLLYVFGPNLSDPVSNSMPIQAISQAFGGNYSTLLRTLGYAPEDFDDLLESDSITGQIIYVDLSSYYIIVRVYFPNGSGPLTKDIVIKMIPNVSNMSQCTGSVMENYKTRLNGILTPIKGALEIYKNNCHDGGGSGGSGGSGGSGGGMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLR

SEQ ID NO: 39 - DNA, 132 bp, Artificial Sequence
  Leader sequence
atggactggacctggagaatcctgttcctggtggccgccgccaccggcacacacgccgatacacacttccccatctgcatcttttgctgtggctgttgccataggtccaagtgtgggatgtgctgcaaaact

SEQ ID NO: 40 - protein, 19 aa, Artificial Sequence
  Leader sequence
MDWTWRILFLVAAATGTHA

SEQ ID NO: 41 - DNA, 315 bp, West Nile virus
atgtctaagaaaccaggagggcccggcaagagccgggctgtcaatatgctaaaacgcggaatgccccgcgtgttgtccttgattggactgaagagggctatgttgaacctgatcgacggcaaggggccaatacgatttgtgttggctctcttggcgttcttcaggttcacagcaattgctccgacccgagcagtgctggatcgatggagaggtgtgaacaaacaaacagcgatgaaacaccttttgagttttaagaaggaactagggaccttgaccagtgctatcaatcggcggagctcaaaacaaaagaaaaga

SEQ ID NO: 42 - DNA, 1518 bp, Human papillomavirus type 10
atgtcactttggcttccatcagaagctactgtttaccttccaccagttccagtttcaaaagttgtttcaactgatgaatacgttgctaggactaatatttactaccatgctggaacttcaaggcttcttgctgttggacatccatactttccaattaaaaaaccaaataataataaaattcttgttccaaaagtttcaggacttcaatacagggtttttaggattcatcttccagatccaaataaatttggatttccagatacttcattttacaatccagatactcaaaggcttgtttgggcttgtgttggagttgaagttggaaggggacaaccacttggagttggaatttcaggacatccacttcttaataaacttgatgatactgaaaatgcttcagcttacgctgctaatgctggagttgataatagggaatgtatttcaatggattacaaacaaactcaactttgtcttattggatgtaaaccaccaattggagaacattggggaaaaggatcaccatgtactaatgttgctgttaatccaggagattgtccaccacttgaacttattaatactgttattcaagatggagatatggttgatactggatttggagctatggattttactactcttcaagctaataaatcagaagttccacttgatatctgtacttcaatttgtaaatacccagattacattaaaatggtttcagaaccatacggagattcactttttttttaccttaggagggaacaaatgtttgttaggcatctttttaatagggctggagctgttggagaaaatgttccagatgatctttacattaaaggatcaggatcaactgctaatcttgcttcatcaaattactttccaactccatcaggatcaatggttacttcagatgctcaaatttttaataaaccatactggcttcaaagggctcaaggacataataatggaatttgttggggaaatcaactttttgttactgttgttgatactactaggtcaactaatatgtcactttgtgctgctatttcaacttcagaaactacttacaaaaatactaattttaaagaataccttaggcatggagaagaatacgatcttcaatttatttttcaactttgtaaaattactcttactgctgatgttatgacttacattcattcaatgaattcaactattcttgaagattggaattttggacttcaaccaccaccaggaggaactcttgaagatacttacaggtttgttacttcacaagctattgcttgtcaaaaacatactccaccagctccaaaagaggatccacttaaaaaatacactttttgggaagttaatcttaaagaaaaattttcagcagatcttgatcaatttccacttggaaggaaatttcttcttcaagctggacttaaagctaaaccaaaatttactcttggaaaaaggaaagctactccaactacttcatcaacttcaactactgctaaaaggaaaaaaaggaaactttga

SEQ ID NO: 43 - DNA, 1422 bp, Human papillomavirus type 10
atgaggcacaagaggagcgccaagaggaccaagagggccagcgccacccagctgtacaagacctgcaagcaggccggcacctgcccccccgacatcatccccaaggtggagggcaagaccatcgccgaccagatcctgcagtacggcagcatgggcgtgttcttcggcggcctgggcatcggcaccggcagcggcaccggcggcaggaccggctacatccccctgggcaccaggccccccaccgccaccgacaccctggcccccgtgaggccccccctgaccgtggaccccgtgggccccagcgaccccagcatcgtgagcctggtggaggagaccagcttcatcgacgccggcgcccccaccagcgtgcccagcatcccccccgacgtgagcggcttcagcatcaccaccagcaccgacaccacccccgccatcctggacatcaacaacaccgtgaccaccgtgaccacccacaacaaccccaccttcaccgaccccagcgtgctgcagccccccacccccgccgagaccggcggccacttcaccctgagcagcagcaccatcagcacccacaactacgaggagatccccatggacaccttcatcgtgagcaccaaccccaacaccgtgaccagcagcacccccatccccggcagcaggcccgtggccaggctgggcctgtacagcaggaccacccagcaggtgaaggtggtggaccccgccttcgtgaccacccccaccaagctgatcacctacgacaaccccgcctacgagggcatcgacgtggacaacaccctgtacttcagcagcaacgacaacagcatcaacatcgcccccgaccccgacttcctggacatcgtggccctgcacaggcccgccctgaccagcaggaggaccggcatcaggtacagcaggatcggcaacaagcagaccctgaggaccaggagcggcaagagcatcggcgccaaggtgcactactactacgacctgagcaccatcgaccccgccgaggagatcgagctgcagaccatcacccccagcacctacaccaccaccagccacgccgccagccccaccagcatcaacaacggcctgtacgacatctacgccgacgacttcatcaccgacaccagcaccacccccgtgcccagcgtgcccagcaccagcctgagcggctacatccccgccaacaccaccatccccttcggtggcgcctacaacatccccctggtgagcggccccgacatccccatcaacatcaccgaccaggcccccagcctgatccccatcgtgcccggcagcccccagtacaccatcatcgccgacgccggcgacttctacctgcaccccagctactacatgctgaggaagaggaggaagaggctgccctacttcttcagcgacgtgagcctggccgcctga

SEQ ID NO: 44 - DNA, 1725 bp, respiratory syncytial virus
  misc_feature (1026)..(1026): n is either a or t or g or c
  misc_feature (1062)..(1062): n is either a or t or g or c
  misc_feature (1182)..(1182): n is either a or t or g or c
gagctgcccatcctgaaaacaaacgccatcaccaccatcctggccgccgtgaccctgtgcttcgccagcagccagaacatcaccgaggaattctaccagagcacctgtagcgccgtgtccaagggctacctgtctgccctgcggaccggctggtacaccagcgtgatcaccatcgagctgagcaacatcaaagaaaacaagtgcaacggcaccgacgccaaagtgaagctgatcaagcaggaactggacaagtacaagaacgccgtgaccgagctgcagctgctgatgcagagcacccctgccgccaacaacagagccagacgcgagctgccccggttcatgaactacaccctgaacaacaccaagaacaccaacgtgaccctgagcaagaagcggaagcggcggttcctgggattcctgctgggcgtgggcagcgccattgcctctggaatcgctgtgtctaaggtgctgcacctggaaggcgaagtgaacaagatcaagtccgccctgctgagcaccaacaaggccgtggtgtccctgagcaacggcgtgtccgtgctgaccagcaaggtgctggatctgaagaactacatcgacaagcagctgctgcctatcgtgaacaagcagagctgcagcatcagcaacatcgagacagtgatcgagttccagcagaagaacaaccggctgctggaaatcacccgcgagttcagcgtgaacgccggcgtgaccacccccgtgtccacctacatgctgaccaacagcgagctgctgagcctgatcaacgacatgcccatcaccaacgaccagaaaaagctgatgagcaacaacgtgcagatcgtgcggcagcagagctactccatcatgtccatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgngataatgccggcagcgtgtcattctttccacaggcngagacatgcaaggtgcagagcaaccgggtgttctgcgacaccatgaacagcctgaccctgccctccgaagtgaacctgtgcaacatcgacatcttcaaccctaagtacgactgcaagatnatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcgacgagttcgacgccagcatcagccaggtgaacgagaagatcaaccagagcctggccttcatcagaaagagcgacgagctgctgcacaatgtgaatgccggcaagagcaccaccaatatcatgatcaccacaatcatcatcgtgatcattgtgatcctgctgtccctgatcgccgtgggcctgctgctgtactgcaaggcccggtccacccctgtgaccctgtccaaggaccagctgagcggaatcatcaacaatatcgccttctccaactga

SEQ ID NO: 45 - protein, 479 aa, respiratory syncytial virus
  MISC_FEATURE (247)..(247): Xaa is unknown
  MISC_FEATURE (259)..(259): Xaa is unknown
  MISC_FEATURE (299)..(299): Xaa is unknown
MQSTPAANNRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYXDNAGSVSFFPQXETCKVQSNRVFCDTMNSLTLPSEVNLCNIDIFNPKYDCKXMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGIINNIAFSN

SEQ ID NO: 46 - DNA, 897 bp, respiratory syncytial virus
atgtccaagaataaggatcagaggaccgcgaaaacgcttgagaggacgtgggacacgctgaaccacctcctgttcatctcctcgtgtctctacaagctcaaccttaagtccatcgcgcagatcaccttgagcattctcgccatgatcatctccaccagccttatcattgccgcaatcatcttcatcgcatccgccaaccataaggtgacattgactacagcgattatccaagacgctactagccagatcaagaataccacgccgacctatttgacgcaaaatcctcagttgggaattagcttctcgaatctctcggaaaccacgtcgcagccgactacaattcttgcgtcaacgactccatcggccaaatcaacaccacaatcgactaccgtaaaaacgaagaacacgactacaacacagattcagccttcaaagcccacgaccaaacagagacagaataagccgcccaacaagcccaacaatgattttcacttcgaggtgtttaacttcgtgccctgttcgatttgcagcaataaccccacgtgctgggcgatttgcaagcgaatcccgaataagaagcccgggaaaaagaccacgacgaaaccgacaaagaagccgacaatcaagacaacgaaaaaggatcttaaacctcagacgacaaagcctaaggaagtcttgacaacgaagcctacggaaaaacccactatcaatactaccaagactaacatccggacaacactgctgacgagcaataccacgggaaacccggagctcacatcgcagaaagagacactccattcgacatcctccgagggtaacccttcgcccagccaggtgtatacgacgtcagaataccctagccaaccctcatcgccctcaaatacgacccggcaatga

SEQ ID NO: 47 - protein, 298 aa, respiratory syncytial virus
MSKNKDQRTAKTLERTWDTLNHLLFISSCLYKLNLKSIAQITLSILAMIISTSLIIAAIIFIASANHKVTLTTAIIQDATSQIKNTTPTYLTQNPQLGISFSNLSETTSQPTTILASTTPSAKSTPQSTTVKTKNTTTTQIQPSKPTTKQRQNKPPNKPNNDFHFEVFNFVPCSICSNNPTCWAICKRIPNKKPGKKTTTKPTKKPTIKTTKKDLKPQTTKPKEVLTTKPTEKPTINTTKTNIRTTLLTSNTTGNPELTSQKETLHSTSSEGNPSPSQVYTTSEYPSQPSSPSNTTRQ

SEQ ID NO: 48 - DNA, 879 bp, respiratory syncytial virus
  misc_feature (37)..(37): N is unknown
  misc_feature (173)..(173): N is unknown
  misc_feature (285)..(285): N is unknown
atgagcaaaaacaaaaaccaaaggacggctcggacgnttgagaaaacatgggacacgcttaatcaccttattgtgatctcatcgtgtttgtaccggttgaatctcaagagcatcgcccagattgcgctgtcagtcctggccatgattatctcgacatcactcatcatcgcagncatcatctttatcatttcagcgaatcacaaggtaacgcttacaacagtcacggtgcagaccatcaagaatcataccgaaaagaatatcacaacctacctcacccaagtcagnccggagagagtaagcccctcaaaacagcctactacgacacctcccatccacacgaactcggcgaccatctcaccgaataccaaatcagaaacgcatcatacgaccgcacagacaaagggacgaaccactacacccacacagaacaacaaacccagcaccaagccgaggccaaagaatccgcccaagaagccgaaagatgactatcactttgaagtgttcaacttcgtaccgtgttcgatttgcgggaataatcagttgtgcaaatccatttgcaagacgatcccatccaacaaaccgaagaagaaacctaccatcaagcccacaaacaagccaacgacaaaaacaacgaacaagcgcgatcccaaaacgctcgcgaaaacgttgaagaaggaaacgacgacaaaccctacgaagaaacccacgcccaagaccactgagagagacacctccacctcgcaatcgacggtacttgacacgactacgagcaagcacactatccagcaacagtccctgcactcaaccacgcccgagaatacaccaaactcaacacagactccgacagcttcagagccttccacttcgaattccacatga

SEQ ID NO: 49 - protein, 292 aa, respiratory syncytial virus
  misc_feature (13)..(13): Xaa can be any naturally occurring amino acid
  MISC_FEATURE (20)..(20): Xaa is unknown
  MISC_FEATURE (58)..(58): Xaa is unknown
  MISC_FEATURE (95)..(95): Xaa is unknown
MSKNKNQRTARTXEKTWDTXNHLIVISSCLYRLNLKSIAQIALSVLAMIISTSLIIAXIIFIISANHKVTLTTVTVQTIKNHTEKNITTYLTQVXPERVSPSKQPTTTPPIHTNSATISPNTKSETHHTTAQTKGRTTTPTQNNKPSTKPRPKNPPKKPKDDYHFEVFNFVPCSICGNNQLCKSICKTIPSNKPKKKPTIKPTNKPTTKTTNKRDPKTLAKTLKKETTTNPTKKPTPKTTERDTSTSQSTVLDTTTSKHTIQQQSLHSTTPENTPNSTQTPTASEPSTSNST

SEQ ID NO: 50 - DNA, 2034 bp, Ebola virus
  misc_feature (675)..(675): n is either a or t or g or c
  misc_feature (1492)..(1492): n is either a or t or g or c
  misc_feature (1752)..(1752): n is either a or t or g or c
  misc_feature (1802)..(1802): n is either a or t or g or c
atgggggtcactgggattctgcagctgcctagagatcgcttcaagcgaacctctttctttctgtgggtcatcattctgttccagaggacttttagtatccctctgggcgtcattcacaattctaccctgcaggtgagtgacgtcgataagctggtgtgtcgggacaaactgagctccaccaaccagctgagatctgtcggcctgaatctggaggggaacggagtggctaccgatgtcccaagtgcaacaaagagatgggggtttcgctcaggagtgccccctaaagtggtcaattacgaggccggggaatgggctgagaattgctataacctggaaatcaagaaacccgacggatcagagtgtctgccagccgctcccgatgggattcgcggattccctagatgcagatacgtgcacaaggtcagcggcaccgggccatgtgcaggagacttcgcctttcataaagaaggcgccttctttctgtacgatagactggcttccaccgtgatctatagggggaccacattcgccgagggagtggtcgcttttctgattctgcctcaggccaagaaagacttcttttctagtcatcctctgcgggaaccagtgaacgctaccgaggaccccagcagcggctactattccactaccatcagataccaggccacaggattnggcaccaatgagacagaatacctgtttgaagtggacaacctgacatatgtccagctggagtctaggttcactccccagtttctgctgcagctgaatgaaactatctataccagtggcaagcgctcaaatacaactgggaagctgatttggaaagtgaaccctgagatcgataccacaattggcgaatgggccttttgggagaccaagaaaaacctgacacggaagatcagaagcgaggaactgtccttcaccgcagtgagtaatagggccaaaaacatttcaggccagagcccagcacgaacttcctctgaccccgggaccaatactaccacagaagatcacaagatcatggccagcgagaacagttcagctatggtgcaggtccactcccagggaagggaggcagccgtgtctcatctgactaccctggccacaatctctactagtccccagagccccacaactaagcccgggcctgacaatagcacccataacacacctgtgtacaaactggatatctccgaagccacccaggtcgagcagcaccatcggagaacagacaatgattccactgcatctgacacccctccagcaaccacagctgcaggaccccccaaggctgagaatactaacaccagcaaaagcaccgacctgctggaccccgcaactaccacatcaccacagaaccacagcgagacagccgggaacaataacactcaccatcaggacaccggagaggaatccgccagctccggcaagctggggctgatcacaaatactattgctggagtggcaggactgatcacaggcgggaggngaactcgacgagaagctattgtgaacgcacagcccaaatgcaatcctaacctgcactattggactacccaggacgagggagcagctatcggactggcatggattccatactttgggcccgcagccgaaggaatctataccgagggcctgatgcataatcaggatggactgatctgtggcctgcggcagctggctaacgaaacaactcaggcactgcagctgttcctgcgagctaccacagagctgcggacctttagcatnctgaatcgcaaggcaattgacttcctgctgcagcgatggggaggcacatnccacatcctgggaccagactgctgtattgagcctcatgattggacaaagaacatcactgacaaaattgatcagatcattcacgacttcgtggataaaacactgccagatcagggggacaatgataactggtggactggatggagacagtggattcccgccggcattggcgtcaccggcgtcattattgccgtcattgctctgttctgtatttgtaagttcgtgttctgataa

SEQ ID NO: 51 - protein, 676 aa, Ebola virus
  MISC_FEATURE (225)..(225): Xaa is unknown
  MISC_FEATURE (498)..(498): Xaa is unknown
  MISC_FEATURE (584)..(584): Xaa is unknown
  MISC_FEATURE (601)..(601): Xaa is unknown
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGXGTNETEYLFEVDNLTYVQLESRFTPQFLLQLNETIYTSGKRSNTTGKLIWKVNPEIDTTIGEWAFWETKKNLTRKIRSEELSFTAVSNRAKNISGQSPARTSSDPGTNTTTEDHKIMASENSSAMVQVHSQGREAAVSHLTTLATISTSPQSPTTKPGPDNSTHNTPVYKLDISEATQVEQHHRRTDNDSTASDTPPATTAAGPPKAENTNTSKSTDLLDPATTTSPQNHSETAGNNNTHHQDTGEESASSGKLGLITNTIAGVAGLITGGRXTRREAIVNAQPKCNPNLHYWTTQDEGAAIGLAWIPYFGPAAEGIYTEGLMHNQDGLICGLRQLANETTQALQLFLRATTELRTFSXLNRKAIDFLLQRWGGTXHILGPDCCIEPHDWTKNITDKIDQIIHDFVDKTLPDQGDNDNWWTGWRQWIPAGIGVTGVIIAVIALFCICKFVF

SEQ ID NO: 52 - DNA, 2034 bp, Ebola virus
  misc_feature (202)..(202): n is either a or t or g or c
  misc_feature (422)..(422): n is either a or t or g or c
  misc_feature (510)..(510): n is either a or t or g or c
  misc_feature (720)..(720): n is either a or t or g or c
  misc_feature (1042)..(1042): n is either a or t or g or c
atggagggactgtcactgctgcagctgcctagagataagttcaggaaaagctccttctttgtgtgggtcatcattctgttccagaaggccttttcaatgcccctgggcgtggtcactaatagcaccctggaagtgacagagatcgatcagctggtctgtaaggaccacctggcttcaactgatcagctgaaaagcgtggggntgaacctggagggatcaggcgtcagcactgatattccttctgcaaccaagagatggggatttcgcagcggagtgccccctaaagtggtctcctacgaagcaggggagtgggccgaaaattgctataacctggagatcaagaaaccagatggcagcgaatgtctgccaccccctccagacggggtgcgcggattccccagatgcagatacgtccacaaggnccaggggaccggaccttgtccaggagactatgcctttcataaagatggcgctttctttctgtacgaccgcctggctagtacagtgatntatcgaggcgtcaatttcgccgagggcgtgatcgcttttctgattctggcaaagccaaaagaaaccttcctgcagagccctcccattagggaggccgtgaactacacagaaaacacttctagttactacgctacatcctacctggagtatgaaatcgagaactttggcgctcagcactctaccacactgttcaagattaacaataacacntttgtgctgctggatcgccctcatacaccacagttcctgtttcagctgaacgacactatccacctgcatcagcagctgagcaatactaccggaaaactgatttggacactggacgctaatatcaacgcagatattggcgagtgggccttctgggaaaataagaaaaacctgtccgagcagctgcggggagaggaactgagctttgaaacactgtccctgaatgaaactgaggacgatgacgccacctcaagccgaacaactaagggccggatctctgatcgggctaccagaaagtacagtgatctggtgccaaaagactctnccggcatggtgagtctgcacgtccctgaaggggagaccacactgccatcccagaactctactgagggccggagagtggacgtcaatacccaggagactatcaccgaaactaccgcaacaatcattggcactaacgggaataacatgcagatcagcaccattggcacagggctgtcctctagtcagattctgtcaagctcccctaccatggccccctcccctgagacacagacttctacaacttatacacccaagctgcctgtgatgaccacagaggaacccactaccccacccagaaacagtcctgggtcaacaactgaggcacccaccctgaccacacctgaaaatatcactaccgccgtgaaaacagtcctgcctcaggagtctactagtaacggactgatcaccagcacagtgactggaattctgggcagtctggggctgcgcaagcgatcaaggcgccaagtgaatactcgggctaccggcaaatgcaatccaaacctgcactactggaccgcacaggagcagcataacgccgctgggatcgcttggattccttacttcggaccaggcgcagaggggatctataccgaaggactgatgcataatcagaacgccctggtgtgtggcctgagacagctggcaaatgagacaactcaggccctgcagctgttcctgagagcaaccacagaactgaggacctatacaatcctgaaccggaaggccattgattttctgctgcgacgatggggcgggacctgcagaatcctgggaccagactgctgtattgagccccacgattggaccaagaacatcacagacaagatcaaccagatcattcatgatttcatcgacaacccactgcccaatcaggacaacgatgacaattggtggaccggatggcgacagtggattcccgcaggaattggaatcaccggaattattattgccattattgctctgctgtgtgtctgtaagctgctgtgttgataa

SEQ ID NO: 53 - protein, 676 aa, Ebola virus
  MISC_FEATURE (68)..(68): Xaa is unknown
  MISC_FEATURE (141)..(141): Xaa is unknown
  MISC_FEATURE (170)..(170): Xaa is unknown
  MISC_FEATURE (240)..(240): Xaa is unknown
  MISC_FEATURE (348)..(348): Xaa is unknown
MEGLSLLQLPRDKFRKSSFFVWVIILFQKAFSMPLGVVTNSTLEVTEIDQLVCKDHLASTDQLKSVGXNLEGSGVSTDIPSATKRWGFRSGVPPKVVSYEAGEWAENCYNLEIKKPDGSECLPPPPDGVRGFPRCRYVHKXQGTGPCPGDYAFHKDGAFFLYDRLASTVXYRGVNFAEGVIAFLILAKPKETFLQSPPIREAVNYTENTSSYYATSYLEYEIENFGAQHSTTLFKINNNXFVLLDRPHTPQFLFQLNDTIHLHQQLSNTTGKLIWTLDANINADIGEWAFWENKKNLSEQLRGEELSFETLSLNETEDDDATSSRTTKGRISDRATRKYSDLVPKDSXGMVSLHVPEGETTLPSQNSTEGRRVDVNTQETITETTATIIGTNGNNMQISTIGTGLSSSQILSSSPTMAPSPETQTSTTYTPKLPVMTTEEPTTPPRNSPGSTTEAPTLTTPENITTAVKTVLPQESTSNGLITSTVTGILGSLGLRKRSRRQVNTRATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNQDNDDNWWTGWRQWIPAGIGITGIIIAIIALLCVCKLLC

SEQ ID NO: 54 - DNA, 2049 bp, Marburg virus
  misc_feature (21)..(21): n is either a or t or g or c
atgaaaaccacttgtctgctnatctcactgattctgattcagggcgtcaaaacactgcccattctggaaattgcctctaacatccagccacagaacgtggactccgtctgttctgggaccctgcagaagacagaggatgtgcacctgatgggcttcaccctgagcgggcagaaggtcgcagactcacccctggaagccagcaaacgatgggcatttcgggccggagtgccccctaagaacgtcgagtacaccgaaggcgaggaagccaaaacatgctataatatctccgtgactgatcctagtggcaagtcactgctgctggacccacccaccaacattagggattaccctaagtgtaaaacaatccaccatattcagggccagaatccacacgctcaggggatcgcactgcatctgtggggagccttctttctgtacgacaggattgctagcaccacaatgtatcgcgggaaagtgttcaccgagggaaacatcgccgctatgattgtgaataagacagtccacaaaatgatcttttctcgccagggccaggggtaccgacatatgaacctgaccagtacaaataagtattggaccagctccaacggcactcagaccaatgacactgggtgcttcggaaccctgcaggagtacaacagtactaaaaatcagacctgtgctccatcaaagaaaccactgccactgcctaccgcacacccagaggtgaagctgacaagtacttcaaccgacgccacaaaactgaacactaccgaccccaatagtgacgatgaagatctgacaactagcggatccggctctggggagcaggaaccttataccacatccgatgcagccaccaagcagggcctgtctagtacaatgcctccaactccatctccccagcctagtactccccagcagggcgggaacaataccaaccattcccagggcgtggtcacagagccagggaagactaacactaccgcccagccctctatgccccctcacaatacaactaccatctccaccaacaatacatctaaacataacctgagcacaccttccgtgccaatccagaacgctactaactacaacactcagtctaccgcacccgagaatgaacagacttctgcccctagtaagacaactctgctgcccaccgagaaccctaccacagccaagtcaacaaatagcactaaatcccctactaccacagtgccaaacactaccaataagtacagtacctcaccaagccccacccctaactccacagcacagcacctggtctatttccggagaaaaagaaatatcctgtggagggagggcgacatgttcccttttctggatgggctgatcaacgctccaattgacttcgatccagtgcccaatacaaagactatctttgacgaatcaagctcctctggcgcctctgctgaggaagatcagcacgcctcacccaacattagcctgacactgtcctactttcctaaagtgaacgagaatactgcccatagcggggagaacgaaaatgactgcgatgctgagctgcggatctggagcgtccaggaagacgatctggctgcaggactgtcctggatcccattctttggacccggcattgagggactgtataccgccggcctgattaagaaccagaacaacctggtgtgcagactgaggcgcctggccaatcagaccgctaaatcactggaactgctgctgcgggtcacaactgaggaaagaacattcagcctgatcaaccgacatgctattgactttctgctggcacgctggggaggcacctgcaaggtgctgggaccagactgctgtatcggcattgaggatctgtctcgcaatatcagtgaacagatcgaccagattaagaaagatgagcagaaggaaggaaccggatggggactgggcggcaagtggtggaccagcgattggggcgtgctgacaaacctgggaatcctgctgctgctgtccatcgccgtcctgattgctctgtcctgtatttgtcggattttcactaagtatattgggtgataa

SEQ ID NO: 55 - protein, 681 aa, Marburg virus
  MISC_FEATURE (7)..(7): Xaa is unknown
  MISC_FEATURE (200)..(200): Xaa is unknown
MKTTCLXISLILIQGVKTLPILEIASNIQPQNVDSVCSGTLQKTEDVHLMGFTLSGQKVADSPLEASKRWAFRAGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTXSNGTQTNDTGCFGTLQEYNSTKNQTCAPSKKPLPLPTAHPEVKLTSTSTDATKLNTTDPNSDDEDLTTSGSGSGEQEPYTTSDAATKQGLSSTMPPTPSPQPSTPQQGGNNTNHSQGVVTEPGKTNTTAQPSMPPHNTTTISTNNTSKHNLSTPSVPIQNATNYNTQSTAPENEQTSAPSKTTLLPTENPTTAKSTNSTKSPTTTVPNTTNKYSTSPSPTPNSTAQHLVYFRRKRNILWREGDMFPFLDGLINAPIDFDPVPNTKTIFDESSSSGASAEEDQHASPNISLTLSYFPKVNENTAHSGENENDCDAELRIWSVQEDDLAAGLSWIPFFGPGIEGLYTAGLIKNQNNLVCRLRRLANQTAKSLELLLRVTTEERTFSLINRHAIDFLLARWGGTCKVLGPDCCIGIEDLSRNISEQIDQIKKDEQKEGTGWGLGGKWWTSDWGVLTNLGILLLLSIAVLIALSCICRIFTKYIG

SEQ ID NO: 56 - DNA, 2999 bp, Artificial Sequence
  pVAX1 backbone sequence
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 57 - DNA, 1221 bp, Artificial Sequence
  Influenza Construct 1 - NC99_60mer_pVax (DLnano_LS_HA(NC99)
ggatccgccaccatggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgcccctctgcagctgggaaactgctccgtggcaggatggattctgggcaatccagagtgtgagctgctgatctctaaggagtcctggtcttacatcgtggagaccccaaaccccgagaatggcacatgctttcccggctacttcgccgactatgaggagctgagggagcagctgagctccgtgtctagcttcgagagatttgagatcttccctaaggagtcctcttggccaaaccacaccgtgacaggcgtgagcgcctcctgttctcacaacggcaagagctccttttataggaatctgctgtggctgaccggcaagaacggcctgtaccctaatctgagcaagtcctatgtgaacaataaggagaaggaggtgctggtgctgtggggcgtgcaccaccctcccaacatcggcaatcagagggccctgtaccacaccgagaacgcctacgtgagcgtggtgtctagccactacagcaggagattcacacccgagatcgccaagaggcctaaggtgcgcgaccaggagggacggatcaattactattggaccctgctggagccaggcgatacaatcatctttgaggccaacggcaatctgatcgccccctggtatgccttcgccctgtcccgcggctgataactcgag

SEQ ID NO: 58 - protein, 399 aa, Artificial Sequence
  Expressed self assembly viral antigen NC99_60mer_pVax
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRG

SEQ ID NO: 59 - DNA, 1230 bp, Artificial Sequence
  NC99_g6_60mer_pVax
ggatccgccaccatggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgcccctctgcagctgggaaactgcagcgtggcaggatggattctgggcaatccagagtgtgagctgctgatctccaaggagtcctggtcttacatcgtggagaccccaaaccccgagaatggcacatgctttcccggcaacttctctgactatgaggagctgagggagcagctgagctccgtgtctagcttcgagagatttgagatcttccctaaggagtcctcttggccaaatcacaccgtgacaggcgtgagcgcctcctgttctcacaacggcaagagctccttttacaggaatctgctgtggctgaccggcaagaacggcctgtaccctaatctgagcaagtcctataacaatacaaaggagaaggaggtgctggtgctgtggggcgtgcaccaccctcccaacatcggcaatcagagggccctgtaccacaccgagaacgcctacgtgagcgtggtgtctagccactactctaggagattcacacccaacatcagcaagaggcctaaggtgcgcgaccaggagggacggatcaattactattggaccctgctggagccaggcgatacaatcatctttgaggccaacggcaatctgatcgccccctggtatgccttcgccctgtctcgcggcaacggcagctgataactcgag

SEQ ID NO: 60 - protein, 402 aa, Artificial Sequence
  Expressed self assembly viral antigen NC99_g6_60mer_pVax
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGAPLQLGNCSVAGWILGNPECELLISKESWSYIVETPNPENGTCFPGNFSDYEELREQLSSVSSFERFEIFPKESSWPNHTVTGVSASCSHNGKSSFYRNLLWLTGKNGLYPNLSKSYNNTKEKEVLVLWGVHHPPNIGNQRALYHTENAYVSVVSSHYSRRFTPNISKRPKVRDQEGRINYYWTLLEPGDTIIFEANGNLIAPWYAFALSRGNGS

SEQ ID NO: 61 - DNA, 1227 bp, Artificial Sequence
  CA09(175L)_Ferritin_pVax
atggactggacttggattctgtttctggtcgccgctgccactcgcgtgcattctgccccactgcacctgggcaagtgcaacatcgccggctggattctgggcaatcccgagtgtgagagcctgtccaccgccagctcctggagctacatcgtggagaccccttctagcgacaacggcacatgctttccaggcgacttcatcgattatgaggagctgagggagcagctgtcctctgtgagctccttcgagagatttgagatcttccccaagacctctagctggcctaaccacgattccaataagggagtgacagcagcatgtcctcacgcaggcgccaagagcttttacaagaacctgatctggctggtgaagaagggcaattcctacccaaagctgtctaagagctatatcaacgacaagggcaaggaggtgctggtgctgtggggcatccaccacccatccacctctgccgaccagcagtctctgtaccagaatgccgatacatacgtgttcgtgggctcctctcggtactccaagaagttcaagccagagatcgccatcaggcccaaggtgagagaccaggagggccgcatgaattactattggacactggtggagcccggcgataagatcacctttgaggccacaggcaacctggtggtgcctcggtatgccttcgccatggagcgcaatgcaagcggggaaagccaggtgcgacagcagttctccaaagacatcgaaaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcaggaagc

SEQ ID NO: 62 - protein, 409 aa, Artificial Sequence
  Expressed self assembly viral antigen CA09(175L)_Ferritin_pVax
MDWTWILFLVAAATRVHSAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 63 - DNA, 2112 bp, Artificial Sequence
  H1_CA04/09_FL_HA_3BVE_pVAX
atggactggacttggattctgttcctggtcgccgccgcaacccgcgtgcattctatgaaggctattctggtcgtgctgctgtatactttcgccaccgccaacgccgacacactgtgcatcggctaccacgccaacaattctaccgacacagtggataccgtgctggagaagaatgtgaccgtgacacacagcgtgaacctgctggaggataagcacaatggcaagctgtgcaagctgaggggagtggcaccactgcacctgggcaagtgcaacatcgccggctggattctgggcaatcccgagtgtgagtccctgtctacagccagctcctggtcctacatcgtggagacaccctctagcgacaacggcacatgctaccctggcgactttatcgattatgaggagctgcgggagcagctgagcagcgtgagcagcttcgagaggttcgagatcttccccaagacctctagctggcctaaccacgatagcaataagggagtgacagcagcatgtccacacgcaggcgccaagagcttctataagaacctgatctggctggtgaagaagggcaattcctaccctaagctgagcaagtcctatatcaacgacaagggcaaggaggtgctggtgctgtggggcatccaccacccatctaccagcgccgaccagcagtccctgtaccagaatgccgatacatacgtgttcgtgggctcctctcggtactctaagaagttcaagccagagatcgccatcaggccaaaggtgagggaccaggagggacgcatgaactactattggaccctggtggagcccggcgataagatcacctttgaggccacaggcaacctggtggtgcctagatatgccttcgccatggagagaaatgccggctccggcatcatcatctctgacacccctgtgcacgattgcaacaccacatgtcagaccccaaagggcgccatcaacacatccctgccttttcagaatatccacccaatcacaatcggcaagtgccctaagtacgtgaagagcaccaagctgaggctggcaacaggcctgcgcaatatcccatctatccagagcaggggcctgtttggagcaatcgcaggcttcatcgagggaggatggaccggaatggtggacggctggtacggctatcaccaccagaacgagcagggcagcggatatgcagcagacctgaagtccacccagaatgccatcgatgagatcacaaacaaggtcaattccgtgatcgagaagatgaacacccagtttacagccgtgggcaaggagttcaatcacctggagaagagaatcgagaacctgaataagaaggtggacgatggcttcctggacatctggacctacaacgccgagctgctggtgctgctggagaatgagaggacactggactaccacgattccaacgtgaagaatctgtatgagaaggtgagatctcagctgaagaacaatgccaaggagatcggcaacggctgcttcgagttttaccacaagtgcgacaacacctgtatggagagcgtgaagaatggcacatacgattatcctaagtattccgaggaggccaagctgaaccgcgaggagatcgactctggcggcgatatcatcaagctgctgaacgagcaagtgaataaggagatgcagagctccaatctgtacatgtctatgtctagctggtgttatacccacagcctggacggagcaggcctgttcctgtttgatcacgccgccgaggagtacgagcacgccaagaagctgatcatctttctgaacgagaacaatgtgccagtgcagctgacctccatctctgcccccgagcacaagtttgagggcctgacacagatcttccagaaggcctacgagcacgagcagcacatcagcgagtccatcaacaatatcgtggaccacgccatcaagagcaaggatcacgccaccttcaactttctgcagtggtacgtggccgagcagcacgaggaggaggtgctgttcaaggacatcctggataagatcgagctgatcggcaacgagaatcacgggctgtacctggcagaccagtatgtcaagggcatcgcaaagtcacggaagagcgggagc

SEQ ID NO: 64 - protein, 704 aa, Artificial Sequence
  Expressed self assembly viral antigen H1_CA04/09_FL_HA_3BVE_pVAX
MDWTWILFLVAAATRVHSMKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 65 - protein, 327 aa, Influenza A virus
FTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIH

SEQ ID NO: 66 - protein, 566 aa, Influenza A virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 67 - protein, 566 aa, Influenza A virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 68 - DNA, 1620 bp, Artificial Sequence
  GT8_180mer_pVAX (DLnano_PfV_GT8)
ctgagcattgcccccacactgattaaccgggacaaaccctacaccaaagaggaactgatggagattctgagactggctattatcgctgagctggacgccatcaacctgtacgagcagatggcccggtattctgaggacgagaatgtgcgcaagatcctgctggatgtggccagggaggagaaggcacacgtgggagagttcatggccctgctgctgaacctggaccccgagcaggtgaccgagctgaagggcggctttgaggaggtgaaggagctgacaggcatcgaggcccacatcaacgacaataagaaggaggagagcaacgtggagtatttcgagaagctgagatccgccctgctggatggcgtgaataagggcaggagcctgctgaagcacctgcctgtgaccaggatcgagggccagagcttcagagtggacatcatcaagtttgaggatggcgtgcgcgtggtgaagcaggagtacaagcccatccctctgctgaagaagaagttctacgtgggcatcagggagctgaacgacggcacctacgatgtgagcatcgccacaaaggccggcgagctgctggtgaaggacgaggagtccctggtcatccgcgagatcctgtctacagagggcatcaagaagatgaagctgagctcctgggacaatccagaggaggccctgaacgatctgatgaatgccctgcaggaggcatctaacgcaagcgccggaccattcggcctgatcatcaatcccaagagatacgccaagctgctgaagatctatgagaagtccggcaagatgctggtggaggtgctgaaggagatcttccggggcggcatcatcgtgaccctgaacatcgatgagaacaaagtgatcatctttgccaacacccctgccgtgctggacgtggtggtgggacaggatgtgacactgcaggagctgggaccagagggcgacgatgtggcctttctggtgtccgaggccatcggcatcaggatcaagaatccagaggcaatcgtggtgctggagggcggctctggcggaagtggcggaagtgggggaagtggaggcggcggaagcgggggaggcagcgggggaggggacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccacc

SEQ ID NO: 69 - protein, 540 aa, Artificial Sequence
  Expressed self assembly viral antigen GT8_180mer_pVAX (DLnano_PfV_GT8)
LSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGGSGGSGGSGGSGGGGSGGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNST

SEQ ID NO: 70 - DNA, 1146 bp, Artificial Sequence
  LS_HA_CA09 (DLnano_CD4MutLS_GT8)
atgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgccccactgcacctgggcaagtgcaacatcgccggctggattctgggcaatcccgagtgtgagagcctgtccaccgccagctcctggagctacatcgtggagaccccttctagcgacaacggcacatgctttccaggcgacttcatcgattatgaggagctgagggagcagctgtcctctgtgagctccttcgagagatttgagatcttccccaagacctctagctggcctaaccacgattccaataagggagtgacagcagcatgtcctcacgcaggcgccaagagcttttacaagaacctgatctggctggtgaagaagggcaattcctacccaaagctgtctaagagctatatcaacgacaagggcaaggaggtgctggtgctgtggggcatccaccacccatccacctctgccgaccagcagtctatctaccagaatgccgatacatacgtgttcgtgggctcctctcggtactccaagaagttcaagccagagatcgccatcaggcccaaggtgagagaccaggagggccgcatgaattactattggacactggtggagcccggcgataagatcacctttgaggccacaggcaacctggtggtgcctcggtatgccttcgccatggagcgcaat

SEQ ID NO: 71 - protein, 382 aa, Artificial Sequence
  Expressed self assembly viral antigen LS_HA_CA09 (DLnano_CD4MutLS_GT8)
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGAPLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSIYQNADTYVFVGSSRYSKKFKPEIAIRPKVRDQEGRMNYYWTLVEPGDKITFEATGNLVVPRYAFAMERN

SEQ ID NO: 72 - DNA, 780 bp, Artificial Sequence
  GT8_7mer-pVAX (DL_GT8_IMX313P)
atggactggacctggattctgttcctggtggccgccgccacaagggtgcacagcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccaccggcgggagcggagggagtggcggatctggcgggagcggcggaggcaaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaa

SEQ ID NO: 73 - protein, 261 aa, Artificial Sequence
  Expressed GT8_7mer-pVAX (DL_GT8_IMX313P)
MDWTWILFLVAAATRVHSMDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNSTGGSGGSGGSGGSGGGKKQGDADVCGEVAYIQSVVSDCHVPTAELRTLLEIRKLFLEIQKLKVELQGLSKE

SEQ ID NO: 74 - DNA, 1101 bp, Artificial Sequence
  GT8_CD4Mut1LS-60mer (DLnano_CD4MutLS_GT8)
ggatccgccaccatggactggacctggattctgttcctggtggccgccgccacaagggtgcacagcatgcagatctacgaaggaaaactgaccgctgagggactgaagttcggcatcgtgggcagcaggtttaaccacggcctggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccacctgataactcgag

SEQ ID NO: 75 - protein, 359 aa, Artificial Sequence
  Expressed GT8_CD4Mut1LS-60mer (DLnano_CD4MutLS_GT8)
MDWTWILFLVAAATRVHSMQIYEGKLTAEGLKFGIVGSRFNHGLVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNST

SEQ ID NO: 76 - DNA, 5428 bp, Artificial Sequence
  pcDNA3.1(+) backbone
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 77 - DNA, 4006 bp, Artificial Sequence
  pCI mammalian expression vector backbone
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 78 - DNA, 3632 bp, Artificial Sequence
  pSI vector backbone
gcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 79 - DNA, 3515 bp, Artificial Sequence
  pZeoSV2(+) backbone
ggatcgatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgaactagatctagagtccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacggaccgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacactcgacctcgaaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 80 - DNA, 4239 bp, Artificial Sequence
  phCMV1 backbone
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaattaatacgactcactatagggagacagactgttcctttcctgggtcttttctgcaggcaccgtcgtcgacttaacagatctcgagctcaagcttcgaattctgcagtcgacggtaccgcgggcccgggatccaccgggtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 81 - DNA, 4930 bp, Artificial Sequence
  pTCP backbone
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtggatcccgtcgcttaccgattcagaatggttgatatccgccattctgaatcggtaagcgacgaagcttaataaaggatcttttattttcattggatctgtgtgttggttttttgtgtgcggccgccctcgactgtgccttctagaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggatgaccgagtacaagcccacggtgcgcctcgccacccgcgacgacgtcccgcgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgattcgaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgac

SEQ ID NO: 82 - DNA, 6105 bp, Artificial Sequence
  pIRES backbone
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtcgagcatgcatctagggcggccaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 83 - DNA, 1029 bp, Artificial Sequence
  LS_GT8 without IgE
atgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccacctgataa

SEQ ID NO: 84 - protein, 341 aa, Artificial Sequence
  Expressed LS_GT8 without IgE
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNST

SEQ ID NO: 85 - DNA, 1050 bp, Artificial Sequence
  IgE-GLT1-3BVE without IgE
gacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccaccggcggaagcggcggaagcggcgggtctgggctgagtaaggacattatcaagctgctgaacgaacaggtgaacaaagagatgcagtctagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaatgagaaccacgggctgtacctggcagatcagtatgtcaagggcatcgctaagtcaaggaaaagctgataa

SEQ ID NO: 86 - protein, 348 aa, Artificial Sequence
  Expressed IgE-GLT1-3BVE without IgE
DTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNSTGGSGGSGGSGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 87 - DNA, 1203 bp, Artificial Sequence
  IgE-GLT1-I3 without IgE
gacaccatcacactgccatgccgccctgcaccacctccacattgtagctccaacatcaccggcctgattctgacaagacaggggggatatagtaacgataataccgtgattttcaggccctcaggaggggactggagggacatcgcacgatgccagattgctggaacagtggtctctactcagctgtttctgaacggcagtctggctgaggaagaggtggtcatccgatctgaagactggcgggataatgcaaagtcaatttgtgtgcagctgaacacaagcgtcgagatcaattgcactggcgcagggcactgtaacatttctcgggccaaatggaacaataccctgaagcagatcgccagtaaactgagagagcagtacggcaataagacaatcatcttcaagccttctagtggaggcgacccagagttcgtgaaccatagctttaattgcgggggagagttcttttattgtgattccacacagctgttcaacagcacttggtttaattccaccggcggcagcggcggcagcggcgggagcggaggaagtgagaaagcagccaaagcagaggaagcagcacggaagatggaagaactgttcaagaagcacaagatcgtggccgtgctgagggccaactccgtggaggaggccaagaagaaggccctggccgtgttcctgggcggcgtgcacctgatcgagatcacctttacagtgcccgacgccgataccgtgatcaaggagctgtctttcctgaaggagatgggagcaatcatcggagcaggaaccgtgacaagcgtggagcagtgcagaaaggccgtggagagcggcgccgagtttatcgtgtcccctcacctggacgaggagatctctcagttctgtaaggagaagggcgtgttttacatgccaggcgtgatgacccccacagagctggtgaaggccatgaagctgggccacacaatcctgaagctgttccctggcgaggtggtgggcccacagtttgtgaaggccatgaagggccccttccctaatgtgaagtttgtgcccaccggcggcgtgaacctggataacgtgtgcgagtggttcaaggcaggcgtgctggcagtgggcgtgggcagcgccctggtgaagggcacacccgtggaagtcgctgagaaggcaaaggcattcgtggaaaagattagggggtgtactgagtgataa

SEQ ID NO: 88 - protein, 399 aa, Artificial Sequence
  Expressed IgE-GLT1-I3 without IgE
DTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNSTGGSGGSGGSGGSEKAAKAEEAARKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 89 - DNA, 1200 bp, Artificial Sequence
  NivFtop_stab2_gMax_Nt_60mer without IgE
atgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcggggtcacttgtgccggacgagccatcggaaatgctaccgccgcccagattactgccggagtcgccctgtatgaagccatgaagaatgccgacaacatcaataagctgaagagctccatcgagagcaccaacgaggccgtggtgaagctgcaggagacagccgagaagacagtgtacgtgctgacagccctgcaggactatatcaacaccaatctggtgcccacaatcgataagatcagctgcaagcagaccgaggcatccctggacgccgccctgtccaagtacctgtctgatctgctgtacgtgttcggccccaacctgagcgaccccgtgagcaattctatgcctatccaggccatctctcaggccttcggcggcaactacagcaccctgctgaggacactgggctatgccccagaggactttgacgatctgctggagagcgattccatcacaggccagatcatctacgtggacctgtctagctactatatcatcgtgagagtgtattttccaaatggctccggccccctgaccaaggatatcgtgatcaagatgatccccaacgtgtctaatatgagccagtgtacaggctctgtgatggagaactacaagaccaggctgaatggcatcctgacacctatcaagggcgccctggagatctataagaataactgtcacgatggatgataa

SEQ ID NO: 90 - protein, 398 aa, Artificial Sequence
  Expressed NivFtop_stab2_gMax_Nt_60mer without IgE
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGGVTCAGRAIGNATAAQITAGVALYEAMKNADNINKLKSSIESTNEAVVKLQETAEKTVYVLTALQDYINTNLVPTIDKISCKQTEASLDAALSKYLSDLLYVFGPNLSDPVSNSMPIQAISQAFGGNYSTLLRTLGYAPEDFDDLLESDSITGQIIYVDLSSYYIIVRVYFPNGSGPLTKDIVIKMIPNVSNMSQCTGSVMENYKTRLNGILTPIKGALEIYKNNCHDG

SEQ ID NO: 91 - DNA, 1200 bp, Artificial Sequence
  NivFtop_stab2_gMax_Ct_60mer without IgE
ggggtcacttgtgccggacgagccatcggaaatgctaccgccgcccagattactgccggagtcgccctgtatgaagccatgaagaatgccgacaacatcaataagctgaagagctccatcgagagcaccaacgaggccgtggtgaagctgcaggagacagccgagaagacagtgtacgtgctgacagccctgcaggactatatcaacaccaatctggtgcccacaatcgataagatcagctgcaagcagaccgaggcatccctggacgccgccctgtccaagtacctgtctgatctgctgtacgtgttcggccccaacctgagcgaccccgtgagcaattctatgcctatccaggccatctctcaggccttcggcggcaactacagcaccctgctgaggacactgggctatgccccagaggactttgacgatctgctggagagcgattccatcacaggccagatcatctacgtggacctgtctagctactatatcatcgtgagagtgtattttccaaatggctccggccccctgaccaaggatatcgtgatcaagatgatccccaacgtgtctaatatgagccagtgtacaggctctgtgatggagaactacaagaccaggctgaatggcatcctgacacctatcaagggcgccctggagatctataagaataactgtcacgatggaggaggctccggaggatctggagggagtggaggctcaggaggaggcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgatgataa

SEQ ID NO: 92 - protein, 398 aa, Artificial Sequence
  Expressed NivFtop_stab2_gMax_Ct_60mer without IgE
GVTCAGRAIGNATAAQITAGVALYEAMKNADNINKLKSSIESTNEAVVKLQETAEKTVYVLTALQDYINTNLVPTIDKISCKQTEASLDAALSKYLSDLLYVFGPNLSDPVSNSMPIQAISQAFGGNYSTLLRTLGYAPEDFDDLLESDSITGQIIYVDLSSYYIIVRVYFPNGSGPLTKDIVIKMIPNVSNMSQCTGSVMENYKTRLNGILTPIKGALEIYKNNCHDGGGSGGSGGSGGSGGGMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLR

SEQ ID NO: 93 - DNA, 1146 bp, Artificial Sequence
  NC99_60mer_pVax without IgE
atgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgcccctctgcagctgggaaactgctccgtggcaggatggattctgggcaatccagagtgtgagctgctgatctctaaggagtcctggtcttacatcgtggagaccccaaaccccgagaatggcacatgctttcccggctacttcgccgactatgaggagctgagggagcagctgagctccgtgtctagcttcgagagatttgagatcttccctaaggagtcctcttggccaaaccacaccgtgacaggcgtgagcgcctcctgttctcacaacggcaagagctccttttataggaatctgctgtggctgaccggcaagaacggcctgtaccctaatctgagcaagtcctatgtgaacaataaggagaaggaggtgctggtgctgtggggcgtgcaccaccctcccaacatcggcaatcagagggccctgtaccacaccgagaacgcctacgtgagcgtggtgtctagccactacagcaggagattcacacccgagatcgccaagaggcctaaggtgcgcgaccaggagggacggatcaattactattggaccctgctggagccaggcgatacaatcatctttgaggccaacggcaatctgatcgccccctggtatgccttcgccctgtcccgcggctga

SEQ ID NO: 94 - protein, 381 aa, Artificial Sequence
  Expressed NC99_60mer_pVax without IgE
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRG

SEQ ID NO: 95 - DNA, 1155 bp, Artificial Sequence
  NC99_g6_60mer_pVax without IgE
atgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcgcccctctgcagctgggaaactgcagcgtggcaggatggattctgggcaatccagagtgtgagctgctgatctccaaggagtcctggtcttacatcgtggagaccccaaaccccgagaatggcacatgctttcccggcaacttctctgactatgaggagctgagggagcagctgagctccgtgtctagcttcgagagatttgagatcttccctaaggagtcctcttggccaaatcacaccgtgacaggcgtgagcgcctcctgttctcacaacggcaagagctccttttacaggaatctgctgtggctgaccggcaagaacggcctgtaccctaatctgagcaagtcctataacaatacaaaggagaaggaggtgctggtgctgtggggcgtgcaccaccctcccaacatcggcaatcagagggccctgtaccacaccgagaacgcctacgtgagcgtggtgtctagccactactctaggagattcacacccaacatcagcaagaggcctaaggtgcgcgaccaggagggacggatcaattactattggaccctgctggagccaggcgatacaatcatctttgaggccaacggcaatctgatcgccccctggtatgccttcgccctgtctcgcggcaacggcagctga

SEQ ID NO: 96 - protein, 384 aa, Artificial Sequence
  Expressed NC99_g6_60mer_pVax without IgE
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGAPLQLGNCSVAGWILGNPECELLISKESWSYIVETPNPENGTCFPGNFSDYEELREQLSSVSSFERFEIFPKESSWPNHTVTGVSASCSHNGKSSFYRNLLWLTGKNGLYPNLSKSYNNTKEKEVLVLWGVHHPPNIGNQRALYHTENAYVSVVSSHYSRRFTPNISKRPKVRDQEGRINYYWTLLEPGDTIIFEANGNLIAPWYAFALSRGNGS

SEQ ID NO: 97 - DNA, 1173 bp, Artificial Sequence
  CA09(175L)_Ferritin_pVax without IgE
gccccactgcacctgggcaagtgcaacatcgccggctggattctgggcaatcccgagtgtgagagcctgtccaccgccagctcctggagctacatcgtggagaccccttctagcgacaacggcacatgctttccaggcgacttcatcgattatgaggagctgagggagcagctgtcctctgtgagctccttcgagagatttgagatcttccccaagacctctagctggcctaaccacgattccaataagggagtgacagcagcatgtcctcacgcaggcgccaagagcttttacaagaacctgatctggctggtgaagaagggcaattcctacccaaagctgtctaagagctatatcaacgacaagggcaaggaggtgctggtgctgtggggcatccaccacccatccacctctgccgaccagcagtctctgtaccagaatgccgatacatacgtgttcgtgggctcctctcggtactccaagaagttcaagccagagatcgccatcaggcccaaggtgagagaccaggagggccgcatgaattactattggacactggtggagcccggcgataagatcacctttgaggccacaggcaacctggtggtgcctcggtatgccttcgccatggagcgcaatgcaagcggggaaagccaggtgcgacagcagttctccaaagacatcgaaaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcaggaagc

SEQ ID NO: 98 - protein, 391 aa, Artificial Sequence
  Expressed CA09(175L)_Ferritin_pVax without IgE
APLHLGKCNIAGWILGNPECESLSTASSWSYIVETPSSDNGTCFPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 99 - DNA, 2058 bp, Artificial Sequence
  H1_CA04/09_FL_HA_3BVE_pVAX without IgE
atgaaggctattctggtcgtgctgctgtatactttcgccaccgccaacgccgacacactgtgcatcggctaccacgccaacaattctaccgacacagtggataccgtgctggagaagaatgtgaccgtgacacacagcgtgaacctgctggaggataagcacaatggcaagctgtgcaagctgaggggagtggcaccactgcacctgggcaagtgcaacatcgccggctggattctgggcaatcccgagtgtgagtccctgtctacagccagctcctggtcctacatcgtggagacaccctctagcgacaacggcacatgctaccctggcgactttatcgattatgaggagctgcgggagcagctgagcagcgtgagcagcttcgagaggttcgagatcttccccaagacctctagctggcctaaccacgatagcaataagggagtgacagcagcatgtccacacgcaggcgccaagagcttctataagaacctgatctggctggtgaagaagggcaattcctaccctaagctgagcaagtcctatatcaacgacaagggcaaggaggtgctggtgctgtggggcatccaccacccatctaccagcgccgaccagcagtccctgtaccagaatgccgatacatacgtgttcgtgggctcctctcggtactctaagaagttcaagccagagatcgccatcaggccaaaggtgagggaccaggagggacgcatgaactactattggaccctggtggagcccggcgataagatcacctttgaggccacaggcaacctggtggtgcctagatatgccttcgccatggagagaaatgccggctccggcatcatcatctctgacacccctgtgcacgattgcaacaccacatgtcagaccccaaagggcgccatcaacacatccctgccttttcagaatatccacccaatcacaatcggcaagtgccctaagtacgtgaagagcaccaagctgaggctggcaacaggcctgcgcaatatcccatctatccagagcaggggcctgtttggagcaatcgcaggcttcatcgagggaggatggaccggaatggtggacggctggtacggctatcaccaccagaacgagcagggcagcggatatgcagcagacctgaagtccacccagaatgccatcgatgagatcacaaacaaggtcaattccgtgatcgagaagatgaacacccagtttacagccgtgggcaaggagttcaatcacctggagaagagaatcgagaacctgaataagaaggtggacgatggcttcctggacatctggacctacaacgccgagctgctggtgctgctggagaatgagaggacactggactaccacgattccaacgtgaagaatctgtatgagaaggtgagatctcagctgaagaacaatgccaaggagatcggcaacggctgcttcgagttttaccacaagtgcgacaacacctgtatggagagcgtgaagaatggcacatacgattatcctaagtattccgaggaggccaagctgaaccgcgaggagatcgactctggcggcgatatcatcaagctgctgaacgagcaagtgaataaggagatgcagagctccaatctgtacatgtctatgtctagctggtgttatacccacagcctggacggagcaggcctgttcctgtttgatcacgccgccgaggagtacgagcacgccaagaagctgatcatctttctgaacgagaacaatgtgccagtgcagctgacctccatctctgcccccgagcacaagtttgagggcctgacacagatcttccagaaggcctacgagcacgagcagcacatcagcgagtccatcaacaatatcgtggaccacgccatcaagagcaaggatcacgccaccttcaactttctgcagtggtacgtggccgagcagcacgaggaggaggtgctgttcaaggacatcctggataagatcgagctgatcggcaacgagaatcacgggctgtacctggcagaccagtatgtcaagggcatcgcaaagtcacggaagagcgggagc

SEQ ID NO: 100 - protein, 686 aa, Artificial Sequence
  Expressed H1_CA04/09_FL_HA_3BVE_pVAX without IgE
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 101 - protein, 20 aa, Artificial Sequence
  Leader sequence
MRRMQLLLLIALSLALVTNS

SEQ ID NO: 102 - DNA, 1822 bp, Artificial Sequence
  X68550.1 H.sapiens TRAP mRNA for ligand of CD40
catgctgcctctgccaccttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggacgtgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacatt

SEQ ID NO: 103 - protein, 261 aa, Artificial Sequence
  P29965|CD40L_HUMAN CD40 ligand OS=Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 104 - protein, 211 aa, Artificial Sequence
  a fragment of the human CD40 ligand
DKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 105 - protein, 149 aa, Artificial Sequence
  a fragment of the human CD40 ligand
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 106 - protein, 140 aa, Artificial Sequence
  a fragment of the human CD40 ligand
IAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 107 - DNA, 1250 bp, Artificial Sequence
  NM_011616.2 Mus musculus CD40 ligand (Cd40lg), mRNA
ctttcagtcagcatgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctgaacagtgcgctgtcctaggctgcagcagggctgatgctggcagtcttccctatacagcaagtcagttaggacctgccctgtgttgaactgcctatttataaccctaggatcctcctcatggagaactatttattatgtacccccaaggcacatagagctggaataagagaattacagggcaggcaaaaatcccaagggaccctgctccctaagaacttacaatctgaaacagcaaccccactgattcagacaaccagaaaagacaaagccataatacacagatgacagagctctgatgaaacaacagataactaatgagcacagttttgttgttttatgggtgtgtcgttcaatggacagtgtacttgacttaccagggaagatgcagaagggcaactgtgagcctcagctcacaatctgttatggttgacctgggctccctgcggccctagtagg

SEQ ID NO: 108 - protein, 260 aa, Artificial Sequence
  P27548|CD40L_MOUSE CD40 ligand OS=Mus musculus
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 109 - protein, 210 aa, Artificial Sequence
  mouse CD40 ligand
DKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 110 - protein, 149 aa, Artificial Sequence
  mouse CD40 ligand
MQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 111 - protein, 140 aa, Artificial Sequence
  mouse CD40 ligand
IAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 112 - protein, 377 aa, Artificial Sequence
  Expressed gp100_25_GT60mer (DLnano_LS_Gp100_25)
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGGSEGPRNQDWL

SEQ ID NO: 113 - DNA, 1131 bp, Artificial Sequence
  gp100_25_GT60mer (DLnano_LS_Gp100_25)
atggactggacctggatcctgttcctggtggccgccgccaccagggtgcacagcatgcagatctacgagggcaagctgaccgccgagggcctgaggttcggcatcgtggccagcagggccaaccacgccctggtggacaggctggtggagggcgccatcgacgccatcgtgaggcacggcggcagggaggaggacatcaccctggtgagggtgtgcggcagctgggagatccccgtggccgccggcgagctggccaggaaggaggacatcgacgccgtgatcgccatcggcgtgctgtgcaggggcgccacccccagcttcgactacatcgccagcgaggtgagcaagggcctggccgacctgagcctggagctgaggaagcccatcaccttcggcgtgatcaccgccgacaccctggagcaggccatcgaggccgccggcacctgccacggcaacaagggctgggaggccgccctgtgcgccatcgagatggccaacctgttcaagagcctgaggggcggcagcggcggcagcggcggcagcggcggcagcggcggcggcgacaccatcaccctgccctgcaggcccgccccccccccccactgcagcagcaacatcaccggcctgatcctgaccaggcagggcggctacagcaacgacaacaccgtgatcttcaggcccagcggcggcgactggagggacatcgccaggtgccagatcgccggcaccgtggtgagcacccagctgttcctgaacggcagcctggccgaggaggaggtggtgatcaggagcgaggactggagggacaacgccaagagcatctgcgtgcagctgaacaccagcgtggagatcaactgcaccggcgccggccactgcaacatcagcagggccaagtgggacaacaccctgaagcagatcgccagcaagctgagggagcagtacggcaacaagaccatcatcttcaagcccagcagcggcggcgaccccgagttcgtgaaccacagcttcaactgcggcggcgagttcttctactgcgacagcacccagctgttcgacagcacctggttcgacagcaccggcggcagcggcagcggcggcggcagcgagggccccaggaaccaggactggctg

SEQ ID NO: 114 - protein, 377 aa, Artificial Sequence
  Expressed TRP2_188_GT60mer (DLnano_LS_Trp2_188)
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGGSSVYDFFVWL

SEQ ID NO: 115 - DNA, 1131 bp, Artificial Sequence
  TRP2_188_GT60mer (DLnano_LS_Trp2_188)
atggactggacctggatcctgttcctggtggccgccgccaccagggtgcacagcatgcagatctacgagggcaagctgaccgccgagggcctgaggttcggcatcgtggccagcagggccaaccacgccctggtggacaggctggtggagggcgccatcgacgccatcgtgaggcacggcggcagggaggaggacatcaccctggtgagggtgtgcggcagctgggagatccccgtggccgccggcgagctggccaggaaggaggacatcgacgccgtgatcgccatcggcgtgctgtgcaggggcgccacccccagcttcgactacatcgccagcgaggtgagcaagggcctggccgacctgagcctggagctgaggaagcccatcaccttcggcgtgatcaccgccgacaccctggagcaggccatcgaggccgccggcacctgccacggcaacaagggctgggaggccgccctgtgcgccatcgagatggccaacctgttcaagagcctgaggggcggcagcggcggcagcggcggcagcggcggcagcggcggcggcgacaccatcaccctgccctgcaggcccgccccccccccccactgcagcagcaacatcaccggcctgatcctgaccaggcagggcggctacagcaacgacaacaccgtgatcttcaggcccagcggcggcgactggagggacatcgccaggtgccagatcgccggcaccgtggtgagcacccagctgttcctgaacggcagcctggccgaggaggaggtggtgatcaggagcgaggactggagggacaacgccaagagcatctgcgtgcagctgaacaccagcgtggagatcaactgcaccggcgccggccactgcaacatcagcagggccaagtgggacaacaccctgaagcagatcgccagcaagctgagggagcagtacggcaacaagaccatcatcttcaagcccagcagcggcggcgaccccgagttcgtgaaccacagcttcaactgcggcggcgagttcttctactgcgacagcacccagctgttcgacagcacctggttcgacagcaccggcggcagcggcagcggcggcggcagcagcgtgtacgacttcttcgtgtggctg

SEQ ID NO: 116 - protein, 382 aa, Artificial Sequence
  Expressed TC1_HPV_E743_GT60mer
  MISC_FEATURE (375)..(375): Xaa is unknown
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGGSRAHYNMXDIFIEDF

SEQ ID NO: 117 - DNA, 1131 bp, Artificial Sequence
  TC1_HPV_E743_GT60mer
atggactggacctggatcctgttcctggtggccgccgccaccagggtgcacagcatgcagatctacgagggcaagctgaccgccgagggcctgaggttcggcatcgtggccagcagggccaaccacgccctggtggacaggctggtggagggcgccatcgacgccatcgtgaggcacggcggcagggaggaggacatcaccctggtgagggtgtgcggcagctgggagatccccgtggccgccggcgagctggccaggaaggaggacatcgacgccgtgatcgccatcggcgtgctgtgcaggggcgccacccccagcttcgactacatcgccagcgaggtgagcaagggcctggccgacctgagcctggagctgaggaagcccatcaccttcggcgtgatcaccgccgacaccctggagcaggccatcgaggccgccggcacctgccacggcaacaagggctgggaggccgccctgtgcgccatcgagatggccaacctgttcaagagcctgaggggcggcagcggcggcagcggcggcagcggcggcagcggcggcggcgacaccatcaccctgccctgcaggcccgccccccccccccactgcagcagcaacatcaccggcctgatcctgaccaggcagggcggctacagcaacgacaacaccgtgatcttcaggcccagcggcggcgactggagggacatcgccaggtgccagatcgccggcaccgtggtgagcacccagctgttcctgaacggcagcctggccgaggaggaggtggtgatcaggagcgaggactggagggacaacgccaagagcatctgcgtgcagctgaacaccagcgtggagatcaactgcaccggcgccggccactgcaacatcagcagggccaagtgggacaacaccctgaagcagatcgccagcaagctgagggagcagtacggcaacaagaccatcatcttcaagcccagcagcggcggcgaccccgagttcgtgaaccacagcttcaactgcggcggcgagttcttctactgcgacagcacccagctgttcgacagcacctggttcgacagcaccggcggcagcggcagcggcggcggcagcagggcccactacaacatcgtgaccttc

SEQ ID NO: 118 - protein, 407 aa, Artificial Sequence
  Expressed gp100_3pep_link6_GT60mer_pVax
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGGSTGRAMLGTHTMEVTVYHGGIMDQVPFSVGGEGPRNQDWL

SEQ ID NO: 119 - DNA, 1221 bp, Artificial Sequence
  gp100_3pep_link6_GT60mer_pVax
atggactggacctggatcctgttcctggtggccgccgccaccagggtgcacagcatgcagatctacgagggcaagctgaccgccgagggcctgaggttcggcatcgtggccagcagggccaaccacgccctggtggacaggctggtggagggcgccatcgacgccatcgtgaggcacggcggcagggaggaggacatcaccctggtgagggtgtgcggcagctgggagatccccgtggccgccggcgagctggccaggaaggaggacatcgacgccgtgatcgccatcggcgtgctgtgcaggggcgccacccccagcttcgactacatcgccagcgaggtgagcaagggcctggccgacctgagcctggagctgaggaagcccatcaccttcggcgtgatcaccgccgacaccctggagcaggccatcgaggccgccggcacctgccacggcaacaagggctgggaggccgccctgtgcgccatcgagatggccaacctgttcaagagcctgaggggcggcagcggcggcagcggcggcagcggcggcagcggcggcggcgacaccatcaccctgccctgcaggcccgccccccccccccactgcagcagcaacatcaccggcctgatcctgaccaggcagggcggctacagcaacgacaacaccgtgatcttcaggcccagcggcggcgactggagggacatcgccaggtgccagatcgccggcaccgtggtgagcacccagctgttcctgaacggcagcctggccgaggaggaggtggtgatcaggagcgaggactggagggacaacgccaagagcatctgcgtgcagctgaacaccagcgtggagatcaactgcaccggcgccggccactgcaacatcagcagggccaagtgggacaacaccctgaagcagatcgccagcaagctgagggagcagtacggcaacaagaccatcatcttcaagcccagcagcggcggcgaccccgagttcgtgaaccacagcttcaactgcggcggcgagttcttctactgcgacagcacccagctgttcgacagcacctggttcgacagcaccggcggcagcggcagcggcggcggcagcaccggcagggccatgctgggcacccacaccatggaggtgaccgtgtaccacggcggcatcatggaccaggtgcccttcagcgtgggcggcgagggccccaggaaccaggactggctg

SEQ ID NO: 120 - protein, 377 aa, Artificial Sequence
  Expressed P53_link6_GT60mer_pVax
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGGSKYICNSSCM

SEQ ID NO: 121 - DNA, 1131 bp, Artificial Sequence
  P53_link6_GT60mer_pVax
atggactggacctggatcctgttcctggtggccgccgccaccagggtgcacagcatgcagatctacgagggcaagctgaccgccgagggcctgaggttcggcatcgtggccagcagggccaaccacgccctggtggacaggctggtggagggcgccatcgacgccatcgtgaggcacggcggcagggaggaggacatcaccctggtgagggtgtgcggcagctgggagatccccgtggccgccggcgagctggccaggaaggaggacatcgacgccgtgatcgccatcggcgtgctgtgcaggggcgccacccccagcttcgactacatcgccagcgaggtgagcaagggcctggccgacctgagcctggagctgaggaagcccatcaccttcggcgtgatcaccgccgacaccctggagcaggccatcgaggccgccggcacctgccacggcaacaagggctgggaggccgccctgtgcgccatcgagatggccaacctgttcaagagcctgaggggcggcagcggcggcagcggcggcagcggcggcagcggcggcggcgacaccatcaccctgccctgcaggcccgccccccccccccactgcagcagcaacatcaccggcctgatcctgaccaggcagggcggctacagcaacgacaacaccgtgatcttcaggcccagcggcggcgactggagggacatcgccaggtgccagatcgccggcaccgtggtgagcacccagctgttcctgaacggcagcctggccgaggaggaggtggtgatcaggagcgaggactggagggacaacgccaagagcatctgcgtgcagctgaacaccagcgtggagatcaactgcaccggcgccggccactgcaacatcagcagggccaagtgggacaacaccctgaagcagatcgccagcaagctgagggagcagtacggcaacaagaccatcatcttcaagcccagcagcggcggcgaccccgagttcgtgaaccacagcttcaactgcggcggcgagttcttctactgcgacagcacccagctgttcgacagcacctggttcgacagcaccggcggcagcggcagcggcggcggcagcaagtacatctgcaacagcagctgcatg

SEQ ID NO: 122 - protein, 385 aa, Artificial Sequence
  Expressed gp100_pep174_L6GT60_pVax
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGGSTGRAMLGTHTMEVTVYH

SEQ ID NO: 123 - DNA, 1155 bp, Artificial Sequence
  gp100_pep174_L6GT60_pVax
atggactggacctggatcctgttcctggtggccgccgccaccagggtgcacagcatgcagatctacgagggcaagctgaccgccgagggcctgaggttcggcatcgtggccagcagggccaaccacgccctggtggacaggctggtggagggcgccatcgacgccatcgtgaggcacggcggcagggaggaggacatcaccctggtgagggtgtgcggcagctgggagatccccgtggccgccggcgagctggccaggaaggaggacatcgacgccgtgatcgccatcggcgtgctgtgcaggggcgccacccccagcttcgactacatcgccagcgaggtgagcaagggcctggccgacctgagcctggagctgaggaagcccatcaccttcggcgtgatcaccgccgacaccctggagcaggccatcgaggccgccggcacctgccacggcaacaagggctgggaggccgccctgtgcgccatcgagatggccaacctgttcaagagcctgaggggcggcagcggcggcagcggcggcagcggcggcagcggcggcggcgacaccatcaccctgccctgcaggcccgccccccccccccactgcagcagcaacatcaccggcctgatcctgaccaggcagggcggctacagcaacgacaacaccgtgatcttcaggcccagcggcggcgactggagggacatcgccaggtgccagatcgccggcaccgtggtgagcacccagctgttcctgaacggcagcctggccgaggaggaggtggtgatcaggagcgaggactggagggacaacgccaagagcatctgcgtgcagctgaacaccagcgtggagatcaactgcaccggcgccggccactgcaacatcagcagggccaagtgggacaacaccctgaagcagatcgccagcaagctgagggagcagtacggcaacaagaccatcatcttcaagcccagcagcggcggcgaccccgagttcgtgaaccacagcttcaactgcggcggcgagttcttctactgcgacagcacccagctgttcgacagcacctggttcgacagcaccggcggcagcggcagcggcggcggcagcaccggcagggccatgctgggcacccacaccatggaggtgaccgtgtaccac

SEQ ID NO: 124 - protein, 377 aa, Artificial Sequence
  Expressed gp100_pep209_L6GT60_pVax
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGGSIMDQVPFSV

SEQ ID NO: 125 - DNA, 1131 bp, Artificial Sequence
  gp100_pep209_L6GT60_pVax
atggactggacctggatcctgttcctggtggccgccgccaccagggtgcacagcatgcagatctacgagggcaagctgaccgccgagggcctgaggttcggcatcgtggccagcagggccaaccacgccctggtggacaggctggtggagggcgccatcgacgccatcgtgaggcacggcggcagggaggaggacatcaccctggtgagggtgtgcggcagctgggagatccccgtggccgccggcgagctggccaggaaggaggacatcgacgccgtgatcgccatcggcgtgctgtgcaggggcgccacccccagcttcgactacatcgccagcgaggtgagcaagggcctggccgacctgagcctggagctgaggaagcccatcaccttcggcgtgatcaccgccgacaccctggagcaggccatcgaggccgccggcacctgccacggcaacaagggctgggaggccgccctgtgcgccatcgagatggccaacctgttcaagagcctgaggggcggcagcggcggcagcggcggcagcggcggcagcggcggcggcgacaccatcaccctgccctgcaggcccgccccccccccccactgcagcagcaacatcaccggcctgatcctgaccaggcagggcggctacagcaacgacaacaccgtgatcttcaggcccagcggcggcgactggagggacatcgccaggtgccagatcgccggcaccgtggtgagcacccagctgttcctgaacggcagcctggccgaggaggaggtggtgatcaggagcgaggactggagggacaacgccaagagcatctgcgtgcagctgaacaccagcgtggagatcaactgcaccggcgccggccactgcaacatcagcagggccaagtgggacaacaccctgaagcagatcgccagcaagctgagggagcagtacggcaacaagaccatcatcttcaagcccagcagcggcggcgaccccgagttcgtgaaccacagcttcaactgcggcggcgagttcttctactgcgacagcacccagctgttcgacagcacctggttcgacagcaccggcggcagcggcagcggcggcggcagcatcatggaccaggtgcccttcagcgtg

SEQ ID NO: 126 - protein, 377 aa, Artificial Sequence
  Expressed NYESO1_A2_L6GT60_pVax
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGGSSLLMWITQC

SEQ ID NO: 127 - DNA, 1131 bp, Artificial Sequence
  NYESO1_A2_L6GT60_pVax
atggactggacctggatcctgttcctggtggccgccgccaccagggtgcacagcatgcagatctacgagggcaagctgaccgccgagggcctgaggttcggcatcgtggccagcagggccaaccacgccctggtggacaggctggtggagggcgccatcgacgccatcgtgaggcacggcggcagggaggaggacatcaccctggtgagggtgtgcggcagctgggagatccccgtggccgccggcgagctggccaggaaggaggacatcgacgccgtgatcgccatcggcgtgctgtgcaggggcgccacccccagcttcgactacatcgccagcgaggtgagcaagggcctggccgacctgagcctggagctgaggaagcccatcaccttcggcgtgatcaccgccgacaccctggagcaggccatcgaggccgccggcacctgccacggcaacaagggctgggaggccgccctgtgcgccatcgagatggccaacctgttcaagagcctgaggggcggcagcggcggcagcggcggcagcggcggcagcggcggcggcgacaccatcaccctgccctgcaggcccgccccccccccccactgcagcagcaacatcaccggcctgatcctgaccaggcagggcggctacagcaacgacaacaccgtgatcttcaggcccagcggcggcgactggagggacatcgccaggtgccagatcgccggcaccgtggtgagcacccagctgttcctgaacggcagcctggccgaggaggaggtggtgatcaggagcgaggactggagggacaacgccaagagcatctgcgtgcagctgaacaccagcgtggagatcaactgcaccggcgccggccactgcaacatcagcagggccaagtgggacaacaccctgaagcagatcgccagcaagctgagggagcagtacggcaacaagaccatcatcttcaagcccagcagcggcggcgaccccgagttcgtgaaccacagcttcaactgcggcggcgagttcttctactgcgacagcacccagctgttcgacagcacctggttcgacagcaccggcggcagcggcagcggcggcggcagcagcctgctgatgtggatcacccagtgc

SEQ ID NO: 128 - protein, 385 aa, Artificial Sequence
  Expressed NYESO1_B18C3_L6GT60_pVax
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFDSTWFDSTGGSGSGGGSLEFYLAMPFATPMEAEL

SEQ ID NO: 129 - DNA, 1155 bp, Artificial Sequence
  NYESO1_B18C3_L6GT60_pVax
atggactggacctggatcctgttcctggtggccgccgccaccagggtgcacagcatgcagatctacgagggcaagctgaccgccgagggcctgaggttcggcatcgtggccagcagggccaaccacgccctggtggacaggctggtggagggcgccatcgacgccatcgtgaggcacggcggcagggaggaggacatcaccctggtgagggtgtgcggcagctgggagatccccgtggccgccggcgagctggccaggaaggaggacatcgacgccgtgatcgccatcggcgtgctgtgcaggggcgccacccccagcttcgactacatcgccagcgaggtgagcaagggcctggccgacctgagcctggagctgaggaagcccatcaccttcggcgtgatcaccgccgacaccctggagcaggccatcgaggccgccggcacctgccacggcaacaagggctgggaggccgccctgtgcgccatcgagatggccaacctgttcaagagcctgaggggcggcagcggcggcagcggcggcagcggcggcagcggcggcggcgacaccatcaccctgccctgcaggcccgccccccccccccactgcagcagcaacatcaccggcctgatcctgaccaggcagggcggctacagcaacgacaacaccgtgatcttcaggcccagcggcggcgactggagggacatcgccaggtgccagatcgccggcaccgtggtgagcacccagctgttcctgaacggcagcctggccgaggaggaggtggtgatcaggagcgaggactggagggacaacgccaagagcatctgcgtgcagctgaacaccagcgtggagatcaactgcaccggcgccggccactgcaacatcagcagggccaagtgggacaacaccctgaagcagatcgccagcaagctgagggagcagtacggcaacaagaccatcatcttcaagcccagcagcggcggcgaccccgagttcgtgaaccacagcttcaactgcggcggcgagttcttctactgcgacagcacccagctgttcgacagcacctggttcgacagcaccggcggcagcggcagcggcggcggcagcctggagttctacctggccatgcccttcgccacccccatggaggccgagctg

SEQ ID NO: 130 - protein, 244 aa, Artificial Sequence
  Expressed CD40L-FoldOn (Trimer 1)
DKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGSGSGGYIPEAPRDGQAYVRKDGEWVLLSTFLG

SEQ ID NO: 131 - protein, 249 aa, Artificial Sequence
  Expressed CD40L-GCN4 (Trimer 2)
DKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGSGSGRMKQIEDKIEEILSKIYHIENEIARIKKLIGER

SEQ ID NO: 132 - protein, 373 aa, Artificial Sequence
  Expressed CD40L-1HQK (60mer)
MQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLADLSLELRKPITFGVITADTLEQAIERAGTKHGNKGWEAALSAIEMANLFKSLRGGSGSGGGSDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 133 - protein, 310 aa, Artificial Sequence
  Expressed CD40L_g12
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGIAAHVVSEANSNATSVLQWAKKGYYTMKSNLVMLENGSQLTVNRSGLYYVYTQVTFCSNREPSSQRPFIVGLWLKPSSGSERILLKAANTHSSSQLCEQQSVHLGGVFELQAGASVFVNVTEASQVIHRVGFSSFGLLKLG

SEQ ID NO: 134 - protein, 491 aa, Artificial Sequence
  Expressed CD40L_4mer_mC
DKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGSGGSGGSGGSGGGGELKAIAQELKAIAKECKAIAYELKAIAQGAGGGSGGSGGSGGSGGGDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGHHHHHH

SEQ ID NO: 135 - protein, 438 aa, Artificial Sequence
  Expressed CD40L_SC_mC
GIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGHHHHHH

SEQ ID NO: 136 - protein, 931 aa, Artificial Sequence
  Expressed CD40L_SC_14mer_mC
GIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGSGGSGGSGGSGGGMKVKQLADAVEELASANYHLANAVARLAKAVGERGGSGGSGGSGGSGGGGIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGHHHHHH

SEQ ID NO: 137 - protein, 560 aa, Artificial Sequence
  Expressed CD40L-GT60v1_m
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNSTGGSGSGGGSDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 138 - protein, 561 aa, Artificial Sequence
  Expressed CD40L-dGly_GT60v1_m
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWDNTLKQIASKLREQYGNKTIIFKPSSGGDPEFVNHSFNCGGEFFYCDSTQLFNASTWFASTGGSGSGGGSDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 139 - protein, 566 aa, Artificial Sequence
  Expressed CD40L-GT60v2_m
DTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNSTGGSGGSGGSGGSGGGMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 140 - protein, 780 aa, Artificial Sequence
  Expressed CD40L_SC_GT60_m
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNSTGGSGSGGGSGIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLG

SEQ ID NO: 141 - protein, 598 aa, Artificial Sequence
  Expressed CD40L_SC_g9_m
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGIAAHVVSEANSNAASVLQWAKKGYYTMKSNLVMLENGSQLTVNRSGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGIAAHVVSEANSNAASVLQWAKKGYYTMKSNLVMLENGSQLTVNRSGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGIAAHVVSEANSNAASVLQWAKKGYYTMKSNLVMLENGSQLTVNRSGLYYVYTQVTFCSNREPSSQRPFIVGLWLKPSSGSERILLKAANTHSSSQLCEQQSVHLGGVFELQAGASVFVNVTEASQVIHRVGFSSFGLLKLG

SEQ ID NO: 142 - protein, 598 aa, Artificial Sequence
  Expressed CD40L_SC_g12_m
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGIAAHVVSEANSNATSVLQWAKKGYYTMKSNLVMLENGSQLTVNRSGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGIAAHVVSEANSNATSVLQWAKKGYYTMKSNLVMLENGSQLTVNRSGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGIAAHVVSEANSNATSVLQWAKKGYYTMKSNLVMLENGSQLTVNRSGLYYVYTQVTFCSNREPSSQRPFIVGLWLKPSSGSERILLKAANTHSSSQLCEQQSVHLGGVFELQAGASVFVNVTEASQVIHRVGFSSFGLLKLG

SEQ ID NO: 143 - protein, 310 aa, Artificial Sequence; identical to SEQ ID NO: 133
  Expressed CD40L_g12_m

SEQ ID NO: 144 - protein, 573 aa, Artificial Sequence
  Fusion protein CD40L-(Gly3Ser1)4linker-pembroPD1-VH-Linker(C4S1)3-PembroPD1-VL- Hig Tag
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGIAAHVVSEANSNATSVLQWAKKGYYTMKSNLVMLENGSQLTVNRSGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGSGGGSGGGSGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 145 - protein, 282 aa, Artificial Sequence
  Fusion protein CD40L-Tri
MRRMQLLLLIALSLALVTNSGDRMKQIEDKIEEILSKIYHIENEIARIKKLIGERTSGGSGGTGGSGGTGGSDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 146 - protein, 550 aa, Artificial Sequence
  Fusion protein mCD40L-Tri-PD-1ScFv
MRRMQLLLLIALSLALVTNSGDRMKQIEDKIEEILSKIYHIENEIARIKKLIGERTSGGSGGTGGSGGTGGSDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGGSGGGSGGGSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSGGGGSGGGGSGGGGSDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKHHHHHH

SEQ ID NO: 147 - protein, 120 aa, Artificial Sequence
  PD1 scFv variable heavy chain
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSS

SEQ ID NO: 148 - protein, 111 aa, Artificial Sequence
  PD1 scFv variable light chain
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELK